(19) United States
(12) Patent Application Publication
(10) Pub. No.: US 2023/0190798 A1
(43) Pub. Date: Jun. 22, 2023

(54) CD19-DIRECTED CHIMERIC ANTIGEN RECEPTOR T CELL COMPOSITIONS AND METHODS AND USES THEREOF

(71) Applicant: Juno Therapeutics, Inc., Seattle, WA (US)

(72) Inventors: Matthew WESTOBY, Seattle, WA (US); Adrian Wrangham BRIGGS, Seattle, WA (US); David G. KUGLER, Seattle, WA (US); Robert Guy CASPARY, Seattle, WA (US); Calvin CHAN, Seattle, WA (US); Divya VARUN, Seattle, WA (US); Lothar GERMEROTH, Munich (DE); Christian STEMBERGER, Munich (DE); Mateusz Pawel POLTORAK, Munich (DE); Keenan BASHOUR, Seattle, WA (US); Oleksandr BATUREVYCH, Seattle, WA (US); Nurgul KILAVUZ, Summit, NJ (US); Kristen HEGE, Summit, NJ (US); Michael BURGESS, Summit, NJ (US); Kaida WU, Summit, NJ (US); Ruth Amanda SALMON, Seattle, WA (US)

(73) Assignee: Juno Therapeutics, Inc., Seattle, WA (US)

(21) Appl. No.: 17/799,254

(22) PCT Filed: Feb. 11, 2021
(86) PCT No.: PCT/US2021/017739

§ 371 Date: Aug. 11, 2022

Related U.S. Application Data
(60) Provisional application No. 62/975,724, filed on Feb. 12, 2020.

(51) Int. Cl. A61K 35/17; A61P 35/00; A61K 31/7076; A61K 31/675; C07K 16/28; C07K 14/725; C07K 14/705; C12N 5/0783; A61K 39/395; A61K 38/17

(52) U.S. Cl. CPC A61K 35/17; A61P 35/00; A61K 31/7076; A61K 31/675; C07K 16/2803; C07K 14/7051; C07K 14/70578; C07K 14/70521; C12N 5/0636; A61K 39/3955; A61K 38/177; A61K 38/1774; A61K 2039/5156

(57) ABSTRACT

Provided in some aspects are compositions of cells for treating subjects with disease and conditions such as non-Hodgkin's lymphoma (NHL), and related methods, compositions, uses and articles of manufacture. In some embodiments, the disease or condition is a B-cell non-Hodgkin lymphoma (B-cell NHL). The cells generally express recombinant receptors such as chimeric antigen receptors (CARs) for targeting an antigen, such as CD 19, on cells of the lymphoma.

### CROSS-REFERENCE TO RELATED APPLICATIONS

This application is a national stage application under 35 U.S.C. § 371 of international application No. PCT/US2021/017739, filed internationally on Feb. 11, 2021, which claims priority from U.S. provisional application No. 62/975,724 filed Feb. 12, 2020, entitled “CD19-DIRECTED CHIMERIC ANTIGEN RECEPTOR T CELL COMPOSITIONS AND METHODS AND USES THEREOF,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042022700SubSeqList.txt, created Jan. 23, 2023, which is 58,727 bytes in size. The information in the electronic format of the Sequence Listing is incorporated by reference in its entirety.

### FIELD

The present disclosure relates in some aspects to adoptive cell therapy involving the administration of compositions of cells for treating subjects with disease and conditions such as non-Hodgkin's lymphoma (NHL), and related methods, compositions, uses and articles of manufacture.

### BACKGROUND

Various immunotherapy and/or cell therapy methods are available for treating diseases and conditions. For example, adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be beneficial in the treatment of cancer or other diseases or disorders. Improved approaches are needed. Provided are methods, uses and articles of manufacture that meet such needs.

### SUMMARY

In one aspect, provided herein is a method of treating a B-cell non-Hodgkin lymphoma (B-cell NHL), wherein the method includes administering to a subject having or suspected of having a B-cell NHL a composition including engineered T cells expressing a chimeric antigen receptor (CAR) that targets CD19, wherein the composition includes CD4⁺ T cells expressing the CAR and CD8⁺ T cells expressing the CAR, and the composition includes between at or about 5×10⁶ CAR-expressing T cells and at or about 25×10⁶ CAR-expressing T cells, inclusive; and at least or at least about 80% of the cells in the composition are CD3⁺ cells.

In one aspect, provided herein is a method of treating a B-cell non-Hodgkin lymphoma (B-cell NHL), wherein the method includes administering to a subject having or suspected of having a B-cell NHL a composition including engineered T cells expressing a chimeric antigen receptor (CAR) that targets CD19, wherein the composition includes CD4⁺ T cells expressing the CAR and CD8⁺ T cells expressing the CAR at a ratio between about 1:2.5 and about 2.5:1, and the composition includes between at or about 5×10⁶ CAR-expressing T cells and at or about 100×10⁶ CAR-expressing T cells, inclusive, and at least or at least about 90% of the cells in the composition are CD3⁺ cells.

In one aspect, provided herein is a method of treating a B-cell non-Hodgkin lymphoma (B-cell NHL), the method including administering to a subject having or suspected of having a B-cell NHL, a composition including engineered T cells expressing a chimeric antigen receptor (CAR) that targets CD19, wherein the composition includes CD4⁺ T cells expressing the CAR and CD8⁺ T cells expressing the CAR at a ratio between about 1:1 and about 2.5:1, and the composition includes between at or about 5×10⁶ CAR-expressing T cells and at or about 50×10⁶ CAR-expressing T cells, inclusive, and at least or at least about 80% of the cells in the composition are CD3⁺ cells.

In one aspect, provided herein is a method of treating a B-cell non-Hodgkin lymphoma (r/r B-cell NHL), the method including administering to a subject having or suspected of having a B-cell NHL a composition including engineered T cells expressing a chimeric antigen receptor (CAR) that targets CD19, wherein the composition includes CD4+ T cells expressing the CAR and CD8+ T cells expressing the CAR, and the composition includes between at or about 5×10⁶ CAR-expressing T cells and at or about 100×10⁶ CAR-expressing T cells, inclusive, and at least or at least about 80% of the cells in the composition are CD3⁺ cells, and at least or at least about 80% of the CAR⁺ T cells in the composition are of a naïve-like or central memory phenotype.

In one aspect, provided herein is a method of treating a B-cell non-Hodgkin lymphoma (r/r B-cell NHL), the method including administering to a subject having or suspected of having a B-cell NHL a composition including engineered T cells expressing a chimeric antigen receptor (CAR) that targets CD19, wherein the composition includes CD4+ T cells expressing the CAR and CD8+ T cells expressing the CAR, and the composition includes between at or about 5×10⁶ CAR-expressing T cells and at or about 100×10⁶ CAR-expressing T cells, inclusive, and at least or at least about 80% of the cells in the composition are CD3⁺ cells; and at least or at least about 50% of the CD4⁺CAR⁺ T cells in the composition are CD27⁺CCR7⁺ and/or at least or at least about 50% of the CD8⁺CAR⁺ T cells in the composition are CD27⁺CCR7⁺.

In one aspect, provided herein is a method of treating a B-cell non-Hodgkin lymphoma (B-cell NHL), the method including administering to a subject having or suspected of having a B-cell NHL a composition including engineered T cells expressing a chimeric antigen receptor (CAR) that targets CD19, wherein the composition includes CD4+ T cells expressing the CAR and CD8+ T cells expressing the CAR, and the composition includes between at or about 5×10⁶ CAR-expressing T cells and at or about 50×10⁶ CAR-expressing T cells, inclusive, and at least or at least about 96% of the cells in the composition are CD3⁺ cells, and at least or at least about 80% of the CAR⁺ T cells in the composition are of a naïve-like or central memory phenotype.

In one aspect, provided herein is a method of treating a B-cell non-Hodgkin lymphoma (r/r B-cell NHL), the method including administering to a subject having or suspected of having a B-cell NHL a composition including engineered T cells expressing a chimeric antigen receptor (CAR) that targets CD19, wherein the composition includes CD4+ T cells expressing the CAR and CD8+ T cells expressing the CAR, and the composition includes between at or about 5×10⁶ CAR-expressing T cells and at or about 100×10⁶ CAR-expressing T cells, inclusive, and at least or at least about 80% of the cells in the composition are CD3⁺ cells; and the fraction of integrated vector copy number (iVCN) to total VCN in the CAR⁺ T cells in the composition, on average, is less than or less than about 0.9.

In one aspect, provided herein is a method of treating a B-cell non-Hodgkin lymphoma (r/r B-cell NHL), the method including administering to a subject having or suspected of having a B-cell NHL a composition including engineered T cells expressing a chimeric antigen receptor (CAR) that targets CD19, wherein the composition includes CD4+ T cells expressing the CAR and CD8+ T cells expressing the CAR, and the composition includes between at or about 5×10⁶ CAR-expressing T cells and at or about 100×10⁶ CAR-expressing T cells, inclusive, and at least or at least about 80% of the cells in the composition are CD3⁺ cells; and the integrated vector copy number (iVCN) of the CAR⁺ T cells in the composition, on average, is between or between about 0.4 copies per diploid genome and 3.0 copies per diploid genome, inclusive.

In any of the embodiments herein, the composition comprises CD4⁺ T cells expressing the CAR and CD8⁺ T cells expressing the CAR at a ratio between about 1:2.5 and about 2.5:1.

In any of the embodiments herein, the composition comprises between at or about 5×10⁶ CAR-expressing T cells and at or about 50×10⁶ CAR-expressing T cells, inclusive. In any of the embodiments herein, the composition comprises between at or about 5×10⁶ CAR-expressing T cells and at or about 25×10⁶ CAR-expressing T cells, inclusive.

In any of the embodiments herein, the composition may include CD4⁺ T cells expressing the CAR and CD8⁺ T cells expressing the CAR at a ratio between about 1:2 and about 2:1, between about 1:1.5 and about 1.5:1, or at or at about 1:1. In any of the embodiments herein, the composition may include CD4⁺ T cells expressing the CAR and CD8⁺ T cells expressing the CAR at a ratio between about 1:1 and about 2.5:1, between about 1.5:1 and about 2:1, at or at about 1.5:1, or at or at about 2:1. In any of the embodiments herein, the composition may include between at or about 5×10⁶ CAR-expressing T cells and at or about 10×10⁶ CAR-expressing T cells, inclusive. In any of the embodiments herein, the composition may include between at or about 10×10⁶ CAR-expressing T cells and at or about 25×10⁶ CAR-expressing T cells, inclusive. In any of the embodiments herein, the composition may include at or about 5×10⁶ CAR-expressing T cells. In any of the embodiments herein, the composition may include at or about 10×10⁶ CAR-expressing T cells. In any of the embodiments herein, the composition may include at or about 25×10⁶ CAR-expressing T cells. In any of the embodiments herein, the composition may include at or about 50×10⁶ CAR-expressing T cells. In any of the embodiments herein, the composition may include at or about 100×10⁶ CAR-expressing T cells.

In any of the embodiments herein, at least or at least about 90% of the cells in the composition are CD3⁺ cells.

In any of the embodiments herein, at least or at least about 80% of the CAR⁺ T cells in the composition are of a naïve-like or central memory phenotype.

In any of the embodiments herein, at least or at least about 91%, at least or at least about 92%, at least or at least about 93%, at least or at least about 94%, at least or at least about 95%, or at least or at least about 96% of the cells in the composition are CD3⁺ cells. In any of the embodiments herein, between at or about 5% and at or about 30%, optionally between at or about 5% and at or about 30%, of the CAR⁺ T cells in the composition express a marker of apoptosis, optionally Annexin V or active Caspase 3. In any of the embodiments herein, between at or about 10% and at or about 15% of the CAR⁺ T cells in the composition express a marker of apoptosis, optionally Annexin V or active Caspase 3. In any of the embodiments herein, between at or about 15% and at or about 20% of the CAR⁺ T cells in the composition express a marker of apoptosis, optionally Annexin V or active Caspase 3. In any of the embodiments herein, between at or about 20% and at or about 25% of the CAR⁺ T cells in the composition express a marker of apoptosis, optionally Annexin V or active Caspase 3. In any of the embodiments herein, between at or about 25% and at or about 30% of the CAR⁺ T cells in the composition express a marker of apoptosis, optionally Annexin V or active Caspase 3. In any of the embodiments herein, at or about 5%, at or about 10%, at or about 15%, at or about 20%, at or about 25%, or at or about 30% of the CAR⁺ T cells in the composition express a marker of apoptosis, optionally Annexin V or active Caspase 3. In any of the embodiments herein, between at or about 80% and at or about 85% of the CAR⁺ T cells in the composition are of a naïve-like or central memory phenotype. In any of the embodiments herein, between at or about 85% and at or about 90% of the CAR⁺ T cells in the composition are of a naïve-like or central memory phenotype. In any of the embodiments herein, between at or about 90% and at or about 95% of the CAR⁺ T cells in the composition are of a naïve-like or central memory phenotype. In any of the embodiments herein, between at or about 95% and at or about 99% of the CAR⁺ T cells in the composition are of a naïve-like or central memory phenotype. In any of the embodiments herein, at or about 85%, at or about 90%, at or about 95%, or at or about 99% of the CAR⁺ T cells in the composition are of a naïve-like or central memory phenotype.

In some of any of the embodiments herein, the marker of apoptosis is Annexin V. In some of any of the embodiments herein, the marker of apoptosis is active Caspase 3.

In any of the embodiments herein, the at least or at least about 80% of the CAR⁺ T cells in the composition that are of a naïve-like or central memory phenotype are surface positive for a marker expressed on naïve-like or central memory T cells. In some embodiments, the marker expressed on naïve-like or central memory T cell is selected from the group consisting of CD45RA, CD27, CD28, and CCR7.

In some of any embodiments, the at least or at least about 80% of the CAR⁺ T cells in the composition that are of a naïve-like or central memory phenotype have a phenotype selected from CCR7⁺CD45RA⁺, CD27⁺CCR7⁺, or CD62L⁻CCR7⁺. In some of any embodiments, between at or about 80% and at or about 85%, between at or about 85% and at or about 90%, between at or about 90% and at or about 95%, between at or about 95% and at or about 99% of the CAR⁺ T cells in the composition are of a naïve-like or central memory phenotype selected from CCR7⁺CD45RA⁺, CD27⁺CCR7⁺, or CD62L⁻CCR7⁺. In some of any embodiments, at or about 80%, at or about 85%, at or about 90%, at or about 95%, or at or about 99% of the CAR⁺ T cells in the composition are of a naïve-like or central memory phenotype selected from CCR7⁺CD45RA⁺, CD27⁺CCR7⁺, or CD62L⁻CCR7⁺. In some of any embodiments, at or about 80%, at or about 85%, at or about 90%, at or about 95%, or at or about 99% of the CAR⁺ T cells in the composition are of a naïve-like or central memory phenotype that is CD27⁺CCR7⁺.

In some of any embodiments, at least or at least about 50% of the CD4⁺CAR⁺ T cells in the composition are of a naïve-like or central memory phenotype that is CCR7⁺CD45RA⁺ or CCR7⁺CD45RA⁻. In some of any embodiments, at least or at least about 60% of the CD4⁺CAR⁺ T cells in the composition are of a naïve-like or central memory phenotype that is CCR7⁺CD45RA⁺ or CCR7⁺CD45RA⁻. In some of any embodiments, at least or at least about 70% of the CD4⁺CAR⁺ T cells in the composition are of a naïve-like or central memory phenotype that is CCR7⁺CD45RA⁺ or CCR7⁺CD45RA⁻. In some of any embodiments, at least or at least about 80% of the CD4⁺CAR⁺ T cells in the composition are of a naïve-like or central memory phenotype that is CCR7⁺CD45RA⁺ or CCR7⁺CD45RA⁻. In some of any embodiments, at least or at least about 85% of the CD4⁺CAR⁺ T cells in the composition are of a naïve-like or central memory phenotype that is CCR7⁺CD45RA⁺ or CCR7⁺CD45RA⁻.

In some of any embodiments, at least or at least about 50% of the CD4⁺CAR⁺ T cells in the composition are of a naïve-like or central memory phenotype that is CD27⁺CCR7⁺. In some of any embodiments, at least or at least about 60% of the CD4⁺CAR⁺ T cells in the composition are of a naïve-like or central memory phenotype that is CD27⁺CCR7⁺. In some of any embodiments, at least or at least about 70% of the CD4⁺CAR⁺ T cells in the composition are of a naïve-like or central memory phenotype that is CD27⁺CCR7⁺. In some of any embodiments, at least or at least about 80% of the CD4⁺CAR⁺ T cells in the composition are of a naïve-like or central memory phenotype that is CD27⁺CCR7⁺. In some of any embodiments, at least or at least about 85% of the CD4⁺CAR⁺ T cells in the composition are of a naïve-like or central memory phenotype that is CD27⁺CCR7⁺.

In some of any embodiments, at least or at least about 50% of the CD8⁺CAR⁺ T cells in the composition are of a naïve-like or central memory phenotype that is CCR7⁺CD45RA⁺ or CCR7⁺CD45RA⁻. In some of any embodiments, at least or at least about 60% of the CD8⁺CAR⁺ T cells in the composition are of a naïve-like or central memory phenotype that is CCR7⁺CD45RA⁺ or CCR7⁺CD45RA⁻. In some of any embodiments, at least or at least about 70% of the CD8⁺CAR⁺ T cells in the composition are of a naïve-like or central memory phenotype that is CCR7⁺CD45RA⁺ or CCR7⁺CD45RA⁻. In some of any embodiments, at least or at least about 80% of the CD8⁺CAR⁺ T cells in the composition are of a naïve-like or central memory phenotype that is CCR7⁺CD45RA⁺ or CCR7⁺CD45RA⁻. In some of any embodiments, at least or at least about 85% of the CD8⁺CAR⁺ T cells in the composition are of a naïve-like or central memory phenotype that is CCR7⁺CD45RA⁺ or CCR7⁺CD45RA⁻.

In some of any embodiments, at least or at least about 50% of the CD8⁺CAR⁺ T cells in the composition are of a naïve-like or central memory phenotype that is CD27⁺CCR7⁺. In some of any embodiments, at least or at least about 60% of the CD8⁺CAR⁺ T cells in the composition are of a naïve-like or central memory phenotype that is CD27⁺CCR7⁺. In some of any embodiments, at least or at least about 70% of the CD8⁺CAR⁺ T cells in the composition are of a naïve-like or central memory phenotype that is CD27⁺CCR7⁺. In some of any embodiments, at least or at least about 80% of the CD8⁺CAR⁺ T cells in the composition are of a naïve-like or central memory phenotype that is CD27⁺CCR7⁺. In some of any embodiments, at least or at least about 85% of the CD8⁺CAR⁺ T cells in the composition are of a naïve-like or central memory phenotype that is CD27⁺CCR7⁺.

In any of the embodiments herein, at least or at least about 80% of the CAR⁺ T cells in the composition are surface positive for a marker expressed on naïve-like or central memory T cells. In any of the embodiments herein, the marker expressed on naïve-like or central memory T cell is selected from the group consisting of CD45RA, CD27, CD28, and CCR7. In any of the embodiments herein, at least or at least about 80% of the CAR⁺ T cells in the composition are CCR7⁺CD45RA⁺, CD27⁺CCR7⁺, and/or CD62L⁻CCR7⁺. In any of the embodiments herein, between at or about 80% and at or about 85%, between at or about 85% and at or about 90%, between at or about 90% and at or about 95%, between at or about 95% and at or about 99% of the CAR⁺ T cells in the composition are CCR7⁺CD45RA⁺, CD27⁺CCR7⁺, and/or CD62L⁻CCR7⁺. In any of the embodiments herein, at or about 80%, at or about 85%, at or about 90%, at or about 95%, or at or about 99% of the CAR⁺ T cells in the composition are CCR7⁺CD45RA⁺, CD27⁺CCR7⁺, and/or CD62L⁻CCR7⁺. In any of the embodiments herein, at or about 80%, at or about 85%, at or about 90%, at or about 95%, or at or about 99% of the CAR⁺ T cells in the composition are CD27⁺CCR7⁺. In any of the embodiments herein, at least or at least about 50% of the CD4⁺CAR⁺ T cells in the composition are CCR7⁺CD45RA⁺ or CCR7⁺CD45RA⁻. In any of the embodiments herein, at least or at least about 60% of the CD4⁺CAR⁺ T cells in the composition are CCR7⁺CD45RA⁺ or CCR7⁺CD45RA⁻. In any of the embodiments herein, at least or at least about 70% of the CD4⁺CAR⁺ T cells in the composition are CCR7⁺CD45RA⁺ or CCR7⁺CD45RA⁻. In any of the embodiments herein, at least or at least about 80% of the CD4⁺CAR⁺ T cells in the composition are CCR7⁺CD45RA⁺ or CCR7⁺CD45RA⁻. In any of the embodiments herein, at least or at least about 85% of the CD4⁺CAR⁺ T cells in the composition are CCR7⁺CD45RA⁺ or CCR7⁺CD45RA⁻. In any of the embodiments herein, at least or at least about 50% of the CD4⁺CAR⁺ T cells in the composition are CD27⁺CCR7⁺. In any of the embodiments herein, at least or at least about 60% of the CD4⁺CAR⁺ T cells in the composition are CD27⁺CCR7⁺. In any of the embodiments herein, at least or at least about 70% of the CD4⁺CAR⁺ T cells in the composition are CD27⁺CCR7⁺. In any of the embodiments herein, at least or at least about 80% of the CD4⁺CAR⁺ T cells in the composition are CD27⁺CCR7⁺. In any of the embodiments herein, at least or at least about 85% of the CD4⁺CAR⁺ T cells in the composition are CD27⁺CCR7⁺.

In any of the embodiments herein, at least or at least about 50% of the CD8⁺CAR⁺ T cells in the composition are CCR7⁺CD45RA⁺ or CCR7⁺CD45RA⁻. In any of the embodiments herein, at least or at least about 60% of the CD8⁺CAR⁺ T cells in the composition are CCR7⁺CD45RA⁺ or CCR7⁺CD45RA⁻. In any of the embodiments herein, at least or at least about 70% of the CD8⁺CAR⁺ T cells in the composition are CCR7⁺CD45RA⁺ or CCR7⁺CD45RA⁻. In any of the embodiments herein, at least or at least about 80% of the CD8⁺CAR⁺ T cells in the composition are CCR7⁺CD45RA⁺ or CCR7⁺CD45RA⁻. In any of the embodiments herein, at least or at least about 85% of the CD8⁺CAR⁺ T cells in the composition are CCR7⁺CD45RA⁺ or CCR7⁺CD45RA⁻. In any of the embodiments herein, at least or at least about 50% of the CD8⁺CAR⁺ T cells in the composition are CD27⁺CCR7⁺. In any of the embodiments herein, at least or at least about 60% of the CD8⁺CAR⁺ T cells in the composition are CD27⁺CCR7⁺. In any of the embodiments herein, at least or at least about 70% of the CD8⁺CAR⁺ T cells in the composition are CD27⁺CCR7⁺. In any of the embodiments herein, at least or at least about 80% of the CD8⁺CAR⁺ T cells in the composition are CD27⁺CCR7⁺. In any of the embodiments herein, at least or at least about 85% of the CD8⁺CAR⁺ T cells in the composition are CD27⁺CCR7⁺.

In any of the embodiments herein, the fraction of integrated vector copy number (iVCN) to total VCN in the CAR⁺ T cells in the composition, on average, is less than or less than about 0.9.

In any of the embodiments herein, the fraction of integrated vector copy number (iVCN) to total VCN in the CAR⁺ T cells in the composition, on average, is between at or about 0.9 and at or about 0.8. In any of the embodiments herein, the fraction of integrated vector copy number (iVCN) to total VCN in the CAR⁺ T cells in the composition, on average, is less than or less than about 0.8. In any of the embodiments herein, the fraction of integrated vector copy number (iVCN) to total VCN in the CAR⁺ T cells in the composition, on average, is between at or about 0.8 and at or about 0.7. In any of the embodiments herein, the fraction of integrated vector copy number (iVCN) to total VCN in the CAR⁺ T cells in the composition, on average, is between at or about 0.7 and at or about 0.6. In any of the embodiments herein, the fraction of integrated vector copy number (iVCN) to total VCN in the CAR⁺ T cells in the composition, on average, is between at or about 0.6 and at or about 0.5. In any of the embodiments herein, the fraction of integrated vector copy number (iVCN) to total VCN in the CAR⁺ T cells in the composition, on average, is between at or about 0.5 and at or about 0.4.

In any of the embodiments herein, the integrated vector copy number (iVCN) of the CAR⁺ T cells in the composition, on average, is between or between about 0.4 copies per diploid genome and 3.0 copies per diploid genome, inclusive.

In any of the embodiments herein, the integrated vector copy number (iVCN) of the CAR⁺ T cells in the composition, on average, is between or between about 0.8 copies per diploid genome and 2.0 copies per diploid genome, inclusive. In any of the embodiments herein, the integrated vector copy number (iVCN) of the CAR⁺ T cells in the composition, on average, is between or between about 0.8 copies per diploid genome and 1.0 copies per diploid genome, inclusive. In any of the embodiments herein, the integrated vector copy number (iVCN) of the CAR⁺ T cells in the composition, on average, is between or between about 1.0 copies per diploid genome and 1.5 copies per diploid genome, inclusive. In any of the embodiments herein, the integrated vector copy number (iVCN) of the CAR⁺ T cells in the composition, on average, is between or between about 1.5 copies per diploid genome and 2.0 copies per diploid genome, inclusive.

In any of the embodiments herein, the r/r B-cell NHL is selected from the group consisting of a diffuse large B-cell lymphoma (DLBCL), optionally DLBCL not otherwise specified (DLBCL NOS; including de novo or transformed DLBCL, e.g., from follicular lymphoma or marginal zone lymphoma); high grade B-cell lymphoma (HGBCL), optionally HGBCL with MYC and BCL2 and/or BCL6 rearrangements with DLBCL histology; primary mediastinal large B cell lymphoma (PMBCL); and follicular lymphoma (FL), optionally follicular lymphoma grade 3b (FL3B).

In any of the embodiments herein, the B-cell NHL is selected from the group consisting of: diffuse large B-cell lymphoma (DLBCL), optionally DLBCL not otherwise specified; transformed DLBCL, optionally transformed DLBCL from follicular lymphoma or marginal zone lymphoma; high grade B-cell lymphoma (HGBCL), optionally HGBCL with MYC and BCL2 and/or BCL6 rearrangements with DLBCL histology; primary mediastinal large B cell lymphoma (PMBCL); and follicular lymphoma (FL), optionally follicular lymphoma grade 3b (FL3B).

In any of the embodiments herein, the B-cell NHL is a diffuse large B-cell lymphoma. In any of the embodiments herein, the B-cell NHL is a diffuse large B-cell lymphoma not otherwise specified.

In any of the embodiments herein, the B-cell NHL is a de novo diffuse large B-cell lymphoma. In any of the embodiments herein, the B-cell NHL is a diffuse large B-cell lymphoma transformed from a follicular lymphoma. In any of the embodiments herein, the B-cell NHL is a diffuse large B-cell lymphoma transformed from a marginal zone lymphoma. In any of the embodiments herein, the B-cell NHL is a high grade B-cell lymphoma (HGBCL). In any of the embodiments herein, the B-cell NHL is a high grade B-cell lymphoma with MYC and BCL2 and/or BCL6 rearrangements. In any of the embodiments herein, the B-cell NHL is a high grade B-cell lymphoma with DLBCL histology. In any of the embodiments herein, the B-cell NHL is a double-hit lymphoma or triple-hit lymphoma. In any of the embodiments herein, the B-cell NHL is a primary mediastinal large B cell lymphoma. In any of the embodiments herein, the B-cell NHL is a follicular lymphoma. In any of the embodiments herein, the B-cell NHL is a follicular lymphoma grade 3b. In any of the embodiments herein, the B-cell NHL is histologically confirmed.

In any of the embodiments herein, at or immediately prior to the time of the administration of the composition comprising engineered T cells, the subject has relapsed following remission after treatment with, or become refractory to: (i) two or more prior therapies for the B-cell NHL and/or (ii) an autologous stem cell transplant (ASCT) therapy.

In any of the embodiments herein, at or immediately prior to the time of the administration of the composition comprising engineered T cells, the subject has relapsed following remission after treatment with, or become refractory to, two or more prior therapies for the B-cell NHL. In any of the embodiments herein, at or immediately prior to the time of the administration of the composition comprising engineered T cells, the subject has relapsed following remission after treatment with, or become refractory to, three or more prior therapies for the B-cell NHL. In any of the embodiments herein, the two or more prior therapies for the r/r B-cell NHL does not comprise another dose of cells expressing the CAR.

In any of the embodiments herein, the two or more prior therapies for the B-cell NHL comprise an anthracycline and a CD20-targeted agent. In any of the embodiments herein, the two or more prior therapies for the B-cell NHL do not include those given for prior indolent lymphoma. In any of the embodiments herein, the two or more prior therapies for the B-cell NHL do not include anthracycline given for indolent DLBCL. In any of the embodiments herein, the two or more prior therapies for the B-cell NHL comprise a CD20-targeted agent, and the two or more prior therapies for the B-cell NHL excludes an anthracycline if it is given for prior indolent lymphoma. In any of the embodiments herein, the CD20-targeted agent comprises an anti-CD20 monoclonal antibody. In any of the embodiments herein, the CD20-targeted agent comprises rituximab.

In any of the embodiments herein, at or immediately prior to the time of the administration of the composition comprising engineered T cells, the subject has relapsed following remission after treatment with, or become refractory to, an autologous stem cell transplant (ASCT) therapy. In some embodiments, the subject has relapsed and/or refractory DLBCL. In some embodiments, the ASCT fails to achieve an objective response (partial response (PR) or better). In any of the embodiments herein, after the ASCT, the disease of the subject has progressed.

In any of the embodiments herein, at or prior to the administration of the composition comprising engineered T cells, the subject has been identified as having an aggressive disease or a high-risk disease, or as having poor prognosis. In any of the embodiments herein, at or prior to the administration of the composition comprising engineered T cells, the subject has been identified as having a chemorefractory disease or as having a persistent or relapsed disease following chemotherapy.

In any of the embodiments herein, the subject has a pathologically-confirmed secondary central nervous system (CNS) involvement by malignancy. In any of the embodiments herein, the subject does not have a central nervous system (CNS)-only involvement by malignancy.

In any of the embodiments herein, the subject has not received a prior CAR T cell or genetically-modified T cell therapy. In any of the embodiments herein, the subject has not received prior CD19-targeted therapy such as an anti-CD19 monoclonal antibody or bispecific antibody.

In any of the embodiments herein, the method further comprises obtaining a leukapheresis sample from the subject for manufacturing the composition comprising engineered T cells. In some embodiments, the subject has not received a therapeutic dose of a corticosteroid, optionally within at or about 14 days prior to the time of leukapheresis. In some embodiments, the subject has not received a cytotoxic chemotherapeutic agent, which is not a lymphotoxic chemotherapeutic agent or intrathecal therapy, within at or about 7 days prior to the time of leukapheresis. In any of the embodiments herein, the subject has not received a lymphotoxic chemotherapeutic agent within at or about 4 weeks prior to the time of leukapheresis. In any of the embodiments herein, the subject has not received an immunosuppressive therapy within at or about 4 weeks prior to the time of leukapheresis. In any of the embodiments herein, the subject has not received radiation within at or about 6 weeks prior to the time of leukapheresis. In any of the embodiments herein, the subject has not received an autologous stem-cell transplant within at or about 3 months prior to the time of leukapheresis.

In any of the embodiments herein, the subject has not achieved complete remission (CR) in response to a prior therapy. In any of the embodiments herein, the subject has not achieved an objective response (partial response (PR) or better) in response to a prior therapy.

In any of the embodiments herein, at or prior to the administration of the composition comprising engineered T cells, the subject is evaluated for a lymphoma associated with or involving central nervous system (CNS) involvement or a secondary CNS lymphoma. In any of the embodiments herein, at or prior to the administration of the composition comprising engineered T cells, the subject is or has been identified as having: an adequate cardiac function, optionally with a left ventricular ejection fraction (LVEF) at or about 40%, greater than 40%, or great than about 40%; and/or an adequate renal function, optionally with a calculated creatinine clearance of at or about 45 mL/min, greater than 45 mL/min, or greater than about 45 mL/min; and/or an adequate hepatic function, optionally with an aspartate aminotransferase (AST) and alanine aminotransferase (ALT) of or less than 2.5 times the upper limit of normal (ULN) and total bilirubin less than 1.5 times the ULN; and/or an adequate pulmonary function, optionally with ≤CTCAE Grade 1 dyspnea and saturated oxygen (e.g., SaO₂≥92%) on room air. In any of the embodiments herein, at or prior to the administration of the composition comprising engineered T cells, the subject is or has been identified as having an absolute neutrophil count (ANC) of at or about 1.0×10⁹ cells/L, greater than 1.0×10⁹ cells/L, or greater than about 1.0×10⁹ cells/L without growth factor support. In any of the embodiments herein, at or prior to the administration of the composition comprising engineered T cells, the subject is or has been identified as having platelets of at or about 50×10⁹ cells/L, greater than 50×10⁹ cells/L, or greater than about 50×10⁹ cells/L without transfusion support.

In any of the embodiments herein, at or prior to the administration of the composition comprising engineered T cells, the subject has received a bridging chemotherapy between the time of leukapheresis to produce the composition comprising engineered T cells and the administration of the composition comprising engineered T cells. In any of the embodiments herein, at or prior to the administration of the composition comprising engineered T cells, the subject has received a bridging chemotherapy for disease control after a prior therapy.

In any of the embodiments herein, the subject is or has been identified as having an Eastern Cooperative Oncology Group Performance Status (ECOG PS) of 0 or 1. In any of the embodiments herein, prior to the administration of the composition comprising engineered T cells, the subject is or has been identified as having a high baseline tumor burden, such as measured by the sum of product of the perpendicular diameters (SPD), or high serum lactate dehydrogenase (LDH), such as LDH≥500 U/L. In any of the embodiments herein, at or prior to the administration of the composition comprising engineered T cells, the subject has a positron emission tomography (PET)-positive disease.

In any of the embodiments herein, the method further comprises, prior to administration of the composition comprising engineered T cells, identifying or selecting the subject for administration of the composition comprising engineered T cells.

In any of the embodiments herein, prior to the administration, the subject has been preconditioned with a lymphodepleting therapy. In any of the embodiments herein, the method further comprises, immediately prior to the administration of the composition comprising engineered T cells, administering a lymphodepleting therapy to the subject, wherein the lymphodepleting therapy comprises the administration of fludarabine and/or cyclophosphamide.

In any of the embodiments herein, the administration of the composition comprising engineered T cells and/or the lymphodepleting therapy is carried out via outpatient delivery. In some of any embodiments, the administration of the composition is carried out on an outpatient basis, optionally unless or until the subject exhibits a sustained fever or a fever that is or has not been reduced or not reduced by more than 1° C. after treatment with an antipyretic. In some of any embodiments, the administration of the composition is without admitting the subject to a hospital and/or without an overnight stay at a hospital, optionally unless or until the subject exhibits a sustained fever or a fever that is or has not been reduced or not reduced by more than 1° C. after treatment with an antipyretic. In some of any embodiments, the administration of the composition is without requiring admission to or an overnight stay at a hospital, optionally unless or until the subject exhibits a sustained fever or a fever that is or has not been reduced or not reduced by more than 1° C. after treatment with an antipyretic.

In any of the embodiments herein, the lymphodepleting therapy comprises the administration of fludarabine at 30 mg/m² body surface area of the subject, daily, and cyclophosphamide at 300 mg/m² body surface area of the subject, daily, each for 3 days. In any of the embodiments herein, the composition comprising engineered T cells is administered between at or about 48 hours and at or about 9 days, inclusive, after completion of the lymphodepleting therapy. In any of the embodiments herein, the subject has a creatinine clearance of at least or at least about 30 mL/min when receiving the lymphodepleting therapy.

In any of the embodiments herein, prior to initiation of administration of the composition comprising engineered T cells, the subject has not been administered an agent or treatment for the treatment, prevention, reduction, or attenuation of a neurotoxicity and/or a cytokine release syndrome or risk thereof. In any of the embodiments herein, the method further comprises administering to the subject an agent or treatment for the treatment, prevention, reduction, or attenuation of a neurotoxicity and/or a cytokine release syndrome or risk thereof. In any of the embodiments herein, the agent is or comprises an anti-IL-6 antibody, an anti-IL-6 receptor antibody, or a steroid. In any of the embodiments herein, the agent is or comprises tocilizumab or methylprednisolone.

In any of the embodiments herein, the T cells are primary T cells obtained from a subject.

In any of the embodiments herein, the T cells are autologous to the subject.

In any of the embodiments herein, at least 35%, at least 40% or at least 50% of subjects treated according to the method achieve a complete response (CR); at least 60%, 70%, 80%, 90%, or 95% of subjects achieving a CR exhibit a CR that is durable for at or greater than 3 months or at or greater than 6 months; and/or at least 60%, 70%, 80%, 90%, or 95% of subjects achieving a CR by one month and/or by 3 months remain in response, remain in CR, and/or survive or survive without progression, for at or greater than 3 months and/or at or greater than 6 months and/or at greater than 9 months after achieving the CR; and/or at least 50%, at least 60% or at least 70% of the subjects treated according to the method achieve objective response (OR); at least 60%, 70%, 80%, 90%, or 95% of subjects achieving an OR exhibit an OR that is durable for at or greater than 3 months or at or greater than 6 months; and/or at least 35%, at least 40%, or at least 50% of subjects achieving an OR remain in response or survive for at or greater than 3 months and/or at or greater than 6 months after achieving the OR.

In any of the embodiments herein, at least 50% of subjects treated according to the method achieve a complete response (CR); at least 60% of subjects achieving a CR exhibit a CR that is durable for at or greater than 6 months; and/or at least 60% of subjects achieving a CR by 1 month and/or by 3 months remain in response, remain in CR, and/or survive or survive without progression, for at or greater than 6 months after achieving the CR; and/or at least 70% of the subjects treated according to the method achieve objective response (OR); at least 60% of subjects achieving an OR exhibit an OR that is durable for at or greater than 6 months; and/or at least 50% of subjects achieving an OR remain in response or survive for at or greater than 6 months after achieving the OR.

In any of the embodiments herein, the CR or the OR is durable for greater than 3 months or greater than 6 months; and/or at least 20%, at least 25%, at least 35%, at least 40% or at least 50% of subjects treated according to the method achieve a CR that is durable for greater than 3 months or greater than 6 months; and/or at least 60%, 70%, 80%, 90%, or 95% of subjects treated with the method and who achieve a CR, remain in CR or remain in response or remain surviving for at or greater than 3 months or at or greater than 6 months or at or greater than 9 months; and/or at least 60%, 70%, 80%, 90%, or 95% of subjects treated with the method who achieve a CR by one month and/or by 3 months remain in response, remain in CR, and/or survive or survive without progression, for greater at or greater than 3 months and/or at or greater than 6 months and/or at greater than 9 months; and/or at least 50%, at least 60% or at least 70% of the subjects treated according to the method achieve objective response (OR); at least 60%, 70%, 80%, 90%, or 95% of subjects achieve an OR that is durable for at or greater than 3 months or at or greater than 6 months; and/or at least at least 35%, at least 40%, or at least 50% of subjects treated with the method and achieving an OR remain in response or survive for at or greater than 3 months and/or at or greater than 6 months.

In any of the embodiments herein, the cells are autologous to the subject, and no minimum absolute lymphocyte count (ALC) for apheresis is required and/or specified for production of the therapy; and/or the cells are produced by a process which, for at least 90%, 91%, 92%, 93%, 94%, 95%, 96%, 97%, 98%, 99%, or 100% of subjects having the B-cell NHL, is capable of generating a cell product for administration according to the method.

In any of the embodiments herein, at least 35%, at least 40% or at least 50% of subjects treated according to the method achieve a complete response (CR) or remission of CNS disease; at least 60%, 70%, 80%, 90%, or 95% of subjects who achieve a CR remain in CR for at or greater than 3 months or at or greater than 6 months; and/or at least 60%, 70%, 80%, 90%, or 95% of subjects achieving a CR or remission of CNS disease by one month and/or by 3 months remain in response, remain in CR, and/or survive or survive without progression, for greater at or greater than 3 months and/or at or greater than 6 months and/or at greater than 9 months; and/or at least 50%, at least 60% or at least 70% of the subjects treated according to the method achieve objective response (OR) or remission of CNS disease; at least 60%, 70%, 80%, 90%, or 95% of subjects achieving the OR, for at or greater than 3 months or at or greater than 6 months; and/or at least 60%, 70%, 80%, 90%, or 95% of subjects achieving OR or remission of CNS disease remain in response or survive for at or greater than 3 months and/or at or greater than 6 months; and/or the brain lesion is reduced in size or volume by greater than or greater than about 25%, 50%, 75% or more; and/or reduction or remission or clearance of CNS disease is achieved in at least 35%, at least 40% or at least 50% of subjects treated according to the method.

In any of the embodiments herein, greater than or greater than about 50%, about 60%, about 70%, or about 80% of the subjects treated according to the method do not exhibit a grade 3 or greater cytokine release syndrome (CRS) and/or do not exhibit a grade 3 or greater neurotoxicity and/or greater than 40% or 50% or 55% of the subjects treated according to the method do not exhibit any neurotoxicity or CRS. In any of the embodiments herein, greater than or greater than about 80% of the subjects treated according to the method do not exhibit a grade 3 or greater cytokine release syndrome (CRS) and/or do not exhibit a grade 3 or greater neurotoxicity. In any of the embodiments herein, greater than 95% of the subjects treated according to the method do not exhibit grade 3 or greater CRS. In any of the embodiments herein, greater than 85% of the subjects treated according to the method do not exhibit grade 3 or greater neurotoxicity.

In any of the embodiments herein, greater than or greater than about 30%, 35%, 40%, or 50% of the subjects treated according to the method do not exhibit any grade of cytokine release syndrome (CRS) or neurotoxicity; and/or at least at or about 45%, 50%, 60%, 65%, 70%, 75%, 80%, 85%, 90%, or 95% of subjects treated according to the method do not exhibit onset of CRS earlier than 3 days following initiation of the administration and/or do not exhibit onset of neurotoxicity earlier than 5 days following initiation of the administration; and/or the median onset of neurotoxicity among subjects treated according to the method is at or after the median peak of, or median time to resolution of, CRS in subjects treated according to the method and/or the median onset of neurotoxicity among subjects treated according to the method is greater than at or about 8, 9, 10, or 11 days.

In any of the embodiments herein, greater than or greater than about 50% of the subjects treated according to the method do not exhibit any grade of cytokine release syndrome (CRS) or neurotoxicity; and/or at least at or about 45% of subjects treated according to the method do not exhibit onset of CRS earlier than 3 days following initiation of the administration and/or do not exhibit onset of neurotoxicity earlier than 5 days following initiation of the administration; and/or the median onset of neurotoxicity among subjects treated according to the method is at or after the median peak of, or median time to resolution of, CRS in subjects treated according to the method and/or the median onset of neurotoxicity among subjects treated according to the method is greater than at or about 8 days.

In any of the embodiments herein, at least 50% of subjects treated according to the method achieve a complete response (CR); at least 70% of the subjects treated according to the method achieve objective response (OR); and greater than or greater than about 50% of the subjects treated according to the method do not exhibit any grade of cytokine release syndrome (CRS) or neurotoxicity; and greater than or greater than about 80% of the subjects treated according to the method do not exhibit a grade 3 or greater cytokine release syndrome (CRS) and/or do not exhibit a grade 3 or greater neurotoxicity.

In any of the embodiments herein, the CAR may include an extracellular antigen-binding domain specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta; in some any embodiments, the CAR may include, in order, an extracellular antigen-binding domain specific for the antigen, a transmembrane domain, a cytoplasmic signaling domain derived from a costimulatory molecule, and a cytoplasmic signaling domain derived from a primary signaling ITAM-containing molecule; or the CAR may include an extracellular antigen-recognition domain that specifically binds to the antigen and an intracellular signaling domain including a CD3-zeta (CD3ζ) chain and a costimulatory signaling region that is a signaling domain of 4-1BB. In any of the embodiments herein, the CAR may include an extracellular antigen-binding domain specific for CD19, a transmembrane domain, a cytoplasmic signaling domain derived from a 4-1BB, and a cytoplasmic signaling domain derived from a CD3zeta.

In any of the embodiments herein, the CAR comprises an extracellular antigen-binding domain specific for CD19, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta; the CAR comprises, in order, an extracellular antigen-binding domain specific for CD19, a transmembrane domain, a cytoplasmic signaling domain derived from a costimulatory molecule, and a cytoplasmic signaling domain derived from a primary signaling ITAM-containing molecule; or the CAR comprises an extracellular antigen-recognition domain that specifically binds to CD19 and an intracellular signaling domain comprising a CD3-zeta (CD3ζ) chain and a costimulatory signaling region that is a signaling domain of 4-1BB.

In any of the embodiments herein, the CAR comprises an extracellular antigen-binding domain specific for CD19, a transmembrane domain, a cytoplasmic signaling domain derived from a costimulatory molecule that is 4-1BB, and a cytoplasmic signaling domain derived from a primary signaling ITAM-containing molecule that is CD3zeta.

In any of the embodiments herein, the extracellular antigen-binding domain is an scFv. In any of the embodiments herein, the scFv may include an amino acid sequence of RASQDISKYLN (SEQ ID NO: 35), an amino acid sequence of SRLHSGV (SEQ ID NO: 36), and/or an amino acid sequence of GNTLPYTFG (SEQ ID NO: 37) and/or an amino acid sequence of DYGVS (SEQ ID NO: 38), an amino acid sequence of VIWGSETTYYNSALKS (SEQ ID NO: 39), and/or an amino acid sequence of YAMDYWG (SEQ ID NO: 40). In any of the embodiments herein, the six CDR sequences (CDRL1-L3 and CDRH1-H3) of the scFv may include an amino acid sequences RASQDISKYLN (SEQ ID NO: 35), SRLHSGV (SEQ ID NO: 36), GNTLPYTFG (SEQ ID NO: 37), DYGVS (SEQ ID NO: 38), VIWGSETTYYNSALKS (SEQ ID NO: 39), and YAMDYWG (SEQ ID NO: 40). In some embodimetnts, the scFv includes a variable heavy chain region of FMC63 and a variable light chain region of FMC63. In some embodiments, the six CDR sequences (CDRL1-L3 and CDRH1-H3) of the scFv may include a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63. In some embodiments, the scFv may be an scFv that binds to the same epitope as or competes for binding with any one of the foregoing. In some embodiments, the scFv includes, in order, a VH, a linker, optionally including a linker set forth in SEQ ID NO: 24, and a VL. In any of the embodiments herein, the scFv includes, in order, a VL, a linker, optionally including a linker set forth in SEQ ID NO: 24, and a VH. In some embodiments, the scFv includes a flexible linker. In some embodiments, the scFv includes the amino acid sequence set forth as SEQ ID NO: 43. In any of the embodiments herein, the scFv is set forth as SEQ ID NO: 43.

In any of the embodiments herein, the scFv may comprise an amino acid sequence of RASQDISKYLN (SEQ ID NO: 35), an amino acid sequence of SRLHSGV (SEQ ID NO: 36), and/or an amino acid sequence of GNTLPYTFG (SEQ ID NO: 37) and/or an amino acid sequence of DYGVS (SEQ ID NO: 38), an amino acid sequence of VIWGSETTYYNSALKS (SEQ ID NO: 39), and/or an amino acid sequence of YAMDYWG (SEQ ID NO: 40). In some embodiments, the scFv comprises a variable heavy chain region of FMC63 and a variable light chain region of FMC63. In some embodiments, the scFv comprises a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63. In some embodiments, the scFv comprises, in order, a VH, a linker SEQ ID NO: 24, and a VL. In some embodiments, the scFv comprises a flexible linker. In some embodiments, the scFv comprises the amino acid sequence set forth as SEQ ID NO: 43. In some embodiments, the CD19-directed scFv binds to the same epitope as or competes for binding with any one of the foregoing.

In any of the embodiments herein, the scFv may include a variable heavy chain region of FMC63 and a variable light chain region of FMC63.

In some embodiments, the transmembrane domain is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8. In some embodiments, the transmembrane-domain is a transmembrane domain that comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In any of the embodiments herein, the costimulatory signaling region is a signaling domain of 4-1BB. In any of the embodiments herein, the costimulatory domain may include SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto. In any of the embodiments herein, the primary signaling domain is a CD3zeta signaling domain. In any of the embodiments herein, the primary signaling domain may include SEQ ID NO: 13, 14 or 15 having at least 85%, 86%, 87%, 88%, 89%, 90%, 91%, 92%, 93%, 94%, 95%, 96%, 97%, 98%, 99% or more sequence identity thereto.

In any of the embodiments herein, the cytoplasmic signaling domain derived from a costimulatory molecule is a signaling domain of 4-1BB. In any of the embodiments herein, the cytoplasmic signaling domain derived from a costimulatory molecule comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto.

In any of the embodiments herein, the cytoplasmic signaling domain derived from a primary signaling ITAM-containing molecule is a CD3zeta signaling domain. In any of the embodiments herein, the cytoplasmic signaling domain derived from a primary signaling ITAM-containing molecule comprises SEQ ID NO: 13, 14 or 15 having at least 85%, 86%, 87%, 88%, 89%, 90%, 91%, 92%, 93%, 94%, 95%, 96%, 97%, 98%, 99% or more sequence identity thereto.

In any of the embodiments herein, the CAR further comprises a spacer between the transmembrane domain and the extracellular antigen-binding domain. In some embodiments, the spacer comprises or consists of the sequence of SEQ ID NO: 1, a sequence encoded by SEQ ID NO: 2, the sequence of SEQ ID NO: 30, the sequence of SEQ ID NO: 31, the sequence of SEQ ID NO: 32, the sequence of SEQ ID NO: 33, the sequence of SEQ ID NO: 34, or a variant of any one of the foregoing having at least 85%, 86%, 87%, 88%, 89%, 90%, 91%, 92%, 93%, 94%, 95%, 96%, 97%, 98%, 99% or more sequence identity thereto. In some embodiments, the spacer comprises or consists of the formula X₁PPX₂P, where X₁ is glycine, cysteine or arginine and X₂ is cysteine or threonine

In any of the embodiments herein, the CAR may further include a spacer between the transmembrane domain and the scFv. In any of the embodiments herein, the spacer is a polypeptide spacer that includes or consists of all or a portion of an immunoglobulin hinge or a modified version thereof. In some embodiments, the spacer is an IgG4 hinge, or a modified version thereof. In any of the embodiments herein, the spacer is at or about 12 amino acids in length. In any of the embodiments herein, the spacer has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any one of the foregoing having at least 85%, 86%, 87%, 88%, 89%, 90%, 91%, 92%, 93%, 94%, 95%, 96%, 97%, 98%, 99% or more sequence identity thereto. In some embodiments, the spacer may include or consist of the formula X₁PPX₂P, where X₁ is glycine, cysteine or arginine and X₂ is cysteine or threonine. In any of the embodiments herein, the spacer is a polypeptide spacer that may include or consist of all or a portion of an immunoglobulin hinge or a modified version thereof or includes about 15 amino acids or less. In some embodiments, the spacer does not include a CD28 extracellular region or a CD8 extracellular region, and may include or consist of all or a portion of an immunoglobulin hinge, optionally an IgG4 hinge, or a modified version thereof and/or includes about 15 amino acids or less, and does not include a CD28 extracellular region or a CD8 extracellular region.

In some embodiments, the spacer is at or about 12 amino acids in length and/or includes or consists of all or a portion of an immunoglobulin hinge, optionally an IgG4, or a modified version thereof. In some embodiments, the spacer has or may consist of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any one of the foregoing having at least 85%, 86%, 87%, 88%, 89%, 90%, 91%, 92%, 93%, 94%, 95%, 96%, 97%, 98%, 99% or more sequence identity thereto. In some embodiments, the spacer may include or consist of the formula X₁PPX₂P, where X₁ is glycine, cysteine or arginine and X₂ is cysteine or threonine. In some of any of the embodiments, the costimulatory domain may include SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto. In some of any of the provided embodiments, the primary signaling domain may include SEQ ID NO: 13, 14 or 15 having at least 85%, 86%, 87%, 88%, 89%, 90%, 91%, 92%, 93%, 94%, 95%, 96%, 97%, 98%, 99% or more sequence identity thereto. In some of any embodiments, the scFv may include an amino acid sequence of RASQDISKYLN (SEQ ID NO: 35), an amino acid sequence of SRLHSGV (SEQ ID NO: 36), an amino acid sequence of GNTLPYTFG (SEQ ID NO: 37), an amino acid sequence of DYGVS (SEQ ID NO: 38), an amino acid sequence of VIWGSETTYYNSALKS (SEQ ID NO: 39), and an amino acid sequence of YAMDYWG (SEQ ID NO: 40). In some embodiments, the scFv includes a variable heavy chain region of FMC63 and a variable light chain region of FMC63. In some embodiments, the scFv includes six CDRs that includes a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63. In some embodiments, the scFv is an scFv that binds to the same epitope as or competes for binding with any one of the foregoing. In some embodiments, the scFv includes, in order, a VH, a linker, optionally a linker including SEQ ID NO: 24, and a VL. In some embodiments, the scFv includes, in order, a V, a linker, optionally a linker including SEQ ID NO: 24, and a VH. In some embodiments, the scFv includes a flexible linker. In some embodiments, the scFv amino acid sequence is set forth as SEQ ID NO: 43.

In any of the embodiments herein, (i) the scFv may comprise an amino acid sequence of RASQDISKYLN (SEQ ID NO: 35), an amino acid sequence of SRLHSGV (SEQ ID NO: 36), an amino acid sequence of GNTLPYTFG (SEQ ID NO: 37), an amino acid sequence of DYGVS (SEQ ID NO: 38), an amino acid sequence of VIWGSETTYYNSALKS (SEQ ID NO: 39), and an amino acid sequence of YAMDYWG (SEQ ID NO: 40), such as the scFv may comprise the amino acid sequence set forth as SEQ ID NO: 43; (ii) the spacer may be a polypeptide spacer that (a) comprises or consists of all or a portion of an immunoglobulin hinge or a modified version thereof is about 15 amino acids or less, and comprises or consists of all or a portion of an immunoglobulin hinge, optionally an IgG4 hinge, or a modified version thereof, optionally, such as wherein the spacer may comprise or consist of the sequence of SEQ ID NO: 1, or a sequence encoded by SEQ ID NO: 2; (iii) the costimulatory domain may comprise SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and (iv) the primary signaling domain may comprise SEQ ID NO: 13, 14 or 15 having at least 85%, 86%, 87%, 88%, 89%, 90%, 91%, 92%, 93%, 94%, 95%, 96%, 97%, 98%, 99% or more sequence identity thereto.

In any of the embodiments herein, the CAR is one in which (i) the scFv comprises the amino acid sequence set forth as SEQ ID NO: 43; (ii) the spacer is a polypeptide spacer that comprises or consists of the sequence of SEQ ID NO: 1, or a sequence encoded by SEQ ID NO: 2; (iii) the cytoplasmic signaling domain derived from a costimulatory molecule is a costimulatory domain that comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and (iv) the cytoplasmic signaling domain derived from a primary signaling ITAM-containing molecule is a cytoplasmic signaling domain that comprises SEQ ID NO: 13, 14 or 15 or a variant thereof having at least 85%, 86%, 87%, 88%, 89%, 90%, 91%, 92%, 93%, 94%, 95%, 96%, 97%, 98%, 99% or more sequence identity thereto.

In any of the embodiments herein, the CAR is one in which (i) the extracellular antigen binding domain comprises an scFv that comprises a variable heavy chain region of FMC63 and a variable light chain region of FMC63; the spacer is a polypeptide spacer that comprises the sequence of SEQ ID NO: 1; the cytoplasmic signaling domain derived from a costimulatory molecule comprises SEQ ID NO: 12; and the cytoplasmic signaling domain derived from a primary signaling ITAM-containing molecule comprises SEQ ID NO: 13, 14 or 15.

In any of the embodiments herein, the spacer is a polypeptide spacer that may include the sequence of SEQ ID NO: 1; the costimulatory domain may include SEQ ID NO: 12; the primary signaling domain may include SEQ ID NO: 13, 14 or 15; the antigen binding domain may include an scFv that includes a variable heavy chain region of FMC63 and a variable light chain region of FMC63. In any of the embodiments herein, the composition of cells is administered parenterally, optionally intravenously. In any of the embodiments herein, the subject is a human subject.

In particular embodiments of any of the provided methods, the CAR contains in order from N-terminus to C-terminus: an extracellular antigen-binding domain that is the scFv set forth in SEQ ID NO: 43, the spacer set forth in SEQ ID NO:1, the transmembrane domain set forth in SEQ ID NO:8, the 4-1BB costimulatory signaling domain set forth in SEQ ID NO:12, and the signaling domain of a CD3-zeta (CD3ζ) chain set forth in SEQ ID NO:13.

In some of any embodiments, the composition comprising engineered T cells is produced by a manufacturing process comprising: (i) exposing an input composition comprising primary T cells with a stimulatory reagent comprising an oligomeric particle reagent comprising a plurality of streptavidin mutein molecules under conditions to stimulate T cells, thereby generating a stimulated population, wherein: the oligomeric particle reagent comprises a first agent comprising an anti-CD3 antibody or antigen binding fragment thereof and a second agent comprising an anti-CD28 antibody or antigen binding fragment thereof; (ii) introducing into T cells of the stimulated population, a heterologous polynucleotide encoding the CAR that targets CD19, thereby generating a population of transformed cells; (iii) incubating the population of transformed cells for up to 96 hours; and (iv) harvesting T cells of the population of transformed cells, thereby producing a composition of engineered cells, wherein the harvesting is carried out at a time between 24 and 120 hours, inclusive, after the exposing to the stimulatory reagent is initiated. In some embodiments, the input composition comprises autologous T cells selected from the subject, such as enriched by immunoaffinity-based selection for CD3 T cells or CD4 and CD8 T cells from a blood or apheresis (e.g. leukarephesis) sample from the subject.

In any of the embodiments herein, the composition including engineered T cells is produced by a manufacturing process including exposing an input composition including primary T cells with a stimulatory reagent including an oligomeric particle reagent including a plurality of avidin, streptavidin, avidin mutein, or streptavidin mutein molecules under conditions to stimulate T cells, thereby generating a stimulated population, wherein the stimulatory reagent is capable of activating one or more intracellular signaling domains of one or more components of a TCR complex and one or more intracellular signaling domains of one or more costimulatory molecules. In any of the embodiments herein, the manufacturing process further may include introducing into T cells of the stimulated population, a heterologous polynucleotide encoding the CAR that targets CD19, thereby generating a population of transformed cells.

In some of any embodiments, the oligomeric particle reagent comprises a first agent comprising an anti-CD3 antibody or antigen binding fragment thereof and a second agent comprising an anti-CD28 antibody or antigen binding fragment thereof. In some of any embodiments, the anti-CD3 antibody or antigen binding fragment is a Fab and the anti-CD28 antibody or antigen binding fragment is a Fab. In some of any embodiments, the first agent and the second agent each comprise a streptavidin-binding peptide that reversibly binds the first agent and the second agent to the oligomeric particle reagent, optionally wherein the streptavidin-binding peptide comprises the sequence of amino acids set forth in any of SEQ ID NOS:78-82. In some of any embodiments, the streptavidin mutein molecule is a tetramer of a streptavidin mutein comprising amino acid residues Val44-Thr45-Ala46-Arg47 or Ile44-Gly45-Ala46-Arg47, optionally wherein the streptavidin mutein comprises the sequence set forth in any of SEQ ID NOS: 69, 84, 87, 88, 90, 85 or 59. In some of any embodiments, the oligomeric particle reagent comprises between 1,000 and 5,000 streptavidin mutein tetramers, inclusive. In some of any embodiments, the method further comprises, prior to harvesting the cells, adding biotin or a biotin analog after or during the incubation.

In any of the embodiments herein, the manufacturing process may further include incubating the population of transformed cells for up to 96 hours. In any of the embodiments herein, the incubating is carried out in basal media lacking one or more recombinant cytokines. In any of the embodiments herein, the manufacturing process may further include harvesting T cells of the population of transformed cells, thereby producing a composition of engineered cells. In any of the embodiments herein, the harvesting is carried out at a time between 24 and 120 hours, inclusive, after the exposing to the stimulatory reagent is initiated. In any of the embodiments herein, the harvesting is carried out at a time between 48 and 120 hours, inclusive, after the exposing to the stimulatory reagent is initiated. In any of the embodiments herein, the harvesting is carried out at a time when integrated vector is detected in the genome but prior to achieving a stable integrated vector copy number (iVCN) per diploid genome. In any of the embodiments herein, the harvesting is carried out at a time before the total number of viable cells at the harvesting is more than or more than about three times as the number of total viable cells of the stimulated population. In any of the embodiments herein, the harvesting is carried out at a time when the total number of viable cells at the harvesting is at or about three times, at or about two times, or the same or about the same as the number of total viable cells of the stimulated population. In any of the embodiments herein, the harvesting is carried out at a time when the percentage of CD27⁺CCR7⁺ cells is greater than or greater than about 50% among total T cells in the population of transformed cells, total CD3⁺ T cells in the population, total CD4⁺ T cells in the population of transformed cells, or total CD8⁺ T cells in the population of transformed cells, or of CAR-expressing cells thereof, in the population of transformed cells. In any of the embodiments herein, the harvesting is carried out at a time when the percentage of CD45RA⁺CCR7⁺ and CD45RA-CCR7⁺ cells is greater than or greater than about 60% among total T cells in the population of transformed cells, total CD3⁺ T cells in the population of transformed cells, total CD4⁺ T cells in the population of transformed cells, or total CD8⁺ T cells, or of CAR-expressing cells thereof, in the population of transformed cells.

In any of the embodiments herein, the cells in the administered composition are produced by a manufacturing process to produce an output composition (i) comprising engineered CD4+ T cells and engineered CD8+ T cells and (ii) exhibiting a predetermined feature, wherein iterations of the manufacturing process produce a plurality of the output compositions, optionally from human biological samples, when carried out among a plurality of different individual subjects, in which the predetermined feature of the output composition among the plurality of output compositions is selected from: the mean percentage of cells of a memory phenotype in the plurality of the output compositions is between about 40% and about 65%, between about 40% and about 45%, between about 45% and about 50%, between about 50% and about 55%, between about 55% and about 60%, or between about 60% and about 65%; the mean percentage of cells of a central memory phenotype in the plurality of the output compositions is between about 40% and about 65%, between about 40% and about 45%, between about 45% and about 50%, between about 50% and about 55%, between about 55% and about 60%, or between about 60% and about 65%; the mean percentage of cells that are CD27+, CD28+, CCR7+, CD45RA−, CD45RO+, CD62L+, CD3+, CD95+, granzyme B−, and/or CD127+ in the plurality of the output compositions is between about 40% and about 65%, between about 40% and about 45%, between about 45% and about 50%, between about 50% and about 55%, between about 55% and about 60%, or between about 60% and about 65%; the mean percentage of cells that are CCR7+/CD45RA− or CCR7+/CD45RO+ in the plurality of the output compositions is between about 40% and about 65%, between about 40% and about 45%, between about 45% and about 50%, between about 50% and about 55%, between about 55% and about 60%, or between about 60% and about 65%; the mean percentage of central memory CD4+ T cells in the engineered CD4+ T cells, optionally CAR+CD4+ T cells, of the plurality of the output compositions is between about 40% and about 65%, between about 40% and about 45%, between about 45% and about 50%, between about 50% and about 55%, between about 55% and about 60%, or between about 60% and about 65%; the mean percentage of central memory CD8+ T cells in the engineered CD8+ T cells, optionally CAR+CD8+ T cells, of the plurality of the output compositions is between about 40% and about 65%, between about 40% and about 45%, between about 45% and about 50%, between about 50% and about 55%, between about 55% and about 60%, or between about 60% and about 65%; and/or the mean percentage of central memory T cells, optionally CD4+ central memory T cells and CD8+ central memory T cells, in the engineered T cells, optionally CAR+ T cells, of the plurality of the output compositions is between about 40% and about 65%, between about 40% and about 45%, between about 45% and about 50%, between about 50% and about 55%, between about 55% and about 60%, or between about 60% and about 65%. In some embodiments, the plurality of output compositions are produced from human biological samples.

In any of the embodiments herein, the administered composition is produced by a manufacturing process to produce an output composition exhibiting a predetermined feature, optionally a threshold number of cells expressing the CAR in the output composition, in at least about 80%, about 90%, about 95%, about 97%, about 99%, about 100%, or is 100% of the human biological samples in which it is carried out among a plurality of different individual subjects. In any of the embodiments herein, the composition including genetically engineered cells does not contain residual beads from the manufacturing process. In any of the embodiments herein, the B-cell NHL is a relapsed and/or refractory B-cell non-Hodgkin lymphoma (B-cell NHL). In any of the embodiments herein, the predetermined feature is a threshold number of cells expressing the CAR in the output composition.

Also provided herein is an article of manufacture including a composition including genetically engineered cells expressing a chimeric antigen receptor (CAR) that targets CD19, and instructions for administering the composition of the cells in accordance with any of the methods provided herein.

### BRIEF DESCRIPTION OF THE DRAWINGS

FIG. 1 shows exemplary quantifications determined by flow cytometry of cell purity of T cell compositions produced from non-expanded engineering processes using different donor types (Reference, Patient). Cells were engineered to express an anti-CD19 CAR (CD19) or were mock transduced (mock). Percentages of CD3+ cells of live CD45+ cells (left panel), percentages of NK cells of live CD45+ cells (middle panel), and percentages of CD19+ cells of live CD45+ cells (right panel) were determined.

FIGS. 2-3 shows exemplary quantifications of cell phenotypes determined by flow cytometry for expanded and non-expanded engineering processes using different donor types (Reference, Patient). Cells were engineered to express an anti-CD19 CAR (CD19) or were mock transduced (mock). FIG. 2 shows percentages of CD3+CD8+ and CD3+CD4+ cells of live CD45+ cells (left panel) and percentages of CD8+CAR+ and CD4+CAR+ cells of live CD45+ cells (right panel). FIG. 3 shows ratios of CD4+ cells to CD8+ cells, and CD4+CAR+ cells to CD8+CAR+ cells.

FIG. 4 shows exemplary quantifications determined by flow cytometry of cell viability of T cell compositions produced from non-expanded engineering processes using different donor types (Reference, Patient). Cells were engineered to express an anti-CD19 CAR (CD19) or were mock transduced (mock). Percentages of aCas3+ cells of CD3+ cells were determined.

FIG. 5A shows exemplary relationship between copy number per cell among total cells as assessed by standard VCN (without pulse field gel electrophoresis, or PFGE) and iVCN (with PFGE), in cell compositions produced from primary T cells from different human donors that had been engineered to express a CAR using an expanded process (∘) or a non-expanded process (●). FIGS. 5B-5C show the relationship between the copy number per cell in the cell compositions as assessed by standard VCN (FIG. 5B) or iVCN (FIG. 5C) and the surface expression of the CAR, as indicated by the percentage of CAR-expressing CD3+ cells (% CD3+CAR+) among viable CD45+ cells assessed by flow cytometry.

FIGS. 6A-6B show exemplary percentages of cell phenotypes resulting from expanded and non-expanded engineering processes using different donor types (Reference, Patient). Cells were engineered to express an anti-CD19 CAR (CD19) or were mock transduced (mock). FIG. 6A shows exemplary percentages of CD45RA+CCR7+ cells of aCas-CD8+CAR+ and aCas-CD4+CAR+ cells (left top panel), CD45RA-CCR7+ cells of aCas-CD8+CAR+ and aCas-CD4+CAR+ cells (right top panel), CD45RA-CCR7− cells of aCas-CD8+CAR+ and aCas-CD4+CAR+ cells (left bottom panel), and CD45RA+CCR7− cells of aCas-CD8+CAR+ and aCas-CD4+CAR+ cells (right bottom panel). FIG. 6B shows exemplary percentages of CD27+CCR7+ cells of aCas-CD8+CAR+ and aCas-CD4+CAR+ cells.

FIGS. 7A-7D show exemplary quantifications of cell phenotypes as indicated determined by flow cytometry for donor-matched expanded and non-expanded engineering processes where cells were engineered to express an anti-CD19 CAR, before the long-term CAR-dependent stimulation (primary) and after the long-term CAR-dependent stimulation (after 9 to 14 days of chronic stimulation with an agonistic anti-idiotype antibody) (secondary). DP1=diffuse large B-cell lymphoma (DLBCL) patient 1; DP2=DLBCL patient 2; HD1=healthy donor 1; MP1=mantle cell lymphoma patient 1.

FIGS. 8A-8F show exemplary in vitro proliferative capacity of cells generated from non-expanded and expended processes following long-term anti-CD19 CAR-dependent stimulation with an anti-ID antibody. Viability (FIG. 8A) and cell size (FIG. 8B) were measured. Fold change in expansion was calculated as the daily counts divided by the starting number of cells (FIG. 8C), and then transformed into an area under the curve (AUC) for comparison either for individual arms (FIG. 8D) or by manufacturing process (FIG. 8E, statistical significance with a Mann-Whitney test; *p<0.05). Individual donor fold expansion between the manufacturing platforms was also directly measured by taking the daily fold expansion of compositions generated from the non-expanded process and dividing them by the donor-matched control values for compositions generated from the expanded process (FIG. 8F).

FIGS. 9A-9B show exemplary cytolytic potential of anti-CD19 CAR T cells engineered by non-expanded or expanded processes before (FIG. 9A) and after chronic stimulation (FIG. 9B) at different effector to target ratios. Area under the curve (AUC) values were compared either for individual arms (left panels of FIG. 9A and FIG. 9B) or by manufacturing process (right panels of FIG. 9A and FIG. 9B, statistical significance with a Mann-Whitney test; *p<0.05).

FIG. 10 shows exemplary cytokine productions by cells engineered by non-expanded or expanded processes, measured by a polyfunctional score using flow cytometry, at Day 0 and Day 10 of chronic antigen stimulation. Statistical significance was assessed by Mann-Whitney, *p<0.05.

FIGS. 11A-11B show exemplary tumor burden and circulating CAR-T cells in the Nalm6 leukemia model over time following treatment with anti-CD19 CAR-T cell compositions generated from non-expanded and expanded matched-donor engineering processes. FIG. 11A shows tumor growth from Day −1 (before treatment) to about Day 25 post-treatment calculated from area under the curve (AUC) of bioluminescence (BLI) for each group. FIG. 11B shows circulating anti-CD19 CAR-T cell counts per 1 μl of blood between about Day 5 and about Day 20 post-treatment for each group.

FIGS. 12A-12B show exemplary tumor burden and circulating CAR-T cells in Raji lymphoma model over time following treatment with anti-CD19 CAR-T cell compositions generated from non-expanded and expanded matched-donor engineering processes. FIG. 12A shows tumor growth from Day −1 (before treatment) to about Day 80 post-treatment calculated from area under the curve (AUC) of BLI for each group, with higher (left panel) and lower (right panel) doses of anti-CD19 CAR-T cell compositions generated from non-expanded and expanded matched-donor engineering processes. Changes in BLI radiance (photons/second; y-axis) are shown for all groups (tumor burden). FIG. 12B shows circulating anti-CD19 CAR-T cell counts per 1 μl of blood at the indicated time points post-treatment for each group (non-expanded, NE; expanded, E). Absolute counts at each time point are shown as group means. Differences were compared using Mann-Whitney U test; *p<0.05.

### DETAILED DESCRIPTION

Provided herein are methods and uses of engineered cells (e.g., T cells) and/or compositions thereof, for the treatment of subjects having a disease or condition, which generally is or includes a cancer or a tumor, such as a lymphoma, most particularly a B cell malignancy that is a non-Hodgkin lymphoma (NHL), including aggressive subtypes of NHL. In particular aspects, the subject has an aggressive or high-risk NHL. In embodiments of the provided methods, the therapeutic T cell compositions containing the engineered cells are administered to a subject having NHL, e.g., via adoptive cell therapy, such as adoptive T cell therapy. In particular embodiments of any of the provided methods, the T cells are engineered with a chimeric antigen receptor (CAR) that is directed against cluster of differentiation 19 (CD19). In some aspects, the disease or condition is a B cell lymphoma. In some aspects, the disease or condition is a large B cell lymphoma. In some aspects, the disease or condition is a diffuse large B-cell lymphoma (DLBCL) or a subtype thereof. In some aspects, the methods and uses provide for or achieve improved response and/or more durable responses or efficacy and/or a reduced risk of toxicity or other side effects, e.g., in particular groups of subjects treated, as compared to certain alternative methods. In some embodiments, the methods are advantageous by virtue of the administration of specified numbers or relative numbers of the engineered cells, the administration of defined ratios of particular types of the cells, the administration of cells of a particular high percentage of less differentiated cells (e.g. naïve-like or central memory cells or cells of an early differentiation state, such as CCR7+CD27+ cells), treatment of particular patient populations, such as those having a particular risk profile, staging, and/or prior treatment history, and/or combinations thereof.

In some embodiments, the methods and uses include administering to the subject T cells expressing genetically engineered (recombinant) cell surface receptors in adoptive cell therapy, which generally are chimeric receptors such as chimeric antigen receptors (CARs), recognizing CD19 expressed by, associated with and/or specific to the NHL and/or cell type from which it is derived. The cells are generally administered in a composition formulated for administration; the methods generally involve administering one or more doses of the cells to the subject, which dose(s) may include a particular number or relative number of cells or of the engineered cells. In some cases, the CD19-directed CAR+ engineered cells in the composition include a defined ratio or compositions of two or more sub-types within the composition, such as CD4 vs. CD8 T cells. In particular embodiments, the compositions of cells for use or administration in the provided methods include primary T cells engineered to express a CD19-directed CAR that (i) contain a low percentage (e.g. less than 40%, less than 30%, less than 20%, or less than 10%) of exhausted cells and/or cells that display markers or phenotypes associated with exhaustion; and/or (ii) contain a relatively high percentage (e.g. greater than 50%, greater than 60%, greater than 70%, greater than 80% or greater than 90%) of memory-like T cells, such as naïve-like T cells, central memory T cells or long-lived memory T cells. In provided embodiments, the features of the compositions and provided methods result in improved or enhanced survival, expansion, persistence, and/or anti-tumor activity compared to methods involving administration other CD19-directed CAR T cell therapies that contain a higher percentage of exhausted cells and/or a higher number of cells that display phenotypes associated with exhaustion and/or that contain a lower percentage of certain T cells, such as naïve-like T cells, central memory T cells or long-lived memory T cells. In provided embodiments, the features of the compositions and provided methods result in improved therapeutic efficacy, e.g. increased percentage of patients achieving a complete response (CR), compared to methods involving administration of other CD19-directed CAR T cell therapies that contain a higher percentage of exhausted cells and/or a higher number of cells that display phenotypes associated with exhaustion and/or that contain a lower percentage of certain T cells, such as naïve-like T cells, central memory T cells or long-lived memory T cells. In provided methods, the features of the compositions and provided methods result in improved clinical durability of therapeutic response, such as CR, e.g., response that persists after a period of time from initiation of therapy, compared to methods involving administration of other CD19-directed CAR T cell therapies that contain a higher percentage of exhausted cells and/or a higher number of cells that display phenotypes associated with exhaustion and/or that contain a lower percentage of memory-like T cells, such as naïve-like T cells, central memory T cells or long-lived memory T cells. In particular embodiments, the use or administration of the provided CD19-directed CAR T cell compositions in the provided methods can be achieved with doses of cells that are more than 2-fold lower, such as 5-fold or 10-fold, lower than doses of reference CD19-directed CAR T cell compositions (e.g. engineered with the same or similar CAR, such as with the same antigen-binding domain) but in which the reference CD19-directed CAR T cell composition contain a higher percentage of exhausted cells and/or a higher number of cells that display phenotypes associated with exhaustion and/or that contains a lower percentage of memory-like T cells, such as naïve-like T cells, central memory T cells or long-lived memory T cells. In some embodiments, the reference CD19-directed CAR T cell composition is a composition that is produced ex vivo by processes that involve steps of cultivating the cells under conditions for expansion, such as resulting in proliferation of cells or population doubling of cells (e.g. 2, 3, 4, 5, 6, 7, 8, 9, 10 or more doublings of cells in the population compared to the start of the process) during the process for producing the cells.

In some embodiments, the CD19-directed CAR T cell compositions for use in the provided methods and uses are produced by a relatively short process that do not include a step for cultivating the cells under conditions for expansion designed for expanding or proliferating the cells. Different processes are available for generating compositions containing genetically engineered T cell populations, including for generating engineered T cells that express a CAR, which typically include a step designed for or for the purpose of cultivating the cells to expand or increase proliferation of the cells. However, in particular aspects, some of these processes may require a long or a relatively long amount of time to generate the engineered cells. In addition, in various aspects, some existing processes may vary in the amount of time required to successfully produce engineered T cells suitable for cell therapy, making it difficult to coordinate that administration of the cell therapy. In certain aspects, some of these processes may produce populations of cells that include a relatively high percentage or amount of exhausted cells, differentiated cells, or cells with a low potency. The provided CD19-directed CAR T cell compositions for use in the provided methods address one or more of these problems.

In particular embodiments, the provided methods are used in connection with a process for efficiently producing or generating engineered cells that are suitable for use in a cell therapy. In some embodiments, provided compositions containing CD19-directed CAR engineered T cells are produced by a process without the need for any additional steps for expanding the cells, e.g. without an expansion unit operation and/or without steps intended to cause expansion of cells. In aspects of processes for producing CD19-directed CAR T cell composition, the processes include one or more steps for stimulating and genetically engineering (e.g., transforming, transducing or transfecting) T cells to produce a population of engineered T cells that may be collected or formulated for use as a composition for cell therapy. In particular embodiments, the processes include a step of transducing cells with a viral vector (e.g. lentiviral vector) that contains a nucleic acid encoding the CD19-directed CAR. In some aspects, the provided processes result in the stable integration of the heterologous nucleic acid (expressed from the viral vector) into the genome of the cells. In some aspects, the provided processes generate engineered CD19-directed CAR T cells with enhanced potency as compared to engineered T cell compositions produced from alternative processes, such as those that involve expanding the cells.

In particular aspects, the durations of the processes for producing the provided compositions can be measured from when cells, e.g., T cells of an input cell population or input composition, are first contacted or exposed to stimulating conditions (e.g., as described herein such as in Section II-C), referred to herein as the initiation of the stimulation or stimulating and also referred to herein as the exposing to the stimulatory reagent, e.g., as in when the exposing to the stimulatory reagent is initiated. In some embodiments, the duration of time required to harvest or collect an output population (also referred to herein as an output composition or as a composition of engineered cells, e.g., engineered T cells) containing engineered cells is measured from initiation of the stimulation. In particular embodiments, the duration of the process is, is about, or is less than 120 hours, 108 hours, 96 hours, 84 hours, 72 hours, 60 hours, 48 hours, 36 hours, or 30 hours. In particular embodiments, the duration of the process is, is about, or is less than 5 days, 4 days, 3 days, 2 days, or one day. In particular embodiments, the engineered cells, e.g., the cells of the output composition or population, are more potent, persistent or naïve-like than cells that are engineered with processes that require longer amounts of time. In some aspects, the duration, e.g., the amount of time required to generate or produce an engineered population of T cells, of the provided processes are shorter than those of some existing processes by, by about, or by at least 2 days, 3 days, 4 days, 5 days, 6 days, 7 days, or more than 7 days. In some embodiments, the duration of the provided process is, is about, or is less than 75%, 60%, 50%, 40%, 30%, 25%, 15%, or 10% of alternative or existing processes.

In certain embodiments, the provided processes are performed on a population of cells, e.g., CD3+, CD4+, and/or CD8+ T cells, that are isolated, enriched, or selected from a biological sample. In some aspects, the provided methods can produce or generate a composition of engineered T cells from when a biological sample is collected from a subject within a shortened amount of time as compared to other methods or processes. In some embodiments, the provided methods can produce or generate engineered T cells, including any or all times where biological samples, or enriched, isolated, or selected cells are cryopreserved and stored, within or within about 10 days, 9 days, 8 days, 7 days, 6 days, 5 days, 4 days, 3 days, or 2 days, or within or within about 120 hours, 96 hours, 72 hours, or 48 hours, from when a biological sample is collected from a subject to when the engineered T cells are collected, harvested, or formulated (e.g., for cryopreservation or administration).

In particular embodiments, the processes for producing or engineering T cell populations include a step of stimulating the cells, such as prior to transduction with a viral vector. In aspects of the provided processes, stimulation is carried out with an oligomeric stimulatory reagent, such as a streptavidin mutein oligomer, to which is immobilized or attached a stimulatory binding agent(s), e.g. anti-CD3/anti-CD28. Existing reagents for use in stimulating T cells in vitro, such as in the absence of exogenous growth factors or low amounts of exogenous growth factors, are known (see e.g. U.S. Pat. No. 6,352,694 B1 and European Patent EP 0 700 430 B1). In general, such reagents may employ beads, e.g., magnetic beads, of greater than 1 μm in diameter to which various binding agents (e.g. anti-CD3 antibody and/or anti-CD28 antibody) are immobilized. However, in some cases, such magnetic beads are, for example, difficult to integrate into methods for stimulating cells under conditions required for clinical trials or therapeutic purposes since it has to be made sure that these magnetic beads are completely removed before administering the expanded T cells to a subject. In some aspects, such removal, such as by exposing the cells to a magnetic field, may decrease the yield of viable cells available for the cell therapy. In certain cases, such reagents, e.g., stimulatory reagents containing magnetic beads, must be incubated with the cells for a minimal amount of time to allow a sufficient amount of detachment of the T cells from the stimulatory reagent.

The provided processes utilizing oligomeric stimulatory reagents, e.g. streptavidin mutein polymer, overcome such potential limitations. For example, in some embodiments, the provided processes avoid or reduce risk of residual stimulatory reagent, e.g., reagents containing magnetic beads, in the output cells generated or produced by the processes. In some embodiments, this also means that a process that is compliant with GMP standards can be more easily established compared to other methods, such as those where additional measures have to be taken to ensure that the final engineered T cell population is free of beads. In some embodiments, this may be readily accomplished in the present embodiments by the addition of a substance, e.g., a competition reagent, that dissociates the oligomeric stimulatory reagents from the cells, e.g., by simply rinsing or washing the cells. e.g., by centrifugation. Thus, in some aspects, removal or separation of oligomeric stimulatory reagent from cells, such as by the addition of a substance or competition reagent, results in little or no cell loss as compared to removal or separation of bead based stimulatory reagents. In some aspects, the timing of the oligomeric stimulatory reagent removal or separation is not limited or is less limited than the removal or separation of bead based stimulatory reagents. Thus, in some aspects, the oligomeric stimulatory reagent may be removed or separated from the cells at any time or stage during the provided processes.

In some aspects, the use of oligomeric stimulatory reagents (e.g. anti-CD3/anti-CD28 streptavidin mutein oligomers) can result in an overall reduced stimulatory signal compared to alternative stimulatory reagents, such as anti-CD3/anti-CD28 paramagnetic beads. The provided process, which can involve a weaker or reduced stimulation, can generate engineered CAR+ T cells that are as, or even more, potent, persistent, or efficacious as CAR+ T cells generated by processes that involve stronger stimulatory conditions or higher amounts or concentrations of stimulatory reagent, such as may occur following stimulation with anti-CD3/anti-CD28 paramagnetic beads. In addition, in some embodiments, stimulating cells with a lower amount or relatively low amount of oligomeric stimulatory reagents may increase the potency, efficacy, or persistency of the resulting engineered cell population, as compared to processes using higher amounts of oligomeric stimulatory reagent. Such embodiments contemplate that such effects may persist even at doses sufficiently low enough to reduce the expression of activation markers or the portion of cells positive for the activation markers during and after the process.

In certain embodiments, the engineered T cells, e.g., output composition or populations of T cells containing T cells expressing a recombinant receptor, such as a chimeric antigen receptor, produced or generated by the provided processes are particularly effective or potent when utilized as cells for a cell therapy. For example, in some aspects, an output composition containing engineered T cells, e.g., CAR+ T cells, that are generated from the provided processes have a much higher degree of potency and/or proliferative capacity than engineered T cells generated or produced by alternative existing processes. In some aspects, an output composition containing engineered T cells, e.g., CAR+ T cells, produced by the provided processes have enhanced anti-tumor or anti-cancer cell activity than engineered T cells, e.g., CAR+ T cells, produced by alternative or existing methods.

In particular embodiments, the processes for producing the provided CD19-directed T cell compositions that do not contain steps where the cells are expanded to a threshold amount or concentration have further advantages. In some aspects, protocols that do not rely on expanding the cells to increase the number or concentration of cells from a starting cell population, e.g., an input population, do not require incubations or cultivations that may vary between cell populations. For example, some embodiments contemplate that cell populations obtained from different subjects, such as subjects having different diseases or disease subtypes, particularly as is the case for patients with NHL, including high-risk, aggressive and/or R/R NHL, may divide or expand at different rates. In certain aspects, eliminating potentially variable steps requiring cell expansion allows for the duration of the whole process to be tightly controlled. In certain embodiments, the variability of the process duration is reduced or eliminated which may, in some aspects, allow for improved coordination for appointments and treatment between doctors, patients, and technicians to facilitate autologous cell therapies.

In some embodiments, the provided methods involve treating a specific group or subset of subjects, e.g., subjects identified as having high-risk disease, e.g., high-risk NHL or a high-risk large B cell lymphoma. In some aspects, the methods treat subjects having a form of aggressive and/or poor prognosis B-cell non-Hodgkin lymphoma (NHL), such as NHL that has relapsed or is refractory (R/R) to standard therapy and/or has a poor prognosis. In some aspects, the methods treat subjects having a large B cell lymphoma that has relapsed or is refractory (R/R) to standard therapy. In particular aspects, the engineered cells are autologous to the subject and are administered following generation by ex vivo processes that are shortened compared to existing methods, that do not include or involve a cultivation step for expanding the cells during the methods of producing the engineered cells, and/or that are able to produce a CAR-engineered T cell composition that is less differentiated permitting administration of lower doses. As a result, the provided methods are advantageous compared to existing methods because they can shorten the time until the engineered T cell therapy is available to the patient, particularly among patients who are in need of treatment, such as subjects that have relapsed to or are refractory to treatment following one or more other prior therapies for treating the disease or condition. In some aspects, the provided methods, compositions, uses and articles of manufacture achieve improved and superior responses to available therapies. In some embodiments, the improved or superior responses are to current standard of care (SOC).

Non-Hodgkin lymphoma (NHL) is a group of lymphoid malignant neoplasms with diverse biological and clinical behavior. It is estimated there will be approximately 74,200 new cases diagnosed and 19,970 deaths from NHL in the United States (US) in 2019 (Siegel et al., CA Cancer J Clin. 2019; 69(1):7-34). Non-Hodgkin lymphoma can be divided into 2 prognostic groups: the indolent lymphoma (slowly growing with waxing and waning lymphadenopathy for years) and the aggressive lymphoma (rapidly growing and resulting death within a few weeks if not treated). The most common aggressive lymphoma is diffuse large B-cell lymphoma (DLBCL) accounting for 30 to 40% of all NHL (Li et al., Pathology 2018; 50(1):74-87). Other aggressive lymphomas include but are not limited to high grade B-cell lymphoma (HGBCL) so-called as double hit (DHL) or triple hit lymphoma (THL), mantle cell lymphoma (MCL), primary mediastinal large B-cell lymphoma (PMBCL), and follicular lymphoma grade 3b (FL3B). Prognosis and therapy of other aggressive lymphoma subtypes are similar to DLBCL. About 50% of newly diagnosed patients with DLBCL can be cured with first-line R-CHOP immunochemotherapy (rituximab, cyclophosphamide, doxorubicin, vincristine and prednisone). However, approximately half of the patients treated with R-CHOP will relapse, mostly within the first 2 years after therapy (Coiffier et al., Blood 2010; 116(12):2040-5; Vitolo et al., J Clin Oncol. 2017; 35(31):3529-37). Salvage high-dose immunochemotherapy followed by autologous stem-cell transplantation (ASCT) is the standard secondline therapy for relapsed or refractory (R/R) DLBCL. Approximately half of the R/R patients will not be transplant eligible due to no response to salvage therapy or medical condition, and significant portion of patients will relapse even after ASCT. A multicohort retrospective NHL research study in refractory DLBCL (SCHOLAR-1; n=636), demonstrated very poor outcomes in this patient population with an overall response rate (ORR) of 26% to the next line of therapy and a median overall survival (OS) of 6.3 months (Crump et al., Blood 2017; 130(16):1800-8; Gisselbrecht et al., Br J Haematol. 2018; 182(5):633-43).

CD19 is a member of the immunoglobulin superfamily and a component of the B-cell surface signal transduction complex that positively regulates signal transduction through the B-cell receptor. It is expressed by most B-cell malignancies from early development until differentiation into plasma cells (Stamenkovic et al., J Exp Med. 1988; 168(3):1205-10). CD19 is an attractive therapeutic target because it is expressed by most B-cell malignancies, including B-cell NHL (Davila et al., Oncoimmunology. 2012;(9):1577-83). Importantly, the CD19 antigen is not expressed on hematopoietic stem cells or on any normal tissue apart from those of the B-cell lineage. Additionally, CD19 is not shed in the circulation, which limits off-target adverse effects (Shank et al., Pharmacotherapy. 2017; 37(3):334-45).

In particular embodiments, the methods provided herein are based on administration of a CD19-directed CAR T cell therapy in which the CAR contains a CD19-directed scFv antigen binding domain (e.g. from FMC63). The CAR further contains an intracellular signaling domain containing a signaling domain from CD3zeta, and also incorporates a 4-1BB costimulatory domain, which has been associated with lower incidence of cytokine release syndrome (CRS) and neurotoxicity (NE) compared with CD28-containing constructs (Lu et al. J Clin Oncol. 2018; 36:3041).

A challenge in CAR T cell development is to generate a product that consistently expands, persists, and mediates durable antitumor responses after infusion. Certain CD19-directed CAR-T cell therapies are available for the treatment of B cell lymphoma, including Kymriah™ (tisagenlecleucel) (Kymriah PI) and Yescarta® (axicabtagene ciloleucel) (Yescarta PI). Both CAR T cell therapies have initial 50 to 60% of complete response rates (CRR) in this difficult to treat patient population; however, at 6 months the response rate drops to the 40 to 50% range with responders more likely to have ongoing durable responses (Locke et al., Mol Ther. 2017; 25(1):285-95; Schuster et al., N Engl J Med. 2017; 377(26):2545-54). Thus, more than half of the patients who receive these therapies will not have a long lasting, durable response, and the prognosis for overall survival (OS) is very poor.

The provided methods are based on findings that a lower differentiation state of adoptively transferred T cells can influence the ability of these cells to persist and promote durable antitumor immunity. In some embodiments, the provided CD19-directed CAR+ engineered T cell compositions are produced by a method in which the cells are not cultivated under conditions of expansion, thereby limiting or reducing the number of population doublings of the final engineered output composition and resulting in a less differentiated product. Yet, the provided compositions also are produced via processes that result in stably integrated vector copy number (iVCN) to ensure consistent and reliable expression of the CAR, thereby resulting in a consistent cell product for administration to subjects and low variability among CAR-expressing cells in administered doses. In contrast, most protocols for T cell engineering routinely expand T cells ex vivo for 9 to 14 days or more. Provided data exemplified herein support a model in which CAR T cell products with an increased composition of less differentiated memory T cells may exhibit enhanced durable antitumor activity. These findings reveal that strategies aimed at minimizing effector differentiation in CAR T cell products could result in improved clinical efficacy. Provided herein are embodiments that can meet such aims.

The observations herein support treating subjects with high-risk disease with a CD19-directed CAR T cell therapy in accord with the provided methods. For example, subjects with NHL, including patients with aggressive NHL or certain high-risk features, such as those with relapsed/refractory (R/R) NHL, can be treated in accord with the provided methods. In some embodiments, the provided methods can be used to treat subjects that have been heavily pretreated (e.g. with one, two, three, four, or more prior therapies for treating the disease).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Methods and Uses of CD19-Targeted Cell Therapy in B Cell Non-Hodgkin's Lymphoma

Provided herein are methods of treatment that involve administering engineered cells or compositions containing engineered cells, such as engineered T cells. Also provided are methods and uses of provided CD19-directed CAR engineered cells (e.g., T cells) and/or compositions thereof, including methods for the treatment of subjects having a B cell non-Hodgkin's lymphoma (NHL), including aggressive or high-risk NHL, such as R/R NHL, that involves administration of the engineered cells and/or compositions thereof. In some embodiments, the methods and use of provided CD19-directed CAR engineered cells (e.g., T cells) and/or compositions thereof, including methods for the treatment of subjects with R/R NHL that have failed at least two or more prior therapies. In particular embodiments, the method includes administering to the subject a dose of T cells that includes CD4+ and CD8+ T cells, wherein the T cells comprises a chimeric antigen receptor (CAR) that specifically binds to CD19.

In some embodiments, the methods and uses include administering to the subject cells expressing genetically engineered (recombinant) cell surface receptors in adoptive cell therapy, which generally are chimeric receptors such as chimeric antigen receptors (CARs), recognizing CD19 expressed by, associated with and/or specific to the lymphoma and/or cell type from which it is derived. The cells are generally administered in a composition formulated for administration. In some embodiments, cells are collected from the subject prior to treatment for the purpose of engineering the cells with the CD19-directed recombinant receptor (e.g. CAR). In some embodiments, the cells are collected by leukapheresis. In some aspects, the cells are engineered by ex vivo methods that do not involve cultivating the cells for expansion (hereinafter also called non-expanded process). Exemplary non-expanded processes for engineering the provided CAR-expressing therapeutic compositions are described in Section II-C.

In some embodiments, the disease or condition is an aggressive NHL. In some embodiments, the disease or condition is an aggressive NHL that was previously indolent. In some embodiments, the NHL is non-indolent, such is an aggressive lymphoma that is fast moving. Among the most common aggressive NHL subtype is Diffuse large B-cell lymphoma (DLBCL).

In some embodiments, the disease or condition is confirmed using positron emission tomography (PET). In some embodiments, the disease or condition is confirmed using PET and staged based on the Lugano Classification (see, e.g., Cheson et al., (2014) JCO 32(27):3059-3067; Cheson, B. D. (2015) Chin Clin Oncol 4(1):5). In some embodiments, the disease or condition is histologically confirmed. In any embodiments disclosed herein, the disease or conditions, or subtype or state, can be determined prior to leukapheresis in connection with obtaining T cells for autologous T cell therapy.

In some embodiments, the disease or condition is a diffuse large B cell lymphoma (DLBCL).

In some embodiments, the disease or condition is a DLBCL not otherwise specified (NOS). In some embodiments, the disease or condition is a high-grade B cell lymphoma with MYC and BCL2 and/or BCL6 rearrangements with DLBCL histology (HGBCL). In some embodiments, the disease or condition is a DLBCL transformed from follicular lymphoma (tFL). In some embodiments, the disease or condition is a DLBCL transformed from marginal zone lymphoma (tMZL). In some embodiments, the disease or condition is a primary mediastinal B cell lymphoma (PMBCL). In some embodiments, the disease or condition is a follicular lymphoma (FL). In some embodiments, the disease or condition is a follicular lymphoma grade 3B (FL3B).

In some embodiments, the subject prior to leukapheresis has an Eastern Cooperative Oncology Group (ECOG) performance status of 0 or 1 (see, e.g., Oken et al., (1982) Am J Clin Oncol. 5:649-655). In some embodiments, the Eastern Cooperative Oncology Group (ECOG) performance status indicator can be used to assess or select subjects for treatment, e.g., subjects who have had poor performance from prior therapies (see, e.g., Oken et al., (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of self-care; see e.g., Ssrensen et al., (1993) Br J Cancer 67(4) 773-775. The criteria reflective of the ECOG performance status are described in Table 1 below:

TABLE 1
ECOG Performance Status Criteria

| Grade | ECOG performance status |
|---|---|
| 0 | Fully active, able to carry on all pre-disease |
| | performance without restriction |
| 1 | Restricted in physically strenuous activity but |
| | ambulatory and able to carry out work of a |
| | light or sedentary nature, e.g., light house |
| | work, office work |
| 2 | Ambulatory and capable of all self-care but |
| | unable to carry out any work activities; |
| | up and about more than 50% of waking hours |
| 3 | Capable of only limited self-care; confined |
| | to bed or chair more than 50% of waking hours |
| 4 | Completely disabled; cannot carry on any |
| | self-care; totally confined to bed or chair |
| 5 | Dead |

In some embodiments, prior to, such as at the time of, administration of the provided CD19-directed CAR T cell compositions, the subject has relapsed following remission after treatment with, or become refractory to, one or more lines of prior therapy for the NHL. In any embodiments, at a time prior to leukapheresis in connection with engineering the CD19-directed CAR T cell composition, the subject has relapsed following remission after treatment with, or become refractory to, one or more lines of prior therapy for treating the NHL. Thus, in particular embodiments, prior to the time of treatment, such as prior to leukapheresis, the subject has a R/R NHL. In some embodiments, the subject has been previously treated with a therapy or a therapeutic agent targeting the disease or condition, e.g., a NHL or a, prior to administration of the cells expressing the recombinant receptor. In some embodiments, the subject has been previously treated with a hematopoietic stem cell transplantation (HSCT), e.g., allogeneic HSCT or autologous HSCT. In some embodiments, the subject has had poor prognosis after treatment with standard therapy and/or has failed one or more lines of previous therapy. In some embodiments, the subject has been treated or has previously received at least or at least about or about 1, 2, 3, 4, or more other therapies for treating the NHL, such as an aggressive or high-risk NHL, such as DLBCL or a subtype thereof. In some embodiments, the subject has been treated or has previously received a therapy that includes a CD20 targeted agent (e.g. anti-CD20 antibody) and an alkylating agent. In some aspects, the subject has relapsed after an initial response of complete response (CR) or partial response (PR) to the prior therapy. In some embodiments, the subject is refractory to treatment with the at least one or more prior therapy, and the refractory treatment is a best response of stable disease (SD) or progressive disease (PD) after the prior therapy.

In some embodiments, the subject has relapsed following remission after treatment with, or become refractory to, at least two lines of prior systemic therapy for the disease or condition. In some embodiments, at least one said line of prior systemic therapy comprises anthracycline and anti-CD20 monoclonal antibody treatment. Exemplary anti-CD20 antibodies include but are not limited to rituximab, ofatumumab, ocrelizumab (also known as GA101 or R05072759), veltuzumab, obinutuzumab, TRU-015 (Trubion Pharmaceuticals), ocaratuzumab (also known as AME-133v or ocaratuzumab), and Pro131921 (Genentech). See, e.g., Lim et al. Haematologica. (2010) 95(1):135-43. Rituximab is a chimeric mouse/human monoclonal antibody IgG1 kappa that binds to CD20 and causes cytolysis of a CD20 expressing cell. In some embodiments, at least one said line of prior systemic therapy comprises anthracycline and rituximab treatment.

In some embodiments, the subject has relapsed following remission after treatment with, or become refractory to, autologous stem-cell transplantation (ASCT). In some embodiments, relapse following remission after treatment with or becoming refractory to ASCT is failure to achieve an objective response after ASCT. In some embodiments, relapse following remission after treatment with or becoming refractory to ASCT is failure to achieve a partial response (PR) or better after ASCT. In some embodiments, relapse following remission after treatment with or becoming refractory to ASCT includes disease progression after ASCT.

In some embodiments, the subject has relapsed following remission after treatment with, or become refractory to, at least two lines of prior systemic therapy and ASCT for the disease or condition. In some embodiments, at least one said line of prior systemic therapy comprises anthracycline and anti-CD20 monoclonal antibody treatment. In some embodiments, at least one said line of prior systemic therapy comprises anthracycline and rituximab treatment.

In some embodiments, prior to leukapheresis, the subject does not show central nervous system (CNS)-only involvement by the disease or condition. In some embodiments, the subject has pathologically-confirmed secondary CNS involvement by the disease or condition.

In some embodiments, the subject has not previously received CAR T cell therapy prior to administration of the CD19-directed engineered CAR T cells in accord with the provided methods. In some embodiments, the subject has not received genetically-modified T cell therapy. In some embodiments, the subject has not received CD19-targeted therapy. Exemplary CD19-targeted therapies include, but are not limited to, anti-CD19 monoclonal antibodies or anti-CD19 bispecific antibodies. In some embodiments, the subject does not have hypersensitivity to fludarabine and/or cyclophosphamide. In some embodiments, the subject does not have an active autoimmune disease requiring immunosuppressive therapy.

In some embodiments, the subject has not received therapeutic doses of corticosteroids less than 14 days prior to leukapheresis. In some embodiments, a therapeutic dose of corticosteroids is defined as greater than 20 mg/day of prednisone or equivalent. In some embodiments, the subject has not received cytotoxic chemotherapeutic agents that are not considered lymphotoxic less than 7 days prior to leukapheresis. In some embodiments, the subject has not received intrathecal therapy (IT) less than 7 days prior to leukapheresis. Exemplary cytotoxic chemotherapeutic agents include, but are not limited to, doxorubicin, vincristine, gemcitabine, oxaliplatin, carboplatin, and etoposide. In some embodiments, the subject has not received an oral chemotherapeutic agent less than 5 half-lives prior to leukapheresis. Exemplary oral chemotherapeutic agents include, but are not limited to, lenalidomide and ibrutinib. In some embodiments, the subject has not received lymphotoxic chemotherapeutic agents less than 4 weeks prior to leukapheresis. Exemplary lymphotoxic chemotherapeutic agents include, but are not limited to, cyclophosphamide, ifosfamide, and bendamustine. In some embodiments, the subject has not received any experimental therapy less than 8 weeks (for biologics) or 5 half-lives (for small molecules) prior to leukapheresis treatment. In some embodiments, the subject has not received immunosuppressive therapies less than 4 weeks prior to leukapheresis. Exemplary immunosuppressive therapies include, but are not limited to, calcineurin inhibitors, methotrexate or other chemotherapeutics, mycophenolate, rapamycin, immunosuppressive antibodies such as anti-TNF, anti-IL6, or anti-IL6R. In some embodiments, the subject has not received donor lymphocyte infusions less than 6 weeks prior to leukapheresis. In some embodiments, the subject has not received radiation therapy for multiple lesions less than 6 weeks of leukapheresis. In some embodiments, the subject has not received radiation therapy for a single lesion, if additional non-irradiated PET-positive lesions are present, less than 14 days prior to leukapheresis. In some embodiments, the subject has not received an autologous stem-cell transplant (SCT) less than 3 months prior to leukapheresis. In some embodiments, the subject has not received an allogenic SCT less than 6 months prior to leukapheresis.

In some embodiments, the eligibility of subjects for treatment involving administering engineered cells is determined prior to leukapheresis. In some embodiments, the subject prior to leukapheresis has adequate vascular access for leukapheresis. In some embodiments, the subject prior to leukapheresis has adequate organ function. In some embodiments, adequate organ function is indicated by, among other factors, an absolute neutrophil count (ANC) greater than 1.0×10⁹ cells/L without growth factor support within 7 days of determination of eligibility; a platelet count greater than 50×10⁹ cells/L without transfusion support within 7 days of determination of eligibility; a calculated creatinine clearance rate (CrCl, Cockcroft-Gault formula) greater than 45 mL/min; an aspartate aminotransferase (AST) level less than or equal to 2.5 times the upper limit of normal (ULN); an alanine aminotransferase (ALT) level less than or equal to 2.5 times the ULN; a total bilirubin level less than 1.5 times the ULN; a direct bilirubin level less than 1.5 times the ULN, in the case of Gilbert's syndrome or lymphomatous infiltration of the liver; adequate pulmonary function, for instance less than or equal to CTCAE Grade 1 dyspnea and saturated oxygen (SaO2 greater than 92%) on room air; adequate cardiac function, for instance left ventricular ejection fraction (LVEF) greater than or equal to 40% as assessed by echocardiogram (ECHO) or a multiple uptake gated acquisition (MUGA) scan performed within 30 days of determination of eligibility; adequate organ function to receive lymphodepleting (LD) chemotherapy; or a combination of any of the foregoing.

In particular embodiments, prior to administration of the dose of CD19-directed engineered CAR T cells, the subject is administered or has received a lymohodepleting chemotherapy.

Lymphodepletion may improve the engraftment and activity of CAR T cells through homeostatic cytokines, reduction of CD4+CD25+ regulatory T cells, increase of SDF-1 within bone marrow microenvironment, and stimulatory effects on antigen presenting cells (Grossman et al., Nat Rev Immunol. 2004; 4(5):387-395; Stachel et al., Pediatr Blood Cancer 2004; 43(6):644-50; Pinthus et al., J Clin Invest 2004; 114(12):1774-81; Turk et al., J Exp Med 2004; 200(6):771-82). In addition, LD chemotherapy may further reduce the subject's tumor burden and potentially lower the risk and severity of cytokine release syndrome (CRS).

Thus, in some embodiments, the methods include administering a preconditioning agent, such as a lymphodepleting or chemotherapeutic agent, such as cyclophosphamide, fludarabine, or combinations thereof, to a subject prior to the administration of engineered cells. For example, the subject may be administered a preconditioning agent at least 2 days prior, such as at least 3, 4, 5, 6, 7, 8, or 9 days prior, to the administration of engineered cells. In some embodiments, the subject is administered a preconditioning agent no more than 9 days prior, such as no more than 8, 7, 6, 5, 4, 3, or 2 days prior, to the administration of engineered cells.

In some embodiments, the subject is preconditioned with cyclophosphamide at a dose between or between about 20 mg/kg and 100 mg/kg body weight of the subject, such as between or between about 40 mg/kg and 80 mg/kg. In some aspects, the subject is preconditioned or administered with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m² body surface area of the subject, such as between or between about 200 mg/m² and 400 mg/m², or 250 mg/m² and 350 mg/m², inclusive. In some instances, the subject is administered about 100 mg/m² of cyclophosphamide. In some instances, the subject is administered about 150 mg/m² of cyclophosphamide. In some instances, the subject is administered about 200 mg/m² of cyclophosphamide. In some instances, the subject is administered about 250 mg/m² of cyclophosphamide. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² body surface area of the subject, of cyclophosphamide, daily for 3 days, prior to initiation of the cell therapy. In some embodiments, the subject is administered a total of at or about 300 mg/m², 400 mg/m², 500 mg/m², 600 mg/m², 700 mg/m², 800 mg/m², 900 mg/m², 1000 mg/m², 1200 mg/m², 1500 mg/m², 1800 mg/m², 2000 mg/m², 2500 mg/m², 2700 mg/m², 3000 mg/m², 3300 mg/m², 3600 mg/m², 4000 mg/m² or 5000 mg/m² cyclophosphamide, or a range defined by any of the foregoing, prior to initiation of the cell therapy.

In some embodiments, where the lymphodepleting agent comprises fludarabine, the subject is administered fludarabine at a dose between at or about 1 mg/m² and at or 100 mg/m², such as between at or about 10 mg/m² and at or about 75 mg/m², at or about 15 mg/m² and at or about 50 mg/m², at or about 20 mg/m² and at or about 40 mg/m², at or about or 24 mg/m² and at or about 35 mg/m², inclusive. In some instances, the subject is administered at or at or about 10 mg/m² of fludarabine. In some instances, the subject is administered at or about 15 mg/m² of fludarabine. In some instances, the subject is administered at or about 20 mg/m² of fludarabine. In some instances, the subject is administered at or about 25 mg/m² of fludarabine. In some instances, the subject is administered at or about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered at or about 30 mg/m² body surface area of the subject, of fludarabine, daily for 3 days, prior to initiation of the cell therapy. In some embodiments, the subject is administered a total of at or about 10 mg/m², 20 mg/m², 25 mg/m², 30 mg/m², 40 mg/m², 50 mg/m², 60 mg/m², 70 mg/m², 80 mg/m², 90 mg/m², 100 mg/m², 120 mg/m², 150 mg/m², 180 mg/m², 200 mg/m², 250 mg/m², 270 mg/m², 300 mg/m², 330 mg/m², 360 mg/m², 400 mg/m² or 500 mg/m² cyclophosphamide, or a range defined by any of the foregoing, prior to initiation of the cell therapy.

In some embodiments, the lymphodepleting agent comprises a single agent, such as cyclophosphamide or fludarabine. In some embodiments, the subject is administered cyclophosphamide only, without fludarabine or other lymphodepleting agents. In some embodiments, prior to the administration, the subject has received a lymphodepleting therapy comprising the administration of cyclophosphamide at or about 200-400 mg/m² body surface area of the subject, optionally at or about 300 mg/m², daily, for 2-4 days. In some embodiments, the subject is administered fludarabine only, for example, without cyclophosphamide or other lymphodepleting agents. In some embodiments, prior to the administration, the subject has received a lymphodepleting therapy comprising the administration of fludarabine at or about 20-40 mg/m² body surface area of the subject, optionally at or about 30 mg/m², daily, for 2-4 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered at or about 60 mg/kg (˜2 g/m²) of cyclophosphamide and 3 to 5 doses of 25 mg/m² fludarabine prior to the first or subsequent dose. In some the subject is administered fludarabine (30 mg/m²/day for 3 days) and cyclophosphamide (300 mg/m²/day for 3 days) (flu/cy) concurrently, intravenously, prior to administration of the cells. In some embodiments, the subject is administered a reduced, delayed or eliminated dose of one or more doses of the lymphodepleting agent(s).

In some embodiments, after collecting the cells from the subject and prior to administering lymphodepleting (LD) chemotherapy, the subject can receive bridging therapy for disease control. Any of a variety of therapies can be administered as part of a bridging therapy based on the judgment of a skilled practitioner for treating the particular disease or condition, including based on factors such as the age of the patient, severity or extent of the disease, potential for side effects, timing of the administration prior to the LD chemotherapy, previous therapies and other factors. Exemplary therapies that can be given as a bridge prior to the lymphodepleting therapy include, but are not limited to, corticosteroids, vincristine, cyclophosphamide, rituximab, dexamethasone, prednisone, lenalidomide, gemcitabine, oxaliplatin, Brentuximab vedotin, ibrutininb, methotrexate, cytosine arabinoside, cytarabine, bendamustine, or any combination of any of the foregoing.

In some embodiments, the subjects are premedicated, e.g., to minimize the risk of infusion reaction. In some aspects, the premedication includes administering pain reliever and/or an antihistamine. In some embodiments, the premedication includes administering an acetaminophen and/or a diphenhydramine, or another H1-antihistamine. In some embodiments, the patient with acetaminophen (e.g., 650 mg orally) and diphenhydramine (e.g., 25-50 mg, IV or orally), or another H1-antihistamine, at or about 30 to 60 minutes prior to treatment with the cell therapy.

In some embodiments, the subject is at least 18 years of age. In embodiments of any of the provided methods, the subject is a human subject.

A. Dosing

In some embodiments, a dose of engineered cells is administered to subjects in accordance with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some of any of the provided embodiments, the dose of T cells, such as engineered T cells expressing a recombinant receptor, includes is enriched for, or comprises a cell composition or a cell population that is enriched for, CD3⁺ T cells, CD4⁺ T cells, CD8⁺ T cells or CD4⁺ T cells and CD8⁺ T cells. In some of any such embodiments, greater than at or about 70%, 75%, 80%, 85%, 90%, 95% or 98% of the cells in the dose of T cells are CD3⁺ T cells, CD4⁺ T cells, CD8⁺ T cells or CD4⁺ T cells and CD8⁺ T cells. In some of any such embodiments, greater than at or about 70%, 75%, 80%, 85%, 90%, 95% or 98% of the cells in the dose of T cells are CD3⁺ T cells. In some of any of the provided embodiments, the dose of T cells comprises both CD4⁺ cells and CD8⁺ cells. In some of any such embodiments, greater than at or about 70%, 75%, 80%, 85%, 90%, 95% or 98% of the cells in the dose of T cells are CD4⁺ T cells and CD8⁺ T cells.

In some embodiments, the dose of cells comprises between at or about 0.1×10⁵ of the CD19-directed CAR engineered cells per kilogram body weight of the subject (cells/kg) and at or about 2×10⁶ cells/kg, such as between at or about 0.1×10⁵ cells/kg and at or about 0.5×10⁵ cells/kg, between at or about 0.5×10⁵ cells/kg and at or about 1×10⁵ cells/kg, between at or about 1×10⁵ cells/kg and at or about 1.5×10⁵ cells/kg, between at or about 1.5×10⁵ cells/kg and at or about 2×10⁵ cells/kg, between at or about 2×10⁵ cells/kg and at or about 2.5×10⁵ cells/kg, between at or about 2.5×10⁵ cells/kg and at or about 3×10⁵ cells/kg, between at or about 3×10⁵ cells/kg and at or about 3.5×10⁵ cells/kg, between at or about 3.5×10⁵ cells/kg and at or about 4×10⁵ cells/kg, between at or about 4×10⁵ cells/kg and at or about 4.5×10⁵ cells/kg, between at or about 4.5×10⁵ cells/kg and at or about 5×10⁵ cells/kg, between at or about 5×10⁵ cells/kg and at or about 5.5×10⁵ cells/kg, between at or about 5.5×10⁵ cells/kg and at or about 6×10⁵ cells/kg, between at or about 6×10⁵ cells/kg and at or about 6.5×10⁵ cells/kg, between at or about 6.5×10⁵ cells/kg and at or about 7×10⁵ cells/kg, between at or about 7×10⁵ cells/kg and at or about 7.5×10⁵ cells/kg, between at or about 7.5×10⁵ cells/kg and at or about 8×10⁵ cells/kg, or between at or about 8×10⁵ of the cells/kg and at or about 10×10⁵ of the cells/kg. In some embodiments, the dose of cells comprises no more than 2×10⁵ of the CD19-directed CAR engineered cells per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 0.1×10⁵ of the CD19-directed CAR engineered cells per kilogram body weight of the subject (cells/kg), such as at least or at least about or at or about 0.2×10⁵ cells/kg, at least or at least about or at or about 0.3×10⁵ cells/kg, at least or at least about or at or about 0.4×10⁵ cells/kg, at least or at least about or at or about 0.5×10⁵ cells/kg, at least or at least about or at or about 0.6×10⁵ cells/kg, at least or at least about or at or about 0.7×10⁵ cells/kg, at least or at least about or at or about 0.8×10⁵ cells/kg, at least or at least about or at or about 0.9×10⁵ cells/kg, at least or at least about or at or about 0.1×10⁶ cells/kg, or at least or at least about or at or about 0.2×10⁶ cells/kg. In some embodiments, the number of cells is the number of such cells that are viable cells, e.g., viable T cells such as viable CD3⁺ cells expressing the CD19-directed CAR.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or total cells in the composition administered. In some embodiments, the number of cells is the number of such cells that are viable cells.

In some embodiments, the dose of cells is a flat dose of cells or fixed dose of cells such that the dose of cells is not tied to or based on the body surface area or weight of a subject.

In some embodiments, the dose of genetically engineered cells comprises from at or about 1×10⁵ to at or about 1×10⁸ total T cells expressing the CD19-directed CAR, from at or about 1×10⁵ to at or about 0.8×10⁸ total T cells expressing the CD19-directed CAR, from at or about 1×10⁵ to at or about 0.6×10⁸ total T cells expressing the CD19-directed CAR, from at or about 1×10⁵ to at or about 0.4×10⁸ total T cells expressing the CD19-directed CAR, from at or about 1×10⁵ to at or about 0.2×10⁸ total T cells expressing the CD19-directed CAR, from at or about 1×10⁵ to at or about 1.0×10⁷ total T cells expressing the CD19-directed CAR, from at or about 1×10⁵ to at or about 0.8×10⁷ total T cells expressing the CD19-directed CAR, from at or about 1×10⁵ to at or about 0.6×10⁷ total T cells expressing the CD19-directed CAR, from at or about 1×10⁵ to at or about 0.4×10⁷ total T cells expressing the CD19-directed CAR, from at or about 1×10⁵ to at or about 0.2×10⁷ total T cells expressing the CD19-directed CAR, from at or about 1×10⁵ to at or about 1.0×10⁶ total T cells expressing the CD19-directed CAR, from at or about 1×10⁶ to at or about 1.0×10⁸ total T cells expressing the CD19-directed CAR, from at or about 1×10⁶ to at or about 0.8×10⁸ total T cells expressing the CD19-directed CAR, from at or about 1×10⁶ to at or about 0.6×10⁸ total T cells expressing the CD19-directed CAR, from at or about 1×10⁶ to at or about 0.4×10⁸ total T cells expressing the CD19-directed CAR, from at or about 1×10⁶ to at or about 0.2×10⁸ total T cells expressing the CD19-directed CAR, from at or about 1×10⁶ to at or about 1.0×10⁷ total T cells expressing the CD19-directed CAR, from at or about 1×10⁶ to at or about 0.8×10⁷ total T cells expressing the CD19-directed CAR, from at or about 1×10⁶ to at or about 0.6×10⁷ total T cells expressing the CD19-directed CAR, from at or about 1×10⁶ to at or about 0.4×10⁷ total T cells expressing the CD19-directed CAR, from at or about 1×10⁶ to at or about 0.2×10⁷ total T cells expressing the CD19-directed CAR, from at or about 5×10⁶ to at or about 1.0×10⁸ total T cells expressing the CD19-directed CAR, from at or about 5×10⁶ to at or about 0.8×10⁸ total T cells expressing the CD19-directed CAR, from at or about 5×10⁶ to at or about 0.6×10⁸ total T cells expressing the CD19-directed CAR, from at or about 5×10⁶ to at or about 0.4×10⁸ total T cells expressing the CD19-directed CAR, from at or about 5×10⁶ to at or about 0.2×10⁸ total T cells expressing the CD19-directed CAR, from at or about 5×10⁶ to at or about 1.0×10⁷ total T cells expressing the CD19-directed CAR, from at or about 5×10⁶ to at or about 0.8×10⁷ total T cells expressing the CD19-directed CAR, from at or about 5×10⁶ to at or about 0.6×10⁷ total T cells expressing the CD19-directed CAR, from at or about 10×10⁶ to at or about 1.0×10⁸ total T cells expressing the CD19-directed CAR, from at or about 10×10⁶ to at or about 0.9×10⁸ total T cells expressing the CD19-directed CAR, from at or about 10×10⁶ to at or about 0.8×10⁸ total T cells expressing the CD19-directed CAR, from at or about 10×10⁶ to at or about 0.7×10⁸ total T cells expressing the CD19-directed CAR, from at or about 10×10⁶ to at or about 0.6×10⁸ total T cells expressing the CD19-directed CAR, from at or about 10×10⁶ to at or about 0.5×10⁸ total T cells expressing the CD19-directed CAR, from at or about 10×10⁶ to at or about 0.4×10⁸ total T cells expressing the CD19-directed CAR, from at or about 10×10⁶ to at or about 0.3×10⁸ total T cells expressing the CD19-directed CAR, from at or about 10×10⁶ to at or about 0.2×10⁸ total T cells expressing the CD19-directed CAR, from at or about 10×10⁶ to at or about 15×10⁶ total T cells expressing the CD19-directed CAR, from at or about 15×10⁶ to at or about 1.0×10⁸ total T cells expressing the CD19-directed CAR, from at or about 15×10⁶ to at or about 0.9×10⁸ total T cells expressing the CD19-directed CAR, from at or about 15×10⁶ to at or about 0.8×10⁸ total T cells expressing the CD19-directed CAR, from at or about 15×10⁶ to at or about 0.7×10⁸ total T cells expressing the CD19-directed CAR, from at or about 15×10⁶ to at or about 0.6×10⁸ total T cells expressing the CD19-directed CAR, from at or about 15×10⁶ to at or about 0.5×10⁸ total T cells expressing the CD19-directed CAR, from at or about 15×10⁶ to at or about 0.4×10⁸ total T cells expressing the CD19-directed CAR, from at or about 15×10⁶ to at or about 0.3×10⁸ total T cells expressing the CD19-directed CAR, from at or about 15×10⁶ to at or about 0.2×10⁸ total T cells expressing the CD19-directed CAR, from at or about 20×10⁶ to at or about 1.0×10⁸ total T cells expressing the CD19-directed CAR, from at or about 20×10⁶ to at or about 0.9×10⁸ total T cells expressing the CD19-directed CAR, from at or about 20×10⁶ to at or about 0.8×10⁸ total T cells expressing the CD19-directed CAR, from at or about 20×10⁶ to at or about 0.7×10⁸ total T cells expressing the CD19-directed CAR, from at or about 20×10⁶ to at or about 0.6×10⁸ total T cells expressing the CD19-directed CAR, from at or about 20×10⁶ to at or about 0.5×10⁸ total T cells expressing the CD19-directed CAR, from at or about 20×10⁶ to at or about 0.4×10⁸ total T cells expressing the CD19-directed CAR, from at or about 20×10⁶ to at or about 0.3×10⁸ total T cells expressing the CD19-directed CAR, from at or about 20×10⁶ to at or about 25×10⁶ total T cells expressing the CD19-directed CAR, from at or about 25×10⁶ to at or about 1.0×10⁸ total T cells expressing the CD19-directed CAR, from at or about 25×10⁶ to at or about 0.9×10⁸ total T cells expressing the CD19-directed CAR, from at or about 25×10⁶ to at or about 0.8×10⁸ total T cells expressing the CD19-directed CAR, from at or about 25×10⁶ to at or about 0.7×10⁸ total T cells expressing the CD19-directed CAR, from at or about 25×10⁶ to at or about 0.6×10⁸ total T cells expressing the CD19-directed CAR, from at or about 25×10⁶ to at or about 0.5×10⁸ total T cells expressing the CD19-directed CAR, from at or about 25×10⁶ to at or about 0.4×10⁸ total T cells expressing the CD19-directed CAR, from at or about 25×10⁶ to at or about 0.3×10⁸ total T cells expressing the CD19-directed CAR, from at or about 30×10⁶ to at or about 1.0×10⁸ total T cells expressing the CD19-directed CAR, from at or about 30×10⁶ to at or about 0.9×10⁸ total T cells expressing the CD19-directed CAR, from at or about 30×10⁶ to at or about 0.8×10⁸ total T cells expressing the CD19-directed CAR, from at or about 30×10⁶ to at or about 0.7×10⁸ total T cells expressing the CD19-directed CAR, from at or about 30×10⁶ to at or about 0.6×10⁸ total T cells expressing the CD19-directed CAR, from at or about 30×10⁶ to at or about 0.5×10⁸ total T cells expressing the CD19-directed CAR, from at or about 30×10⁶ to at or about 0.4×10⁸ total T cells expressing the CD19-directed CAR, from at or about 30×10⁶ to at or about 35×10⁶ total T cells expressing the CD19-directed CAR, from at or about 35×10⁶ to at or about 1.0×10⁸ total T cells expressing the CD19-directed CAR, from at or about 35×10⁶ to at or about 0.9×10⁸ total T cells expressing the CD19-directed CAR, from at or about 35×10⁶ to at or about 0.8×10⁸ total T cells expressing the CD19-directed CAR, from at or about 35×10⁶ to at or about 0.7×10⁸ total T cells expressing the CD19-directed CAR, from at or about 35×10⁶ to at or about 0.6×10⁸ total T cells expressing the CD19-directed CAR, from at or about 35×10⁶ to at or about 0.5×10⁸ total T cells expressing the CD19-directed CAR, from at or about 35×10⁶ to at or about 0.4×10⁸ total T cells expressing the CD19-directed CAR, from at or about 40×10⁶ to at or about 1.0×10⁸ total T cells expressing the CD19-directed CAR, from at or about 40×10⁶ to at or about 0.9×10⁸ total T cells expressing the CD19-directed CAR, from at or about 40×10⁶ to at or about 0.8×10⁸ total T cells expressing the CD19-directed CAR, from at or about 40×10⁶ to at or about 0.7×10⁸ total T cells expressing the CD19-directed CAR, from at or about 40×10⁶ to at or about 0.6×10⁸ total T cells expressing the CD19-directed CAR, from at or about 40×10⁶ to at or about 0.5×10⁸ total T cells expressing the CD19-directed CAR, or from at or about 40×10⁶ to at or about 45×10⁶ total T cells expressing the CD19-directed CAR. In some embodiments, the number of cells is the number of such cells that are viable cells, such as viable T cells.

In some embodiments, the dose of genetically engineered cells comprises at least or at least about 1×10⁵ T cells expressing the CD19-directed CAR, at least or at least about 2.5×10⁵ T cells expressing the CD19-directed CAR, at least or at least about 5×10⁵ T cells expressing the CD19-directed CAR, at least or at least about 1×10⁶ T cells expressing the CD19-directed CAR, at least or at least about 2.5×10⁶ T cells expressing the CD19-directed CAR, at least or at least about 5×10⁶ T cells expressing the CD19-directed CAR, at least or at least about 1×10⁷ T cells expressing the CD19-directed CAR, at least or at least about 2.5×10⁷ T cells expressing the CD19-directed CAR, or at least or at least about 5×10⁷ T cells expressing the CD19-directed CAR. In some embodiments, the number of cells is the number of such cells that are viable cells, such as viable T cells.

In some embodiments, the dose of genetically engineered cells comprises less than or less than about 1×10⁵ T cells expressing the CD19-directed CAR, less than or less than about 2.5×10⁵ T cells expressing the CD19-directed CAR, less than or less than about 5×10⁵ T cells expressing the CD19-directed CAR, less than or less than about 1×10⁶ T cells expressing the CD19-directed CAR, less than or less than about 2.5×10⁶ T cells expressing the CD19-directed CAR, less than or less than about 5×10⁶ T cells expressing the CD19-directed CAR, less than or less than about 1×10⁷ T cells expressing the CD19-directed CAR, less than or less than about 1.5×10⁷ T cells expressing the CD19-directed CAR, less than or less than about 2×10⁷ T cells expressing the CD19-directed CAR, less than or less than about 2.5×10⁷ T cells expressing the CD19-directed CAR, less than or less than about 3×10⁷ T cells expressing the CD19-directed CAR, less than or less than about 3.5×10⁷ T cells expressing the CD19-directed CAR, less than or less than about 4×10⁷ T cells expressing the CD19-directed CAR, less than or less than about 4.5×10⁷ T cells expressing the CD19-directed CAR, or less than or less than about 5×10⁷ T cells expressing the CD19-directed CAR. In some embodiments, the number of cells is the number of such cells that are viable cells, such as viable T cells.

In some embodiments, the cell therapy comprises administration of a dose comprising a number of cell from or from about 1×10⁵ to or to about 5×10⁸ total recombinant receptor-expressing cells or total T cells, from or from about 5×10⁵ to or to about 1×10⁷ total recombinant receptor-expressing cells or total T cells, or from or from about 1×10⁶ to or to about 1×10⁷ total recombinant receptor-expressing cells or total T cells, each inclusive. In some embodiments, the cell therapy comprises administration of a dose comprising a number of cell from or from about 1×10⁵ to or to about 1×10⁸ total recombinant receptor-expressing cells, or total T cells, from or from about 5×10⁵ to or to about 1×10⁸ total recombinant receptor-expressing cells, or total T cells, from or from about 1×10⁶ to or to about 50×10⁶ total recombinant receptor-expressing cells, or total T cells, from or from about 5×10⁶ to or to about 45×10⁶ total recombinant receptor-expressing cells or total T cells, or from or from about 10×10⁶ to or to about 25×10⁶ total recombinant receptor-expressing cells or total T cells, each inclusive. In some embodiments, the cell therapy comprises administration of a dose of cells comprising a number of cells at least or at least about 1×10⁵ total recombinant receptor-expressing cells or total T cells, such at least or at least 1×10⁶, at least or at least about 1×10⁷, at least or at least about 1×10⁸ of such cells. In some embodiments, the number of cells is the number of such cells that are viable cells, such as viable T cells.

In some embodiments, the number is with reference to the total number of CD3⁺, CD8⁺, or CD4⁺ and CD8⁺, in some cases also recombinant receptor-expressing (e.g. CAR⁺) cells. In some embodiments, the number of cells is the number of such cells that are viable cells.

In some embodiments, the cell therapy comprises administration of a dose comprising a number of cell from or from about 1×10⁵ to or to about 1×10⁸ CD3⁺, CD8⁺ or CD4⁺ and CD8⁺ total T cells or CD3⁺, CD8⁺ or CD4⁺ and CD8⁺ recombinant receptor (e.g. CAR)-expressing cells, from or from about 5×10⁵ to or to about 5×10⁷ CD3⁺, CD8⁺ or CD4⁺ and CD8⁺ total T cells or CD3⁺, CD8⁺ or CD4⁺ and CD8⁺ recombinant receptor (e.g. CAR)-expressing cells, or from or from about 1×10⁶ to or to about 2.5×10⁷ CD3⁺, CD8⁺ or CD4⁺ and CD8⁺ total T cells or CD3⁺, CD8⁺ or CD4⁺ and CD8⁺ recombinant receptor (e.g. CAR)-expressing cells, each inclusive. In some embodiments, the cell therapy comprises administration of a dose comprising a number of cell from or from about 1×10⁵ to or to about 1×10⁸ total CD3⁺/CAR⁺, CD8⁺/CAR⁺ or CD4⁺/CD8⁺/CAR⁺ cells, from or from about 5×10⁵ to or to about 5×10⁷ total CD3⁺/CAR⁺, CD8⁺/CAR⁺ or CD4⁺/CD8⁺/CAR⁺ cells, or from or from about 1×10⁶ to or to about 2.5×10⁷ total CD3⁺/CAR⁺, CD8⁺/CAR⁺ or CD4⁺/CD8⁺/CAR⁺ cells, each inclusive. In some embodiments, the number of cells is the number of such cells that are viable cells.

In some embodiments, the dose of genetically engineered cells comprises at least or at least about 0.1×10⁷ CD3⁺/CAR⁺, CD8⁺/CAR⁺, or CD4⁺/CD8⁺/CAR⁺ T cells, at least or at least about 0.5×10⁷ CD3⁺/CAR⁺, CD8⁺/CAR⁺, or CD4⁺/CD8⁺/CAR⁺ T cells, at least or at least about 1.0×10⁷ CD3⁺/CAR⁺, CD8⁺/CAR⁺, or CD4⁺/CD8⁺/CAR⁺ T cells, at least or at least about 2.5×10⁷ CD3⁺/CAR⁺, CD8⁺/CAR⁺, or CD4⁺/CD8⁺/CAR⁺ T cells, or at least or at least about 5×10⁷ CD3⁺/CAR⁺, CD8⁺/CAR⁺, or CD4⁺/CD8⁺/CAR⁺ T cells. In some embodiments, the dose of genetically engineered cells comprises less than or less than about 0.1×10⁷ CD3⁺/CAR⁺, CD8⁺/CAR⁺, or CD4⁺/CD8⁺/CAR⁺ T cells, less than or less than about 0.5×10⁷ CD3⁺/CAR⁺, CD8⁺/CAR⁺, or CD4⁺/CD8⁺/CAR⁺ T cells, less than or less than about 1.0×10⁷ CD3⁺/CAR⁺, CD8⁺/CAR⁺, or CD4⁺/CD8⁺/CAR⁺ T cells, less than or less than about 2.5×10⁷ CD3⁺/CAR⁺, CD8⁺/CAR⁺, or CD4⁺/CD8⁺/CAR⁺ T cells, less than or less than about 5×10⁷ CD3⁺/CAR⁺, CD8⁺/CAR⁺, or CD4⁺/CD8⁺/CAR⁺ T cells, less than or less than about 7.5×10⁷ CD3⁺/CAR⁺, CD8⁺/CAR⁺, or CD4⁺/CD8⁺/CAR⁺ T cells, or less than or less than about less than or less than about 10×10⁷ CD3⁺/CAR⁺, CD8⁺/CAR⁺, or CD4⁺/CD8⁺/CAR⁺ T cells. In some embodiments, the dose of genetically engineered cells comprises at or about 0.5×10⁷ CD3⁺/CAR⁺, CD8⁺/CAR⁺, or CD4⁺/CD8⁺/CAR⁺ T cells, at or about 1.0×10⁷ CD3⁺/CAR⁺, CD8⁺/CAR⁺, or CD4⁺/CD8⁺/CAR⁺ T cells, at or about 1.5×10⁷ CD3⁺/CAR⁺, CD8⁺/CAR⁺, or CD4⁺/CD8⁺/CAR⁺ T cells, at or about 2.0×10⁷ CD3⁺/CAR⁺, CD8⁺/CAR⁺, or CD4⁺/CD8⁺/CAR⁺ T cells, at or about 2.5×10⁷ CD3⁺/CAR⁺, CD8⁺/CAR⁺, or CD4⁺/CD8⁺/CAR⁺ T cells, at or about 3.0×10⁷ CD3⁺/CAR⁺, CD8⁺/CAR⁺, or CD4⁺/CD8⁺/CAR⁺ T cells, at or about 3.5×10⁷ CD3⁺/CAR⁺, CD8⁺/CAR⁺, or CD4⁺/CD8⁺/CAR⁺ T cells, at or about 4.0×10⁷ CD3⁺/CAR⁺, CD8⁺/CAR⁺, or CD4⁺/CD8⁺/CAR⁺ T cells, at or about 4.5×10⁷ CD3⁺/CAR⁺, CD8⁺/CAR⁺, or CD4⁺/CD8⁺/CAR⁺ T cells, at or about 5×10⁷ CD3⁺/CAR⁺, CD8⁺/CAR⁺, or CD4⁺/CD8⁺/CAR⁺ T cells. In some embodiments, the number of cells is the number of such cells that are viable cells.

In some embodiments, the dose of T cells comprises: at or about 0.5×10⁷ recombinant receptor (e.g. CAR)-expressing T cells, at or about 1.0×10⁷ recombinant receptor (e.g. CAR)-expressing T cells, at or about 1.5×10⁷ recombinant receptor (e.g. CAR)-expressing T cells, at or about 2.0×10⁷ recombinant receptor (e.g. CAR)-expressing T cells, at or about 2.5×10⁷ recombinant receptor (e.g. CAR)-expressing CD8⁺ T cells, at or about 3.0×10⁷ recombinant receptor (e.g. CAR)-expressing T cells, at or about 3.5×10⁷ recombinant receptor (e.g. CAR)-expressing T cells, at or about 4.0×10⁷ recombinant receptor (e.g. CAR)-expressing T cells, or at or about 4.5×10⁷ recombinant receptor (e.g. CAR)-expressing T cells. In some embodiments, the dose of T cells comprises: at or about 1×10⁸ recombinant receptor (e.g. CAR)-expressing T cells or at or about 5×10⁷ recombinant receptor (e.g. CAR)-expressing CD8⁺ T cells. In some embodiments, the dose of T cells comprises: at or about 1.5×10⁸ recombinant receptor (e.g. CAR)-expressing T cells or at or about 0.75×10⁸ recombinant receptor (e.g. CAR)-expressing CD8⁺ T cells. In some embodiments, the number of cells is the number of such cells that are viable cells.

In some embodiments, the T cells of the dose include CD4⁺ T cells, CD8⁺ T cells or CD4⁺ and CD8⁺ T cells.

In some embodiments, for example, where the subject is human, the CD8⁺ T cells of the dose, including in a dose including CD4⁺ and CD8⁺ T cells, includes between at or about 1×10⁶ and at or about 1×10⁸ total recombinant receptor (e.g., CAR)-expressing CD8⁺ cells, e.g., in the range of from at or about 1×10⁶ to at or about 1×10⁸ such cells, such as 1×10⁶, 2.5×10⁶, 5×10⁶, 7.5×10⁶, 1×10⁷, 2.5×10⁷, 5×10⁷, or 7.5×10⁷ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁶ to or to about 5×10⁷ total recombinant receptor-expressing CD8⁺ T cells, from or from about 5×10⁶ to or to about 2.5×10⁷ total recombinant receptor-expressing CD8⁺ T cells, from or from about 10×10⁶ to or to about 2.5×10⁷ total recombinant receptor-expressing CD8⁺ T cells, each inclusive. In some embodiments, the number of cells is the number of such cells that are viable cells.

In some embodiments, for example, where the subject is human, the CD4⁺ T cells of the dose, including in a dose including CD4⁺ and CD8⁺ T cells, includes between at or about 1×10⁶ and at or about 1×10⁸ total recombinant receptor (e.g., CAR)-expressing CD4⁺ cells, e.g., in the range of from at or about 1×10⁶ to at or about 1×10⁸ such cells, such as 1×10⁶, 2.5×10⁶, 5×10⁶, 7.5×10⁶, 1×10⁷, 2.5×10⁷, 5×10⁷, or 7.5×10⁷ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁶ to or to about 5×10⁷ total recombinant receptor-expressing CD4⁺ T cells, from or from about 5×10⁶ to or to about 2.5×10⁷ total recombinant receptor-expressing CD4⁺ T cells, from or from about 10×10⁶ to or to about 2.5×10⁷ total recombinant receptor-expressing CD4⁺ T cells, each inclusive. In some embodiments, the number of cells is the number of such cells that are viable cells.

In some embodiments, for example, where the subject is a human, the dose includes fewer than about 5×10⁸ total recombinant receptor (e.g., CAR)-expressing cells or T cells, e.g., in the range of at or about 1×10⁶ to at or about 1×10⁸ such cells, such as at or about 2×10⁶, 5×10⁶, 1×10⁷, 5×10⁷, or 1×10⁸ total such cells, or the range between any two of the foregoing values. In some embodiments, the number of cells is the number of such cells that are viable cells.

In some embodiments, the patient is administered multiple doses, and each of the doses or the total dose can be within any of the foregoing values. In some embodiments, the dose of cells comprises the administration of from or from about 1×10⁵ to or to about 1×10⁸ total recombinant receptor (e.g. CAR)-expressing T cells or total T cells, from or from about 1×10⁵ to or to about 0.5×10⁸ total recombinant receptor (e.g. CAR)-expressing T cells or total T cells, from or from about 1×10⁵ to or to about 0.5×10⁸ total recombinant receptor (e.g. CAR)-expressing T cells or total T cells, from or from about 5×10⁵ to or to about 5×10⁷ total recombinant receptor (e.g. CAR)-expressing T cells or total T cells, or from or from about 1×10⁶ to or to about 1×10⁷ total recombinant receptor (e.g. CAR)-expressing T cells or total T cells, each inclusive.

In some embodiments, the T cells of the dose include CD4⁺ T cells, CD8⁺ T cells or CD4⁺ and CD8⁺ T cells.

In some embodiments, the dose of cells, e.g., recombinant receptor-expressing T cells, is administered to the subject as a single dose or is administered only one time within a period of two weeks, one month, 3 months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

In particular embodiments, the numbers and/or concentrations of cells refer to the number of recombinant receptor (e.g., CAR)-expressing cells. In other embodiments, the numbers and/or concentrations of cells refer to the number or concentration of T cells administered.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells is generally large enough to be effective in reducing disease burden.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as at or about 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

B. Response, Efficacy, and Survival

In some embodiments, the administration in accord with the provided methods effectively treats the subject despite the subject having become resistant to another therapy. In some embodiments, at least 30%, at least 35%, at least 40% at least 50%, at least 60%, at least 70%, or at least 80%, of subjects treated according to the method achieve complete remission (CR). In some embodiments, at least about 40%, at least about 50%, at least about 60%, at least about 70%, at least 80%, or at least 90% of the subjects treated according to the method achieve an objective response (OR). In some embodiments, at least or at least about 50% of subjects, at least or at least about 60% of the subjects, at least or at least about 70% of the subjects, at least or at least about 80% of the subjects or at least or at least about 90% of the subjects treated according to the method achieve CR and/or achieve an objective response (OR). In some embodiments, criteria assessed for effective treatment includes overall response rate (ORR; also known in some cases as objective response rate), complete response (CR; also known in some cases as complete remission), duration of response (DOR), progression-free survival (PFS), and/or overall survival (OS).

In some embodiments, at least 40% or at least 50% of subjects treated according to the methods provided herein achieve complete remission (CR; also known in some cases as complete response), exhibit progression-free survival (PFS) and/or overall survival (OS) of greater than at or about 3 months, 6 months or 12 months or greater than 13 months or approximately 14 months; on average, subjects treated according to the method exhibit a median PFS or OS of greater than at or about 6 months, 12 months, or 18 months; and/or the subject exhibits PFS or OS following therapy for at least at or about 6, 12, 18 or more months or longer.

In some embodiments, the subjects treated according to the provided methods exhibits a CRR of at least 50%, at least 60%, at least 70%, at least 80%, or at least 90%. In some embodiments, the complete response rate (CRR) is calculated as the percentage of subjects with the best overall response (BOR) up to 12 months, up to 18 months, up to 24 months, up to 36 months or longer.

In some aspects, response rates in subjects, such as subjects with NHL, are based on the Lugano criteria. (Cheson et al., (2014) JCO 32(27):3059-3067; Johnson et al., (2015) Radiology 2:323-338; Cheson, B. D. (2015) Chin Clin Oncol 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake≤mediastinum; 3, uptake>mediastinum but≤liver; 4, uptake moderately>liver; 5, uptake markedly higher than liver and/or new lesions; X, new areas of uptake unlikely to be related to lymphoma.

In some aspects, a complete response as described using the Lugano criteria involves a complete metabolic response and a complete radiologic response at various measureable sites. In some aspects, these sites include lymph nodes and extralymphatic sites, wherein a CR is described as a score of 1, 2, or 3 with or without a residual mass on the 5-point scale, when PET-CT is used. In some aspects, in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. In some aspects, response is assessed in the lymph nodes using CT, wherein a CR is described as no extralymphatic sites of disease and target nodes/nodal masses must regress to ≤1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology, which if indeterminate should be IHC negative. Further sites may include assessment of organ enlargement, which should regress to normal. In some aspects, non-measured lesions and new lesions are assessed, which in the case of CR should be absent (Cheson et al., (2014) JCO 32(27):3059-3067; Johnson et al., (2015) Radiology 2:323-338; Cheson, B. D. (2015) Chin Clin Oncol 4(1):5).

In some aspects, a partial response (PR; also known in some cases as partial remission) as described using the Lugano criteria involves a partial metabolic and/or radiological response at various measureable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease. In some aspects, response is assessed in the lymph nodes using CT, wherein a PR is described as ≥50% decrease in SPD of up to 6 target measureable nodes and extranodal sites. If a lesion is too small to measure on CT, 5 mm×5 mm is assigned as the default value; if the lesion is no longer visible, the value is 0 mm×0 mm; for a node>5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, non-measured lesions and new lesions are assessed, which in the case of PR should be absent/normal, regressed, but no increase. No response/stable disease (SD) or progressive disease (PD) can also be measured using PET-CT and/or CT based assessments. (Cheson et al., (2014) JCO 32(27):3059-3067; Johnson et al., (2015) Radiology 2:323-338; Cheson, B. D. (2015) Chin Clin Oncol 4(1):5).

In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR; also known in some cases as overall response rate) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months or greater than 6 months.

In some aspects, the RECIST criteria is used to determine objective tumor response; in some aspects, in solid tumors. (Eisenhauer et al., European Journal of Cancer 45 (2009) 228-247.) In some aspects, the RECIST criteria is used to determine objective tumor response for target lesions. In some respects, a complete response as determined using RECIST criteria is described as the disappearance of all target lesions and any pathological lymph nodes (whether target or non-target) must have reduction in short axis to <10 mm. In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

In some embodiments survival rates in subjects with follicular lymphoma (FL) are based on scoring systems developed by the Italian Lymphoma Intergroup (ILI) and/or the International Follicular Lymphoma Prognostic Factor Project (IFLPFP), generally as described above (Luminari et al., (2012) Rev. Brad. Hematol. Hemoter., 34:54-59). In some embodiments, the extent of disease such as a FL may be assessed by the Ann Arbor staging system, tumor burden, bulky disease, number of nodal or extranodal sites of disease, and/or bone marrow involvement, generally as described above.

In some aspects, the administration in accord with the provided methods, and/or with the provided articles of manufacture or compositions, generally reduces or prevents the expansion or burden of the disease or condition in the subject. For example, where the disease or condition is a tumor, the methods generally reduce tumor size, bulk, metastasis, percentage of blasts in the bone marrow or molecularly detectable cancer and/or improve prognosis or survival or other symptom associated with tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood or bone marrow in the context of certain hematological malignancies. Disease burden can include, in some embodiments, the mass of a tumor, the number or extent of metastases and/or the percentage of blast cells present in the bone marrow.

In some aspects, minimal residual disease (MRD) is detected by flow cytometry. Flow cytometry can be used to monitor bone marrow and peripheral blood samples for cancer cells. In particular aspects, flow cytometry is used to detect or monitor the presence of cancer cells in bone marrow. In some aspects, multiparameter immunological detection by flow cytometry is used to detect cancer cells (see for example, Coustan-Smith et al., (1998) Lancet 351:550-554). In some aspects, multiparameter immunological detection by mass cytometry is used to detect cancer cells. In some examples, 1, 2, 3, 4, 5, 6, 7, 8, 9, 10, 11, 12, 13, 14, 15, 20, 25, 30, 35, 40, 45 or 50 parameters can be used to detect cancer cells. The antigens used for detection are selected based on the cancer being detected (Foon and Todd (1986) Blood 68:1-31).

In some examples, bone marrow is harvested by bone marrow aspirates or bone marrow biopsies, and lymphocytes are isolated for analysis. Monoclonal and/or polyclonal antibodies conjugated to a fluorochrome (e.g., fluorescein isothiocyanate (FITC), phycoerythrin, peridinin chlorophyll protein, or biotin) can be used to detect epitopes, such as terminal deoxynucleotidyl transferase (TdT), CD3, CD10, CD11c, CD13, CD14, CD33, CD19, CD20, CD21, CD22, CD23, CD34, CD45, CD56, CD79b, IgM, and/or KORSA3544, on isolated lymphocytes. Labeled cells can then be detected using flow cytometry, such as multiparameter flow cytometry, or mass cytometry, to detect multiple epitopes.

Lymphoid cells can be identified and gated based on a light-scatter dot plot and then secondarily gated to identify cell populations expressing the immunophenotypic features of interest. Exemplary epitopes are set forth in Table 2 below. Other immunologic classification of leukemias and lymphomas are provided by Foon and Todd (Blood (1986) 68(1): 1-31). In some aspects, flow cytometric assessment of MRD can be achieved by quantifying live lymphocytes bearing one or more CLL immunophenotypes (e.g., low forward/side scatter; CD3^{neg}; CD5⁺; CD14^{neg}; CD19⁺; CD23⁺; CD45⁺; CD56^{neg}).

TABLE 2
Exemplary Immnunophenotype and Cytogentics Characteristics

| Disease | Immunophenotype | Cytogenetics |
|---|---|---|
| Chronic Lymphocytic | Pan-B+; CD5+; CD23+; | Trisomy12 |
| Leukemia (CLL) | CD79b/CD22 weak; | del(13)(q14.3) |
| | FMC7−; sIg weak | del 11q22-q23 |
| | | del 17p13 (p53) |
| | | t(11;14)(q13;q32) BCL1/IgH rearrangement |
| | | t(14;19)(q32;q13) |
| | | IgH deletion (14q32) |
| | | del(6q) |
| | | +8q24 |
| | | +3 |
| | | +18 |
| | | del 6q21 |
| Small lymphocytic | Pan-B+; CD5+; CD23+; | del(6)(q21-23) |
| lymphoma (SLL) | CD10−; sIgM+ faint | |
| Lymphoplasmacytic | Pan-B+; CD5−; CD10−; | t(9;14)(p13;q32) PAX5/IgH |
| lymphoma | cyIgM+ | |
| Follicle center cell | Pan-B+; CD10+/−; CD5−; | t(14;18)(q32;q21)/BCL2 Rearr |
| lymphoma | sIg+ | |
| Diffuse large cell | CD19+; CD22+; CD10−/+; | t(14;18) and p53 mutations |
| lymphoma | SIg+ | t(3;V)(q27;V)/BCL6 Rearr |
| | | variants c-MYC Rearr |
| Burkitt's lymphoma | Pan-B+; TdT−; CD10+; | t(8;14)(q24;q32) or variants/c-MYC Rearr |
| | CD5−; sIgM+ | |
| Burkitt-like lymphoma | Pan-B+; TdT−; CD10−/+ | t(8; 14) or variants |
| | CD5−; sIg+ | t(8;14) + t(14;18) |
| Mantle cell lymphoma | Pan-B+; CD5+; CD23−; | t(11;14)(q13;q32)/BCL1 Rearr |
| | CD10−/+; sIgM+ bright | |
| Marginal zone B-cell | pan-B+; CD5−/+; CD10−; | t(11;18)(q21;q21)/PI2/MLT fusion: Extra- |
| lymphoma (MZBCL) | CD23−; CD11c+/−; | nodal low-grade MALT lymphoma; indolent |
| | cyIg+ (40% of the | disease |
| | cells), sIgM+ bright; | t(1;14)(p21;q32): Extra-nodal MALT lymphoma |
| | sIgD− | del(7)(q22-31): Splenic MZBCL/+3q: |
| | | Nodal, extra-nodal and splenic MZBCL |

+: positive in >90% of the cases
+/−: positive in more than 50% of the cases
−/+: positive in less than 50% of cases
−: positive in <10% of the cases
Pan-B markers: e.g., CD19, CD20, CD79a
sIG: surface immunoglobulins
cyIg: cytoplasmic immunoglobulins

In some aspects, deep sequencing of the immunoglobulin heavy chain (IGH) locus of harvested B cells can be used to detect minimal residual disease (MRD). Clonal presence of a particular IgG rearrangement can provide a marker to detect the presence of B cell malignancies, such as CLL or NHL and/or residual presence of malignant cells thereof. In some aspects cells such as a population containing or suspected of containing B cells are harvested and isolated from blood. In some aspects, cells are harvested and isolated from bone marrow, e.g., from bone marrow aspirates or bone marrow biopsies and/or from other biological samples. In some aspects, polymerase chain reaction (PCR) amplification of the complementarity determining region 3 (CDR3) is achieved using primers to highly conserved sequences within the V and J regions of the gene locus, which may be used to identify clonal populations of cells for purposes of assessing minimal residual disease. Other methods for detecting clonal populations, such as single cell sequencing approaches, including those providing information regarding number of cells of a particular lineage and/or expressing a particular variable chain such as variable heavy chain or binding site thereof, such as a clonal population, may be used. In some aspects, the IGH DNA is amplified using a degenerate primers or primers recognizing regions of variable chains shared among different cell clones, such as those recognizing consensus V and degenerate consensus J region of the IGH sequence. An exemplary sequence of the V region is ACACGGCCTCGTGTATTACTGT (SEQ ID NO: 57). An exemplary degenerate consensus sequence of the J region is ACCTGAGGAGACGGTGACC (SEQ ID NO: 58).

The PCR product or sequencing result in some aspects is specific to the rearranged allele and serves as a clonal marker for MRD detection. Following PCR amplification of the CDR3 region, PCR products can be sequenced to yield patient-specific oligonucleotides constructed as probes for allele-specific PCR for sensitive detection of MRD following treatment of B-cell malignancies with CAR-T cell therapy, e.g. CD19 CAR-T cell therapy. In examples where a PCR product is not generated using the consensus primers, V region family-specific primers for the framework region 1 can be used instead.

In some aspects, persistence of PCR-detectable tumor cells such as cells of the B cell malignancy such as the NHL or CLL, such as detectable IGH sequences corresponding to the malignant or clonal IGH sequences, after treatment is associated with increased risk of relapse. In some aspects, patients who are negative for malignant IGH sequences following treatment (in some aspects, even in the context of other criteria indicating progressive disease or only a partial response, such as persistence of enlarged lymph nodes or other criteria that may in some contexts be associated with disease or lack of complete response) may be deemed to have increased likelihood of PFS or to enter into CR or durable CR or prolonged survival, compared to patients with persistent malignant IGH sequences. In some embodiments, such prognostic and staging determinations are particularly relevant for treatments in which clearance of malignant cells is observed within a short period of time following administration of the therapy, e.g., in comparison to resolution of other clinical symptoms such as lymph node size or other staging criteria. For example, in some such aspects, absence of detectable IGH or minimal residual disease in a sample such as the bone marrow may be a preferred readout for response or likelihood of response or durability thereof, as compared to other available staging or prognostic approaches. In some aspects, results from MRD, e.g., IGH deep sequencing information, may inform further intervention or lack thereof. For example, the methods and other provided embodiments in some contexts provide that a subject deemed negative for malignant IGH may in some aspects be not further treated or not be further administered a dose of the therapy provided, or that the subject be administered a lower or reduced dose. Conversely, it may be provided or specified that a subject exhibiting MRD via IGH deep sequencing be further treated, e.g., with the therapy initially administered at a similar or higher dose or with a further treatment. In some aspects, the disease or condition persists following administration of the first dose and/or administration of the first dose is not sufficient to eradicate the disease or condition in the subject.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative dosing regimen, such as one in which the subject receives one or more alternative therapeutic agents and/or one in which the subject does not receive a dose of cells and/or a lymphodepleting agent in accord with the provided methods, and/or with the provided articles of manufacture or compositions. In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified.

In some embodiments, the event-free survival rate or overall survival rate of the subject is improved by the methods, as compared with other methods, for example, methods in which the subject receives one or more alternative therapeutic agents and/or one in which the subject does not receive a dose of cells and/or a lymphodepleting agent in accord with the provided methods, and/or with the provided articles of manufacture or compositions. For example, in some embodiments, event-free survival rate or probability for subjects treated by the methods at 6 months following the dose is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods, for example, methods in which the subject receives one or more alternative therapeutic agents and/or one in which the subject does not receive a dose of cells and/or a lymphodepleting agent in accord with the provided methods, and/or with the provided articles of manufacture or compositions. For example, in some embodiments, the probability of relapse at 6 months following the first dose is less than about 80%, less than about 70%, less than about 60%, less than about 50%, less than about 40%, less than about 30%, less than about 20%, or less than about 10%.

In some cases, the pharmacokinetics of administered cells, e.g., adoptively transferred cells are determined to assess the availability, e.g., bioavailability of the administered cells. Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (a CAR) in transduced cells (see e.g. U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the number of CAR⁺ T cells in a biological sample obtained from the patient, e.g., blood, can be determined at a period of time after administration of the cell therapy, e.g., to determine the pharmacokinetics of the cells. In some embodiments, number of CAR⁺ T cells, optionally CAR⁺CD8⁺ T cells and/or CAR⁺CD4⁺ T cells, detectable in the blood of the subject, or in a majority of subjects so treated by the method, is greater than 1 cells per μL, greater than 5 cells per μL or greater than per 10 cells per μL.

C. Toxicity

In some embodiments, the provided methods are designed to or include features that result in a lower rate and/or lower degree of toxicity, toxic outcome or symptom, toxicity-promoting profile, factor, or property, such as a symptom or outcome associated with or indicative of cytokine release syndrome (CRS) or neurotoxicity (NT), for example, compared to administration of an alternative cell therapy, such as an alternative CAR⁺ T cell composition and/or an alternative dosing of cells, e.g. a dosing of cells that is not administered at a defined ratio. Cytokine release syndrome (CRS) and neurotoxicity) can be graded according to the American Society for Transplantation and Cellular Therapy (ASTCT) Consensus Grading System (see e.g., Lee et al. Biol Blood Marrow Transplant. 2019 April;25(4):625-38)).

In some aspects, although the lower differentiation state of the engineered T cells administered as part of the methods provided herein (e.g., the higher proportion of engineered T cells having a naïve-like or central memory phenotype, such as a phenotype selected from CCR7⁺CD45RA⁺, CD27⁺CCR7⁺, or CD62L⁻CCR7⁺) are expected to be more active than cells that are more differentiated, findings indicate that safety of the cell therapy can be successfully managed. In some aspects, providing a lower dose of the composition, e.g. compared to a cell composition produced by a process in which the cells are more differentiated, such as a process that includes expansion of the cells, achieves robust efficacy and high safety. In some aspects, it is found that even higher doses of cells of the provided anti-BCMA CAR compositions can be administered while maintaining a lower degree of toxicity, such as a severe cytokine release syndrome (CRS) or severe neurotoxicity. Thus, the provided methods in some embodiments include the administration of higher doses of engineered T cells (e.g., greater than 50×10⁶ CAR-expressing T cells, such as at or about 100×10⁶ CAR-expressing T cells), compared to methods that include the administration of an alternative cell therapy, such as an alternative CAR⁺ T cell composition with engineered T cells that are more differentiated than those administered herein.

In some embodiments, the provided methods do not result in a high rate or likelihood of toxicity or toxic outcomes, or reduces the rate or likelihood of toxicity or toxic outcomes, such as neurotoxicity (NT), cytokine release syndrome (CRS), such as compared to certain other cell therapies. In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxicity. In some embodiments, no more than 50% of subjects treated (e.g. at least 60%, at least 70%, at least 80%, at least 90% or more of the subjects treated) exhibit a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells. In some embodiments, parameters assessed to determine certain toxicities include adverse events (AEs), dose-limiting toxicities (DLTs), CRS and NT.

Administration of adoptive T cell therapy, such as treatment with T cells expressing chimeric antigen receptors, can induce toxic effects or outcomes such as cytokine release syndrome and neurotoxicity. In some examples, such effects or outcomes parallel high levels of circulating cytokines, which may underlie the observed toxicity.

In some aspects, the toxic outcome is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure. In some aspects, fever, especially high fever (≥38.5° C. or ≥101.3° F.), is associated with CRS or risk thereof. In some cases, features or symptoms of CRS mimic infection. In some embodiments, infection is also considered in subjects presenting with CRS symptoms, and monitoring by cultures and empiric antibiotic therapy can be administered. Other symptoms associated with CRS can include cardiac dysfunction, adult respiratory distress syndrome, renal and/or hepatic failure, coagulopathies, disseminated intravascular coagulation, and capillary leak syndrome.

CRS may be treated using anti-inflammatory therapy such as an anti-IL-6 therapy, e.g., anti-IL-6 antibody, e.g., tocilizumab, or antibiotics or other agents as described. Outcomes, signs and symptoms of CRS are known and include those described herein. In some embodiments, where a particular dosage regimen or administration effects or does not effect a given CRS-associated outcome, sign, or symptom, particular outcomes, signs, and symptoms and/or quantities or degrees thereof may be specified.

In the context of administering CAR-expressing cells, CRS typically occurs 6-20 days after infusion of cells that express a CAR. See Xu et al., Cancer Letters 343 (2014) 172-78. In some cases, CRS occurs less than 6 days or more than 20 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1β, IL-6, IL-8, and IL-10.

Exemplary outcomes associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly. Other exemplary signs or symptoms associated with CRS include hemodynamic instability, febrile neutropenia, increase in serum C-reactive protein (CRP), changes in coagulation parameters (for example, international normalized ratio (INR), prothrombin time (PTI) and/or fibrinogen), changes in cardiac and other organ function, and/or absolute neutrophil count (ANC).

In some embodiments, outcomes associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, neurotoxicity (NT) can be observed concurrently with CRS.

Exemplary CRS-related outcomes include increased or high serum levels of one or more factors, including cytokines and chemokines and other factors associated with CRS. Exemplary outcomes further include increases in synthesis or secretion of one or more of such factors. Such synthesis or secretion can be by the T cell or a cell that interacts with the T cell, such as an innate immune cell or B cell.

In some embodiments, the CRS-associated serum factors or CRS-related outcomes include inflammatory cytokines and/or chemokines, including interferon gamma (IFN-γ), TNF-α, IL-1β, IL-2, IL-6, IL-7, IL-8, IL-10, IL-12, sIL-2Ra, granulocyte macrophage colony stimulating factor (GM-CSF), macrophage inflammatory protein (MIP)-1, tumor necrosis factor alpha (TNFα), IL-6, and IL-10, IL-13, IL-8, IL-2, MIP-1, Flt-3L, fracktalkine, and/or IL-5. In some embodiments, the factor or outcome includes C reactive protein (CRP). In addition to being an early and easily measurable risk factor for CRS, CRP also is a marker for cell expansion. In some embodiments, subjects that are measured to have high levels of CRP, such as ≥15 mg/dL, have CRS. In some embodiments, subjects that are measured to have high levels of CRP do not have CRS. In some embodiments, a measure of CRS includes a measure of CRP and another factor indicative of CRS.

In some embodiments, one or more inflammatory cytokines or chemokines are monitored before, during, or after CAR treatment. In some aspects, the one or more cytokines or chemokines include IFN-γ, TNF-α, IL-2, IL-1β, IL-6, IL-7, IL-8, IL-10, IL-12, sIL-2Ra, granulocyte macrophage colony stimulating factor (GM-CSF), or macrophage inflammatory protein (MIP). In some embodiments, IFN-γ, TNF-α, and IL-6 are monitored.

CRS criteria that appear to correlate with the onset of CRS to predict which patients are more likely to be at risk for developing sCRS have been developed (see Davilla et al. Science translational medicine. 2014;6(224):224ra25). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. Other guidelines on the diagnosis and management of CRS are known (see e.g., Lee et al, Blood. 2014; 124(2):188-95; Lee et al., Biol Blood Marrow Transplant 2019; 25(4):625-38). In some embodiments, the criteria reflective of CRS grade are those detailed in Table 3 below.

TABLE 3
Exemplary Grading Criteria for CRS

| Grade | Description of Symptoms |
|---|---|
| 1 | Not life-threatening, require only symptomatic |
| Mild | treatment such as antipyretics and anti-emetics |
| | (e.g., fever, nausea, fatigue, headache, |
| | myalgias, malaise) |
| 2 | Require and respond to moderate intervention: |
| Moderate | Oxygen requirement <40%, or |
| | Hypotension responsive to fluids or low dose of a |
| | single vasopressor, or |
| | Grade 2 organ toxicity (by CTCAE v4.0) |
| 3 | Require and respond to aggressive intervention: |
| Severe | Oxygen requirement ≥40%, or |
| | Hypotension requiring high dose of a single |
| | vasopressor (e.g., norepinephrine ≥20 μg/kg/min, |
| | dopamine ≥10 μg/kg/min, phenylephrine ≥200 |
| | μg/kg/min, or epinephrine ≥10 μg/kg/min), or |
| | Hypotension requiring multiple vasopressors (e.g., |
| | vasopressin + one of the above agents, or combination |
| | vasopressors equivalent to ≥20 μg/kg/min norepinephrine), |
| | or |
| | Grade 3 organ toxicity or Grade 4 transaminitis (by |
| | CTCAE v4.0) |
| 4 | Life-threatening: |
| Life- | Requirement for ventilator support, or |
| threatening | Grade 4 organ toxicity (excluding transaminitis) |
| 5 | Death |
| Fatal | |

In some embodiments, a criteria reflective of CRS grade are those detailed in Table 4 below.

TABLE 4
Exemplary Grading Criteria for CRS

| | | | | Grade 4 (life- |
|---|---|---|---|---|
| | Grade | Grade 2 (moderate) | Grade 3 (severe) | threatening) |
| Symptoms/Signs | 1 (mild) | CRS grade is defined by the most severe symptom (excluding fever) | | |
| Temperature ≥38.5° C./ | Any | Any | Any | Any |
| 101.3° F. | | | | |
| Systolic blood | N/A | Responds to fluid or single | Needs high-dose or | Life-threatening |
| pressure ≤90 mm Hg | | low-dose vasopressor | multiple vasopressors | |
| Need for oxygen | N/A | FiO2 < 40% | FiO₂ ≥ 40% | Needs ventilator |
| to reach SaO₂ > 90% | | | | support |
| Organ toxivity | N/A | Grade 2 | Grade 3 or | Grade 4 |
| | | | transaminitis | excluding |
| | | | | transaminitis) |

In some embodiments, high-dose vasopressor therapy include those described in Table 5 below.

TABLE 5
High dose vasopressors (all doses required for ≥3 hours)

| Vasopressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + noreprinephrine equivalent |
| | (NE) of ≥10 μg/min^{a} |
| If on combination | Norepinephrine equvalent of ≥20 μg/min^{a} |
| vasopressors (not vasopressin) | |

^{a}VASST Trial Vasopressor Equivalent Equation: Norepinephrine equivalent dose = [norephinephrine (μg/min)] + [dopamine (μg/kg/min) ÷ 2] + [epinephrine (μg/min)] + [phenylephrine (μg/min) ÷ 10]

In some embodiments, the toxic outcome is a severe CRS. In some embodiments, the toxic outcome is the absence of severe CRS (e.g. moderate or mild CRS). In some embodiments, a subject is deemed to develop “severe CRS” (“sCRS”) in response to or secondary to administration of a cell therapy or dose of cells thereof, if, following administration, the subject displays: (1) fever of at least 38 degrees Celsius for at least three days; (2) cytokine elevation that includes either (a) a max fold change of at least 75 for at least two of the following group of seven cytokines compared to the level immediately following the administration: interferon gamma (IFNγ), GM-CSF, IL-6, IL-10, Flt-3L, fracktalkine, and IL-5 and/or (b) a max fold change of at least 250 for at least one of the following group of seven cytokines compared to the level immediately following the administration: interferon gamma (IFNγ), GM-CSF, IL-6, IL-10, Flt-3L, fracktalkine, and IL-5; and (c) at least one clinical sign of toxicity such as hypotension (requiring at least one intravenous vasoactive pressor) or hypoxia (PO₂<90%) or one or more neurologic disorder(s) (including mental status changes, obtundation, and/or seizures). In some embodiments, severe CRS includes CRS with a grade of 3 or greater, such as set forth in Table 3 and Table 4.

In some embodiments, the level of the toxic outcome, e.g. the CRS-related outcome, e.g. the serum level of an indicator of CRS, is measured by ELISA. In some embodiments, fever and/or levels of C-reactive protein (CRP) can be measured. In some embodiments, subjects with a fever and a CRP≥15 mg/dL may be considered high-risk for developing severe CRS. In some embodiments, the CRS-associated serum factors or CRS-related outcomes include an increase in the level and/or concentration of inflammatory cytokines and/or chemokines, including Flt-3L, fracktalkine, granulocyte macrophage colony stimulating factor (GM-CSF), interleukin-1 beta (IL-1β), IL-2, IL-5, IL-6, IL-7, IL-8, IL-10, IL-12, interferon gamma (IFN-γ), macrophage inflammatory protein (MIP)-1, MIP-1, sIL-2Ra, or tumor necrosis factor alpha (TNFα). In some embodiments, the factor or outcome includes C reactive protein (CRP). In addition to being an early and easily measurable risk factor for CRS, CRP also is a marker for cell expansion. In some embodiments, subjects that are measured to have high levels of CRP, such as ≥15 mg/dL, have CRS. In some embodiments, subjects that are measured to have high levels of CRP do not have CRS. In some embodiments, a measure of CRS includes a measure of CRP and another factor indicative of CRS.

In some embodiments, outcomes associated with severe CRS or grade 3 CRS or greater, such as grade 4 or greater, include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In some embodiments, severe CRS includes CRS that requires management or care in the intensive care unit (ICU).

In some embodiments, the CRS, such as severe CRS, encompasses a combination of (1) persistent fever (fever of at least 38 degrees Celsius for at least three days) and (2) a serum level of CRP of at least at or about 20 mg/dL. In some embodiments, the CRS encompasses hypotension requiring the use of two or more vasopressors or respiratory failure requiring mechanical ventilation. In some embodiments, the dosage of vasopressors is increased in a second or subsequent administration.

In some embodiments, severe CRS or grade 3 CRS encompasses an increase in alanine aminotransferase, an increase in aspartate aminotransferase, chills, febrile neutropenia, headache, left ventricular dysfunction, encephalopathy, hydrocephalus, and/or tremor.

The method of measuring or detecting the various outcomes may be specified.

In some aspects, the toxic outcome is or is associated with neurotoxicity. In some embodiments, symptoms associated with a clinical risk of neurotoxicity include confusion, delirium, aphasia, expressive aphasia, obtundation, myoclonus, lethargy, altered mental status, convulsions, seizure-like activity, seizures (optionally as confirmed by electroencephalogram (EEG)), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute-Common Toxicity Criteria version 4.03 (NCI-CTCAE v4.03).

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, a subject is deemed to develop “severe neurotoxicity” in response to or secondary to administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table 6.

TABLE 6
Exemplary Grading Criteria for neurotoxicity

| Grade | Description of Symptoms |
|---|---|
| 1 | Mild or asymptomatic symptoms |
| Asymptomatic or Mild | |
| 2 | Presence of symptoms that limit instrumental |
| Moderate | activities of daily living (ADL), such as preparing |
| | meals, shopping for groceries or clothes, using the |
| | telephone, managing money |
| 3 | Presence of symptoms that limit self-care ADL, |
| Severe | such as bathing, dressing and undressing, feeding |
| | self, using the toilet, taking medications |
| 4 | Symptoms that are life-threatening, requiring |
| Life-threatening | urgent intervention |
| 5 | Death |
| Fatal | |

In some embodiments, the methods reduce symptoms associated with CRS or neurotoxicity compared to other methods. In some aspects, the provided methods reduce symptoms, outcomes or factors associated with CRS, including symptoms, outcomes or factors associated with severe CRS or grade 3 or higher CRS, compared to other methods. For example, subjects treated according to the present methods may lack detectable and/or have reduced symptoms, outcomes or factors of CRS, e.g. severe CRS or grade 3 or higher CRS, such as any described, e.g. set forth in Table 3 and Table 4. In some embodiments, subjects treated according to the present methods may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods. In some embodiments, subjects treated according to the present methods may have reduced symptoms associated with peripheral motor neuropathy, peripheral sensory neuropathy, dysethesia, neuralgia or paresthesia.

In some embodiments, the methods reduce outcomes associated with neurotoxicity including damages to the nervous system and/or brain, such as the death of neurons. In some aspects, the methods reduce the level of factors associated with neurotoxicity such as beta amyloid (A3), glutamate, and oxygen radicals.

In some embodiments, the toxicity outcome is a dose-limiting toxicity (DLT). In some embodiments, the toxic outcome is a dose-limiting toxicity. In some embodiments, the toxic outcome is the absence of a dose-limiting toxicity. In some embodiments, a dose-limiting toxicity (DLT) is defined as any grade 3 or higher toxicity as assessed by any known or published guidelines for assessing the particular toxicity, such as any described above and including the National Cancer Institute (NCI) Common Terminology Criteria for Adverse Events (CTCAE) version 4.0.

In some embodiments, the low rate, risk or likelihood of developing a toxicity, e.g. CRS or neurotoxicity or severe CRS or neurotoxicity, e.g. grade 3 or higher CRS or neurotoxicity, observed with administering a dose of T cells in accord with the provided methods, and/or with the provided articles of manufacture or compositions, permits administration of the cell therapy on an outpatient basis. In some embodiments, the administration of the cell therapy, e.g. dose of T cells (e.g. CAR⁺ T cells) in accord with the provided methods, and/or with the provided articles of manufacture or compositions, is performed on an outpatient basis or does not require admission to the subject to the hospital, such as admission to the hospital requiring an overnight stay.

In some aspects, subjects administered the cell therapy, e.g. dose of T cells (e.g. CAR⁺ T cells) in accord with the provided methods, and/or with the provided articles of manufacture or compositions, including subjects treated on an outpatient basis, are not administered an intervention for treating any toxicity prior to or with administration of the cell dose, unless or until the subject exhibits a sign or symptom of a toxicity, such as of a neurotoxicity or CRS. Exemplary agents for treating, delaying, attenuating or ameliorating a toxicity are described in Section I-C.

In some embodiments, if a subject administered the cell therapy, e.g. dose of T cells (e.g. CAR⁺ T cells), including subjects treated on an outpatient basis, exhibits a fever the subject is given or is instructed to receive or administer a treatment to reduce the fever. In some embodiments, the fever in the subject is characterized as a body temperature of the subject that is (or is measured at) at or above a certain threshold temperature or level. In some aspects, the threshold temperature is that associated with at least a low-grade fever, with at least a moderate fever, and/or with at least a high-grade fever. In some embodiments, the threshold temperature is a particular temperature or range. For example, the threshold temperature may be at or about or at least at or about 38, 39, 40, 41, or 42 degrees Celsius, and/or may be a range of at or about 38 degrees Celsius to at or about 39 degrees Celsius, a range of at or about 39 degrees Celsius to at or about 40 degrees Celsius, a range of at or about 40 degrees Celsius to at or about 41 degrees, or a range of at or about 41 degrees Celsius to at or about 42 degrees Celsius.

In some embodiments, the treatment designed to reduce fever includes treatment with an antipyretic. An antipyretic may include any agent, e.g., compound, composition, or ingredient, that reduces fever, such as one of any number of agents known to have antipyretic effects, such as NSAIDs (such as ibuprofen, naproxen, ketoprofen, and nimesulide), salicylates, such as aspirin, choline salicylate, magnesium salicylate, and sodium salicylate, paracetamol, acetaminophen, Metamizole, Nabumetone, Phenaxone, antipyrine, febrifuges. In some embodiments, the antipyretic is acetaminophen. In some embodiments, acetaminophen can be administered at a dose of 12.5 mg/kg orally or intravenously up to every four hours. In some embodiments, it is or comprises ibuprofen or aspirin.

In some embodiments, if the fever is a sustained fever, the subject is administered an alternative treatment for treating the toxicity, such as any described in Section I-C. For subjects treated on an outpatient basis, the subject is instructed to return to the hospital if the subject has and/or is determined to or to have a sustained fever. In some embodiments, the subject has, and/or is determined to or considered to have, a sustained fever if he or she exhibits a fever at or above the relevant threshold temperature, and where the fever or body temperature of the subject is not reduced, or is not reduced by or by more than a specified amount (e.g., by more than 1° C., and generally does not fluctuate by about, or by more than about, 0.5° C., 0.4° C., 0.3° C., or 0.2° C.), following a specified treatment, such as a treatment designed to reduce fever such as treatment with an antipyreticm, e.g. NSAID or salicylates, e.g. ibuprofen, acetaminophen or aspirin. For example, a subject is considered to have a sustained fever if he or she exhibits or is determined to exhibit a fever of at least at or about 38 or 39 degrees Celsius, which is not reduced by or is not reduced by more than at or about 0.5° C., 0.4° C., 0.3° C., or 0.2° C., or by at or about 1%, 2%, 3%, 4%, or 5%, over a period of 6 hours, over a period of 8 hours, or over a period of 12 hours, or over a period of 24 hours, even following treatment with the antipyretic such as acetaminophen. In some embodiments, the dosage of the antipyretic is a dosage ordinarily effective in such as subject to reduce fever or fever of a particular type such as fever associated with a bacterial or viral infection, e.g., a localized or systemic infection.

In some embodiments, the subject has, and/or is determined to or considered to have, a sustained fever if he or she exhibits a fever at or above the relevant threshold temperature, and where the fever or body temperature of the subject does not fluctuate by about, or by more than about, 1° C., and generally does not fluctuate by about, or by more than about, 0.5° C., 0.4° C., 0.3° C., or 0.2° C. Such absence of fluctuation above or at a certain amount generally is measured over a given period of time (such as over a 24-hour, 12-hour, 8-hour, 6-hour, 3-hour, or 1-hour period of time, which may be measured from the first sign of fever or the first temperature above the indicated threshold). For example, in some embodiments, a subject is considered to or is determined to exhibit sustained fever if he or she exhibits a fever of at least at or about or at least at or about 38 or 39 degrees Celsius, which does not fluctuate in temperature by more than at or about 0.5° C., 0.4° C., 0.3° C., or 0.2° C., over a period of 6 hours, over a period of 8 hours, or over a period of 12 hours, or over a period of 24 hours.

In some embodiments, the fever is a sustained fever; in some aspects, the subject is treated at a time at which a subject has been determined to have a sustained fever, such as within one, two, three, four, five six, or fewer hours of such determination or of the first such determination following the initial therapy having the potential to induce the toxicity, such as the cell therapy, such as dose of T cells, e.g. CAR⁺ T cells.

In some embodiments, one or more interventions or agents for treating the toxicity, such as a toxicity-targeting therapies, is administered at a time at which or immediately after which the subject is determined to or confirmed to (such as is first determined or confirmed to) exhibit sustained fever, for example, as measured according to any of the aforementioned embodiments. In some embodiments, the one or more toxicity-targeting therapies is administered within a certain period of time of such confirmation or determination, such as within 30 minutes, 1 hour, 2 hours, 3 hours, 4 hours, 6 hours, or 8 hours thereof.

### II. Cell Therapy and Engineering Cells

In some embodiments, the cell therapy (e.g., T cell therapy) methods disclosed herein includes administering engineered cells expressing recombinant receptors (e.g. CAR) designed to recognize and/or specifically bind to antigens associated with the disease or condition, such as r/r/ B-cell NHL. In particular embodiments, the antigen that is bound or recognized by the recombinant receptor (e.g. CAR) is CD19. In some embodiments, binding to the antigen results in a response, such as an immune response against such antigens. In some embodiments, the cells contain or are engineered to contain the recombinant receptor, such as a chimeric antigen receptor (CAR). The recombinant receptor, such as a CAR, generally includes an extracellular antigen (or ligand) binding domain specific to the antigen that is linked to one or more intracellular signaling components, in some aspects via linkers and/or transmembrane domain(s). In some aspects, the engineered cells are provided as pharmaceutical compositions and formulations suitable for administration to a subjects, such as for adoptive cell therapy. Also provided are therapeutic methods for administering the cells and compositions to subjects, e.g., patients.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Chimeric Antigen Receptors

In some embodiments of the provided methods and uses, the engineered cells, such as T cells, express a chimeric receptors, such as a chimeric antigen receptors (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the engineered cells, such as T cells, express a recombinant receptor such as a chimeric antigen receptor (CAR) with specificity for a particular antigen (or marker or ligand), such as an antigen expressed on the surface of a particular cell type. In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30. In particular aspects, the antigen is CD19. In some embodiments, any of such antigens are antigens expressed on human B cells.

The chimeric receptors, such as CARs, generally include an extracellular antigen binding domain that is an antigen-binding portion or portions of an antibody molecule. In some embodiments, the antigen-binding domain is a portion of an antibody molecule, generally a variable heavy (V_{H}) chain region and/or variable light (V_{L}) chain region of the antibody, e.g., an scFv antibody fragment. In some embodiments, the antigen-binding domain is a single domain antibody (sdAb), such as sdFv, nanobody, V_{H}H and V_{NAR}. In some embodiments, an antigen-binding fragment comprises antibody variable regions joined by a flexible linker.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments the antigen-binding domain includes a V_{H} and/or V_{L} derived from FMC63, which, in some aspects, can be an scFv. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NO: 38 and 39, respectively, and CDR-H3 set forth in SEQ ID NO: 40 or 54 and CDR-L1 set forth in SEQ ID NO: 35 and CDR-L2 set forth in SEQ ID NO: 36 or 55 and CDR-L3 sequences set forth in SEQ ID NO: 37 or 56. In some embodiments, the FMC63 antibody comprises the heavy chain variable region (V_{H}) comprising the amino acid sequence of SEQ ID NO: 41 and the light chain variable region (V_{L}) comprising the amino acid sequence of SEQ ID NO: 42.

In some embodiments, the scFv comprises a variable light chain containing the CDR-L1 sequence of SEQ ID NO:35, a CDR-L2 sequence of SEQ ID NO:36, and a CDR-L3 sequence of SEQ ID NO:37 and/or a variable heavy chain containing a CDR-H1 sequence of SEQ ID NO:38, a CDR-H2 sequence of SEQ ID NO:39, and a CDR-H3 sequence of SEQ ID NO:40, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of FMC63 set forth in SEQ ID NO:41 and a variable light chain region of FMC63 set forth in SEQ ID NO:42, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:24. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:25 or a sequence that exhibits at least 85%, 86%, 87%, 88%, 89%, 90%, 91%, 92%, 93%, 94%, 95%, 96%, 97%, 98%, or 99% sequence identity to SEQ ID NO:25. In some embodiments, the scFv comprises the sequence of amino acids set forth in SEQ ID NO:43 or a sequence that exhibits at least 85%, 86%, 87%, 88%, 89%, 90%, 91%, 92%, 93%, 94%, 95%, 96%, 97%, 98%, or 99% sequence identity to SEQ ID NO:43.

In some embodiments the antigen-binding domain includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 47-49, respectively, and CDR-L1, CDR-L2 and CDR-L3 sequences set forth in SEQ ID NOS: 44-46, respectively. In some embodiments, the SJ25C1 antibody comprises the heavy chain variable region (V_{H}) comprising the amino acid sequence of SEQ ID NO: 50 and the light chain variable region (V_{L}) comprising the amino acid sequence of SEQ ID NO: 51. In some embodiments, the svFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:47, a CDR-H2 sequence of SEQ ID NO:48, and a CDR-H3 sequence of SEQ ID NO:49, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of SJ25C1 set forth in SEQ ID NO:50 and a variable light chain region of SJ25C1 set forth in SEQ ID NO:51, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:52. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:53 or a sequence that exhibits at least 85%, 86%, 87%, 88%, 89%, 90%, 91%, 92%, 93%, 94%, 95%, 96%, 97%, 98%, or 99% sequence identity to SEQ ID NO:53.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody, V_{H}H or V_{NAR}) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. In some aspects, the CAR is a bispecific CAR, e.g., containing two antigen-binding domains with different specificities.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

The terms “complementarity determining region,” and “CDR,” synonymous with “hypervariable region” or “HVR,” are known, in some cases, to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known, in some cases, to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January;27(1):55-77 (“IMGT” numbering scheme); Honegger A and Plickthun A, “Yet another numbering scheme for immunoglobulin variable domains: an automatic modeling and analysis tool,” J Mol Biol, 2001 Jun. 8;309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 7, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 7
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32.34 | H26--H35B | H30--H35B |
| (Kabat | | | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAb) are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include sdFv, nanobody, V_{H}H or V_{NAR}.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some aspects, the recombinant receptor, e.g., a chimeric antigen receptor, includes an extracellular portion containing one or more ligand- (e.g., antigen-) binding domains, such as an antibody or fragment thereof, and one or more intracellular signaling region or domain (also interchangeably called a cytoplasmic signaling domain or region). In some aspects, the recombinant receptor, e.g., CAR, further includes a spacer and/or a transmembrane domain or portion. In some aspects, the spacer and/or transmembrane domain can link the extracellular portion containing the ligand- (e.g., antigen-) binding domain and the intracellular signaling region(s) or domain(s)

In some embodiments, the recombinant receptor such as the CAR, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, Hudecek et al. (2015) Cancer Immunol Res. 3(2): 125-135 or international patent application publication number WO2014031687.

In some embodiments, the spacer contains only a hinge region of an IgG, such as only a hinge of IgG4 or IgG1, such as the hinge only spacer set forth in SEQ ID NO: 1, and encoded by the sequence set forth in SEQ ID NO: 2. In some embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 4. In some embodiments, the spacer the spacer is an Ig hinge, e.g., an IgG4 hinge, linked to a C_{H}3 domain only, such as set forth in SEQ ID NO: 3. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 5. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some aspects, the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) consists or comprises the sequence of amino acids set forth in SEQ ID NOS: 1, 3-5, 27-34 or 24, or a variant of any of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 (4-1BB), or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28 or a variant thereof. The extracellular domain and transmembrane can be linked directly or indirectly. In some embodiments, the extracellular domain and transmembrane are linked by a spacer, such as any described herein.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:8. In some embodiments, the transmembrane-domain containing portion of the recombinant receptor comprises the sequence of amino acids set forth in SEQ ID NO: 9 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the recombinant receptor, e.g., CAR, includes at least one intracellular signaling component or components, such as an intracellular signaling region or domain. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components. Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the intracellular signaling regions, e.g., comprising intracellular domain or domains, include the cytoplasmic sequences of a region or domain that is involved in providing costimulatory signal.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8alpha, CD8beta, CD4, CD25, or CD16. For example, in some aspects, the CAR or other chimeric receptor includes a chimeric molecule between CD3-zeta (CD3-ζ) or Fc receptor γ and CD8alpha, CD8beta, CD4, CD25 or CD16.

In some embodiments, the intracellular (or cytoplasmic) signaling region comprises a human CD3 chain, optionally a CD3 zeta stimulatory signaling domain or functional variant thereof, such as an 112 AA cytoplasmic domain of isoform 3 of human CD3ζ (Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 13, 14 or 15 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 13, 14 or 15.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40 (CD134), CD27, DAP10, DAP12, ICOS and/or other costimulatory receptors. In some embodiments, the CAR includes a costimulatory region or domain of CD28 or 4-1BB, such as of human CD28 or human 4-1BB.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 12.

In some aspects, the same CAR includes both the primary (or activating) cytoplasmic signaling regions and costimulatory signaling components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16. An exemplary T2A linker sequence comprises the sequence of amino acids set forth in SEQ ID NO: 6 or 17 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 17.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered.

In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the chimeric antigen receptor includes an extracellular portion containing the antibody or fragment described herein. In some aspects, the chimeric antigen receptor includes an extracellular portion containing the antibody or fragment described herein and an intracellular signaling domain. In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD3ζ) chain. In some embodiments, the CD3-zeta chain is a human CD3-zeta chain. In some embodiments, the intracellular signaling region further comprises a CD28 and CD137 (4-1BB, TNFRSF9) co-stimulatory domains, linked to a CD3 zeta intracellular domain. In some embodiments, the CD28 is a human CD28. In some embodiments, the 4-1BB is a human 4-1BB. In some embodiments, the chimeric antigen receptor includes a transmembrane domain disposed between the extracellular domain and the intracellular signaling region. In some aspects, the transmembrane domain contains a transmembrane portion of CD28. The extracellular domain and transmembrane can be linked directly or indirectly. In some embodiments, the extracellular domain and transmembrane are linked by a spacer, such as any described herein.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. For example, in some embodiments, the CAR includes an antibody such as an antibody fragment, including scFvs, e.g. specific for CD19 such as any described above, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain.

In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer. In some embodiments, the CAR includes an antibody or fragment, such as scFv, e.g. specific for CD19 such as any described above, a spacer such as any of the Ig-hinge containing spacers, a CD28-derived transmembrane domain, a 4-1BB-derived intracellular signaling domain, and a CD3 zeta-derived signaling domain.

In particular embodiments of any of the provided methods, the CAR contains in order from N-terminus to C-terminus: an extracellular antigen-binding domain that is the scFv set forth in SEQ ID NO: 43, the spacer set forth in SEQ ID NO:1, the transmembrane domain set forth in SEQ ID NO:8, the 4-1BB costimulatory signaling domain set forth in SEQ ID NO:12, and the signaling domain of a CD3-zeta (CD3ζ) chain set forth in SEQ ID NO:13.

B. Nucleic Acids, Vectors and Methods for Genetic Engineering

In some embodiments, the cells, e.g., T cells, are genetically engineered to express a recombinant receptor. In some embodiments, the engineering is carried out by introducing polynucleotides that encode the recombinant receptor. Also provided are polynucleotides encoding a recombinant receptor, and vectors or constructs containing such nucleic acids and/or polynucleotides.

In some cases, the nucleic acid sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO:65 and encoded by the nucleotide sequence set forth in SEQ ID NO:66. In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 65 and encoded by the nucleotide sequence set forth in SEQ ID NO:66, or the CD8 alpha signal peptide set forth in SEQ ID NO:67.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor.

In certain cases where nucleic acid molecules encode two or more different polypeptide chains, e.g., a recombinant receptor and a marker, each of the polypeptide chains can be encoded by a separate nucleic acid molecule. For example, two separate nucleic acids are provided, and each can be individually transferred or introduced into the cell for expression in the cell. In some embodiments, the nucleic acid encoding the recombinant receptor and the nucleic acid encoding the marker are operably linked to the same promoter and are optionally separated by an internal ribosome entry site (IRES), or a nucleic acid encoding a self-cleaving peptide or a peptide that causes ribosome skipping, which optionally is a T2A, a P2A, an E2A or an F2A. In some embodiments, the nucleic acids encoding the marker and the nucleic acid encoding the recombinant receptor are operably linked to two different promoters. In some embodiments, the nucleic acid encoding the marker and the nucleic acid encoding the recombinant receptor are present or inserted at different locations within the genome of the cell. In some embodiments, the polynucleotide encoding the recombinant receptor is introduced into a composition containing cultured cells, such as by retroviral transduction, transfection, or transformation.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products ((e.g. encoding the marker and encoding the recombinant receptor) by a message from a single promoter. Alternatively, in some cases, a single promoter may direct expression of an RNA that contains, in a single open reading frame (ORF), two or three genes (e.g. encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No. 20070116690.

Any of the recombinant receptors described herein can be encoded by polynucleotides containing one or more nucleic acid sequences encoding recombinant receptors, in any combinations or arrangements. For example, one, two, three or more polynucleotides can encode one, two, three or more different polypeptides, e.g., recombinant receptors. In some embodiments, one vector or construct contains a nucleic acid sequence encoding marker, and a separate vector or construct contains a nucleic acid sequence encoding a recombinant receptor, e.g., CAR. In some embodiments, the nucleic acid encoding the marker and the nucleic acid encoding the recombinant receptor are operably linked to two different promoters. In some embodiments, the nucleic acid encoding the recombinant receptor is present downstream of the nucleic acid encoding the marker.

In some embodiments, the vector backbone contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR).

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16.

In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy, 2014 Apr. 3. doi: 10.1038/gt.2014.25; Carlens et al. (2000) Exp. Hematol., 28(10): 1137-46; Alonso-Camino et al. (2013) Mol. Ther. Nucl. Acids, 2, e93; Park et al., Trends Biotechnol., 2011 Nov. 29(11): 550-557.

In some embodiments, the viral vector is an adeno-associated virus (AAV).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV) or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

Cells and Preparation of Cells for Genetic Engineering In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood, 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (fluorescence activated cell sorting, FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° C. per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating or stimulating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/mL). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4⁺ and/or CD8⁺ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

C. Methods of Manufacturing Engineered Cells

In particular embodiments, the engineered cells are produced by a process that generates an output composition of enriched T cells from one or more input compositions and/or from a single biological sample. In certain embodiments, the output composition contains cells that express a recombinant receptor, e.g., a CAR, such as an anti-CD19 CAR. In particular embodiments, the cells of the output compositions are suitable for administration to a subject as a therapy, e.g., an autologous cell therapy. In some embodiments, the output composition is a composition of enriched CD3+ T cells, or enriched CD4+ and CD8+ T cells. The T cells are engineered by methods that involve introduction of a nucleic acid encoding the CAR, e.g. anti-CD19 CAR into cells under conditions in which the nucleic acid is integrated into the genome of the cells. In some embodiments, the engineering methods include transduction with viral vectors, such as lentiviral vectors. In particular embodiments, the T cells are activated or stimulated by contacting the cells with an oligomeric reagent, e.g., a streptavidin mutein oligomer. In some embodiments, the cells are engineered by a process that is completed within 96 hours or less, of stimulating the cells with an oligomeric reagent, e.g., a streptavidin mutein oligomer. In some embodiments, the provided methods do not include a step to expand or increase the number of cells during the process. Exemplary methods of manufacturing and engineered cells produced by such methods are disclosed in PCT/US2019/046062, which is incorporated by reference in its entirety.

In particular embodiments, the provided methods are used in connection with an entire process for generating or producing output cells and/or an output populations of engineered T cells, such as a process including some or all of the steps of: stimulating cells from an input population; engineering, transforming, transducing, or transfecting the stimulated cells to express or contain a heterologous or recombinant polynucleotide, e.g., a polynucleotide encoding a recombinant receptor such as a CAR; incubating the cells, removing or separating a stimulatory reagent from the cells, and harvesting and collecting the cells, in some aspects thereby generating an output population of engineered T cells.

In some embodiments, the provided methods are used in connection with an entire process for generating or producing output cells and/or output compositions of enriched T cells, such as a process including some or all of the steps of: collecting or obtaining a biological sample; isolating, selecting, or enriching input cells from the biological sample; cryofreezing and storing and then thawing the input cells; stimulating the cells; genetically engineering the stimulated cells to express or contain a recombinant polynucleotide, e.g., a polynucleotide encoding a recombinant receptor such as a CAR; formulating the engineered cells in an output composition; and cryofreezing and storing the formulated output cells until the cells are released for infusion and or administration to a subject. In some embodiments, the provided methods do not include a step to expand or increase the number of cells during the process, such as by cultivating the cells in a bioreactor under conditions where the cells expand, such as to a threshold amount that is at least 3, 4, 5, or more times the amount, level, or concentration of the cells as compared to the input population. In some embodiments, genetically engineering the cells is or includes steps for transducing the cells with a viral vector, such as by spinoculating the cells in the presence of viral particles and then incubating the cells under static conditions in the presence of the viral particles.

In certain embodiments, the total duration of the provided process for generating engineered cells, from the initiation of the stimulation to collecting, harvesting, or formulating the cells is, is about, or is less than 36 hours, 42 hours, 48 hours, 54 hours, 60 hours, 72 hours, 84 hours, 96 hours, 108 hours, or 120 hours. In certain embodiments, the total duration of the provided process for generating engineered cells, from the initiation of the stimulation to collecting, harvesting, or formulating the cells is, is about, or is less than 1.5 days, 2 days, 3 days, 4 days, or 5 days. In some embodiments, the total duration of the provided process for generating engineered cells, from the initiation of the stimulation to collecting, harvesting, or formulating the cells is between or between about 36 hours and 120 hours, 48 hours and 96 hours, or 48 hours and 72 hours, inclusive, or between or between about 1.5 days and 5 days, 2 days and 4 days, or 2 days and 3 days, inclusive. In particular embodiments, the amount of time to complete the provided process as measured from the initiation of incubation to harvesting, collecting, or formulating the cells is, is about, or is less than 48 hours, 72 hours, or 96 hours, or is, is about, or is less than 2 days, 3 days, or 4 days. In particular embodiments, the amount of time to complete the provided process as measured from the initiation of incubation to harvesting, collecting, or formulating the cells is 48 hours±6 hours, 72 hours±6 hours, or 96 hours±6 hours.

In some embodiments, the incubation, e.g., as disclosed in Section II-C-5, is completed between or between about 24 hour and 120 hours, 36 hour and 108 hours, 48 hours and 96 hours, or 48 hours and 72 hours, inclusive, after the initiation of the stimulation. In some embodiments, the incubation is completed at, about, or within 120 hours, 108 hours, 96 hours, 72 hours, 48 hours, or 36 hours from the initiation of the stimulation. In particular embodiments, the incubation are completed after 24 hours±6 hours, 48 hours±6 hours, or 72 hours±6 hours. In some embodiments, the incubation is completed between or between about one day and 5 days, 1.5 days and 4.5 days, 2 days and 4 days, or 2 day and 3 days, inclusive, after the initiation of the stimulation. In some embodiments, the incubation is completed at, about, or within 5 days, 4 days, 3 days, 2 days, or 1.5 days from the initiation of the stimulation.

In some embodiments, the entire process is performed with a single population of enriched T cells, e.g., CD4+ and CD8+ T cells. In certain embodiments, the process is performed with two or more input populations of enriched T cells (e.g., CD4 and CD8 cells) that are combined prior to and/or during the process to generate or produce a single output population of enriched T cells. In some embodiments, the enriched T cells are or include engineered T cells, e.g., T cells transduced to express a recombinant receptor.

In some embodiments, an output population, e.g., a population of engineered T cells, is generated by (i) incubating an input population of or containing T cells under stimulating conditions for between or between about 18 and 30 hours, inclusive, (ii) introducing a heterologous or recombinant polynucleotide encoding a recombinant receptor into T cells of the stimulated population, (iii) incubating the cells, and then (iv) collecting or harvesting the incubated cells.

In some embodiments, the cells are collected or harvested within between 36 and 108 hours or between 1.5 days and 4.5 days after the incubation under stimulatory conditions is initiated. In particular embodiments, the cells are collected or harvested within 48 hours or two days after the transformed (e.g., genetically engineered, transduced, or transfected) T cells achieve a stable integrated vector copy number (iVCN) per genome that does not increase or decrease by more than 20% within a span of 24-48 hours or one to two days. In some embodiments, the integration is considered stable when the measured iVCN of a cell population is within or within about 20%, 15%, 10%, or 5% of the total vector copy number (VCN) measured in the population. Particular embodiments contemplate that to achieve a stable integration, the cells must be incubated for, for about, or for at least 48 hours, 60 hours, or 72 hours, or one day, 2 days, or 3 days, after the viral vector is contacted or introduced to the cells. In some embodiments, the stable integration occurs within or with about 72 hours of the incubation. In some embodiments, the cells are collected or harvested at a time when the total number of transformed T cells is at or less than the total number of cells of the input population. In various embodiments, the cells are collected or harvested at a time before the cells of the input population have doubled more than three, two, or one time(s). Exemplary methods and compositions for the VCN and iVCN assays are disclosed in PCT/US2019/046048, which is incorporated herein by reference in its entirety.

In certain embodiments, an output population, e.g., a population of engineered T cells, is generated by (i) incubating an input population comprising T cells under stimulating conditions for between 18 and 30 hours, inclusive, in the presence of a stimulatory reagent, e.g., a stimulatory reagent described herein, such as in Section II-C-2, (ii) transducing the stimulated T cells with a viral vector encoding a recombinant receptor, such as by spinoculating the stimulated T cells in the presence of the viral vector, (iii) incubating the transduced T cells under static conditions for between or between 18 hours and 96 hours, inclusive, and (iv) harvesting T cells of the transformed population within between or between about 36 and 108 hours after the incubation under stimulatory conditions is initiated.

In some embodiments, the process associated with the provided methods is compared to an alternative process. For example, in some embodiments, the provided methods herein are compared an alternative process that contains a step for expanding the cells. In particular embodiments, the alternative process may differ in one or more specific aspects, but otherwise contains similar or the same features, aspects, steps, stages, reagents, and/or conditions of the process associated with the provided methods. In some embodiments, the alternative process is similar as the process associated with the provided methods, e.g., lacks or does not include expansion, but differs in a manner that includes, but is not limited to, one or more of; different reagents and/or media formulations; presence of serum during the incubation, transduction, transfection, and/or incubation of the engineered cells; different cellular makeup of the input population, e.g., ratio of CD4+ to CD8+ T cells; different stimulating conditions and/or a different stimulatory reagent; different ratio of stimulatory reagent to cells; different vector and/or method of transduction; different timing or order for incubating, transducing, and/or transfecting the cells; absence or difference of one or more recombinant cytokines present during the incubation or transduction (e.g., different cytokines or different concentrations), or different timing for harvesting or collecting the cells.

In some embodiments, the duration or amount of time required to complete the provided process, as measured from the isolation, enrichment, and/or selection input cells (e.g., CD4+ or CD8+ T cells) from a biological sample to the time at which a the output cells are collected, formulated, and/or cryoprotected is, is about, or is less than 48 hours, 72 hours, 96 hours, 120 hours, 2 days, 3 days, 4 days, 5 days, 7 days, or 10 days. In some embodiments, isolated, selected, or enriched cells are not cryoprotected prior to the stimulation, and the duration or amount of time required to complete the provided process, as measured from the isolation, enrichment, and/or selection input cells (to the time at which a the output cells are collected, formulated, and/or cryoprotected is, is about, or is less than 48 hours, 72 hours, 96 hours, or 120 hours, or 2 days, 3 days, 4 days, or 5 days.

In certain embodiments, the provided processes are performed on a population of cells, e.g., CD4+ and CD8+ T cells, that were isolated, enriched, or selected from a biological sample. In some aspects, the provided methods can produce or generate a composition of engineered T cells from when a biological sample is collected from a subject within a shortened amount of time as compared to other methods or processes. In some embodiments, the provided methods can produce or generate engineered T cells, including any or all times where biological samples, or enriched, isolated, or selected cells are cryopreserved and stored prior to steps for stimulation or transduction, within or within about 10 days, 9 days, 8 days, 7 days, 6 days, 5 days, 4 days, 3 days, 2 days, or within or within about 120 hours, 96 hours, 72 hours, or 48 hours, from when a biological sample is collected from a subject to when the engineered T cells are collected, harvested, or formulated (e.g., for cryopreservation or administration). In particular embodiments, the provided methods can produce or generate engineered T cells, including any or all times where biological samples, or enriched, isolated, or selected cells are cryopreserved and stored prior to steps for stimulation or transduction, within between or between about 6 days and 8 days, inclusive, from when the biological sample is collected from a subject to when the engineered T cells are collected, harvested, or formulated.

In certain embodiments, the provided methods are used in connection with a process for generating or producing output cells and/or output populations of enriched T cells. In particular embodiments, the output cells and/or output populations of enriched T cells are or include cells that were collected, obtained, isolated, selected, and/or enriched from the biological sample, such as a blood sample or leukapheresis sample; incubated under stimulating conditions; engineered, e.g., transduced, to express or contain a recombinant polynucleotide, e.g., a polynucleotide encoding a recombinant receptor such as a CAR; incubated to a threshold cell amount or density; and/or formulated. In some embodiments, the of the output population have been previously cryoprotected and thawed, e.g., during, prior to, and/or after one or more steps of the process. In some embodiments, the output population contains T cells, e.g., CD4+ T cells and CD8+ T cells, that express a recombinant receptor, e.g., a CAR.

In some embodiments, at least 30%, at least 40%, at least 45%, at least 50%, at least 55%, at least 60%, at least 65%, at least 70%, at least 75%, at least 80%, at least 85%, or at least 90%, at least 95%, of the cells of the output population express the recombinant receptor. In certain embodiments, at least 50% of the cells of the output composition express the recombinant receptor. In certain embodiments, at least 30%, at least 40%, at least 45%, at least 50%, at least 55%, at least 60%, at least 65%, at least 70%, at least 75%, at least 80%, at least 85%, at least 90%, or at least 95%, of the CD3+ T cells of the output composition express the recombinant receptor. In some embodiments, at least 50% of the CD3+ T cells of the output composition express the recombinant receptor. In particular embodiments, at least at least 30%, at least 40%, at least 45%, at least 50%, at least 55%, at least 60%, at least 65%, at least 70%, at least 75%, at least 80%, at least 85%, at least 90%, at least 95%, at least 97%, at least 99%, or more than 99% of the CD4+ T cells of the output composition express the recombinant receptor. In particular embodiments, at least 50% of the CD4+ T cells of the output composition express the recombinant receptor. In some embodiments, at least at least 30%, at least 40%, at least 45%, at least 50%, at least 55%, at least 60%, at least 65%, at least 70%, at least 75%, at least 80%, at least 85%, at least 90%, at least 95%, at least 97%, at least 99%, or more than 99% of the CD8+ T cells of the output composition express the recombinant receptor. In certain embodiments, at least 50% of the CD8+ T cells of the output composition express the recombinant receptor.

In particular embodiments, the cells of the output composition have improved cytolytic activity towards cells expressing an antigen bound by and/or recognized by the recombinant receptor (e.g., target cells) as compared output cells produced by an alternative process, e.g., a process that includes one or more steps of expanding the cells. In some embodiments, when the cells of the output composition are exposed to the cells that express the antigen, e.g., the target cells, the cells of the output composition kill, kill about, or kill at least 25%, 30%, 35%, 40%, 45%, 50%, 55%, 60%, 70%, 75%, 80%, 85%, 90%, 95%, or 100% of cells that express the antigen. In certain embodiments, the cells of the output composition kill at least 25%, 50%, 75%, 100%, 150%, or 1-fold, 2-fold, 3-fold, 4-fold, or 5-fold greater amount of cells that express the antigen, e.g., target cells, than output cells produced by the alternative process under similar or the same conditions.

In particular embodiments, the cells of the output population have improved anti-tumor activity in vivo as compared to output cells produced by an alternative process, e.g., a process that includes one or more steps of expanding the cells. In some embodiments, when the cells of the output composition are administered to a subject, e.g., a subject having a tumor or cancer, the cells of the output population kill, kill about, or kill at least 25%, 30%, 35%, 40%, 45%, 50%, 55%, 60%, 70%, 75%, 80%, 85%, 90%, 95%, or 100% of the tumor cells, e.g., cancer or tumor cells expressing the antigen, in the subject. In certain embodiments, the cells of the output composition kill at least 25%, 50%, 75%, 100%, 150%, or 1-fold, 2-fold, 3-fold, 4-fold, or 5-fold greater amount of tumor cells in vivo than output cells produced by the alternative process under similar or the same conditions.

In particular embodiments, a majority of the cells of the output population are naïve-like, central memory, and/or effector memory cells. In particular embodiments, a majority of the cells of the output population are naïve-like or central memory cells. In some embodiments, a majority of the cells of the output population are positive for one or more of CCR7 or CD27 expression. In certain embodiments, the cells of the output population have a greater portion of naïve-like or central memory cells that output populations generated from alternative processes, such as processes that involve expansion.

In certain embodiments, the cells of the output population have a low portion and/or frequency of cells that are exhausted and/or senescent. In particular embodiments, the cells of the output population have a low portion and/or frequency of cells that are exhausted and/or senescent. In some embodiments, less than 40%, less than 35%, less than 30%, less than 25%, less than 20%, less than 15%, less than 10%, less than 5%, or less than 1% of the cells of the output population are exhausted and/or senescent. In certain embodiments, less than 25% of the cells of the output population are exhausted and/or senescent. In certain embodiments, less than less than 10% of the cells of the output population are exhausted and/or senescent. In particular embodiments, the cells have a low portion

In some embodiments, the cells of the output population have a low portion and/or frequency of cells that are negative for CD27 and CCR7 expression, e.g., surface expression. In particular embodiments, the cells of the output population have a low portion and/or frequency of CD27−CCR7− cells. In some embodiments, less than 40%, less than 35%, less than 30%, less than 25%, less than 20%, less than 15%, less than 10%, less than 5%, or less than 1% of the cells of the output population are CD27−CCR7− cells. In certain embodiments, less than 25% of the cells of the output population are CD27−CCR7− cells. In certain embodiments, less than less than 10% of the cells of the output population are CD27−CCR7− cells. In embodiments, less than 5% of the cells of the output population are CD27−CCR7− cells.

In some embodiments, the cells of the output population have a high portion and/or frequency of cells that are positive for one or both of CD27 and CCR7 expression, e.g., surface expression. In some embodiments, the cells of the output population have a high portion and/or frequency of cells that are positive for one or both of CD27 and CCR7. In some embodiments, at least 50%, at least 60%, at least 70%, at least 75%, at least 80%, at least 85%, at least 90%, at least 95%, or greater than 95% of the cells of the output population are positive for one or both of CD27 and CCR7. In various embodiments, at least 50%, at least 60%, at least 70%, at least 75%, at least 80%, at least 85%, at least 90%, at least 95% or greater than 95% of the CD4+CAR+ cells of the output population are positive for one or both of CD27 and CCR7. In some embodiments, at least 50%, at least 60%, at least 70%, at least 75%, at least 80%, at least 85%, at least 90%, at least 95% or greater than 95% of the CD8+CAR+ cells of the output population are positive for one or both of CD27 and CCR7.

In certain embodiments, the cells of the output population have a high portion and/or frequency of cells that are positive for CD27 and CCR7 expression, e.g., surface expression. In some embodiments, the cells of the output population have a high portion and/or frequency of CD27+CCR7+ cells. In some embodiments, at least 50%, at least 60%, at least 70%, at least 75%, at least 80%, at least 85%, at least 90%, at least 95%, or greater than 95% of the cells of the output population are CD27+CCR7+ cells. In various embodiments, at least 50%, at least 60%, at least 70%, at least 75%, at least 80%, at least 85%, at least 90%, at least 95% or greater than 95% of the CD4+CAR+ cells of the output population are CD27+CCR7+ cells. In some embodiments, at least 50%, at least 60%, at least 70%, at least 75%, at least 80%, at least 85%, at least 90%, at least 95% or greater than 95% of the CD8+CAR+ cells of the output population are CD27+CCR7+ cells.

In certain embodiments, the cells of the output population have a low portion and/or frequency of cells that are negative for CCR7 and positive for CD45RA expression, e.g., surface expression. In some embodiments, the cells of the output population have a low portion and/or frequency of CCR7−CD45RA+ cells. In particular embodiments, less than 40%, less than 35%, less than 30%, less than 25%, less than 20%, less than 15%, less than 10%, less than 5%, or less than 1% of the cells of the output population are CCR7−CD45RA+ cells. In some embodiments, less than 25% of the cells of the output population are CCR7−CD45RA+ cells. In particular embodiments, less than less than 10% of the cells of the output population are CCR7−CD45RA+ cells. In certain embodiments, less than 5% of the cells of the output population are CCR7−CD45RA+ cells.

In particular embodiments, the cells are harvested prior to, prior to about, or prior to at least one, two, three, four, five, six, eight, ten, twenty, or more cell doublings of the cell population, e.g., doublings that occur during the incubating. In certain embodiments, the cells are harvested prior to any doubling of the population, e.g., doubling that occurs during the incubation. In some aspects, reducing the doubling that may occur during an engineering process will, in some embodiments, increase the portion of engineered T cells that are naïve-like. In some embodiments, increasing the doubling during an engineering process increases T cell differentiation that may occur during the engineering process.

In some aspects, it is contemplated that, for a process for generating or producing engineered cell compositions, reducing the expansion or cell doublings that occur during the process, e.g., during the incubation, increases the amount or portion of naïve-like T cells of the resulting engineered cell composition. In particular aspects, increasing the expansion or cell doublings that occur during the process increases the amount or portion of differentiated T cells of the resulting engineered cell composition. In some aspects, it is contemplated that process, such as the processes provided herein, that increase or enlarge the portion of naïve-like cells in the resulting engineered cell composition may increase the potency, efficacy, and persistence, e.g., in vivo after administration, of the engineered cell composition.

1. Cells and Preparation of Cells for Genetic Engineering

In some embodiments, cells, such as T cells, used in connection with the provided methods, uses, articles of manufacture or compositions are cells have been genetically engineered to express a recombinant receptor, e.g., a CAR described herein. In some embodiments, the engineered cells are used in the context of cell therapy, e.g., adoptive cell therapy. In some embodiments, the engineered cells are immune cells. In some embodiments, the engineered cells are T cells, such as CD4+ or CD8+ T cells.

In particular embodiments, the provided methods are used in connection with isolating, selecting, or enriching cells from a biological sample to generate one or more input populations of enriched cells, e.g., T cells. In some embodiments, the provided methods include isolation of cells or populations thereof from biological samples, such as those obtained from or derived from a subject, such as one having a particular disease or condition or in need of a cell therapy or to which cell therapy will be administered. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the sample containing cells (e.g., an apheresis product or a leukapheresis product) is washed in order to remove one or more anti-coagulants, such as heparin, added during apheresis or leukapheresis.

In some embodiments, the sample containing cells (e.g., a whole blood sample, a buffy coat sample, a peripheral blood mononuclear cells (PBMC) sample, an unfractionated T cell sample, a lymphocyte sample, a white blood cell sample, an apheresis product, or a leukapheresis product) is cryopreserved and/or cryoprotected (e.g., frozen) and then thawed and optionally washed prior to any steps for isolating, selecting, activating, stimulating, engineering, transducing, transfecting, incubating, culturing, harvesting, formulating a population of the cells, and/or administering the formulated cell population to a subject.

In some embodiments, a sample containing autologous Peripheral Blood Mononuclear Cells (PBMCs) from a subject is collected in a method suitable to ensure appropriate quality for manufacturing. In one aspect, the sample containing PBMCs is derived from fractionated whole blood. In some embodiments, whole blood from a subject is fractionated by leukapheresis using a centrifugal force and making use of the density differences between cellular phenotypes, when autologous mononuclear cells (MNCs) are preferentially enriched while other cellular phenotypes, such as red blood cells, are reduced in the collected cell composition. In some embodiments, autologous plasma is concurrently collected during the MNC collection, which in some aspects can allow for extended leukapheresis product stability. In one aspect, the autologous plasma is added to the leukapheresis product to improve the buffering capacity of the leukapheresis product matrix. In some aspects, a total volume of whole blood processed in order to generate the leukapheresis product is or is about 2 L, 4 L, 6 L, 8 L, 10 L, 12 L, 14 L, 16 L, 18 L, or 20 L, or is any value between any of the foregoing. In some embodiments, the volume of autologous plasma collected is or is about 10 mL, 50 mL, 100 mL, 150 mL, 200 mL, 250 mL, or 300 mL, or more, or is a volume between any of the foregoing. In some embodiments, the leukapheresis product is subjected to a procedure, e.g., washing and formulation for in-process cryopreservation, within about 48 hours of the leukapheresis collection completion. In some embodiments, the leukapheresis product is subjected to one or more wash steps, e.g., within about 2 hours, 6 hours, 12 hours, 18 hours, 24 hours, 36 hours, or 48 hours of the leukapheresis collection completion. In some aspects, the one or more wash step removes the anticoagulant during leukapheresis collection, cellular waste that may have accumulated in the leukapheresis product, residual platelets and/or cellular debris. In some embodiments, one or more buffer exchange is performed during the one or more wash step.

In particular embodiments, an apheresis product or a leukapheresis product is cryopreserved and/or cryoprotected (e.g., frozen) and then thawed before being subject to a cell selection or isolation step (e.g., a T cell selection or isolation step) as described infra. In some embodiments, after a cryopreserved and/or cryoprotected apheresis product or leukapheresis product is subject to a T cell selection or isolation step, no additional cryopreservation and/or cryoprotection step is performed during or between any of the subsequent steps, such as the steps of activating, stimulating, engineering, transducing, transfecting, incubating, culturing, harvesting, formulating a population of the cells, and/or administering the formulated cell population to a subject. For example, T cells selected from a thawed cryopreserved and/or cryoprotected apheresis product or leukapheresis product are not again cryopreserved and/or cryoprotected before being thawed and optionally washed for a downstream process, such as T cell activation/stimulation or transduction.

In particular embodiments, an apheresis product or a leukapheresis product is cryopreserved and/or cryoprotected (e.g., frozen) at a density of, of about, or at least 5×10⁶ cells/mL, 10×10⁶ cells/mL, 20×10⁶ cells/mL, 30×10⁶ cells/mL, 40×10⁶ cells/mL, 50×10⁶ cells/mL, 60×10⁶ cells/mL, 70×10⁶ cells/mL, 80×10⁶ cells/mL, 90×10⁶ cells/mL, 100×10⁶ cells/mL, 110×10⁶ cells/mL, 120×10⁶ cells/mL, 130×10⁶ cells/mL, 140×10⁶ cells/mL, or 150×10⁶ cells/mL, or any value between any of the foregoing, in a cryopreservation solution or buffer. In some embodiments, the cryopreservation solution or buffer is or contains, for example, a DMSO solution optionally comprising human serum albumin (HSA), or other suitable cell freezing media.

In particular embodiments, the cryopreserved and/or cryoprotected apheresis product or leukapheresis product is banked (e.g., without T cell selection before freezing the sample), which, in some aspects, can allow more flexibility for subsequent manufacturing steps. In some aspects, the cryopreserved and/or cryoprotected apheresis product or leukapheresis product is aliquoted into multiple cryopreservation container such as bags, which can each individually or in combination be used in processing of the product. For example, when the total number of viable cells in the apheresis product or leukapheresis product is less than 15×10⁹ cells, the cryopreserved and/or cryoprotected apheresis product or leukapheresis product is aliquoted into four cryopreservation container such as bags. In some embodiments, when the total number of viable cells in the apheresis product or leukapheresis product is 15-30×10⁹ cells, the cryopreserved and/or cryoprotected apheresis product or leukapheresis product is aliquoted into eight cryopreservation container such as bags.

In one aspect, banking cells before selection increases cell yields for a downstream process, and banking cells earlier may mean they are healthier and may be easier to meet manufacturing success criteria. In another aspect, once thawed, the cryopreserved and/or cryoprotected apheresis product or leukapheresis product can be subject to one or more different selection methods. Advantages of this approach are, among other things, to enhance the availability, efficacy, and/or other aspects of cells of a cell therapy for treatment of a disease or condition of a subject, such as in the donor of the sample and/or another recipient.

In some embodiments, the sample (e.g. apheresis or leukapheresis sample) is collected and cryopreserved and/or cryoprotected prior to or without prior cell selection (e.g., without prior T cell selection, such as selection by chromatography), at a time after the donor is diagnosed with a disease or condition. In some aspects, the time of cryopreservation also is before the donor has received one or more of the following: any initial treatment for the disease or condition, any targeted treatment or any treatment labeled for treatment for the disease or condition, or any treatment other than radiation and/or chemotherapy. In some embodiments, the sample is collected after a first relapse of a disease following initial treatment for the disease, and before the donor or subject receives subsequent treatment for the disease. The initial and/or subsequent treatments may be a therapy other than a cell therapy. In some embodiments, the collected cells may be used in a cell therapy following initial and/or subsequent treatments. In one aspect, the cryopreserved and/or cryoprotected sample without prior cell selection may help reduce up-front costs, such as those associated with non-treatment patients in a randomized clinic trial who may crossover and require treatment later.

In some embodiments, the sample (e.g. apheresis or leukapheresis sample) is collected and cryopreserved and/or cryoprotected prior to or without prior cell selection (e.g., without prior T cell selection, such as selection by chromatography), at a time after a second relapse of a disease following a second line of treatment for the disease, and before the donor or subject receives subsequent treatment for the disease. In some embodiments, patients are identified as being likely to relapse after a second line of treatment, for example, by assessing certain risk factors. In some embodiments, the risk factors are based on disease type and/or genetics, such as double-hit lymphoma, primary refractory cancer, or activated B-cell lymphoma. In some embodiments, the risk factors are based on clinical presentation, such as early relapse after first-line treatment, or other poor prognostic indicators after treatment (e.g., IPI (International Prognostic Index)>2).

In some embodiments, the sample (e.g. apheresis or leukapheresis sample) is collected and cryopreserved and/or cryoprotected prior to or without prior cell selection (e.g., without prior T cell selection, such as selection by chromatography), at a time before the donor or subject is diagnosed with a disease. In some aspects, the donor or subject may be determined to be at risk for developing a disease. In some aspects, the donor or subject may be a healthy subject. In certain cases, the donor or subject may elect to bank or store cells without being deemed at risk for developing a disease or being diagnosed with a disease in the event that cell therapy is required at a later stage in life. In some embodiments, a donor or subject may be deemed at risk for developing a disease based on factors such as genetic mutations, genetic abnormalities, genetic disruptions, family history, protein abnormalities (such as deficiencies with protein production and/or processing), and lifestyle choices that may increase the risk of developing a disease. In some embodiments, the cells are collected as a prophylactic.

In some embodiments, the cryopreserved and/or cryoprotected sample of cells (e.g. apheresis or leukapheresis sample), such as a sample of cells that has not been subjected to a prior cell selection (e.g., without prior T cell selection, such as selection by chromatography) is stored, or banked, for a period of time greater than or equal to 12 hours, 24 hours, 36 hours, or 48 hours, or greater than or equal to 0.5 days, one day, 1.5 days, or two days. In some embodiments, the sample is stored or banked for a period of time greater than or equal to 1 week, 2 weeks, 3 weeks, or 4 weeks. In some embodiments, the sample is placed into long-term storage or long-term banking. In some aspects, the sample is stored for a period of time greater than or equal to 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, or more.

In some embodiments, an apheresis or leukapheresis sample taken from a donor is shipped in a cooled environment to a storage or processing facility, and/or cryogenically stored at the storage facility or processed at the processing facility. In some embodiments, before shipping, the sample is processed, for example, by selecting T cells, such as CD3+ T cells, CD4+ T cells, and/or CD8+ T cells. In some embodiments, such processing is performed after shipping and before cryogenically storing the sample. In some embodiments, the processing is performed after thawing the sample following cryogenically storage.

By allowing donors to store their cells at a stage when the donors, and thus their cells, have not undergone extensive treatment for a disease and/or prior to contracting of a disease or condition or diagnosis thereof, such cells may have certain advantages for use in cell therapy compared to cells harvested after one or after multiple rounds of treatment. For example, cells harvested before one or more rounds of treatment may be healthier, may exhibit higher levels of certain cellular activities, may grow more rapidly, and/or may be more receptive to genetic manipulation than cells that have undergone several rounds of treatment. Another example of an advantage according to embodiments described herein may include convenience. For example, by collecting, optionally processing, and storing a donor's cells before they are needed for cell therapy, the cells would be readily available if and when a recipient later needs them. This could increase apheresis lab capacity, providing technicians with greater flexibility for scheduling the apheresis collection process.

Exemplary methods and systems for cryogenic storage and processing of cells from a sample, such as an apheresis sample, can include those described in WO2018170188. In some embodiments, the method and systems involve collecting apheresis before the patient needs cell therapy, and then subjecting the apheresis sample to cryopreservation for later use in a process for engineering the cells, e.g. T cells, with a recombinant receptor (e.g. CAR). In some cases, such processes can include those described herein. In some embodiments, an apheresis sample is collected from a subject and cryopreserved prior to subsequent T cell selection, activation, stimulation, engineering, transduction, transfection, incubation, culturing, harvest, formulation of a population of the cells, and/or administration of the formulated cell population to a subject. In such examples, the cryopreserved apheresis sample is thawed prior to subjecting the sample to one or more selection steps, such as any as described herein.

In some embodiments, the cryopreserved and/or cryoprotected sample of cells (e.g. apheresis or leukapheresis sample), such as a sample of cells that has not been subject to a prior cell selection (e.g., without prior T cell selection, such as selection by chromatography) is thawed prior to its use for downstream processes for manufacture of a cell population for cell therapy, for example, a T cell population containing CAR+ T cells. In some embodiments, such a cryopreserved and/or cryoprotected sample of cells (e.g. apheresis or leukapheresis sample) is used in connection with the process provided herein for engineered a T cell therapy, such as a CAR+ T cell therapy. In particular examples, no further step of cryopreservation is carried out prior to or during the harvest/formulation steps.

In some embodiments, selection, isolation, or enrichment of the cells or populations includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include density-based cell separation methods, such as the preparation of white blood cells from peripheral blood by lysing the red blood cells and centrifugation through a Percoll or Ficoll gradient. In some embodiments, cells, e.g., T cells, are isolated, selected, or enriched by chromatographic isolation, such as by column chromatography including affinity chromatography or gel permeations chromatography. In some embodiments, the method employs a receptor binding reagent that binds to a receptor molecule that is located on the surface of a target cell, e.g., the cell to be isolated, selected, or enriched. Such methods may be described as (traceless) cell affinity chromatography technology (CATCH). In certain embodiments, methods, techniques, and reagents for selection, isolation, and enrichment are described, for example, in WO2013124474 and WO2015164675, which are hereby incorporated by reference in their entirety.

Cell selection may be performed using one or more chromatography columns. In some embodiments the one or more chromatography columns are included in a closed system. In some embodiments, the closed system is an automated closed system, for example requiring minimal or no user (e.g., human) input. In some embodiments, cell selection is performed sequentially (e.g., a sequential selection technique). In some embodiments, the one or more chromatography columns are arranged sequentially. For example, a first column may be oriented such that is the output of the column (e.g., eluant) can be fed, e.g., via connected tubing, to a second chromatography column. In some embodiments, a plurality of chromatography columns may be arranged sequentially. In some embodiments, cell selection may be achieved by carrying out sequential positive and negative selection steps, the subsequent step subjecting the negative and/or positive fraction from the previous step to further selection, where the entire process is carried out in the same tube or tubing set. In some embodiments, a sample containing target cells is subjected to a sequential selection in which a first selection is effected to enrich for one of the CD4+ or CD8+ populations, and the non-selected cells from the first selection are used as the source of cells for a second selection to enrich for the other of the CD4+ or CD8+ populations. In some embodiments, a further selection or selections can be effected to enrich for sub-populations of one or both of the CD4+ or CD8+ population, for example, central memory T (T_{CM}) cells, naïve T cells, and/or cells positive for or expressing high levels of one or more surface markers, e.g., CD28+, CD62L+, CCR7+, CD27+, CD127+, CD4+, CD8+, CD45RA+, and/or CD45RO+. In some embodiments, a sample containing target cells is subjected to a sequential selection in which a first selection is effected to enrich for a CD3+ population, and the selected cells are used as the source of cells for a second selection to enrich for CD3+ populations. In some embodiments, a sample containing target cells is subjected to a sequential selection in which a first selection is effected to enrich for a CD3+ population on a first stationary phase (e.g., in a first chromatograph column), and the flowthrough containing unbound cells is used as the source of cells for a second selection to enrich for a CD3+ population on a second stationary phase (e.g., in a second chromatograph column), wherein the first and second stationary phases are arranged sequentially. In some embodiments, the selection is a positive selection for CD3+ T cells (e.g., by using an antibody or antigen binding fragment thereof that specifically binds to cell surface CD3). In some embodiments, a further selection or selections can be effected to enrich for sub-populations of the CD3+ population, for example, central memory T (T_{CM}) cells, naïve T cells, and/or cells positive for or expressing high levels of one or more surface markers, e.g., CD28+, CD62L+, CCR7+, CD27+, CD127+, CD4+, CD8+, CD45RA+, and/or CD45RO+. In some embodiments, a sample containing target cells is subjected to sequential selection in which a first selection is effected to enrich for a CD3+ population, and the selected cells are used as the source of cells for a second selection to enrich for CD4+ populations. In some embodiments, a further selection or selections can be effected to enrich for sub-populations of the CD3+CD4+ population, for example, central memory T (T_{CM}) cells, naïve T cells, and/or cells positive for or expressing high levels of one or more surface markers, e.g., CD28+, CD62L+, CCR7+, CD27+, CD127+, CD4+, CD8+, CD45RA+, and/or CD45RO+. In some embodiments, a sample containing target cells is subjected to sequential selection in which a first selection is effected to enrich for a CD3+ population, and the selected cells are used as the source of cells for a second selection to enrich for CD8+ populations. In some embodiments, a further selection or selections can be effected to enrich for sub-populations of the CD3+CD8+ population, for example, central memory T (T_{CM}) cells, naïve T cells, and/or cells positive for or expressing high levels of one or more surface markers, e.g., CD28+, CD62L+, CCR7+, CD27+, CD127+, CD4+, CD8+, CD45RA+, and/or CD45RO+. It is contemplated that in some aspects, specific subpopulations of T cells (e.g., CD3+ cells), such as cells positive or expressing high levels of one or more surface markers, e.g., CD28+, CD62L+, CCR7+, CD27+, CD127+, CD4+, CD8+, CD45RA+, and/or CD45RO+ T cells, are selected by positive or negative sequential selection techniques. In some embodiments, cell selection is performed in parallel (e.g., parallel selection technique). In some embodiments, the one or more chromatography columns are arranged in parallel. For example, two or more columns may be arranged such that a sample is loaded onto two or more columns at the same time via tubing that allows for the sample to be applied to each column without the need for the sample to traverse through a first column. For example, using a parallel selection technique, cell selection may be achieved by carrying out positive and/or negative selection steps simultaneously, for example in a closed system where the entire process is carried out in the same tube or tubing set. In some embodiments, a sample containing target cells is subjected to a parallel selection in which the sample is load onto two or more chromatography columns, where each column effects selection of a cell population. In some embodiments, the two or more chromatography columns effect selection of CD3+, CD4+, or CD8+ populations individually. In some embodiments, the two or more chromatography columns, including affinity chromatography or gel permeation chromatography, independently effect selection of the same cell population. For example, the two or more chromatography columns may effect selection of CD3+ cells. In some embodiments, the two or more chromatography columns, including affinity chromatography or gel permeation chromatography, independently effect selection of different cell populations. For example, the two or more chromatography columns independently may effect selection of CD3+ cells, CD4+ cells, and CD8+ cells. In some embodiments, a further selection or selections, for example using sequential selection techniques, can be effected to enrich for sub-populations of one or all cell populations selected via parallel selection. For example, selected cells may be further selected for central memory T (T_{CM}) cells, naïve T cells, and/or cells positive for or expressing high levels of one or more surface markers, e.g., CD28+, CD62L+, CCR7+, CD27+, CD127+, CD4+, CD8+, CD45RA+, and/or CD45RO+. In some embodiments, a sample containing target cells is subjected to a parallel selection in which parallel selection is effected to enrich for a CD3+ population on the two or more columns. In some embodiments, a further selection or selections can be effected to enrich for sub-populations of the CD3⁺ population, for example, central memory T (T_{CM}) cells, naïve T cells, and/or cells positive for or expressing high levels of one or more surface markers, e.g., CD28+, CD62L+, CCR7+, CD27+, CD127+, CD4+, CD8+, CD45RA+, and/or CD45RO+. In some embodiments, a sample containing target cells is subjected to a parallel selection in which a selection is effected to enrich for a CD3+ population and a CD4+ population on the two or more columns, independently. In some embodiments, a further selection or selections can be effected to enrich for sub-populations of the CD3+ and CD4+ populations, for example, central memory T (T_{CM}) cells, naïve T cells, and/or cells positive for or expressing high levels of one or more surface markers, e.g., CD28+, CD62L+, CCR7+, CD27+, CD127+, CD4+, CD8+, CD45RA+, and/or CD45RO+. In some embodiments, a sample containing target cells is subjected to a parallel selection in which parallel selection is effected to enrich for a CD3+ population and a CD8+ population. In some embodiments, a further selection or selections can be effected to enrich for sub-populations of the CD3+ and CD8+ populations, for example, central memory T (T_{CM}) cells, naïve T cells, and/or cells positive for or expressing high levels of one or more surface markers, e.g., CD28+, CD62L+, CCR7+, CD27+, CD127+, CD4+, CD8+, CD45RA+, and/or CD45RO+. In some embodiments, a sample containing target cells is subjected to a parallel selection in which parallel selection is effected to enrich for a CD4+ population and a CD8+ population. In some embodiments, a further selection or selections can be effected to enrich for sub-populations of the CD4+ and CD8+ populations, for example, central memory T (T_{CM}) cells, naïve T cells, and/or cells positive for or expressing high levels of one or more surface markers, e.g., CD28+, CD62L+, CCR7+, CD27+, CD127+, CD4+, CD8+, CD45RA+, and/or CD45RO+. It is contemplated that in some aspects, specific subpopulations of T cells (e.g., CD3+, CD4+, CD8+ T cells), such as cells positive or expressing high levels of one or more surface markers, e.g., CD28+, CD62L+, CCR7+, CD27+, CD127+, CD4+, CD8+, CD45RA+, and/or CD45RO+ T cells, are selected by positive or negative parallel selection techniques. In some embodiments, sequential and parallel selection techniques can be used in combination.

2. Activation/Stimulation

In some embodiments, the cells are incubated and/or cultured prior to or in connection with genetic engineering. The incubation steps can include culture, stimulation, activation, and/or propagation. The incubation and/or engineering may be carried out in a culture vessel, such as a unit, chamber, well, column, tube, tubing set, valve, vial, culture dish, bag, or other container for culture. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

In particular embodiments, the stimulatory reagent contains an oligomeric reagent, e.g., a streptavidin mutein reagent, that is conjugated, linked, or attached to one or more agent, e.g., ligand, which is capable of activating an intracellular signaling domain of a TCR complex. In some embodiments, the one or more agents have an attached binding domain or binding partner (e.g., a binding partner C) that is capable of binding to oligomeric reagent at a particular binding sites (e.g., binding site Z). In some embodiments, a plurality of the agent is reversibly bound to the oligomeric reagent. In various embodiments, the oligomeric reagent has a plurality of the particular binding sites which, in certain embodiments, are reversibly bound to a plurality of agents at the binding domain (e.g., binding partner C). In some embodiments, the amount of bound agents are reduced or decreased in the presence of a competition reagent, e.g., a reagent that is also capable of binding to the particular binding sites (e.g., binding site Z).

In some embodiments, the stimulatory reagent is or includes a reversible systems in which at least one agent (e.g., an agent that is capable of producing a signal in a cell such as a T cell) is associated, e.g., reversibly associated, with the oligomeric reagent. In some embodiments, the reagent contains a plurality of binding sites capable of binding, e.g., reversibly binding, to the agent. In some cases, the reagent is a oligomeric particle reagent having at least one attached agent capable of producing a signal in a cell such as a T cell. In some embodiments, the agent contains at least one binding site, e.g., a binding site B, that can specifically bind an epitope or region of the molecule and also contains a binding partner, also referred to herein as a binding partner C, that specifically binds to at least one binding site of the reagent, e.g., binding site Z of the reagent. In some embodiments, the binding interaction between the binding partner C and the at least one binding site Z is a non-covalent interaction. In some cases, the binding interaction between the binding partner C and the at least one binding site Z is a covalent interaction. In some embodiments, the binding interaction, such as non-covalent interaction, between the binding partner C and the at least one binding site Z is reversible.

Substances that may be used as oligomeric reagents in such reversible systems are known, see e.g., U.S. Pat. Nos. 5,168,049; 5,506,121; 6,103,493; 7,776,562; 7,981,632; 8,298,782; 8,735,540; 9,023,604; and International published PCT Appl. Nos. WO2013/124474 and WO2014/076277. Non-limiting examples of reagents and binding partners capable of forming a reversible interaction, as well as substances (e.g. competition reagents) capable of reversing such binding, are described below.

In some embodiments, the oligomeric reagent is an oligomer of streptavidin, streptavidin mutein or analog, avidin, an avidin mutein or analog (such as neutravidin) or a mixture thereof, in which such oligomeric reagent contains one or more binding sites for reversible association with the binding domain of the agent (e.g., a binding partner C). In some embodiments, the binding domain of the agent can be a biotin, a biotin derivative or analog, or a streptavidin-binding peptide or other molecule that is able to specifically bind to streptavidin, a streptavidin mutein or analog, avidin or an avidin mutein or analog.

In certain embodiments, one or more agents (e.g., agents that are capable of producing a signal in a cell such as a T cell) associate with, such as are reversibly bound to, the oligomeric reagent, such as via the plurality of the particular binding sites (e.g., binding sites Z) present on the oligomeric reagent. In some cases, this results in the agents being closely arranged to each other such that an avidity effect can take place if a target cell having (at least two copies of) a cell surface molecule that is bound by or recognized by the agent is brought into contact with the agent.

In some embodiments, the oligomeric reagent is a streptavidin oligomer, a streptavidin mutein oligomer, a streptavidin analog oligomer, an avidin oligomer, an oligomer composed of avidin mutein or avidin analog (such as neutravidin) or a mixture thereof. In particular embodiments, the oligomeric reagents contain particular binding sites that are capable of binding to a binding domain (e.g., the binding partner C) of an agent. In some embodiments, the binding domain can be a biotin, a biotin derivative or analog, or a streptavidin-binding peptide or other molecule that is able to specifically bind to streptavidin, a streptavidin mutein or analog, avidin or an avidin mutein or analog. In some embodiments, the streptavidin can be wild-type streptavidin, streptavidin muteins or analogs, such as streptavidin-like polypeptides. Likewise, avidin, in some aspects, includes wild-type avidin or muteins or analogs of avidin such as neutravidin, a deglycosylated avidin with modified arginines that typically exhibits a more neutral pi and is available as an alternative to native avidin. Generally, deglycosylated, neutral forms of avidin include those commercially available forms such as “Extravidin”, available through Sigma Aldrich, or “NeutrAvidin” available from Thermo Scientific or Invitrogen, for example

In some embodiments, the reagent is a streptavidin or a streptavidin mutein or analog. In some embodiments, wild-type streptavidin (wt-streptavidin) has the amino acid sequence disclosed by Argarana et al, Nucleic Acids Res. 14 (1986) 1871-1882 (SEQ ID NO: 68). In general, streptavidin naturally occurs as a tetramer of four identical subunits, i.e. it is a homo-tetramer, where each subunit contains a single binding site for biotin, a biotin derivative or analog or a biotin mimic. An exemplary sequence of a streptavidin subunit is the sequence of amino acids set forth in SEQ ID NO: 68, but such a sequence also can include a sequence present in homologs thereof from other Streptomyces species. In particular, each subunit of streptavidin may exhibit a strong binding affinity for biotin with a dissociation constant (K_{d}) on the order of about 10⁻¹⁴ M. In some cases, streptavidin can exist as a monovalent tetramer in which only one of the four binding sites is functional (Howarth et al. (2006) Nat. Methods, 3:267-73; Zhang et al. (2015) Biochem. Biophys. Res. Commun., 463:1059-63)), a divalent tetramer in which two of the four binding sites are functional (Fairhead et al. (2013) J. Mol. Biol., 426:199-214), or can be present in monomeric or dimeric form (Wu et al. (2005) J. Biol. Chem., 280:23225-31; Lim et al. (2010) Biochemistry, 50:8682-91).

In some embodiments, streptavidin may be in any form, such as wild-type or unmodified streptavidin, such as a streptavidin from a Streptomyces species or a functionally active fragment thereof that includes at least one functional subunit containing a binding site for biotin, a biotin derivative or analog or a biotin mimic, such as generally contains at least one functional subunit of a wild-type streptavidin from Streptomyces avidinii set forth in SEQ ID NO: 68 or a functionally active fragment thereof. For example, in some embodiments, streptavidin can include a fragment of wild-type streptavidin, which is shortened at the N- and/or C-terminus. Such minimal streptavidins include any that begin N-terminally in the region of amino acid positions 10 to 16 of SEQ ID NO: 68 and terminate C-terminally in the region of amino acid positions 133 to 142 of SEQ ID NO: 68. In some embodiments, a functionally active fragment of streptavidin contains the sequence of amino acids set forth in SEQ ID NO: 69. In some embodiments, streptavidin, such as set forth in SEQ ID NO: 69, can further contain an N-terminal methionine at a position corresponding to Ala13 with numbering set forth in SEQ ID NO: 68. Reference to the position of residues in streptavidin or streptavidin muteins is with reference to numbering of residues in SEQ ID NO: 68.

Examples of streptavidins or streptavidin muteins are mentioned, for example, in WO 86/02077, DE 19641876 A1, U.S. Pat. No. 6,022,951, WO 98/40396 or WO 96/24606. Examples of streptavidin muteins are known in the art, see e.g., U.S. Pat. Nos. 5,168,049; 5,506,121; 6,022,951; 6,156,493; 6,165,750; 6,103,493; or 6,368,813; or International published PCT App. No. WO2014/076277.

In some embodiments, a streptavidin mutein can contain amino acids that are not part of an unmodified or wild-type streptavidin or can include only a part of a wild-type or unmodified streptavidin. In some embodiments, a streptavidin mutein contains at least one subunit that can have one more amino acid substitutions (replacements) compared to a subunit of an unmodified or wild-type streptavidin, such as compared to the wild-type streptavidin subunit set forth in SEQ ID NO: 68 or a functionally active fragment thereof, e.g. set forth in SEQ ID NO: 69.

In some embodiments, the binding affinity, such as dissociation constant (K_{d}), of streptavidin or a streptavidin mutein for a binding domain is less than 1×10⁻⁴ M, 5×10⁻⁴ M, 1×10⁻⁵ M, 5×10⁻⁵ M, 1×10⁻⁶ M, 5×10⁻⁶ M or 1×10⁻⁷ M, but generally greater than 1×10⁻¹³ M, 1×10⁻¹² M or 1×10⁻¹¹ M. For example, peptide sequences (e.g., Strep-tags), such as disclosed in U.S. Pat. No. 5,506,121, can act as biotin mimics and demonstrate a binding affinity for streptavidin, e.g., with a K_{d} of approximately between 10⁻⁴ and 10⁻⁵ M. In some cases, the binding affinity can be further improved by making a mutation within the streptavidin molecule, see e.g. U.S. Pat. No. 6,103,493 or WO2014/076277. In some embodiments, binding affinity can be determined by methods known in the art, such as any described herein.

In some embodiments, the reagent, such as a streptavidin or streptavidin mutein, exhibits binding affinity for a peptide ligand binding partner, which peptide ligand binding partner can be the binding partner C present in the agent (e.g., receptor-binding agent or selection agent). In some embodiments, the peptide sequence contains a sequence with the general formula His-Pro-Xaa, where Xaa is glutamine, asparagine, or methionine, such as contains the sequence set forth in SEQ ID NO: 71. In some embodiments, the peptide sequence contains a sequence set forth in SEQ ID NO: 70. In some embodiments, the peptide sequence has the general formula set forth in SEQ ID NO: 72, such as set forth in SEQ ID NO: 73. In one example, the peptide sequence is Trp-Arg-His-Pro-Gln-Phe-Gly-Gly (also called Strep-tag®, set forth in SEQ ID NO: 74). In one example, the peptide sequence is Trp-Ser-His-Pro-Gln-Phe-Glu-Lys (also called Strep-tag® II, set forth in SEQ ID NO: 75). In some embodiments, the peptide ligand contains a sequential arrangement of at least two streptavidin-binding modules, wherein the distance between the two modules is at least 0 and not greater than 50 amino acids, wherein one binding module has 3 to 8 amino acids and contains at least the sequence His-Pro-Xaa, where Xaa is glutamine, asparagine, or methionine, and wherein the other binding module has the same or different streptavidin peptide ligand, such as set forth in SEQ ID NO: 72 (see e.g. International Published PCT Appl. No. WO02/077018; U.S. Pat. No. 7,981,632). In some embodiments, the peptide ligand contains a sequence having the formula set forth in any of SEQ ID NO: 76 or 77. In some embodiments, the peptide ligand has the sequence of amino acids set forth in any of SEQ ID NOS: 78-80, and 81-82. In most cases, all these streptavidin binding peptides bind to the same binding site, namely the biotin binding site of streptavidin. If one or more of such streptavidin binding peptides is used as binding partners C, e.g. C1 and C2, the multimerization reagent and/or oligomeric particle reagents bound to the one or more agents via the binding partner C is typically composed of one or more streptavidin muteins.

In some embodiments, the streptavidin mutein is a mutant as described in U.S. Pat. No. 6,103,493. In some embodiments, the streptavidin mutein contains at least one mutation within the region of amino acid positions 44 to 53, based on the amino acid sequence of wild-type streptavidin, such as set forth in SEQ ID NO: 68. In some embodiments, the streptavidin mutein contains a mutation at one or more residues 44, 45, 46, and/or 47. In some embodiments, the streptavidin mutein contains a replacement of Glu at position 44 of wild-type streptavidin with a hydrophobic aliphatic amino acid, e.g. Val, Ala, Ile or Leu, any amino acid at position 45, an aliphatic amino acid, such as a hydrophobic aliphatic amino acid at position 46 and/or a replacement of Val at position 47 with a basic amino acid, e.g. Arg or Lys, such as generally Arg. In some embodiments, Ala is at position 46 and/or Arg is at position 47 and/or Val or Ile is at position 44. In some embodiments, the streptavidin mutant contains residues Val44-Thr45-Ala46-Arg47, such as set forth in exemplary streptavidin muteins containing the sequence of amino acids set forth in SEQ ID NO: 83 or SEQ ID NO: 84 or 85 (also known as streptavidin mutant 1, SAM1). In some embodiments, the streptavidin mutein contains residues Ile44-Gly45-Ala46-Arg47, such as set forth in exemplary streptavidin muteins containing the sequence of amino acids set forth in SEQ ID NO: 86, 87, or 59 (also known as SAM2). In some cases, such streptavidin mutein are described, for example, in U.S. Pat. No. 6,103,493, and are commercially available under the trademark Strep-Tactin®. In some embodiments, the mutein streptavidin contains the sequence of amino acids set forth in SEQ ID NO: 88 or SEQ ID NO: 89. In particular embodiments, the molecule is a tetramer of streptavidin or a streptavidin mutein comprising a sequence set forth in any of SEQ ID NOS: 69, 84, 87, 88, 90, 85 or 59, which, as a tetramer, is a molecule that contains 20 primary amines, including 1 N-terminal amine and 4 lysines per monomer.

In some embodiments, streptavidin mutein exhibits a binding affinity characterized by a dissociation constant (K_{d}) that is or is less than 3.7×10⁻⁵ M for the peptide ligand (Trp-Arg-His-Pro-Gln-Phe-Gly-Gly; also called Strep-tag®, set forth in SEQ ID NO: 74) and/or that is or is less than 7.1×10⁻⁵ M for the peptide ligand (Trp-Ser-His-Pro-Gln-Phe-Glu-Lys; also called Strep-tag® II, set forth in SEQ ID NO: 75) and/or that is or is less than 7.0×10⁻⁵ M, 5.0×10⁻⁵ M, 1.0×10⁻⁵ M, 5.0×10⁻⁶ M, 1.0×10⁻⁶ M, 5.0×10⁻⁷ M, or 1.0×10⁻⁷ M, but generally greater than 1×10⁻¹³ M, 1×10⁻¹² M or 1×10⁻¹¹ M for any of the peptide ligands set forth in any of SEQ ID NOS: 75, 76-77, 81-82, 78-80, 73, 74, 70, 72.

In some embodiments, the resulting streptavidin mutein exhibits a binding affinity characterized by an association constant (Kₐ) that is or is greater than 2.7×10⁴ M⁻¹ for the peptide ligand (Trp-Arg-His-Pro-Gln-Phe-Gly-Gly; also called Strep-tag®, set forth in SEQ ID NO: 74) and/or that is or is greater than 1.4×10⁴ M⁻¹ for the peptide ligand (Trp-Ser-His-Pro-Gln-Phe-Glu-Lys; also called Strep-tag® II, set forth in SEQ ID NO: 75) and/or that is or is greater than 1.43×10⁴ M⁻¹, 1.67×10⁴ M⁻¹, 2×10⁴ M⁻¹, 3.33×10⁴ M⁻¹, 5×10⁴ M⁻¹, 1×10⁵ M⁻¹, 1.11×10⁵ M⁻¹, 1.25×10⁵ M⁻¹, 1.43×10⁵ M⁻¹, 1.67×10⁵ M⁻¹, 2×10⁵ M⁻¹, 3.33×10⁵ M⁻¹, 5×10⁵ M⁻¹, 1×10⁶ M⁻¹, 1.11×10⁶ M⁻¹, 1.25×10⁶ M⁻¹, 1.43×10⁶ M⁻¹, 1.67×10⁶ M⁻¹, 2×10⁶ M⁻¹, 3.33×10⁶ M⁻¹, 5×10⁶ M⁻¹, 1×10⁷ M⁻¹, but generally less than 1×10¹³ M⁻¹, 1×10¹² M⁻¹ or 1×10¹¹ M⁻¹ for any of the peptide ligands set forth in any of SEQ ID NOS: 75, 76-77, 81-82, 78-80, 73, 74, 70, 72.

In particular embodiments, provided herein is an oligomeric particle reagent that is composed of and/or contains a plurality of streptavidin or streptavidin mutein tetramers. In certain embodiments, the oligomeric particle reagent provided herein contains a plurality of binding sites that reversibly bind or are capable of reversibly binding to one or more agents, e.g., a stimulatory agent and/or a selection agent. In some embodiments, the oligomeric particle has a radius, e.g., an average radius, of between 70 nm and 125 nm, inclusive; a molecular weight of between 1×10⁷ g/mol and 1×10¹ g/mol, inclusive; and/or between 1,000 and 5,000 streptavidin or streptavidin mutein tetramers, inclusive. In some embodiments, the oligomeric particle reagent is bound, e.g., reversibly bound, to one or more agents such as an agent that binds to a molecule, e.g. receptor, on the surface of a cell. In certain embodiments, the one or more agents are agents described herein, e.g., in Section II-C-2. In some embodiments, the agent is an anti-CD3 and/or an anti-CD28 antibody or antigen binding fragment thereof, such as an antibody or antigen fragment thereof that contains a binding partner, e.g., a streptavidin binding peptide, e.g. Strep-tag® II. In particular embodiments, the one or more agents bind to a cell surface receptor and/or an accessory molecule to stimulate the cell, and may include an antibody targeting the TCR complex or a component thereof, an antibody targeting a co-stimulatory molecule, anti-CD3 antibodies, anti-CD28 antibodies, or an anti-CD3 and/or an anti CD28 Fab), and the one or more agents contain a binding partner, e.g., a streptavidin binding peptide, e.g. Strep-tag® II. In particular embodiments, the one or more agents comprise a streptavidin-based oligomer, such as a streptavidin mutein oligomer, conjugated to Strep-tagged anti-CD3 and Strep-tagged anti-CD28 Fabs. In some embodiments, the oligomeric particle reagent is any as described in WO2015/158868 or WO2018/197949.

In particular embodiments, an oligomeric reagent is prepared by polymerizing an exemplary streptavidin mutein designated STREP-TACTIN® M2 (see e.g. U.S. Pat. No. 6,103,493 and Voss and Skerra (1997) Protein Eng., 1:975-982, and Argarana et al. (1986) Nucleic Acids Research, 1871-1882). In particular embodiments, to prepare streptavidin muteins for oligomerization, streptavidin muteins containing one or more reactive thiol groups are incubated with maleimide activated streptavidin muteins. In particular embodiments, to prepare the thiolated streptavidin mutein, about 100 mg of streptavidin mutein is thiolated by incubation with 2-iminothiolane hydrochloride at a molar ratio of 1:100 at a pH of about 8.5 at 24° C. for 1 hour in 100 mM Borate buffer in a total volume of 2.6 mL. For the activation reaction, about 400 mg of streptavidin mutein is incubated with Succinimidyl-6-[(β-maleimidopropionamido) hexanoate (SMPH) at a molar ratio of 1:2 at a pH of about 7.2 at 24° C. for 1 hour in a total volume of about 10.4 mL in a sodium phosphate buffer. The thiolation and activation reactions are coordinated to start at about the same time, and the duration of the reactions is controlled. After the reactions, the 2-Iminothiolane hydrochloride and SMPH are removed from the samples by individually carrying out gel filtration of the samples with PD-10 desalting columns (GE Healthcare). For each 2.5 mL volume of sample, a 1 mL PD-10 column is equilibrated and loaded with either thiolated mutein streptavidin or maleimide mutein streptavidin and elution is carried out by adding 3.5 mL of coupling buffer (100 mM NaH₂PO₄, 150 mM NaCl, 5 mM EDTA, pH 7.2). Gel filtration of the maleimide mutein streptavidin is carried out on 4 columns to account for the >10 mL volume and eluates are pooled. The timing of the activation and thiolation reactions and the timing between the end of the activation and thiolation reactions and the start of the oligomerization reactions are controlled. Generally, no more than ten minutes is allowed to pass from the start of gel filtrations, i.e. the end of the activation and thiolation reactions, to when oligomerization reaction is initiated.

In particular embodiments, the maleimide streptavidin mutein and thiolated streptavidin mutein samples are then combined into an overall volume of about 17.5 mL and incubated for 1 hour at a pH of 7.2 at 24° C. under stirring conditions at about 600 rpm. Because four times more streptavidin mutein was incubated with SMPH than with 2-iminothiolane hydrochloride, the molar ratio of thiolated streptavidin mutein and maleimide streptavidin mutein is 1:4 during the oligomerization reaction. After the reaction, remaining SH groups of the oligomerized streptavidin mutein reagent are saturated by incubation with N-Ethylmaleimide (NEM) for 15 min at 24° C. with stirring (about 600 rpm) followed by incubation for a further 16-20 hours at 4° C. After incubation with NEM, the sample containing oligomerized streptavidin mutein is centrifuged and the supernatant is filtered through a 0.45 μm membrane (Millex-HP 0.45 μm from Merck Millopore). The filtered solution is then loaded into a column (Sephacryl 5-300 HR HiPrep 26/60, GE Healthcare) for size exclusion chromatography (SEC) with an AKTA Explorer chromatography system (GE Healthcare). Fractions with a milli absorbance unit (mAU) greater than or equal to 1500 mAU are pooled. The pooled sample containing oligomeric streptavidin mutein is treated with 100 mM hydroxylamine at a pH of 6.35 for 15 minutes at room temperature. To remove the hydroxylamine after treatment, sample is loaded onto a PD10 column (2.5 mL per column) and eluted with 3.5 mL of buffer containing 100 mM NaH₂PO₄, 140 mM NaCl, 1 mM EDTA, pH 7.2. The PD10 elutes are pooled and sterile filtered with a 0.45 μm filter followed by a 0.22 μm filter and then samples are frozen and stored at −80° C. Prior to freezing, the final concentration of the oligomeric streptavidin mutein reagent is measured and the size of the oligomeric streptavidin mutein reagent is determined by dynamic light scattering (DLS).

In some embodiments, stimulatory agents such as an anti-CD3 antibody and an anti-CD28 Fab antibody were multimerized by reversible binding to the oligomeric streptavidin mutein reagent. In some embodiments, the stimulatory agents, e.g., anti-CD3 and anti-CD28 Fab fragments, are reversibly bound to the streptavidin mutein oligomer via a streptavidin peptide-binding partner fused to each stimulatory agent, e.g. each Fab fragment. In some embodiments, the anti-CD3 Fab fragment is derived from the CD3 binding monoclonal antibody produced by the hybridoma cell line OKT3 (ATCC® CRL-8001™; see also U.S. Pat. No. 4,361,549), and contains the heavy chain variable domain and light chain variable domain of the anti-CD3 antibody OKT3 described in Arakawa et al J. Biochem. 120, 657-662 (1996). These sequences are set forth in SEQ ID NOs: 60 and 61, respectively. In some embodiments, the anti-CD28 Fab fragment is derived from antibody CD28.3 (deposited as a synthetic single chain Fv construct under GenBank Accession No. AF451974.1; see also Vanhove et al., BLOOD, 15 Jul. 2003, Vol. 102, No. 2, pages 564-570) and contains the heavy and light chain variable domains of the anti-CD28 antibody CD28.3 set forth in SEQ ID NOS: 62 and 63, respectively. For exemplary peptide-tagged Fab fragments, see International Patent App. Pub. Nos. WO 2013/011011 and WO 2013/124474.

In some embodiments, provided herein is an oligomeric particle reagent that is composed of and/or contains a plurality of streptavidin or streptavidin mutein tetramers. In certain embodiments, the oligomeric particle reagent provided herein contains a plurality of binding sites that reversibly bind or are capable of reversibly binding to one or more agents, e.g., a stimulatory agent and/or a selection agent. In some embodiments, the oligomeric particle has a radius, e.g., an average radius, of between 80 nm and 120 nm, inclusive; a molecular weight, e.g., an average molecular weight of between 7.5×10⁶ g/mol and 2×10⁸ g/mol, inclusive; and/or an amount, e.g., an average amount, of between 500 and 10,000 streptavidin or streptavidin mutein tetramers, inclusive. In some embodiments, the oligomeric particle reagent is bound, e.g., reversibly bound, to one or more agents, such as an agent that binds to a molecule, e.g. receptor, on the surface of a cell. In some embodiments, the agent comprises one or more agents that bind to a cell surface receptor and/or an accessory molecule to stimulate the cell (e.g., such as an antibody targeting the TCR complex or a component thereof, an antibody targeting a co-stimulatory molecule, anti-CD3 antibodies, anti-CD28 antibodies, or anti-CD3/anti-CD28 Fabs). In some embodiments, the agent is an anti-CD3 and/or an anti-CD28 Fab, such as a Fab that contains a binding partner, e.g., a streptavidin binding peptide, e.g. Strep-tag® II. In particular embodiments, the one or more agents is an anti-CD3 and/or an anti CD28 Fab containing a binding partner, e.g., a streptavidin binding peptide, e.g. Strep-tag® II.

In some embodiments, the cells are stimulated or subjected to stimulation in the presence of, of about, or of at least 0.01 μg, 0.02 μg, 0.03 μg, 0.04 μg, 0.05 μg, 0.1 μg, 0.2 μg, 0.3 μg, 0.4 μg, 0.5 μg, 0.75 μg, 1 μg, 1.2 μg, 1.4 μg, 1.6 μg, 1.8 μg, 2 μg, 3 μg, 4 μg, 5 μg, 6 μg, 7 μg, 8 μg, 9 μg, or 10 μg of the oligomeric stimulatory reagent (e.g., the streptavidin-based oligomer, such as a streptavidin mutein oligomer, conjugated to Strep-tagged anti-CD3 and Strep-tagged anti-CD28 Fabs) per 10⁶ cells. In some embodiments, the cells are stimulated or subjected to stimulation in the presence of or of about 4 μg of the oligomeric stimulatory reagent (e.g., the streptavidin-based oligomer, such as a such as a streptavidin mutein oligomer, conjugated to Strep-tagged anti-CD3 and Strep-tagged anti-CD28 Fabs) per 10⁶ cells. In particular embodiments, the cells are stimulated or subjected to stimulation in the presence of or of about 1.2 μg of the oligomeric stimulatory reagent (e.g., the streptavidin-based oligomer, such as a streptavidin mutein oligomer, conjugated to Strep-tagged anti-CD3 and Strep-tagged anti-CD28 Fabs) per 10⁶ cells. In particular embodiments, the cells are stimulated or subjected to stimulation in the presence of or of about 0.8 μg of the oligomeric stimulatory reagent (e.g., the streptavidin-based oligomer, such as a streptavidin mutein oligomer, conjugated to Strep-tagged anti-CD3 and Strep-tagged anti-CD28 Fabs) per 10⁶ cells. In particular embodiments, the cells are stimulated or subjected to stimulation in the presence of or of about 1.8 μg of the oligomeric stimulatory reagent (e.g., the streptavidin-based oligomer, such as a streptavidin mutein oligomer, conjugated to Strep-tagged anti-CD3 and Strep-tagged anti-CD28 Fabs) per 10⁶ cells. In certain aspects, within the oligomeric stimulatory reagent, the mass ratio between the oligomeric particles and the attached agents is about 3:1. In certain aspects, within the oligomeric stimulatory reagent, the mass ratio among the oligomeric particles, the attached anti-CD3 Fabs, and the attached anti-CD28 Fabs is about 3:0.5:0.5. In certain aspects, 4 μg of the oligomeric stimulatory reagent is or includes 3 μg of oligomeric particles and 1 μg of attached agents, e.g., 0.5 μg of anti-CD3 Fabs and 0.5 μg of anti-CD28 Fabs. In other examples, 1.2 μg of the oligomeric stimulatory reagent per 10⁶ cells is or includes 0.9 μg of oligomeric particles and 0.3 μg of attached agents, e.g., 0.15 μg of anti-CD3 Fabs and 0.15 μg of anti-CD28 Fabs, per 10⁶ cells. In some embodiments, the oligomeric stimulatory reagent is added to a serum-free medium and the stimulation is performed in the serum free medium, e.g., as described in PCT/US2018/064627.

In particular embodiments, an amount of or of about 900×10⁶ T cells (e.g., 900×10⁶ CD3+ T cells, or 450×10⁶ CD4⁺ T cells and 450×10⁶ CD8⁺ T cells) of the input population are subjected to stimulation, e.g., cultured under stimulating conditions, in the presence of the oligomeric stimulatory reagent (e.g., the streptavidin-based oligomer, such as a streptavidin mutein oligomer, conjugated to Strep-tagged anti-CD3 and Strep-tagged anti-CD28 Fabs). In certain embodiments, the cells, e.g., cells of the input population, are stimulated or subjected to stimulation e.g., cultured under stimulating conditions such as in the presence of a stimulatory reagent, at a density of, of about, or at least 0.01×10⁶ cells/mL, 0.1×10⁶ cells/mL, 0.5×10⁶ cells/mL, 1.0×10⁶ cells/mL, 1.5×10⁶ cells/mL, 2.0×10⁶ cells/mL, 2.5×10⁶ cells/mL, 3.0×10⁶ cells/mL, 4.0×10⁶ cells/mL, 5.0×10⁶ cells/mL, 10×10⁶ cells/mL, or 50×10⁶ cells/mL. In certain embodiments, the cells, e.g., cells of the input population, are stimulated or subjected to stimulation e.g., cultured under stimulating conditions such as in the presence of a stimulatory reagent, at a density of, of about, or at least 3.0×10⁶ cells/mL.

In some embodiments, an output population, e.g., a population of engineered T cells, is generated by steps that include: incubating an input population of or containing T cells with a oligomeric stimulatory particle reagent, e.g., an oligomer-based stimulatory reagent described herein, for between or between about 18 and 30 hours, inclusive; introducing a heterologous or recombinant polynucleotide encoding a recombinant receptor into T cells of the stimulated population, (iii) incubating the cells under static conditions, (iv) removing or separating the stimulatory reagents from the cells by adding a competition reagent, and (v) collecting or harvesting the incubated cells.

In certain embodiments, an output population, e.g., a population of engineered T cells, is generated by steps that include: incubating an input population comprising T cells under stimulating conditions for between 18 and 30 hours, inclusive, in the presence of a streptavidin mutein oligomer with reversibly attached to one or more agents that bind to a cell surface receptor and/or an accessory molecule to stimulate the cell (e.g., an antibody targeting the TCR complex or a component thereof, an antibody targeting a co-stimulatory molecule, anti-CD3 antibodies, anti-CD28 antibodies, or anti-CD3/anti-CD28 Fabs); transducing the stimulated T cells with a viral vector encoding a recombinant receptor, such as by spinoculating the stimulated T cells in the presence of the viral vector, and then incubating the transduced T cells under static conditions for between or between about 42 hours and 84 hours, inclusive; and harvesting or collecting the T cells.

In some embodiments, the provided methods for producing a population of engineered cells include one or more of stimulating an input population of T cells in the presence of oligomeric streptavidin mutein with reversibly attached anti-CD3/anti-CD28 Fabs in an amount of between or between about 0.4 μg and 8 μg per 10⁶ cells, inclusive, e.g., 1.2 μg per 10⁶ cells, in serum free media containing recombinant IL-2, IL-7, and IL-15 for between 18 and 30 hours, inclusive; transducing the cells with a viral vector encoding a recombinant receptor by first spinoculating the cells in the presence of the viral vector 30 minutes at a force of 693 g and then incubating the spinoculated cells with the viral vector for between 24 hours and 96 hours, inclusive; adding biotin (e.g., D-biotin) to the cells to remove or separate the oligomeric streptavidin mutein with reversibly attached anti-CD3/anti-CD28 Fabs from the cells; and collecting or harvesting the cells.

In some embodiments, the cells are harvested or collected from between or between about 36 hours and 96 hours, inclusive, from the initiation of the stimulation. In various embodiments, the cells are harvested or collected between 36 hours and 108 hours or 48 hours and 96 hours, inclusive, after the initiation of the stimulation. In particular embodiments, the oligomeric streptavidin mutein with reversibly attached anti-CD3/anti-CD28 Fabs are removed or separated from the cells between 36 hours and 96 hours or 48 hours and 72 hours, inclusive, after the initiation of the stimulation.

In some embodiments, the oligomeric streptavidin mutein with reversibly attached anti-CD3/anti-CD28 Fab are removed or separated (e.g. as described in Section II-C-6) from the cells after or after about 48 hours e.g., 48 hours±6 hours from the initiation of the stimulation. In particular embodiments, the oligomeric streptavidin mutein with reversibly attached anti-CD3/anti-CD28 Fabs are removed or separated from the cells after or after about 72 hours, e.g., 72 hours±6 hours, from the initiation of the stimulation. In particular embodiments, the oligomeric streptavidin mutein with reversibly attached anti-CD3/anti-CD28 Fabs are removed or separated from the cells after or after about 96 hours, e.g., 96 hours±6 hours, from the initiation of the stimulation. In particular embodiments, the oligomeric streptavidin mutein with reversibly attached anti-CD3/anti-CD28 Fabs are removed or separated from the cells after the incubation, and cells are collected or harvested after the addition of biotin or a biotin analogue. In certain embodiments, the oligomeric streptavidin mutein with reversibly attached anti-CD3/anti-CD28 Fabs are removed or separated from the cells during the incubation, such that the cells are returned to the incubation after the addition of the biotin or biotin analog.

In some embodiments, the incubation is performed in the presence of recombinant cytokines (e.g. IL-2, IL-7, and IL-15) in serum free media. In certain embodiments, the incubation is performed in the absence of recombinant cytokines. In particular embodiments, the incubation is performed in the presence of basal media. In certain embodiments, incubation in basal media increases the integration, e.g., stable integration of the heterologous or recombinant nucleotide, increases the percentage of cells expressing the recombinant receptor, improves potency, or reduces differentiation of the cells as compared to processes where cells stimulated with oligomeric stimulatory reagents are incubated in the presence of serum free media containing recombinant cytokines.

In particular embodiments, the removal of the oligomeric stimulatory reagent, e.g., the oligomeric streptavidin mutein with reversibly attached anti-CD3/anti-CD28 Fabs, such as by the addition of biotin or a biotin analogue, reduces the amount cell loss that can occur when stimulatory reagents are separated or removed from cells. In some embodiments, less than or less than about 30%, 25%, 20%, 15%, 10%, or 5% of the cells are lost, killed, or separated from the cell population when the oligomeric stimulatory reagent is separated or removed from the cells. In certain embodiments, output populations generated from processes that use oligomeric stimulatory reagents for stimulation have, have about, or have at least 15%, 20%, 25%, 30%, 35%, 40%, 45%, or 50% more total cells than output populations generated from processes that utilize alternative stimulatory reagents, such as antibody conjugated paramagnetic beads.

3. Vectors and Methods for Genetic Engineering

In some embodiments, engineered cells, such as T cells, used in connection with the provided methods, uses, articles of manufacture or compositions are cells have been genetically engineered to express a recombinant receptor, e.g., a CAR described herein. In some embodiments, the cells are engineered by introduction, delivery or transfer of nucleic acid sequences that encode the recombinant receptor and/or other molecules.

In some embodiments, methods for producing engineered cells includes the introduction of a polynucleotide encoding a recombinant receptor (e.g. anti-CD19 CAR) into a cell, e.g., such as a stimulated or activated cell. In particular embodiments, the recombinant proteins are recombinant receptors, such as any described. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation. In some embodiments, the engineering produces one or more engineered compositions of enriched T cells.

In some embodiments, the provided methods include genetically engineering the cells, e.g., introducing a heterologous or recombinant polynucleotide encoding a recombinant protein. Such recombinant proteins may include recombinant receptors, such as any described in Section II-A. Any method of introducing a heterologous or recombinant polynucleotide that would result in integration of the polynucleotide encoding the recombinant receptor into the genome of a cell such as a T cell may be used, including viral and non-viral methods of genetic engineering. Introduction of the polynucleotides, e.g., heterologous or recombinant polynucleotides, encoding the recombinant protein into the cell may be carried out using any of a number of known vectors. Such vectors include viral, including lentiviral and gammaretroviral, systems. Exemplary methods include those for transfer of heterologous polynucleotides encoding the receptors, including via viral, e.g., retroviral or lentiviral, transduction. In some embodiments, a population of stimulated cells is genetically engineered, such as to introduce a heterologous or recombinant polynucleotide encoding a recombinant receptor, thereby generating a population of transformed cells (also referred to herein as a transformed population of cells).

In some embodiments, the provided methods include genetically engineering the cells, e.g., introducing a heterologous or recombinant polynucleotide encoding a recombinant protein, using a non-viral method, such as electroporation, calcium phosphate transfection, protoplast fusion, cationic liposome-mediated transfection, nanoparticles such as lipid nanoparticles, tungsten particle-facilitated microparticle bombardment, strontium phosphate DNA co-precipitation, and other approaches described in, e.g., WO 2014055668, and U.S. Pat. No. 7,446,190. Transposon-based systems also are contemplated.

In particular embodiments, the cells are genetically engineered, transformed, or transduced after the cells have been stimulated, activated, and/or incubated under stimulating conditions, such as by any of the methods provided herein, e.g., in Section II. In particular embodiments, the one or more stimulated populations have been previously cryoprotected and stored, and are thawed and optionally washed prior to genetically engineering, transforming, transfecting, or transducing the cells.

In particular embodiments, the cells are genetically engineered, transformed, or transduced after the cells are stimulated or subjected to stimulation or cultured under stimulatory conditions. In particular embodiments, the cells are genetically engineered, transformed, or transduced at, at about, or within 72 hours, 60 hours, 48 hours, 36 hours, 24 hours, or 12 hours, inclusive, from the initiation of the stimulation. In particular embodiments, the cells are genetically engineered, transformed, or transduced at, at about, or within 3 days, two days, or one day, inclusive, from the initiation of the stimulation. In certain embodiments, the cells are genetically engineered, transformed, or transduced between or between about 12 hours and 48 hours, 16 hours and 36 hours, or 18 hours and 30 hours after the initiation of the stimulation. In particular embodiments, the cells are genetically engineered, transformed, or transduced between or between about 18 hours and 30 hours after the initiation of the stimulation. In particular embodiments, the cells are genetically engineered, transformed, or transduced at or at about 16 hours, 18 hours, 20 hours, 22 hours, or 24 hours after the initiation of the stimulation.

In certain embodiments, methods for genetic engineering are carried out by contacting or introducing one or more cells of a population with a nucleic acid molecule or polynucleotide encoding the recombinant protein, e.g. a recombinant receptor. In certain embodiments, the nucleic acid molecule or polynucleotide is heterologous to the cells. In particular embodiments, heterologous nucleic acid molecule or heterologous polynucleotide is not native to the cells. In certain embodiments, the heterologous nucleic acid molecule or heterologous polynucleotide encodes a protein, e.g., a recombinant protein, that is not natively expressed by the cell. In particular embodiments, the heterologous nucleic acid molecule or polynucleotide is or contains a nucleic acid sequence that is not found in the cell prior to the contact or introduction.

In some embodiments, the cells, e.g., stimulated cells, are engineered, e.g., transduced or in the presence of a transduction adjuvant. Exemplary transduction adjuvants include, but are not limited to, polycations, fibronectin or fibronectin-derived fragments or variants, and RetroNectin. In certain embodiments, the cells are engineered in the presence of polycations, fibronectin or fibronectin-derived fragments or variants, and/or RetroNectin. In particular embodiments, the cells are engineered in the presence of a polycation that is polybrene, DEAE-dextran, protamine sulfate, poly-L-lysine, or a cationic liposome. In particular embodiments, the cells are engineered in the presence of protamine sulfate. In some embodiments, the presence of an oligomeric stimulatory reagent, e.g., as described in Section II-C-2 can act as a transduction adjuvant, see, e.g., WO/2017/068419 which is incorporated herein by reference.

In some embodiments, the genetic engineering, e.g., transduction, is carried out in serum free media, e.g, as described herein or in PCT/US2018/064627. In some embodiments, the serum free media is a defined or well-defined cell culture media. In certain embodiments, the serum free media is a controlled culture media that has been processed, e.g., filtered to remove inhibitors and/or growth factors. In some embodiments, the serum free media contains proteins. In certain embodiments, the serum-free media may contain serum albumin, hydrolysates, growth factors, hormones, carrier proteins, and/or attachment factors.

In particular embodiments, the cells are engineered in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In particular embodiments, cells, e.g., stimulated cells are engineered under stimulating conditions in the presence of IL-2, IL-7, and/or IL-15. In certain embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15. In certain embodiments, the cells are engineered, e.g., transduced or under stimulating conditions in the presence of recombinant IL-2, IL-7, and IL-15, such as recombinant human IL-2 (e.g., 100 IU/mL), recombinant human IL-7 (e.g., 600 IU/mL), and/or recombinant human IL-15 (e.g., 100 IU/mL).

In some embodiments, the cells are genetically engineered, transformed, or transduced in the presence of the same or similar media as was present during the stimulation. In some embodiments, the cells are genetically engineered, transformed, or transduced in media having the same cytokines as the media present during stimulation. In certain embodiments, the cells are genetically engineered, transformed, or transduced, in media having the same cytokines at the same concentrations as the media present during stimulation.

In some embodiments, genetically engineering the cells is or includes introducing the polynucleotide, e.g., the heterologous or recombinant polynucleotide, into the cells by transduction. In some embodiments, the cells are transduced or subjected to transduction with a viral vector. In particular embodiments, the cells are transduced or subjected to transduction with a viral vector. In some embodiments, the virus is a retroviral vector, such as a gammaretroviral vector or a lentiviral vector. Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the transduction is carried out by contacting one or more cells of a population with a nucleic acid molecule encoding the recombinant protein, e.g. recombinant receptor. In some embodiments, the contacting can be effected with centrifugation, such as spinoculation (e.g. centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In particular embodiments, an amount of, of about, or of at least 50×10⁶, 100×10⁶, 150×10⁶, 200×10⁶, 250×10⁶, 300×10⁶, 350×10⁶, 400×10⁶, 450×10⁶, 500×10⁶, 550×10⁶, 600×10⁶, 700×10⁶, 800×10⁶, 900×10⁶, or 1,000×10⁶ cells of the composition that has been subjected to stimulation, e.g., cultured under stimulating conditions, are subjected to genetic engineering, e.g., transduction. In particular embodiments, the total number of cells, e.g., viable T cells comprising both CD4+ T cells and CD8+ T cells, that have been subjected to stimulation and are subsequently subjected to transduction is at or about 50×10⁶ cells, at or about 100×10⁶ cells, at or about 150×10⁶ cells, at or about 200×10⁶ cells, at or about 250×10⁶ cells, at or about 300×10⁶ cells, at or about 350×10⁶ cells, at or about 400×10⁶ cells, at or about 450×10⁶ cells, at or about 500×10⁶ cells, at or about 550×10⁶ cells, at or about 600×10⁶ cells, at or about 700×10⁶ cells, at or about 800×10⁶ cells, at or about 900×10⁶ cells, or at or about 1,000×10⁶ cells, or any value between any of the foregoing. In particular embodiments, up to 900×10⁶ cells of the input population are subjected to stimulation, and an amount of, of about, or up to 600×10⁶ cells of the cells that have been subjected to stimulation are subjected to genetic engineering, e.g., transduction. In particular embodiments, the cell composition subjected to genetic engineering, e.g., transduction, comprises viable CD4+ T cells and viable CD8+ T cells, at a ratio of between 1:10 and 10:1, between 1:5 and 5:1, between 4:1 and 1:4, between 1:3 and 3:1, between 2:1 and 1:2, between 1.5:1 and 1:1.5, between 1.25:1 and 1:1.25, between 1.2:1 and 1:1.2, between 1.1:1 and 1:1.1, or about 1:1, or 1:1 viable CD4+ T cells to viable CD8+ T cells.

In some embodiments, the provided methods are used in connection with transducing a viral vector containing a polynucleotide encoding a recombinant receptor into, into about, or into less than 300×10⁶ cells, e.g., viable T cells of a stimulated cell population. In certain embodiments, at or about 100×10⁶ cells, e.g., viable T cells of a stimulated cell population are transduced or subjected to transduction.

In some embodiments, the provided methods are used in connection with transducing a viral vector containing a polynucleotide encoding a recombinant receptor into, into about, or into less than 600×10⁶ cells, e.g., viable T cells of a stimulated cell population. In certain embodiments, at or about 600×10⁶ cells, e.g., viable T cells of a stimulated cell population are transduced or subjected to transduction. In some embodiments, up to 900×10⁶ cells (e.g., viable CD3+ cells or mixed viable CD4+ and viable CD8+ cells (e.g., mixed at or at about a 1:1 ratio)) are subjected to stimulation, and an amount of, of about, or up to 600×10⁶ cells of the cells that have been subjected to stimulation are subjected to transduction.

In some embodiments, the transduction is performed in serum free media. In some embodiments, the transduction is performed in the presence of IL-2, IL-7, and IL-15. In some embodiments, the viral vector for transduction is frozen and thawed prior to use, and the thawed viral vector is diluted with serum free media. In some embodiments, the serum free media for diluting the viral vector and for transduction are as described herein or in PCT/US2018/064627.

In some embodiments, the serum-free medium comprises a basal medium (e.g. OpTmizer™ T-Cell Expansion Basal Medium (ThermoFisher)), supplemented with one or more supplement. In some embodiments, the one or more supplement is serum-free. In some embodiments, the serum-free medium comprises a basal medium supplemented with one or more additional components for the maintenance, expansion, and/or activation of a cell (e.g., a T cell), such as provided by an additional supplement (e.g. OpTmizer™ T-Cell Expansion Supplement (ThermoFisher)). In some embodiments, the serum-free medium further comprises a serum replacement supplement, for example, an immune cell serum replacement, e.g., ThermoFisher, #A2596101, the CTS™ Immune Cell Serum Replacement, or the immune cell serum replacement described in Smith et al. Clin Transl Immunology. 2015 January; 4(1): e31. In some embodiments, the serum-free medium further comprises a free form of an amino acid such as L-glutamine. In some embodiments, the serum-free medium further comprises a dipeptide form of L-glutamine (e.g., L-alanyl-L-glutamine), such as the dipeptide in Glutamax™ (ThermoFisher). In some embodiments, the serum-free medium further comprises one or more recombinant cytokines, such as recombinant human IL-2, recombinant human IL-7, and/or recombinant human IL-15.

In particular embodiments, the cells, e.g., the cells of the stimulated cell population contain at least 80%, at least 85%, at least 90%, or at least 95% cells that are CD4+ T cells or CD8+ T cells. In some embodiments, the transduction, including post-transduction incubation, is performed for between 24 and 48 hours, between 36 and 12 hours, between 18 and 30 hours, or for or for about 24 hours. In some embodiments, the transduction, including post-transduction incubation, is performed for or for about 24 hours, 48 hours, or 72 hours, or for or for about 1 day, 2 days, or 3 days, respectively. In particular embodiments, the transduction, including post-transduction incubation, is performed for or for about 24 hours±6 hours, 48 hours±6 hours, or 72 hours±6 hours. In particular embodiments, the transduction, including post-transduction incubation, is performed for or for about 72 hours, 72±4 hours, or for or for about 3 days.

In certain embodiments, the transduction step is initiated within two days, within 36 hours, within 30 hours, within 24 hours, within 18 hours, within 16 hours, within 14 hours, or within 12 hours of the start or initiation of the incubation, e.g., the incubation under stimulating conditions. In certain embodiments, the transduction step is initiated at about 20 hours of the start or initiation of the incubation, e.g., the incubation under stimulating conditions. In certain embodiments, the transduction step is initiated at 20±4 hours of the start or initiation of the incubation, e.g., the incubation under stimulating conditions.

In some embodiments, the system is included with and/or placed into association with other instrumentation, including instrumentation to operate, automate, control and/or monitor aspects of the transduction step and one or more various other processing steps performed in the system, e.g. one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or populations during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, the population containing cells and population containing viral vector particles, and optionally air, can be combined or mixed prior to providing the populations to the cavity. In some embodiments, the population containing cells and population containing viral vector particles, and optionally air, are provided separately and combined and mixed in the cavity. In some embodiments, a population containing cells, a population containing viral vector particles, and optionally air, can be provided to the internal cavity in any order. In any of such some embodiments, a population containing cells and viral vector particles is the input population once combined or mixed together, whether such is combined or mixed inside or outside the centrifugal chamber and/or whether cells and viral vector particles are provided to the centrifugal chamber together or separately, such as simultaneously or sequentially.

In some embodiments, intake of the volume of gas, such as air, occurs prior to the incubating the cells and viral vector particles, such as rotation, in the transduction method. In some embodiments, intake of the volume of gas, such as air, occurs during the incubation of the cells and viral vector particles, such as rotation, in the transduction method.

In some embodiments, the liquid volume of the cells or viral vector particles that make up the transduction population, and optionally the volume of air, can be a predetermined volume. The volume can be a volume that is programmed into and/or controlled by circuitry associated with the system.

In some embodiments, intake of the transduction population, and optionally gas, such as air, is controlled manually, semi-automatically and/or automatically until a desired or predetermined volume has been taken into the internal cavity of the chamber. In some embodiments, a sensor associated with the system can detect liquid and/or gas flowing to and from the centrifuge chamber, such as via its color, flow rate and/or density, and can communicate with associated circuitry to stop or continue the intake as necessary until intake of such desired or predetermined volume has been achieved. In some aspects, a sensor that is programmed or able only to detect liquid in the system, but not gas (e.g. air), can be made able to permit passage of gas, such as air, into the system without stopping intake. In some such embodiments, a non-clear piece of tubing can be placed in the line near the sensor while intake of gas, such as air, is desired. In some embodiments, intake of gas, such as air, can be controlled manually.

In aspects of the provided methods, the internal cavity of the centrifuge chamber is subjected to high speed rotation. In some embodiments, rotation is effected prior to, simultaneously, subsequently or intermittently with intake of the liquid input population, and optionally air. In some embodiments, rotation is effected subsequent to intake of the liquid input population, and optionally air. In some embodiments, rotation is by centrifugation of the centrifugal chamber at a relative centrifugal force at the inner surface of side wall of the internal cavity and/or at a surface layer of the cells of at or about or at least at or about 200 g, 300 g, 400 g, 500 g, 600 g, 700 g, 800 g, 1000 g, 1100 g, 1500, 1600 g, 1800 g, 2000 g, 2200 g, 2500 g, 3000 g, 3200 g, 3500 g or 4000 g. In some embodiments, rotation is by centrifugation at a force that is greater than or about 1100 g, such as by greater than or about 1200 g, greater than or about 1400 g, greater than or about 1600 g, greater than or about 1800 g, greater than or about 2000 g, greater than or about 2400 g, greater than or about 2800 g, greater than or about 3000 g or greater than or about 3200 g. In particular embodiments, the rotation by centrifugation is at a force between 600 g and 800 g. In particular embodiments, the rotation by centrifugation is at a force of or of about 693 g. In some embodiments, rotation is by centrifugation at a force that is or is about 1600 g.

In some embodiments, the gas, such as air, in the cavity of the chamber is expelled from the chamber. In some embodiments, the gas, such as air, is expelled to a container that is operably linked as part of the closed system with the centrifugal chamber. In some embodiments, the container is a free or empty container. In some embodiments, the air, such as gas, in the cavity of the chamber is expelled through a filter that is operably connected to the internal cavity of the chamber via a sterile tubing line. In some embodiments, the air is expelled using manual, semi-automatic or automatic processes. In some embodiments, air is expelled from the chamber prior to, simultaneously, intermittently or subsequently with expressing the output population containing incubated cells and viral vector particles, such as cells in which transduction has been initiated or cells have been transduced with a viral vector, from the cavity of the chamber.

In some embodiments, the transduction and/or other incubation is performed as or as part of a continuous or semi-continuous process. In some embodiments, a continuous process involves the continuous intake of the cells and viral vector particles, e.g., the transduction composition (either as a single pre-existing composition or by continuously pulling into the same vessel, e.g., cavity, and thereby mixing, its parts), and/or the continuous expression or expulsion of liquid, and optionally expelling of gas (e.g. air), from the vessel, during at least a portion of the incubation, e.g., while centrifuging. In some embodiments, the continuous intake and continuous expression are carried out at least in part simultaneously. In some embodiments, the continuous intake occurs during part of the incubation, e.g., during part of the centrifugation, and the continuous expression occurs during a separate part of the incubation. The two may alternate. Thus, the continuous intake and expression, while carrying out the incubation, can allow for a greater overall volume of sample to be processed, e.g., transduced.

In some embodiments, the incubation is part of a continuous process, the method including, during at least a portion of the incubation, effecting continuous intake of said transduction composition into the cavity during rotation of the chamber and during a portion of the incubation, effecting continuous expression of liquid and, optionally expelling of gas (e.g. air), from the cavity through the at least one opening during rotation of the chamber.

In some embodiments, the semi-continuous incubation is carried out by alternating between effecting intake of the composition into the cavity, incubation, expression of liquid from the cavity and, optionally expelling of gas (e.g. air) from the cavity, such as to an output container, and then intake of a subsequent (e.g., second, third, etc.) composition containing more cells and other reagents for processing, e.g., viral vector particles, and repeating the process. For example, in some embodiments, the incubation is part of a semi-continuous process, the method including, prior to the incubation, effecting intake of the transduction composition into the cavity through said at least one opening, and subsequent to the incubation, effecting expression of fluid from the cavity; effecting intake of another transduction composition comprising cells and the viral vector particles into said internal cavity; and incubating the another transduction composition in said internal cavity under conditions whereby said cells in said another transduction composition are transduced or subjected to transduction with said vector. The process may be continued in an iterative fashion for a number of additional rounds. In this respect, the semi-continuous or continuous methods may permit production of even greater volume and/or number of cells.

In some embodiments, a portion of the transduction incubation is performed in the centrifugal chamber, which is performed under conditions that include rotation or centrifugation.

In particular embodiments, transduction of the cells with the viral vector is or includes spinoculation, e.g., centrifugation of a mixture containing the cells and the viral particles. In some embodiments, the composition containing cells and viral particles can be rotated, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm). In some embodiments, the rotation is carried at a force, e.g., a relative centrifugal force, of from or from about 100 g to 4000 g (e.g. at or about or at least at or about 100 g, 200 g, 300 g, 400 g, 500 g, 600 g, 700 g, 800 g, 900 g, 1000 g, 1500 g, 2000 g, 2500 g, 3000 g or 3500 g), as measured for example at an internal or external wall of the chamber or cavity.

In some embodiments, the cells are spinoculated with the viral vector at a force, e.g., a relative centrifugal force, of between or between about 100 g and 4000 g, 200 g and 1,000 g, 500 g and 1200 g, 1000 g and 2000 g, 600 g and 800 g, 1200 g and 1800 g, or 1500 g and 1800 g. In certain embodiments, the cells are spinoculated with the viral vector particle for, for at least, or for about 100 g, 200 g, 300 g, 400 g, 500 g, 600 g, 700 g, 800 g, 900 g, 1000 g, 1200 g, 1500 g, 1600 g, 2000 g, 2500 g, 3000 g, 3200 g, or 3500 g. In some embodiments, the cells are transduced or subjected to transduction with the viral vector at a force of or of about 692 g or 693 g. In particular embodiments, the cells are transduced or subjected to transduction with the viral vector at a force of or of about 1600 g. In some embodiments, the force is the force at the internal surface of the side wall of the internal cavity and/or at a surface layer of the cells.

In certain embodiments, the cells are spinoculated, e.g., the cell composition containing cells and viral vector is rotated, for greater than or about 5 minutes, such as greater than or about 10 minutes, greater than or about 15 minutes, greater than or about 20 minutes, greater than or about 30 minutes, greater than or about 45 minutes, greater than or about 60 minutes, greater than or about 90 minutes or greater than or about 120 minutes; or between or between about 5 minutes and 120 minutes, 30 minutes and 90 minutes, 15 minutes and 60 minutes, 15 minutes and 45 minutes, 30 minutes and 60 minutes or 45 minutes and 60 minutes, each inclusive. In some embodiments, the cells are spinoculated with the viral vector for or for about 30 minutes. In certain embodiments, the cells are spinoculated with the viral vector for or for about 60 minutes.

In some embodiments, the method of transduction includes a spinoculation, e.g., a rotation or centrifugation of the transduction composition, and optionally air, in the centrifugal chamber for greater than or about 5 minutes, such as greater than or about 10 minutes, greater than or about 15 minutes, greater than or about 20 minutes, greater than or about 30 minutes, greater than or about 45 minutes, greater than or about 60 minutes, greater than or about 90 minutes or greater than or about 120 minutes. In some embodiments, the transduction composition, and optionally air, is rotated or centrifuged in the centrifugal chamber for greater than 5 minutes, but for no more than 60 minutes, no more than 45 minutes, no more than 30 minutes or no more than 15 minutes. In particular embodiments, the transduction includes rotation or centrifugation for or for about 60 minutes.

In some embodiments, the method of transduction includes rotation or centrifugation of the transduction composition, and optionally air, in the centrifugal chamber for between or between about 10 minutes and 60 minutes, 15 minutes and 60 minutes, 15 minutes and 45 minutes, 30 minutes and 60 minutes or 45 minutes and 60 minutes, each inclusive, and at a force at the internal surface of the side wall of the internal cavity and/or at a surface layer of the cells of, of about, or at 1000 g, 1100 g, 1200 g, 1400 g, 1500 g, 1600 g, 1800 g, 2000 g, 2200 g, 2400 g, 2800 g, 3200 g or 3600 g. In particular embodiments, the method of transduction includes rotation or centrifugation of the transduction composition, e.g., the cells and the viral vector particles, at or at about 1600 g for or for about 60 minutes.

4. Vector Copy Number (VCN)

In some embodiments, genomic integration of transgene sequences, such as transgene sequences encoding a recombinant receptor, e.g. a CAR, can be assessed in cells produced in connection with any of the provided processes for engineering cells. In some embodiments, the integrated copy number is assessed, which is the copy number of the transgene sequence integrated into the chromosomal DNA or genomic DNA of cells.

In some embodiments, methods for assessing genomic integration of a transgene sequence involve separating a high molecular weight fraction of deoxyribonucleic acid (DNA), such as DNA species that are greater than or greater than about 10 kilobases (kb), from DNA isolated from one or more cell. In some aspects, such separation can be carried out by methods such as pulse field gel electrophoresis (PFGE). In some aspects, the one or more cell contains, or is suspected to contain, at least one engineered cell comprising a transgene sequence encoding a recombinant protein. In some aspects, the methods involve determining the presence, absence or amount of the transgene sequence integrated into the genome of the one or more cell, for example, by quantitative methods such as quantitative polymerase chain reaction (qPCR), digital PCR (dPCR) or droplet digital PCR (ddPCR).

In some embodiments, the high molecular weight fraction primarily contain large DNA molecules such as chromosomal or genomic DNA, and contain low or almost no molecules that are smaller than the threshold value for size, such as plasmids, non-integrated DNA fragments, linear complementary DNA (cDNA), autointegrants, long terminal repeat (LTR) circles or other residual species or molecules that have not been integrated into the genome. In some embodiments, by determining the presence, absence or amount of the transgene sequences in the high molecular weight fraction, the detected transgene sequences represent those that have been integrated into the genome of the engineered cell, and minimizes the detection of non-integrated transgene sequences.

In some embodiments, the high molecular weight fraction comprises DNA molecules that are greater than or greater than about 10 kilobases (kb) in size. In some embodiments, the high molecular weight fraction comprises DNA molecules that are greater than or greater than about 10, 11, 12, 12.5, 13, 14, 15, 16, 17, 17.5, 18, 19, 20, 25 or 30 kilobases (kb) or more in size. In some embodiments, the high molecular weight fraction comprises DNA molecules that are greater than or greater than about 10, 12.5, 15, 17.5 or 20 kilobases (kb) or more in size. In some aspects, the high molecular weight fraction contains genomic DNA or genomic DNA fragments, and excludes or separates non-integrated or residual nucleic acid species that can be present in the DNA sample. In some aspects, the high molecular weight fraction, e.g., DNA samples that are above a threshold value such as about 10, 11, 12, 12.5, 13, 14, 15, 16, 17, 17.5, 18, 19, 20, 25 or 30 kilobases (kb) or more. In some embodiments, the threshold value is greater than or greater than about 10, 12.5, 15, 17.5 or 20 kilobases (kb) or more.

In some embodiments, the high molecular weight fraction is separated or isolated using an electrophoresis-based method. In some aspects, electrophoresis separates biomolecules by charge and/or size via mobility through a separating matrix in the presence of an electric field. In some embodiments, electrophoresis systems can be used to fractionate, analyze, and collect particular analytes, including nucleic acid molecules, based on size or molecular weight. In some aspects, a fraction is or includes a subset of the plurality of molecules. In some aspects, a fraction can be defined or determined by size or molecular weight, or in some aspects, by any physical property that causes it to migrate at a faster or slower rate than other molecules or fractions of a plurality when driven to migrate through a buffer composition of the disclosure by the force of an electric field (i.e., electrophoretic mobility).

In some embodiments, the high molecular weight fraction is separated or isolated using pulse field gel electrophoresis (PFGE). In some aspects, PFGE involves introducing an alternating voltage gradient in an electrophoresis system to improve the resolution of larger nucleic acid molecules, such as chromosomal or genomic DNA. In some aspects, the voltage of the electrophoresis system is periodically switched among three directions: one that runs through the central axis of the gel and two that run at an angle of 60 degrees either side. In some aspects, exemplary systems and methods for separating or isolating nucleic acid molecules by PFGE include those described in, e.g., U.S. Pat. No. 9,599,590; US 2017/0240882; or US 2017/0254774.

In some aspects, the electrophoresis, such as PFGE, can be performed using an apparatus or system. In some aspects, the apparatus or system is an automated system or high-throughput system. Exemplary systems for performing PFGE, include, those described in, e.g., U.S. Pat. No. 9,599,590; US 2017/0240882; or US 2017/0254774, or commercially available apparatus or system, such as Pippin Prep, Blue Pippin or Pippin HT (Sage Science); CHEF Mapper® XA System, CHEF-DR® III Variable Angle System, CHEF-DR II System (Bio-Rad); and Biometra Rotaphor 8 System (Analytik Jena AG).

In some aspects, exemplary samples for assessment include a nucleic acid, an oligonucleotide, a DNA molecule, a RNA molecule, or any combination thereof. In some aspects, the sample can include, an amino acid, a peptide, a protein, or any combination thereof. In some aspects, the sample can be a whole cell lysate, or the DNA or protein fraction of a cell lysate, such as lysate of cells engineered for adoptive cell therapy.

In some embodiments, nucleic acids from the samples can include genomic DNA, double-stranded DNA (dsDNA), single-stranded DNA (ssDNA), coding DNA (or cDNA), messenger RNA (mRNA), short interfering RNA (siRNA), short-hairpin RNA (shRNA), microRNA (miRNA), single-stranded RNA, double-stranded RNA (dsRNA), a morpholino, RNA interference (RNAi) molecule, mitochondrial nucleic acid, chloroplast nucleic acid, viral DNA, viral RNA, and other organelles with separate genetic material. In some aspects, the nucleic acids from the sample can also include nucleic acid analogs that contain modified, synthetic, or non-naturally occurring nucleotides or structural elements or other alternative/modified nucleic acid chemistries, such as base analogs such as inosine, intercalators (U.S. Pat. No. 4,835,263) and minor groove binders (U.S. Pat. No. 5,801,115).

In some embodiments, prior to isolating or separating a high- or low-molecular weight fraction, the samples can be combined with a reagent that imparts a net negative charge, denatures a peptide or protein, or digests a DNA or RNA molecule prior to assessment in an electrophoresis system. In some aspects, samples can be combined with agents that impart fluorescent, magnetic, or radioactive properties to the sample or fractions thereof for the purpose of detection. In some examples, a dsDNA sample is mixed with ethidium bromide, applied to the electrophoresis cassette, and fractions of the sample are detected using an ultrabright green LED.

In some aspects, a system for separating or isolating the nucleic acid samples, such as an electrophoresis system, can be automated and/or high-throughput. In some aspects, the electrophoresis system can utilize disposable consumables or reagents, such as an electrophoresis cassette.

In some aspects, determining the presence, absence or amount of the transgene sequence can be performed using methods for determining the presence, absence or amount of a nucleic acid sequence. In particular, methods used to quantitate nucleic acid sequences, such quantitative polymerase chain reaction (qPCR) or related methods, can be employed in determining the copy number of the transgene sequence in a sample containing DNA, or in a particular fraction, such as the high molecular weight fraction, that is separated or isolated from samples containing DNA. In some embodiments, the determining the presence, absence or amount of the transgene sequence comprises determining the copy number, for example, using any one of the exemplary assays below to quantitate nucleic acid molecules.

In some aspects, the presence, absence and/or amount of a particular sequence can be detected using a probe or a primer, that can specifically bind or recognize all or a portion of the transgene sequence. In some embodiments, copy number can be determined using probes that can specifically detect a portion of the transgene sequence, or primer sequences that can specifically amplify a portion of the transgene sequence. In some aspects, the probe or primer sequences can specifically detect, bind or recognize a portion of the transgene sequence, such as a portion of the transgene sequence that is heterologous, exogenous or transgenic to the cell. In some embodiments, the primers or probe used for qPCR or other nucleic acid-based methods are specific for binding, recognizing and/or amplifying nucleic acids encoding the recombinant protein, and/or other components or elements of the plasmid and/or vector, including regulatory elements, e.g., promoters, transcriptional and/or post-transcriptional regulatory elements or response elements, or markers, e.g., surrogate markers. In some aspects, the probes or primers can be used for exemplary methods to determine the presence, absence and/or amount of transgene sequences, such as quantitative PCR (qPCR), digital PCR (dPCR) or droplet digital PCR (ddPCR).

In some aspects, the determining of the presence, absence or amount comprises determining the amount of the transgene sequence, such as determining the mass, weight, concentration or copy number of the transgene sequences, in one or more cells or in a biological sample containing one or more cells. In some aspects, the determining of the presence, absence or amount of a nucleic acid sequence, or assessing the mass, weight, concentration or copy number of the transgene sequences can be performed in a portion of a population of cells or a portion of a biological sample, and can be normalized, averaged, and/or extrapolated to determine the presence, absence or amount in the entire sample or entire population of cells.

In some embodiments, the determining the presence, absence or amount of the transgene sequence comprises determining the mass, weight, concentration or copy number of the transgene sequence per diploid genome or per cell in the one or more cells. In some embodiments, the one or more cell comprises a population of cells in which a plurality of cells of the population comprise the transgene sequence encoding the recombinant protein. In some embodiments, the copy number is an average or mean copy number per diploid genome or per cell among the population of cells.

In some aspects, determining the copy number comprises determining the number of copies of the transgene sequences present in one or more cells, or in a biological sample. In some aspects, the copy number can be expressed as an average or mean copy number. In some aspects, the copy number of a particular integrated transgene includes the number of integrants (containing transgene sequences) per cell. In some aspects, the copy number of a particular integrated transgene includes the number of integrants (containing transgene sequences) per diploid genome. In some aspects, the copy number of transgene sequence is expressed as the number of integrated transgene sequences per cell. In some aspects, the copy number of transgene sequence is expressed as the number of integrated transgene sequences per diploid genome. In some aspects, the one or more cell comprises a population of cells in which a plurality of cells of the population comprise the transgene sequence encoding the recombinant protein. In some embodiments, the copy number is an average or mean copy number per diploid genome or per cell among the population of cells.

In some embodiments, the determining the amount of the transgene sequence comprises assessing the mass, weight, concentration or copy number of the transgene sequence per the one or more cells, optionally per CD3+, CD4+ and/or CD8+ cell, and/or per cell expressing the recombinant protein. In some aspects, surface markers or phenotypes expressed on the cell can be determined using cell-based methods, such as by flow cytometry or immunostaining. In some aspects, the cells expressing the recombinant protein can be determined using cell-based methods, such as by flow cytometry or immunostaining, for example with an anti-idiotypic antibody or staining for a surrogate marker. In some aspects, the amount of transgene sequences can be normalized to the number of particular cells, such as CD3+, CD4+ and/or CD8+ cell, and/or per cell expressing the recombinant protein, or per total number of cells, such as per total number of cells in the sample or per total number of cells undergoing an engineering process.

In some embodiments, the determined copy number is expressed as a normalized value. In some embodiments, the determined copy number is quantified as a number of copy of the transgene sequence per genome or per cell. In some aspects, the per genome value is expressed as copy of the transgene sequence per diploid genome, as a typical somatic cell, such as a T cell, contains a diploid genome. In some aspects, the determined copy number can be normalized against the copy number of a known reference gene in the genome of the cell. In some aspects, the reference gene is RRP30 (encoding ribonuclease P protein subunit p30), or 18S rRNA (18S ribosomal RNA), 28S rRNA (28S ribosomal RNA), TUBA (α-tubulin), ACTB (β-actin), β2M (β2-microglobulin), ALB (albumin), RPL32 (ribosomal protein L32), TBP (TATA sequence binding protein), CYCC (cyclophilin C), EF1A (elongation factor 1a), GAPDH (glyceraldehyde-3-phosphate dehydrogenase), HPRT (hypoxanthine phosphoribosyl transferase) or RPII (RNA polymerase II). In some embodiments, the determined copy number is quantified as copy of the transgene sequence per microgram of DNA.

In some aspects, the copy number is an average, mean, or median copy number from a plurality or population of cells, such as a plurality or population of engineered cells. In some aspects, the copy number is an average or mean copy number from a plurality or population of cells, such as a plurality or population of engineered cells. In some aspects, the average or mean copy number is determined from a plurality or population of cells, such as a plurality or population of cells undergoing one or more steps of the engineering or manufacturing process, or in a cell composition, such as a cell composition for administration to a subject. In some aspects, a normalized average copy number is determined, for example, as an average or mean copy number of the transgene sequences normalized to a reference gene, such as a known gene that is present in two copies in a diploid genome. In some aspects, normalization to a reference gene that is typically present in two copies per diploid genome, can correspond to the copy number in a cell, such as a diploid cell. Thus, in some aspects, the normalized average or mean copy number can correspond to the average or mean copy number of the detected transgene sequences among a plurality or a population of cells, for example, T cells that typically have a diploid genome.

In some embodiments, the determining the presence, absence or amount of the transgene sequence is carried out by polymerase chain reaction (PCR). In some embodiments, the PCR is quantitative polymerase chain reaction (qPCR), digital PCR or droplet digital PCR, such as any described below. In some embodiments, the presence, absence or amount of the transgene sequence is determined by droplet digital PCR. In some embodiments, the PCR is carried out using one or more primers that is complementary to or is capable of specifically amplifying at least a portion of the transgene sequence, and in some cases, one or more primers that is complementary to or is capable of specifically amplifying at least a portion of a reference gene.

In some aspects, qPCR can be used to detect the accumulation of amplification product measured as the reaction progresses, in real time, with product quantification after each cycle. Thus, in some aspects, qPCR can be used to determine the copy number of a particular nucleic acid sequence, such as the transgene sequence, in a sample. In some aspects, qPCR employs fluorescent reporter molecule in each reaction well that yields increased fluorescence with an increasing amount of product DNA. In some aspects, fluorescence chemistries employed include DNA-binding dyes and fluorescently labeled sequence-specific primers or probes. In some aspects, qPCR employs a specialized thermal cycler with the capacity to illuminate each sample at a specified wavelength and detect the fluorescence emitted by the excited fluorophore. In some aspects, the measured fluorescence is proportional to the total amount of amplicon; the change in fluorescence over time is used to calculate the amount of amplicon produced in each cycle.

In some embodiments, dPCR is a method for detecting and quantifying nucleic acids, and permits accurate quantitative analysis and the highly sensitive detection of a target nucleic acid molecule. In some aspects, dPCR involves a limiting dilution of DNA into a succession of individual PCR reactions (or partitions). In some aspects, limiting dilution can employ the principles of partitioning with nanofluidics and emulsion chemistries, based on random distribution of the template nucleic acid to be assessed, e.g., transgene sequences, and Poisson statistics to measure the quantities of DNA present for a given proportion of positive partitions. In some aspects, dPCR is generally linear and are sensitive, capable of detecting or quantifying very small amounts of DNA. In some aspects, dPCR permits absolute quantification of a DNA sample using a single molecule counting method without a standard curve, and absolute quantification can be obtained from PCR for a single partition per well (see Pohl et al., (2004) Expert Rev. Mol. Diagn. 4(1), 41-47).

Exemplary commercially available apparatuses or systems for dPCR include Raindrop™ Digital PCR System (Raindance™ Technologies); QX200™ Droplet Digital™ PCR System (Bio-Rad); BioMark™ HD System and qdPCR 37K™ IFC (Fluidigm Corporation) and QuantStudio™ 3D Digital PCR System (Life Technologies™) (see, e.g., Huggett et al. (2013) Clinical Chemistry 59: 1691-1693; Shuga, et al. (2013) Nucleic Acids Research 41(16): e159; Whale et al. (2013) PLoS One 3: e58177).

In some embodiments, the presence, absence or amount of the transgene sequences, such as transgene sequences encoding a recombinant protein, for integration into the genome of the engineered cell, is determined using droplet digital polymerase chain reaction (ddPCR). ddPCR is a type of digital PCR, in which the PCR solution is divided or partitioned into smaller reactions through a water-oil emulsion chemistry, to generate numerous droplets. In some aspects, particular surfactants can be used to generate the water-in-oil droplets. (see, e.g., Hindson et al., (2011) Anal Chem 83(22): 8604-8610; Pinheiro et al., (2012) Anal Chem 84, 1003-1011). In some aspects, each individual droplet is subsequently run as individual reaction. In some aspects, the PCR sample is partitioned into nanoliter-size samples and encapsulated into oil droplets. In some aspects, the oil droplets are made using a droplet generator that applies a vacuum to each of the wells. In an exemplary case, approximately 20,000 oil droplets for individual reactions can be made from a 20 μL sample volume.

In some aspects, methods assessing integrated copy number can be performed at various time points to determine and compare the timing, extent or progress of genetic engineering, such as integration of the introduced transgene sequences into the genome of the cell into which the transgene sequences are introduced. In some aspects, the methods can be carried out at various stages of an engineering or manufacturing process for engineered cell compositions, such as any of the processes described. For example, the provided methods can be performed at various stages of an expanded engineering process or a non-expanded engineering process.

In some aspects, cells engineered by the provided methods are assessed for genomic integration of a transgene sequence, such as encoding a recombinant receptor, e.g. CAR, using the assays for vector copy number described above. In some embodiments, the methods involve separating a high molecular weight fraction of greater than or greater than about 10 kilobases (kb) from deoxyribonucleic acid (DNA) isolated from a cell, wherein prior to the separating, the cell has been introduced with a polynucleotide comprising the transgene sequence under conditions for integration of the transgene sequence into a genome of the cell, such as by viral transduction; and determining the presence, absence or amount of the transgene sequence in the high molecular weight fraction.

5. Incubation

In some embodiments, the methods for generating the engineered cells, e.g., for cell therapy in accord with any of provided methods, uses, articles of manufacture or compositions, include one or more steps for incubating cells under conditions that do not promote proliferation and/or expansion. In some embodiments, cells are incubated under conditions that do not promote proliferation and/or expansion subsequent to a step of genetically engineering, e.g., introducing a recombinant polypeptide to the cells by transduction or transfection. In particular embodiments, the cells are incubated after the cells have been incubated under stimulating conditions and transduced or transfected with a recombinant polynucleotide, e.g., a polynucleotide encoding a recombinant receptor. Thus, in some embodiments, a composition of CAR-positive T cells that has been engineered by transduction or transfection with a recombinant polynucleotide encoding the CAR, is incubated under conditions that do not promote proliferation and/or expansion.

In particular embodiments, genetic engineering, such as by transforming (e.g. transducing) the cells with a viral vector, further includes one or more steps of incubating the cells after the introducing or contacting of the cells with the viral vector. In some embodiments, cells, e.g., cells of the transformed cell population (also called “transformed cells”), are incubated subsequent to processes for genetically engineering, transforming, transducing, or transfecting the cells to introduce the viral vector into the cells. In particular embodiments, the incubation results in a population of incubated cells (also referred to herein as an incubated cell population).

In some embodiments, the cells, e.g. transformed cells, are incubated after the introducing of the heterologous or recombinant polynucleotide, e.g., viral vector particles is carried out without further processing of the cells. In particular embodiments, prior to the incubating, the cells are washed, such as to remove or substantially remove exogenous or remaining polynucleotides encoding the heterologous or recombinant polynucleotide, e.g. viral vector particles, such as those remaining in the media after the genetic engineering process following the spinoculation.

In some such embodiments, the further incubation is effected under conditions to result in integration of the viral vector into a host genome of one or more of the cells. For example, the further incubation provides time for the viral vector that may be bound to the T cells following transduction, e.g. via spinoculation, to integrate within the genome of the cell to delivery the gene of interest. In some aspects, the further incubation is carried out under conditions to allow the cells, e.g. transformed cells, to rest or recover in which the culture of the cells during the incubation supports or maintains the health of the cells. In particular embodiments, the cells are incubated under static conditions, such as conditions that do not involve centrifugation, shaking, rotating, rocking, or perfusion, e.g., continuous or semi-continuous perfusion of the media.

It is within the level of a skilled artisan to assess or determine if the incubation has resulted in integration of viral vector particles into a host genome, and hence to empirically determine the conditions for a further incubation. In some embodiments, integration of a viral vector into a host genome can be assessed by measuring the level of expression of a recombinant protein, such as a heterologous protein, encoded by a nucleic acid contained in the genome of the viral vector particle following incubation. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, nucleic acid encoding a truncated surface protein is included within the vector and used as a marker of expression and/or enhancement thereof.

In certain embodiments, the incubation is performed under static conditions, such as conditions that do not involve centrifugation, shaking, rotating, rocking, or perfusion, e.g., continuous or semi-continuous perfusion of the media. In some embodiments, either prior to or shortly after, e.g., within 5, 15, or 30 minutes, the initiation of the incubation, the cells are transferred (e.g., transferred under sterile conditions) to a container such as a bag or vial, and placed in an incubator.

In some embodiments, at least a portion of the incubation is carried out in the internal cavity of a centrifugal chamber, such as described in International Publication Number WO2016/073602.

In some embodiments, the cells that have been introduced with a polynucleotide encoding the heterologous or recombinant polypeptide, e.g., the viral vectors, are transferred into a container for the incubation. In some embodiments, the container is a vial. In particular embodiments, the container is a bag. In some embodiments, the cells, and optionally the heterologous or recombinant polypeptide, are transferred into the container under closed or sterile conditions. In some embodiments, the container, e.g., the vial or bag, is then placed into an incubator for all or a portion of the incubation. In particular embodiments, incubator is set at, at about, or at least 16° C., 24° C., or 35° C. In some embodiments, the incubator is set at 37° C., at about at 37° C., or at 37° C.±2° C., ±1° C., 0.5° C., or ±0.1° C.

In some aspects, the conditions for the incubation can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the incubation is performed in serum free media. In some embodiments, the serum free media is a defined and/or well-defined cell culture media. In certain embodiments, the serum free media is a controlled culture media that has been processed, e.g., filtered to remove inhibitors and/or growth factors. In some embodiments, the serum free media contains proteins. In certain embodiments, the serum-free media may contain serum albumin, hydrolysates, growth factors, hormones, carrier proteins, and/or attachment factors.

In particular embodiments, the cells are incubated in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In particular embodiments, the cells are incubated in the presence of IL-2, IL-7, and/or IL-15. In certain embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15. In certain embodiments, the cells are incubated in the presence of recombinant IL-2, IL-7, and IL-15.

In some embodiments, the cells, e.g., the transformed cells, are incubated with a cytokine, e.g., a recombinant human cytokine, at a concentration of between 1 IU/mL and 1,000 IU/mL, between 10 IU/mL and 50 IU/mL, between 50 IU/mL and 100 IU/mL, between 100 IU/mL and 200 IU/mL, between 100 IU/mL and 500 IU/mL, between 250 IU/mL and 500 IU/mL, or between 500 IU/mL and 1,000 IU/mL.

In some embodiments, the cells, e.g., the transformed cells, are incubated with IL-2, e.g., human recombinant IL-2, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, cells, e.g., transformed cells, are incubated with recombinant IL-2 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 100 IU/mL. In some embodiments, the cells, e.g., the transformed cells, are incubated in the presence of or of about 100 IU/mL of recombinant IL-2, e.g., human recombinant IL-2.

In some embodiments, the cells, e.g., the transformed cells, are incubated with recombinant IL-7, e.g., human recombinant IL-7, at a concentration between 100 IU/mL and 2,000 IU/mL, between 500 IU/mL and 1,000 IU/mL, between 100 IU/mL and 500 IU/mL, between 500 IU/mL and 750 IU/mL, between 750 IU/mL and 1,000 IU/mL, or between 550 IU/mL and 650 IU/mL. In particular embodiments, the cells, e.g., the transformed cells, are incubated with IL-7 at a concentration at or at about 50 IU/mL, 100 IU/mL, 150 IU/mL, 200 IU/mL, 250 IU/mL, 300 IU/mL, 350 IU/mL, 400 IU/mL, 450 IU/mL, 500 IU/mL, 550 IU/mL, 600 IU/mL, 650 IU/mL, 700 IU/mL, 750 IU/mL, 800 IU/mL, 750 IU/mL, 750 IU/mL, 750 IU/mL, or 1,000 IU/mL. In particular embodiments, the cells, e.g., the transformed cells, are incubated in the presence of or of about 600 IU/mL of IL-7.

In some embodiments, the cells, e.g., the transformed cells, are incubated with recombinant IL-15, e.g., human recombinant IL-15, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, cells, e.g., transformed cells, are incubated with recombinant IL-15 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 200 IU/mL. In some embodiments, the cells, e.g., the transformed cells, are incubated in the presence of or of about 100 IU/mL of recombinant IL-15, e.g., human recombinant IL-2.

In particular embodiments, the cells, e.g., transformed cells, are incubated in the presence of IL-2, IL-7, and/or IL-15. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15. In certain embodiments, the cells are incubated in the presence of recombinant IL-2, IL-7, and IL-15.

In some embodiments, all or a portion of the incubation, e.g., of the non-expanded process, is performed in a media comprising a basal medium (e.g., a CTS OpTmizer basal media (Thermofisher)), glutamine, and one or more recombinant cytokines, such as recombinant IL-2, IL-7, and/or IL-15. In some embodiments, the media can contain one or more additional components. In some embodiments, the one or more additional components may include a dipeptide form of L-glutamine (e.g., L-alanyl-L-glutamine). In some embodiments, the one or more additional components are provided by an additional supplement, e.g. OpTmizer® supplement (Thermofisher). In some embodiments, the media is a serum-free media and does not contain any serum component. In some aspects, the media can contain one or more serum-substituting proteins, such as one or more of albumin, insulin or transferrin (e.g. CTS™ Immune Cell Serum Replacement).

In some embodiments, the cells are incubated in the presence of the same or similar media as was present during the stimulation of the cells, such as carried out in connection with methods or processes of stimulation described above. In some embodiments, the cells are incubated in media having the same cytokines as the media present during stimulation of the cells, such as carried out in connection with methods or processes of stimulation described above. In certain embodiments, the cells are incubated in media having the same cytokines at the same concentrations as the media present during stimulation of the cells, such as carried out in connection with methods or processes of stimulation described above. In some embodiments, the cells are incubated in the absence of recombinant cytokines. In some embodiments, the cells are incubated in the absence of one or more cytokines as described herein. In some embodiments, the cells are incubated in the absence of all the cytokines described herein.

In some aspects, the further incubation is carried out under conditions to allow the cells to rest or recover that does not include the presence of a stimulating condition, e.g. in the form of recombinant cytokines or other stimulating agents. For example, the incubating is carried out in the presence of a lean media sufficient to support or maintain the culture of health of the cells during the incubation.

In some embodiments, all or a portion of the incubation is performed in basal media, such as a basal media without one or more recombinant cytokines or without any recombinant cytokine. In some embodiments, the medium does not comprise one or more recombinant cytokines, such as recombinant human IL-2, recombinant human IL-7, and/or recombinant human IL-15. In some aspects, the incubation is carried out without any recombinant cytokines. In certain embodiments, the basal media is supplemented with additional additives. In some embodiments, the basal media is not supplemented with any additional additives. Additives to cell culture media may include, but is not limited to nutrients, sugars, e.g., glucose, amino acids, vitamins, or additives such as ATP and NADH. Other additives also can be added but in general the specific additives and amounts are such that the incubation of the media with the cells facilitates maintenance of the cells but minimizes, limits and/or does not induce the metabolic activity of the cells during the incubation.

In particular embodiments, the media is a basal media that does not contain one or more recombinant cytokines and that does not contain a serum component, i.e. is a serum-free media, but may contain one or more additional components. In particular embodiments, use of such a serum-free media in all or a portion of the incubation, e.g., of the non-expanded process, provides for a lean media that provides for maintenance of the cells but does not include certain factors that may activate or render the cells metabolically active thereby fostering the cells in a state that is or is likely to be a resting or a quiescent state. In some aspects, incubation in the presence of such a serum-free media allows the cells to recover or rest after the stimulation and genetic engineering (e.g. transduction). In some aspects, incubation in the presence of such a serum-free media results in an output composition containing cells that are less susceptible to damage or loss of viability, e.g., during or following the manufacturing process and when the harvested/formulated cells are cryopreserved and then thawed immediately prior to use. In some embodiments, cells in the output composition when thawed have lower levels of caspase or other marker of apoptosis than cells that have been incubated in a similar media but containing one or more recombinant cytokines, serum, or other factors that may make the cells more metabolically active at cryopreservation of the output composition.

In some embodiments, the basal medium contains a mixture of inorganic salts, sugars, amino acids, and, optionally, vitamins, organic acids and/or buffers or other well known cell culture nutrients. In addition to nutrients, the medium also helps maintain pH and osmolality. In some aspects, the reagents of the basal media support cell growth, proliferation and/or expansion. A wide variety of commercially available basal media are well known to those skilled in the art, and include Dulbeccos' Modified Eagles Medium (DMEM), Roswell Park Memorial Institute Medium (RPMI), Iscove modified Dulbeccos' medium and Hams medium. In some embodiments, the basal medium is Iscove's Modified Dulbecco's Medium, RPMI-1640, or α-MEM.

In some embodiments, the basal media is a balanced salt solution (e.g., PBS, DPBS, HBSS, EBSS). In some embodiments, the basal media is selected from Dulbecco's Modified Eagle's Medium (DMEM), Minimal Essential Medium (MEM), Basal Medium Eagle (BME), F-10, F-12, RPMI 1640, Glasgow's Minimal Essential Medium (GMEM), alpha Minimal Essential Medium (alpha MEM), Iscove's Modified Dulbecco's Medium, and M199. In some embodiments, the basal media is a complex medium (e.g., RPMI-1640, IMDM). In some embodiments, the basal medium is OpTmizer™ CTS™ T-Cell Expansion Basal Medium (ThermoFisher).

In some embodiments, the basal medium is free of a protein. In some embodiments, the basal medium is free of a human protein (e.g., a human serum protein). In some embodiments, the basal medium is serum-free. In some embodiments, the basal medium is free of serum derived from human. In some embodiments, the basal medium is free of a recombinant protein. In some embodiments, the basal medium is free of a human protein and a recombinant protein. In some embodiments, the basal medium is free of one or more or all cytokines as described herein. In some embodiments, all or a portion of the incubation, e.g., of the non-expanded process, is performed in sbasal medium without any additional additives or recombinant cytokines. In some embodiments, the basal media is a CTS OpTmizer basal media (Thermofisher) without any additional additives or recombinant cytokines.

In some embodiments, all or a portion of the incubation, e.g., of the non-expanded process, is performed in a media comprising a basal medium and glutamine, e.g., a CTS OpTmizer basal media (Thermofisher) with glutamine.

In some embodiments, all or a portion of the incubation, e.g., of the non-expanded process, is performed in a media comprising a basal medium (e.g., a CTS OpTmizer basal media (Thermofisher)) without one or more recombinant cytokines, such as recombinant human IL-2, recombinant human IL-7, and/or recombinant human IL-15. In some embodiments, the medium is supplemented with one or more additional non-serum component. In some embodiments, the one or more supplement is serum-free. In some embodiments, the serum-free medium further comprises a free form of an amino acid such as L-glutamine. In some embodiments, the serum-free medium does not comprise a serum replacement supplement. In some embodiments, the serum-free medium does not comprise a dipeptide form of L-glutamine (e.g., L-alanyl-L-glutamine). In some embodiments, the serum-free medium does not comprise any recombinant cytokine. In some embodiments, the serum-free medium comprises a basal medium supplemented with a T cell supplement and a free form of L-glutamine, and does not contain any immune cell serum replacement, any dipeptide form of L-glutamine, or any recombinant cytokine. In some embodiments, the serum-free medium comprises a basal medium (e.g. OpTmizer™ T-Cell Expansion Basal Medium), L-glutamine and one or more additional components such as provided by a supplement (e.g. OpTmizer™ T-Cell Expansion Supplement).

In particular embodiments, the cells are incubated in the serum free medium at a concentration of or of about 0.25×10⁶ cells/mL, 0.5×10⁶ cells/mL, 0.75×10⁶ cells/mL, 1.0×10⁶ cells/mL, 1.25×10⁶ cells/mL, 1.5×10⁶ cells/mL, 1.75×10⁶ cells/mL, or 2.0×10⁶ cells/mL. In particular embodiments, the cells are incubated in the serum free medium at a concentration of or of about 0.75×10⁶ cells/mL. In some embodiments, the incubating is for or for about between 18 hours and 30 hours. In particular embodiments, the incubating is for or for about 24 hours or for about one day. In some embodiments, the incubating is for or for about 48 hours or 72 hours, or for or for about 2 days or 3 days, respectively. In particular embodiments, the incubating is for or for about 24 hours±6 hours, 48 hours±6 hours, or 72 hours±6 hours. In particular embodiments, the incubating is for or for about 72 hours, 72±4 hours, or for or for about 3 days, e.g., during which time the cells are incubated in the serum free medium at a concentration of or of about 0.75×10⁶ cells/mL. In some embodiments, all or a portion of the incubation is performed in a serum free media comprising a basal medium (e.g., a CTS OpTmizer basal media (Thermofisher)) without one or more recombinant cytokines, such as recombinant human IL-2, recombinant human IL-7, and/or recombinant human IL-15. In some embodiments, the serum-free media is supplemented with L-glutamine and/or one or more cell supplement, e.g. OpTmizer™ T-Cell Expansion Supplement, but does not contain any immune cell serum replacement, any dipeptide form of L-glutamine, or any recombinant cytokine.

In particular embodiments, the cells are incubated in the absence of cytokines. In particular embodiments, the cells are incubated in the absence of any recombinant cytokine. In particular embodiments, the cells are incubated in the absence of one or more recombinant cytokine, such as recombinant IL-2, IL-7, and/or IL-15.

In some embodiments, the basal medium further comprises glutamine, such as L-glutamine. In some aspects, the glutamine is a free form of glutamine, such as L-glutamine. In some embodiments, the concentration of the glutamine, such as L-glutamine, in the basal medium is about or less than about about 0.5 mM-1 mM, 0.5 mM-1.5 mM, 0.5 mM-2 mM, 0.5 mM-2.5 mM, 0.5 mM-3 mM, 0.5 mM-3.5 mM, 0.5 mM-4 mM, 0.5 mM-4.5 mM, 0.5 mM-5 mM, 1 mM-1.5 mM, 1 mM-2 mM, 1 mM-2.5 mM, 1 mM-3 mM, 1 mM-3.5 mM, 1 mM-4 mM, 1 mM-4.5 mM, 1 mM-5 mM, 1.5 mM-2 mM, 1.5 mM-2.5 mM, 1.5 mM-3 mM, 1.5 mM-3.5 mM, 1.5 mM-4 mM, 1.5 mM-4.5 mM, 1.5 mM-5 mM, 2 mM-2.5 mM, 2 mM-3 mM, 2 mM-3.5 mM, 2 mM-4 mM, 2 mM-4.5 mM, 2 mM-5 mM, 2.5 mM-3 mM, 2.5 mM-3.5 mM, 2.5 mM-4 mM, 2.5 mM-4.5 mM, 2.5 mM-5 mM, 3 mM-3.5 mM, 3 mM-4 mM, 3 mM-4.5 mM, 3 mM-5 mM, 3.5 mM-4 mM, 3.5 mM-4.5 mM, 3.5 mM-5 mM, 4 mM-4.5 mM, 4 mM-5 mM, or 4.5 mM-5 mM, each inclusive. In some embodiments, the concentration of glutamine, such as L-glutamine, in the basal medium is at least about 0.5 mM, 1 mM, 1.5 mM, 2 mM, 2.5 mM, 3 mM, 3.5 mM, 4 mM, 4.5 mM, or 5 mM. In some embodiments, the concentration of glutamine, such as L-glutamine, in the basal medium is at most about 2 mM, 2.5 mM, 3 mM, 3.5 mM, 4 mM, 4.5 mM, 5 mM. In some embodiments, the concentration of glutamine, such as L-glutamine, in the basal medium is about 2 mM. In some embodiments, the basal medium further may comprises a protein or a peptide. In some embodiments, the at least one protein is not of non-mammalian origin. In some embodiments, the at least one protein is human or derived from human. In some embodiments, the at least one protein is recombinant. In some embodiments, the at least one protein includes albumin, transferrin, insulin, fibronectin, aprotinin or fetuin. In some embodiments, the protein comprises one or more of albumin, insulin or transferrin, optionally one or more of a human or recombinant albumin, insulin or transferrin.

In some embodiments, the protein is an albumin or albumin substitute. In some embodiments, the albumin is a human derived albumin. In some embodiments, the albumin is a recombinant albumin. In some embodiments, the albumin is a natural human serum albumin. In some embodiments, the albumin is a recombinant human serum albumin. In some embodiments, the albumin is a recombinant albumin from a non-human source. Albumin substitutes may be any protein or polypeptide source. Examples of such protein or polypeptide samples include but are not limited to bovine pituitary extract, plant hydrolysate (e.g., rice hydrolysate), fetal calf albumin (fetuin), egg albumin, human serum albumin (HSA), or another animal-derived albumins, chick extract, bovine embryo extract, AlbuMAX® I, and AlbuMAX® II. In some embodiments, the protein or peptide comprises a transferrin. In some embodiments, the protein or peptide comprises a fibronectin. In some embodiments, the protein or peptide comprises aprotinin. In some embodiments, the protein comprises fetuin.

In some embodiments, the one or more additional protein is part of a serum replacement supplement that is added to the basal medium. Examples of serum replacement supplements include, for example, Immune Cell Serum Replacement (ThermoFisher, #A2598101) or those described in Smith et al. Clin Transl Immunology. 2015 January; 4(1): e31.

In certain embodiments, the cells are incubated after the introducing of the polynucleotide encoding the heterologous or recombinant protein, e.g., viral vector, for, for about, or for at least 18 hours, 24 hours, 30 hours, 36 hours, 40 hours, 48 hours, 54 hours, 60 hours, 72 hours, 84 hours, 96 hours, or more than 96 hours. In certain embodiments, the cells are incubated after the introducing of the polynucleotide encoding the heterologous or recombinant protein, e.g., viral vector, for, for about, or for at least one day, 2 days, 3 days, 4 days, or more than 4 days. In some embodiments, the incubating is performed for an amount of time between 30 minutes and 2 hours, between 1 hour and 8 hours, between 6 hours and 12 hours, between 12 hours and 18 hours, between 16 hours and 24 hours, between 18 hours and 30 hours, between 24 hours and 48 hours, between 24 hours and 72 hours, between 42 hours and 54 hours, between 60 hours and 120 hours between 96 hours and 120 hours, between 90 hours and between 1 days and 7 days, between 3 days and 8 days, between 1 day and 3 days, between 4 days and 6 days, or between 4 days and 5 days prior to the genetic engineering. In some embodiments, the incubating is for or for about between 18 hours and 30 hours. In particular embodiments, the incubating is for or for about 24 hours or for about one day.

In certain embodiments, the total duration of the incubation is, is about, or is at least 12 hours, 18 hours, 24 hours, 30 hours, 36 hours, 42 hours, 48 hours, 54 hours, 60 hours, 72 hours, 84 hours, 96 hours, 108 hours, or 120 hours. In certain embodiments, the total duration of the incubation is, is about, or is at least one day, 2 days, 3 days, 4 days, or 5 days. In particular embodiments, the incubation is completed at, at about, or within 120 hours, 108 hours, 96 hours, 84 hours, 72 hours, 60 hours, 54 hours, 48 hours, 42 hours, 36 hours, 30 hours, 24 hours, 18 hours, or 12 hours. In particular embodiments, the incubation is completed at, at about, or within one day, 2 days, 3 days, 4 days, or 5 days. In some embodiments, the total duration of the incubation is between or between about 12 hour and 120 hours, 18 hour and 96 hours, 24 hours and 72 hours, or 24 hours and 48 hours, inclusive. In some embodiments, the total duration of the incubation is between or about between 1 hour and 48 hours, 4 hours and 36 hours, 8 hours and 30 hours or 12 hours and 24 hours, inclusive. In particular embodiments, the incubation is performed for or for about 24 hours, 48 hours, or 72 hours, or for or for about 1 day, 2 days, or 3 days, respectively. In particular embodiments, the incubation is performed for 24 hours±6 hours, 48 hours±6 hours, or 72 hours±6 hours. In particular embodiments, the incubation is performed for or for about 72 hours or for or for about 3 days.

In particular embodiments, the incubation is initiated at, at about, or is at least 12 hours, 18 hours, 24 hours, 30 hours, 36 hours, 42 hours, 48 hours after the initiation of the stimulation. In particular embodiments, the incubation is initiated at, at about, or is at least 0.5 days, one day, 1.5 days, or 2 days after the initiation of the stimulation. In particular embodiments, the incubation is initiated at, at about, or within 120 hours, 108 hours, 96 hours, 84 hours, 72 hours, 60 hours, 54 hours, 48 hours, 42 hours, 36 hours, 30 hours, 24 hours, 18 hours, or 12 hours of the initiation of the stimulation. In particular embodiments, the incubation is initiated at, at about, or within 5 days, 4 days, 3 days, 2 days, one day, or 0.5 days of the initiation of the stimulation.

In some embodiments, the incubation is completed between or between about 24 hour and 120 hours, 36 hour and 108 hours, 48 hours and 96 hours, or 48 hours and 72 hours, inclusive, after the initiation of the stimulation. In some embodiments, the incubation is completed at, about, or within 120 hours, 108 hours, 96 hours, 72 hours, 48 hours, or 36 hours from the initiation of the stimulation. In some embodiments, the incubation is completed at, about, or within 5 days, 4.5 days, 4 days, 3 days, 2 days, or 1.5 days from the initiation of the stimulation. In particular embodiments, the incubation is completed after hours 24 hours±6 hours, 48 hours±6 hours, or 72 hours±6 hours after the initiation of the stimulation. In some embodiments, the incubation is completed after or after about 72 hours or after or after about 3 days.

In some embodiments, the incubation is carried out for an amount of time sufficient for the heterologous or recombinant polynucleotide to be integrated into the genome. In particular embodiments, the incubation is performed for an amount of time sufficient for at least an integrated viral copy number (iVCN) of, of about, or of at least 0.1, 0.5, 1, 2, 3, 4, 5, or greater than 5 per diploid genome. In particular embodiments, the incubation is performed for an amount of time sufficient for at least an iVCN of, of about, or of at least 0.5 or 1. In particular embodiments, the incubation is carried out for an amount of time sufficient for the heterologous or recombinant polynucleotide to be stably integrated into the genome. In particular embodiments, the heterologous or recombinant polynucleotide is considered to be stably integrated when the iVCN per diploid genome does not change by more than 20%, 15%, 10%, 5%, 1%, or 0.1% over a period of time, e.g. at least 12, 24, or 48 hours. In particular embodiments, the incubation is completed prior to the stable integration.

In certain embodiments, the incubation is performed or carried out at least until the integrated vector is detected in the genome. In some embodiments, the incubation is completed prior to achieving stable integrated vector copy number (iVCN) per diploid genome. In particular embodiments, the incubation is performed or carried out at least until the integrated vector is detected in the genome but prior to achieving a stable iVCN per diploid genome. In certain embodiments, a stable iVCN per diploid genome is achieved when the iVCN peaks and/or remains unchanged, or unchanged within a tolerated error, for a period of time. In some embodiments, the tolerated error is, is within, or is about ±40%, ±35%, ±30%, ±25%, ±20%, ±15%, ±10%, ±5%, ±2%, ±1%, or less than ±1%. In certain embodiments, the period of time is, is about, or is at least 2 hours, 4 hours, 6 hours, 8 hours, 12 hours, 16 hours, 18 hours, 24 hours, 36 hours, 48 hours, 60 hours, or 72 hours. In certain embodiments, the period of time is, is about, or is at least one day, 2 days, or 3 days. In certain embodiments, the stable iVCN per diploid genome is achieved when the iVCN peaks and remains unchanged, or unchanged within a tolerated error, e.g., ±25%, for a period of time that is, is about, or is at least 24 hours or one day. In some embodiments, a stable iVCN per diploid genome is achieved when the fraction of iVCN to total vector copy number (VCN) in the diploid genome of the population of transformed cells, on average, is, is at least or is about 0.6. 0.7. 0.8, 0.9, 1.0, 1.1, 1.2, 1.3, 1.4, or 1.5, or is within a tolerated error thereof, e.g., ±25%, 20%, ±15%, ±10%, ±5%, or ±1%. In certain embodiments, a stable iVCN per diploid genome is achieved when the fraction of iVCN to total vector copy number (VCN) in the diploid genome of the population of transformed cells, on average, is or is about 0.8, or is within a tolerated error thereof. In some embodiments, a stable iVCN per diploid genome is achieved when the fraction of iVCN to total vector copy number (VCN) in the diploid genome of the population of transformed cells, on average, is or is about 1.0 or is within a tolerated error thereof.

In some embodiments, the incubation is completed before the iVCN of reaches, reaches about, or reaches at least 5.0, 4.0, 3.0, 2.5, 2.0, 1.75, 1.5, 1.25, 1.2, 1.1, 1.0, 0.9, 0.8, 0.75, 0.7, 0.6, 0.5, 0.4, 0.3, or 0.25 copies per diploid genome. In certain embodiments, the incubation is completed before the iVCN reaches or about 1.0 copy per diploid genome. In particular embodiments, the incubation is completed before the iVCN reaches or about 0.5 copies per diploid genome.

In certain embodiments, the cells are harvested prior to, prior to about, or prior to at least one, two, three, four, five, six, eight, ten, twenty, or more cell doublings of the cell population, e.g., doublings that occur during the incubating. In particular embodiments, the amount of cell doublings may be calculated by measuring the number of viable cells in a population at different time points, such as at different times or stages of an engineering process. In particular embodiment, the cell doubling can be calculated by comparing the total amount of viable cells at one time point to the total number of viable cells present at an earlier time point. In certain embodiments, the incubation is completed prior to, to about, or to at least one, two, three, four, five, six, eight, ten, twenty, or more cell doublings of the cell population, e.g., doublings that occur during the incubating. In certain aspects, the cell doubling is calculated by determining the total nucleated cell number (TNC) when the incubation is initiated and when the incubation completed, and then determining the natural log of the product of the TNC at the completion divided by the TNC at the initiation, and then dividing said natural log of the product by the natural log of 2.

In some aspects, the number of doublings of that occurs in a population, e.g., during an engineering process, is determined using the following formula:

$$Cell doublings = \frac{ln ( \frac{TNC at harvest}{TNC 3 days post - activation} )}{ln 2} \qquad 1 )$$

In some aspects, the number of doublings of that occurs in a population, e.g., during an engineering process, using the following formula:

$$Cell doublings = \frac{ln ( \frac{TNC at harvest}{TNC at initation of the stimulating} )}{ln 2} \qquad 2 )$$

In certain embodiments, the number of doublings that occurs in a population, e.g., during the engineering process, is determined suing the following formula:

$$Cell doublings = \frac{ln ( \frac{TNC at harvest}{TNC following stimulation} )}{ln 2} \qquad 3 )$$

In various embodiments, the number of doublings that occurs in a population, e.g., during the engineering process, is determined suing the following formula:

$$Cell doublings = \frac{ln ( \frac{TNC at harvest}{TNC at transduction} )}{ln 2} \qquad 4 )$$

In particular embodiments, the number of doublings that occurs in a population, e.g., during the engineering process, is determined suing the following formula:

$$Cell doublings = \frac{ln ( \frac{TNC at harvest}{TNC at the begining of the incubation} )}{ln 2} \qquad 5 )$$

In certain embodiments, the incubation is completed before the total number cells, e.g., total number of incubated cells or cells undergoing the incubation, is greater than or than about one, two, three, four, five, six, eight, ten, twenty, or more than twenty times the number of cells of the input population, e.g., the total number of cells that were contacted with the stimulatory reagent. In various embodiments, the incubation is completed before the total number of incubated cells is greater than or than about one, two, three, four, five, six, eight, ten, twenty, or more than twenty times the total number of cells that were transformed, transduced, or spinoculated, e.g., the total number of cells that were contacted with a viral vector. In certain embodiments, the cells are T cells, viable T cells, CD3+ T cells, CD4+ T cells, CD8+ T cells, CAR expressing T cells, or a combination of any of the foregoing. In some embodiments, the incubation is completed before the total number of cells is greater than the total number of cells of the input population. In some embodiments, the incubation is completed before the total number of viable CD3+ T cells is greater than the total number of viable CD3+ cells of the input population. In certain embodiments, the incubation is completed before the total number of cells is greater than the total number of cells of the transformed, transduced, or spinoculated cells. In some embodiments, the incubation is completed before the total number of viable CD3+ T cells is greater than the total number of viable CD3+ of the transformed, transduced, or spinoculated cells.

In some embodiments, the total cell number or total viable cell number of the cell population remains similar, the same, or essentially the same during the incubation. In particular embodiments, the total cell number or total viable cell number of the cell population does not change during the incubation. In some aspects, the total cell number or total viable cell number decreases during the incubation. In particular aspects, the total viable cell number is, is about, or is less than 95%, 90%, 85%, 80%, 75%, 70%, 65%, 60%, 55%, of 50% of the total cell number or total viable cell number of the input population prior to, e.g., immediately prior to, or at the initiation of the stimulation.

6. Removal of Stimulatory Reagents

In some embodiments, the population of incubated T cells was produced or generated in accord with any of the methods provided herein in which a substance, such as a competition agent, was added to T cells to disrupt, such as to lessen and/or terminate, the signaling of the stimulatory agent or agents. In some embodiments, the population of the incubated T cells contains the presence of a substance, such as a competition agent, e.g. biotin or a biotin analog, e.g. D-Biotin. In some embodiments, the substance, such as a competition agent, e.g. biotin or a biotin analog, e.g. D-Biotin, is present in an amount that is at least 1.5-fold greater, at least 2-fold, at least 3-fold, at least 4-fold, at least 5-fold, at least 10-fold, at least 100-fold, at least 1000-fold or more greater than the amount of the substance in a reference population or preparation of cultured T cells in which the substance was not added exogenously during the incubation. In some embodiments, the amount of the substance, such as a competition agent, e.g. biotin or a biotin analog, e.g. D-Biotin, in the population of cultured T cells is from or from about 10 μM to 100 μM, 100 μM to 1 mM, 100 μM to 500 μM or 10 μM to 100 μM. In some embodiments, 10 μM or about 10 μM of biotin or a biotin analog, e.g., D-biotin, is added to the cells or the cell population to separate or remove the oligomeric stimulatory reagent from the cells or cell population.

In certain embodiments, the one or more agents (e.g., agents that stimulate or activate a TCR and/or a co-receptor) associate with, such as are reversibly bound to, the oligomeric reagent, such as via the plurality of the particular binding sites (e.g., binding sites Z) present on the oligomeric reagent. In some cases, this results in the agents being closely arranged to each other such that an avidity effect can take place if a target cell having (at least two copies of) a cell surface molecule that is bound by or recognized by the agent is brought into contact with the agent. In some aspects, the receptor binding reagent has a low affinity towards the receptor molecule of the cell at binding site B, such that the receptor binding reagent dissociates from the cell in the presence of the competition reagent. Thus, in some embodiments, the agents are removed from the cells in the presence of the competition reagent.

In some embodiments, the oligomeric stimulatory reagent is a streptavidin mutein oligomer with reversibly attached anti-CD3 and anti-CD28 Fabs. In some embodiments, the Fabs are attached contain streptavidin binding domains, e.g., that allow for the reversible attachment to the streptavidin mutein oligomer. In some cases, anti-CD3 and anti-CD28 Fabs are closely arranged to each other such that an avidity effect can take place if a T cell expressing CD3 and/or CD28 is brought into contact with the oligomeric stimulatory reagent with the reversibly attached Fabs. In some aspects, the Fabs have a low affinity towards CD3 and CD28, such that the Fabs dissociate from the cell in the presence of the competition reagent, e.g., biotin or a biotin variant or analogue. Thus, in some embodiments, the Fabs are removed or dissociated from the cells in the presence of the competition reagent, e.g., D-biotin.

In some embodiments, the oligomeric stimulatory reagent, e.g., the oligomeric stimulatory streptavidin mutein reagent, is removed or separated from the cells or cell populations prior to collecting, harvesting, or formulating the cells. In some embodiments, oligomeric stimulatory reagent, e.g., the oligomeric stimulatory streptavidin mutein reagent, is removed or separated from the cells or cell populations by contact or exposure to a competition reagent, e.g., biotin or a biotin analog such as D-biotin, after or during the incubation, e.g., an incubation described herein such as in Section II-C-5. In certain embodiments, the cells or cell population are contacted or exposed to a competition reagent, e.g., biotin or a biotin analog such as D-biotin, to remove oligomeric stimulatory reagent, e.g., the oligomeric stimulatory streptavidin mutein reagent, after the incubation but prior to steps for collecting, harvesting, or formulating the cells. In particular embodiments, the cells or cell population are contacted or exposed to a competition reagent, e.g., biotin or a biotin analog such as D-biotin, to remove the oligomeric stimulatory reagent, e.g., the oligomeric stimulatory streptavidin mutein reagent, after the incubation. In some aspects, when oligomeric stimulatory reagent, e.g., the oligomeric stimulatory streptavidin mutein reagent, is separated or removed from the cells during the incubation, e.g., by contact or exposure to a competition reagent, e.g., biotin or a biotin analog such as D-biotin, the cells are returned to the same incubation conditions as prior to the separation or removal for the remaining duration of the incubation.

In some embodiments, the cells are contacted with, with about, or with at least 0.01 μM, 0.05 μM, 0.1 μM, 0.5 μM, 1 μM, 2 μM, 3 μM, 4 μM, 5 μM, 10 μM, 100 μM, 500 μM, 0.01 μM, 1 mM, or 10 mM of the competition reagent to remove or separate the oligomeric stimulatory reagent from the cells. In various embodiments, the cells are contacted with, with about, or with at least 0.01 μM, 0.05 μM, 0.1 μM, 0.5 μM, 1 μM, 2 μM, 3 μM, 4 μM, 5 μM, 10 μM, 100 μM, 500 μM, 0.01 μM, 1 mM, or 10 mM of biotin or a biotin analog such as D-biotin, to remove or separate the stimulatory streptavidin mutein oligomers with reversibly attached anti-CD3 and anti-CD28 Fabs from the cells. In various embodiments, the cells are contacted with between or between about 100 μM and 10 mM, e.g., 1 mM, of biotin or a biotin analog such as D-biotin, to remove or separate the oligomeric stimulatory reagent, such as streptavidin mutein oligomers with reversibly attached anti-CD3 and anti-CD28 Fabs from the cells. In various embodiments, the cells are contacted with between or between about 100 μM and 10 mM, e.g., 1 mM, of biotin or a biotin analog such as D-biotin for or for about 2 hours, 6 hours, 12 hours, 18 hours, 24 hours, 30 hours, 36 hours, 42 hours, or 48 hours post contact or exposure to D-biotin.

In particular embodiments, the oligomeric stimulatory reagent, e.g., the oligomeric stimulatory streptavidin mutein reagent, is removed or separated from the cells within or within about 120 hours, 108 hours, 96 hours, 84 hours, 72 hours, 60 hours, 48 hours, 36 hours, 24 hours, or 12 hours, inclusive, of the initiation of the stimulation. In particular embodiments, the oligomeric stimulatory reagent, e.g., the oligomeric stimulatory streptavidin mutein reagent, is removed or separated from the cells within or within about 5 days, 4 days, 3 days, 2 days, one day or 0.5 days, inclusive, of the initiation of the stimulation. In particular embodiments, the oligomeric stimulatory reagent, e.g., the oligomeric stimulatory streptavidin mutein reagent, is removed or separated from the cells at or at about 48 hours or at or at about 2 days after the stimulation is initiated. In certain embodiments, the oligomeric stimulatory reagent, e.g., the oligomeric stimulatory streptavidin mutein reagent, is removed or separated from the cells at or at about 72 hours or at or at about 3 days after the stimulation is initiated. In some embodiments, the oligomeric stimulatory reagent, e.g., the oligomeric stimulatory streptavidin mutein reagent is removed or separated from the cells at or at about 96 hours or at or at about 4 days after the stimulation is initiated.

In certain embodiments, the cells or cell population are contacted or exposed to a competition reagent, e.g., biotin or a biotin analog such as D-biotin, to remove oligomeric stimulatory reagent, e.g., the oligomeric stimulatory streptavidin mutein reagent, at or at about 48 hours or at or at about 2 days after the stimulation is initiated, e.g., during or after the incubation described herein such as in Section II-C-5. In some aspects, when oligomeric stimulatory reagent, e.g., the oligomeric stimulatory streptavidin mutein reagent, is separated or removed from the cells during the incubation, e.g., by contact or exposure to a competition reagent, e.g., biotin or a biotin analog such as D-biotin, the cells are returned to the same incubation conditions as prior to the separation or removal for the remaining duration of the incubation. In other aspects, when oligomeric stimulatory reagent, e.g., the oligomeric stimulatory streptavidin mutein reagent, is separated or removed from the cells after the incubation, e.g., by contact or exposure to a competition reagent, e.g., biotin or a biotin analog such as D-biotin, the cells are further incubated for or for about 2 hours, 6 hours, 12 hours, 18 hours, 24 hours, 30 hours, 36 hours, 42 hours, or 48 hours post contact or exposure to the competition reagent. In some embodiments, the transduced cells with D-Biotin treatment are further incubated for or for about 48 hours post D-Biotin addition.

7. Harvesting, Collecting, or Formulating Cells

In some embodiments, the cells are harvested or collected. In particular embodiments, the cells are collected of harvested after the completion of the incubation. In certain embodiments, the collected or harvested cells are the cells of an output population. In some embodiments, the output population includes cells that are viable, CD3+, CD4+, CD8+, and/or positive for a recombinant receptor, e.g., CAR+. In particular embodiments, the harvested CD4+ T cells and formulated CD8+ T cells are the output CD4+ and CD8+ T cells. In particular embodiments, a formulated cell population, e.g., a formulated population of enriched CD4+ and CD8+ cells, is an output cell population, e.g., an output population of enriched CD4+ and CD8+ cells.

In some embodiments, the cells or cell population that is harvested, collected, or formulated have not undergone any expansion, e.g., any conditions where the cells were incubated or cultivated under conditions that increase the amount of viable cells during the incubation or cultivation. For example, in some aspects, the cells that are harvested have not undergone any incubation or cultivation where the amount of total viable cells is increased at the end of the incubation or cultivation as compared to the number of total viable cells at the beginning of the incubation or cultivation. In some embodiments, the cells that are harvested have not undergone any incubation or cultivation step explicitly for the purpose of increasing (e.g., expanding) the total number of viable cells at the end of the incubation or cultivation process compared to the beginning of said incubation or cultivation process. In some embodiments, the cells are incubated under conditions that may result in expansion, but the incubating conditions are not carried out for purposes of expanding the cell population. In some embodiments, the cells that are harvested may have undergone expansion despite having been manufactured in a process that does not include an expansion step. In some embodiments, a manufacturing process that does not include an expansion step is referred to as a non-expanded or minimally expanded process. A “non-expanded” process may also be referred to as a “minimally expanded” process. In some embodiments, a non-expanded or minimally expanded process may result in cells having undergone expansion despite the process not including a step for expansion. In some embodiments, the cells that are harvested may have undergone an incubation or cultivating step that includes a media composition designed to reduce, suppress, minimize, or eliminate expansion of a cell population as a whole. In some embodiments, the collected, harvested, or formulated cells have not previously undergone an incubation or cultivation that was performed in a bioreactor, or under conditions where the cells were rocked, rotated, shaken, or perfused for all or a portion of the incubation or cultivation. Exemplary non-expanded processes of manufacturing and engineered cells produced by such processes are disclosed in PCT/US2019/046062, which is incorporated by reference in its entirety.

In some embodiments, a cell selection, isolation, separation, enrichment, and/or purification step is performed before the cells or cell population is harvested, collected, or formulated. In some embodiments, the cell selection, isolation, separation, enrichment, and/or purification step is carried out using chromatography as disclosed herein. In some embodiments, a T cell selection step by chromatography is performed after T cell transduction, but prior to harvesting, prior to collecting, and/or prior to formulating the cells. In some embodiments, a T cell selection step by chromatography is performed immediately prior to harvesting the cells.

In certain embodiments, the amount of time from the initiation of the stimulation to collecting, harvesting, or formulating the cells is, is about, or is less than 36 hours, 42 hours, 48 hours, 54 hours, 60 hours, 72 hours, 84 hours, 96 hours, 108 hours, or 120 hours. In certain embodiments, the amount of time from the initiation of the stimulation to collecting, harvesting, or formulating the cells is, is about, or is less than 1.5 days, 2 days, 3 days, 4 days, or 5 days. In some embodiments, the amount of time from the initiation of the stimulation to collecting, harvesting, or formulating the cells for generating engineered cells, from the initiation of the stimulation to collecting, harvesting, or formulating the cells is between or between about 36 hours and 120 hours, 48 hours and 96 hours, or 48 hours and 72 hours, inclusive, or between or between about 1.5 days and 5 days, 2 days and 4 days, or 2 day and 3 days, inclusive. In particular embodiments, the amount of time from the initiation of incubation to harvesting, collecting, or formulating the cells is, is about, or is less than 48 hours, 72 hours, or 96 hours. In particular embodiments, the amount of time from the initiation of incubation to harvesting, collecting, or formulating the cells is, is about, or is less than 2 days, 3 days, or 4 days. In particular embodiments, the amount of time from the initiation of incubation to harvesting, collecting, or formulating the cells is 48 hours±6 hours, 72 hours±6 hours, or 96 hours±6 hours. In particular embodiments, the amount of time from the initiation of incubation to harvesting, collecting, or formulating the cells is or is about 96 hours or four days.

In particular embodiments, the cells are harvested, collected, or formulated in a serum-free medium, such as one described herein or in PCT/US2018/064627, which is incorporated herein by reference. In some embodiments, the cells are harvested, collected, or formulated into the same serum-free medium as used during the incubation.

In particular embodiments, the cells are harvested, collected or formulated in a basal media that does not contain one or more recombinant cytokines and that does not contain a serum component, i.e. is a serum-free media, but may contain one or more additional components. In particular embodiments, use of such a serum-free media provides for a lean media that provides for maintenance of cells but does not include certain factors that may activate or render the cells metabolically active thereby fostering the cells in a state that is or is likely to be a resting or a quiescent state. In some aspects, incubation in the presence of such a serum-free media allows the cells to recover or rest after the stimulation and genetic engineering (e.g. transduction). In some aspects, harvesting, collecting or formulating cells in the presence of such a serum-free media results in a formulation of the output composition containing cells that are less susceptible to damage or loss of viability, e.g., when the harvested/formulated cells are cryopreserved and then thawed immediately prior to use. In some embodiments, cells in the output composition when thawed have lower levels of caspase or other marker of apoptosis than cells that have been incubated in a similar media but containing one or more recombinant cytokines, serum, or other factors that may make the cells more metabolically active at cryopreservation of the output composition.

In certain embodiments, one or more populations of enriched T cells are formulated. In particular embodiments, one or more populations of enriched T cells are formulated after the one or more populations have been engineered and/or incubated. In particular embodiments, the one or more populations are input populations. In some embodiments, the one or more input populations have been previously cryoprotected and stored, and are thawed prior to the incubation.

In certain embodiments, the cells are harvested or collected at least when the integrated vector is detected in the genome. In some embodiments, the cells are harvested or collected prior to stable integrated vector copy number (iVCN) per diploid genome. In particular embodiments, the cells are harvested or collected after the integrated vector is detected in the genome but prior to when a stable iVCN per diploid genome is achieved.

In some embodiments, the cells are harvested or collected before the iVCN of reaches, reaches about, or reaches at least 5.0, 4.0, 3.0, 2.5, 2.0, 1.75, 1.5, 1.25, 1.2, 1.1, 1.0, 0.9, 0.8, 0.75, 0.7, 0.6, 0.5, 0.4, 0.3, or 0.25 copies per diploid genome. In particular embodiments, the cells are harvested or collected before the iVCN reaches or about 1.0 copy per diploid genome. In some embodiments, the cells are collected or harvested before the iVCN reaches or about 0.5 copies per diploid genome.

In certain embodiments, the cells are harvested prior to, prior to about, or prior to at least one, two, three, four, five, six, eight, ten, twenty, or more cell doublings of the cell population, e.g., doublings that occur during the incubating.

In particular embodiments, the cells are harvested or collected at a time before the total number cells, e.g., total number of incubated cells or cells undergoing the incubation, is greater than or than about one, two, three, four, five, six, eight, ten, twenty, or more than twenty times the number of cells of the input population, e.g., the total number of cells that were contacted with the stimulatory reagent. In some embodiments, the cells are harvested or collected at a time before the total number of incubated cells is greater than or than about one, two, three, four, five, six, eight, ten, twenty, or more than twenty times the total number of cells that were transformed, transduced, or spinoculated, e.g., the total number of cells that were contacted with a viral vector. In certain embodiments, the cells are T cells, viable T cells, CD3+ T cells, CD4+ T cells, CD8+ T cells, CAR expressing T cells, or a combination of any of the foregoing. In particular embodiments, the cells are harvested or collected at a time before the total number of cells is greater than the total number of cells of the input population. In various embodiments, the cells are harvested or collected at a time before the total number of viable CD3+ T cells is greater than the total number of viable CD3+ cells of the input population. In particular embodiments, the cells are harvested or collected at a time before the total number of cells is greater than the total number of cells of the transformed, transduced, or spinoculated cells. In various embodiments, the cells are harvested or collected at a time before the total number of viable CD3+ T cells is greater than the total number of viable CD3+ cells of the transformed, transduced, or spinoculated cells. In various embodiments, the cells are harvested or collected at a time before the total number of viable CD4+ cells and CD8+ cells is greater than the total number of viable CD4+ cells and CD8+ cells of the input population. In particular embodiments, the cells are harvested or collected at a time before the total number of cells is greater than the total number of cells of the transformed, transduced, or spinoculated cells. In various embodiments, the cells are harvested or collected at a time before the total number of viable CD4+ cells and CD8+ cells is greater than the total number of viable CD4+ cells and CD8+ cells of the transformed, transduced, or spinoculated cells.

In certain embodiments, the process comprises a step of filtering the cell composition during or after the harvesting or collecting, e.g., using a filter (e.g., a 40 μm filter), for example, to remove large particulates. In certain embodiments, the filtering step is performed while the cells are being harvested or collected. For example, a filter may be in-line with between the cells being incubated after transduction and a harvesting/collection device such as the Sepax® or Sepax 2® cell processing systems. In certain embodiments, the cells are harvested or collected and then filtered before the filtered composition is optionally washed. In certain embodiments, the cells are harvested or collected, washed, and the washed cell composition is filtered.

In certain embodiments, the formulated cells are output cells. In some embodiments, a formulated population of enriched T cells is an output population of enriched T cells. In particular embodiments, the formulated CD4+ T cells and formulated CD8+ T cells are the output CD4+ and CD8+ T cells. In particular embodiments, a formulated cell population, e.g., a formulated population of enriched CD4+ and CD8+ cells, is an output cell population, e.g., an output population of enriched CD4+ and CD8+ cells.

In some embodiments, cells can be formulated into a container, such as a bag or vial. In some embodiments, the vial may be an infusion vial. In some embodiments, the vial is formulated with a single unit dose of the engineered cells, such as including the number of cells for administration in a given dose or fraction thereof.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

In some embodiments, the dose of cells administered is in a cryopreserved composition. In some aspects, the composition is administered after thawing the cryopreserved composition. In some embodiments, the composition is administered within at or about 30 minutes, 45 minutes, 60 minutes, 90 minutes, 120 minutes, 150 minutes or 180 minutes after thawing. In some embodiments, the composition is administered within at or about 120 minutes after thawing.

In some embodiments, the dose of cells is administered with a syringe. In some embodiments, the syringe has a volume of at or about 0.5, 1, 2, 2.5, 3, 4, 5, 7.5, 10, 20 or 25 mL, or a range defined by any of the foregoing.

Also provided are articles of manufacture and kits containing engineered cells expressing a recombinant receptor or compositions thereof, and optionally instructions for use, for example, instructions for administering, according to the provided methods. In some embodiments, the instructions specify the criteria for selection or identification of subjects for therapy in accord with any of the provided methods.

In some embodiments, provided are articles of manufacture and/or kits that include a composition comprising a therapeutically effective amount of any of the engineered cells described herein, and instructions for administering, to a subject for treating a disease or condition. In some embodiments, the instructions can specify some or all of the elements of the methods provided herein. In some embodiments, the instructions specify particular instructions for administration of the cells for cell therapy, e.g., doses, timing, selection and/or identification of subjects for administration and conditions for administration. In some embodiments, the articles of manufacture and/or kits further include one or more additional agents for therapy, e.g., lymphodepleting therapy and/or combination therapy, such as any described herein and optionally further includes instructions for administering the additional agent for therapy. In some embodiments, the articles of manufacture and/or kits further comprise an agent for lymphodepleting therapy, and optionally further includes instructions for administering the lymphodepleting therapy. In some embodiments, the instructions can be included as a label or package insert accompanying the compositions for administration.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cell are formulated with a cryopreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryoprotected or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryoprotected or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the composition of enriched T cells, e.g., T cells that have been stimulated, engineered, and/or incubated, are formulated, cryoprotected, and then stored for an amount of time. In certain embodiments, the formulated, cryoprotected cells are stored until the cells are released for infusion. In particular embodiments, the formulated cryoprotected cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryoprotected and stored for, for about, or for less than 1 days, 2 days, 3 days, 4 days, 5 days, 6 days, or 7 days. In certain embodiments, the cells are thawed and administered to a subject after the storage. In certain embodiments, the cells are stored for or for about 5 days. In some embodiments, the formulated cells are not cryopreserved.

In some embodiments, the formulation is carried out using one or more processing step including washing, diluting or concentrating the cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to transduced and/or incubated cells. In some embodiments, the volume of formulation buffer is from or from about 10 mL to 1000 mL, such as at least or about at least or about or 50 mL, 100 mL, 200 mL, 300 mL, 400 mL, 500 mL, 600 mL, 700 mL, 800 mL, 900 mL or 1000 mL.

In some embodiments, such processing steps for formulating a cell composition are carried out in a closed system. Exemplary of such processing steps can be performed using a centrifugal chamber in conjunction with one or more systems or kits associated with a cell processing system, such as a centrifugal chamber produced and sold by Biosafe SA, including those for use with the Sepax® or Sepax 2® cell processing systems. An exemplary system and process is described in International Publication Number WO2016/073602. In some embodiments, the method includes effecting expression from the internal cavity of the centrifugal chamber a formulated composition, which is the resulting composition of cells formulated in a formulation buffer, such as pharmaceutically acceptable buffer, in any of the above embodiments as described. In some embodiments, the expression of the formulated composition is to a container, such as a bag that is operably linked as part of a closed system with the centrifugal chamber. In some embodiments, the container, such as bag, is connected to a system at an output line or output position.

In some embodiments, the closed system, such as associated with a centrifugal chamber or cell processing system, includes a multi-port output kit containing a multi-way tubing manifold associated at each end of a tubing line with a port to which one or a plurality of containers can be connected for expression of the formulated composition. In some aspects, a desired number or plurality of output containers, e.g., bags, can be sterilely connected to one or more, generally two or more, such as at least 3, 4, 5, 6, 7, 8 or more of the ports of the multi-port output. For example, in some embodiments, one or more containers, e.g., bags can be attached to the ports, or to fewer than all of the ports. Thus, in some embodiments, the system can effect expression of the output composition into a plurality of output bags.

In some aspects, cells can be expressed to the one or more of the plurality of output bags in an amount for dosage administration, such as for a single unit dosage administration or multiple dosage administration. For example, in some embodiments, the output bags may each contain the number of cells for administration in a given dose or fraction thereof. Thus, each bag, in some aspects, may contain a single unit dose for administration or may contain a fraction of a desired dose such that more than one of the plurality of output bags, such as two of the output bags, or 3 of the output bags, together constitute a dose for administration.

Thus, the containers, e.g., output bags, generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, each of the containers, e.g., bags, individually comprises a unit dose of the cells. Thus in some embodiments, each of the containers comprises the same or approximately or substantially the same number of cells. In some embodiments, each unit dose contains at least or about at least 1×10⁶, 2×10⁶, 5×10⁶, 1×10⁷, 5×10⁷, or 1×10⁸ engineered cells, total cells, T cells, or PBMCs. In some embodiments, the volume of the formulated cell composition in each bag is 10 mL to 100 mL, such as at least or about at least 20 mL, 30 mL, 40 mL, 50 mL, 60 mL, 70 mL, 80 mL, 90 mL or 100 mL.

In some embodiments, such cells produced by the method, or a composition comprising such cells, are administered to a subject for treating a disease or condition.

### III. Compositions and Formulations

The provided methods and uses involve use or administration of a dose of engineered cells of a composition comprising engineered T cells expressing a chimeric antigen receptor (CAR), e.g., an anti-CD19 CAR such as a CAR targeting human CD19. In certain embodiments, the composition is a therapeutic composition enriched in T cells, e.g., composition enriched in CD3+ T cells or composition enriched in CD4+ and CD8+ T cells, manufactured using a process for generating or producing output engineered cells and/or output compositions comprising engineered T cells disclosed herein, e.g., in Section II-C. In some embodiments, the engineered T cells are provided as a composition, formulation, or dose, such as a pharmaceutical composition, formulation, or dose. Such compositions, formulations, or doses can be used in accord with the provided methods or uses, and/or with the provided articles of manufacture or compositions, such as in the prevention or treatment of diseases, conditions, and disorders, or in detection, diagnostic, and prognostic methods.

In particular embodiments, the composition comprising engineered T cells expressing an anti-CD19 CAR is enriched in CD3+ T cells. In some embodiments, at least or about 50%, at least or about 60%, at least or about 65%, at least or about 70%, at least or about 75%, at least or about 80%, at least or about 85%, at least or about 90%, at least or about 95%, at least or about 96%, at least or about 98%, at least or about 98.5%, at least or about 99%, at least or about 99.5%, at least or about 99.9%, 100%, or about 100% of the total cells, the total viable cells, the total live cell, the total T cells, the total viable T cells, the total live T cell, the total live CD45+ cells, or CAR-expressing cells thereof in the composition, are CD3+, e.g., CD3+ T cells or CAR+CD3+ T cells. In some embodiments, between at or about 75% and at or about 80%, between at or about 80% and at or about 85%, between at or about 85% and at or about 90%, between at or about 90% and at or about 95%, between at or about 95% and at or about 99% of the total cells, the total viable cells, the total live cell, the total T cells, the total viable T cells, the total live T cell, the total live CD45+ cells, or CAR-expressing cells thereof in the composition, are CD3+, e.g., CD3+ T cells or CAR+CD3+ T cells. In some embodiments, at or about 80%, at or about 81%, at or about 82%, at or about 83%, at or about 84%, at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, at or about 99% of the total live CD45+ cells, or CAR-expressing cells thereof in the composition, are CD3+, e.g., CD3+ T cells or CAR+CD3+ T cells. In some embodiments, between about 80% and about 100%, between about 85% and about 99%, between about 88% and about 98%, between about 96% and about 99%, or between about 97% and about 99% of the total live CD45+ cells, or CAR-expressing cells thereof in the composition, are CD3+, e.g., CD3+ T cells or CAR+CD3+ T cells. In some embodiments, the composition consists of or consists essentially of CD3+ T cells. In some embodiments, at least or about 80% of the total cells in the composition are CD3+ T cells and at least or about 30%, at least or about 40%, at least or about 50%, at least or about 60%, at least or about 70%, at least or about 80%, at least or about 90%, or at least or about 95% of the total cells in the composition express the anti-CD19 CAR. In some embodiments, at least or about 80%, at least or about 85%, at least or about 90%, at least or about 95%, at least or about 96%, at least or about 97%, at least or about 98%, or at least or about 99% of the total live CD45+ cells in the composition are CD3+ and at least or about 40% or at least or about 50% of the total cells in the composition express the anti-CD19 CAR.

In some embodiments, less than or less than about 2.5%, less than or less than about 2%, less than or less than about 1.5%, less than or less than about 1%, less than or less than about 0.5%, less than or less than about 0.4%, less than or less than about 0.3%, less than or less than about 0.2%, less than or less than about 0.1%, less than or less than about 0.05%, or at or about 0% of the total cells, the total viable cells, the total live cell, the total T cells, the total viable T cells, the total live T cell, the total live CD45+ cells, or CAR-expressing cells thereof in the composition, are positive for expression of an NK cell marker. In some embodiments, between at or about 2.5% and at or about 2%, between at or about 2% and at or about 1.5%, between at or about 1.5% and at or about 1%, between at or about 1% and at or about 0.5%, between at or about 0.5% and at or about 0.4%, between at or about 0.4% and at or about 0.3%, between at or about 0.3% and at or about 0.2%, between at or about 0.2% and at or about 0.1%, between at or about 0.1% and at or about 0.05%, less than at or about 0.05%, or at or about 0% of the total cells, the total viable cells, the total live cell, the total T cells, the total viable T cells, the total live T cell, the total live CD45+ cells, or CAR-expressing cells thereof in the composition, are NK cells. In some embodiments, between at or about 1.5% and at or about 0%, or between at or about 0.5% and at or about 0%, of the total live CD45+ cells in the composition are NK cells. In some embodiments, the composition is free of or essentially free of NK cells or cells positive for expression of an NK cell marker.

In some embodiments, less than or less than about 0.2%, less than or less than about 0.15%, less than or less than about 0.1%, less than or less than about 0.05%, less than or less than about 0.01%, or at or about 0% of the total cells, the total viable cells, the total live cell, the total T cells, the total viable T cells, the total live T cell, the total live CD45+ cells, or CAR-expressing cells thereof in the composition, are CD19+. In some embodiments, between at or about 0.2% and at or about 0.15%, between at or about 0.15% and at or about 0.1%, between at or about 0.1% and at or about 0.05%, between at or about 0.05% and at or about 0.01%, less than at or about 0.01%, or at or about 0% of the total cells, the total viable cells, the total live cell, the total T cells, the total viable T cells, the total live T cell, the total live CD45+ cells, or CAR-expressing cells thereof in the composition, are CD19+. In some embodiments, between at or about 0.1% and at or about 0%, or between at or about 0.05% and at or about 0%, of the total live CD45+ cells in the composition are CD19+. In some embodiments, the composition is free of or essentially free of CD19+ cells.

In some embodiments, at least or about 80% of the total live CD45+ cells in the composition are CD3+, at least or about 40% of the total cells in the composition express the anti-CD19 CAR, less than about 1.5% of the total live CD45+ cells in the composition are NK cells or cells positive for expression of an NK cell marker, and less than about 0.1% of the total live CD45+ cells in the composition are CD19+. In some embodiments, at least or about 96% of the total live CD45+ cells in the composition are CD3+, at least or about 50% of the total cells in the composition express the anti-CD19 CAR, less than about 0.5% of the total live CD45+ cells in the composition are NK cells or cells positive for expression of an NK cell marker, and less than about 0.05% of the total live CD45+ cells in the composition are CD19+.

In particular embodiments, the composition comprising engineered T cells expressing an anti-CD19 CAR is enriched in CD4+ and CD8+ T cells. In some embodiments, at least or about 50%, at least or about 60%, at least or about 65%, at least or about 70%, at least or about 75%, at least or about 80%, at least or about 85%, at least or about 90%, at least or about 95%, at least or about 96%, at least or about 98%, at least or about 98.5%, at least or about 99%, at least or about 99.5%, at least or about 99.9%, 100%, or about 100% of the total cells, the total viable cells, the total live cell, the total T cells, the total viable T cells, the total live T cell, the total live CD45+ cells, or CAR-expressing cells thereof in the composition, are CD4+ or CD8+. In some embodiments, between at or about 75% and at or about 80%, between at or about 80% and at or about 85%, between at or about 85% and at or about 90%, between at or about 90% and at or about 95%, between at or about 95% and at or about 99% of the total cells, the total viable cells, the total live cell, the total T cells, the total viable T cells, the total live T cell, the total live CD45+ cells, or CAR-expressing cells thereof in the composition, are CD4+ or CD8+. In some embodiments, at or about 80%, at or about 81%, at or about 82%, at or about 83%, at or about 84%, at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, at or about 99% of the total live CD45+ cells, or CAR-expressing cells thereof in the composition, are CD4+ or CD8+. In some embodiments, between about 80% and about 100%, between about 85% and about 99%, between about 88% and about 98%, between about 96% and about 99%, or between about 97% and about 99% of the total live CD45+ cells, or CAR-expressing cells thereof in the composition, are CD4+ or CD8+. In some embodiments, the composition consists of or consists essentially of CD4+ T cells and CD8+ T cells. In some embodiments, at least or about 80% of the total cells in the composition are CD4+ T cells and CD8+ T cells and at least or about 30%, at least or about 40%, at least or about 50%, at least or about 60%, at least or about 70%, at least or about 80%, at least or about 90%, or at least or about 95% of the total cells in the composition express the anti-CD19 CAR. In some embodiments, at least or about 80%, at least or about 85%, at least or about 90%, at least or about 95%, at least or about 96%, at least or about 97%, at least or about 98%, or at least or about 99% of the total live CD45+ cells in the composition are CD4+ T cells and CD8+ T cells and at least or about 40% or at least or about 50% of the total cells in the composition express the anti-CD19 CAR.

In particular embodiments, CD3+CD4+ cells account for at least or about 50%, at least or about 60%, at least or about 65%, at least or about 70%, at least or about 75%, at least or about 80%, at least or about 85%, at least or about 90%, at least or about 95%, at least or about 96%, at least or about 98%, at least or about 98.5%, at least or about 99%, at least or about 99.5%, at least or about 99.9%, 100%, or about 100% of the total cells, the total viable cells, the total live cell, the total T cells, the total viable T cells, the total live T cell, the total live CD45+ cells, or CAR-expressing cells thereof in the composition. In particular embodiments, CD3+CD4+ cells account for between at or about 50% and at or about 70%, between at or about 50% and at or about 55%, between at or about 55% and at or about 60%, between at or about 60% and at or about 65%, or between at or about 65% and at or about 70% of the total live CD45+ cells in the composition.

In particular embodiments, CD3+CD8+ cells account for at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 98%, at least or about 98.5%, at least or about 99%, at least or about 99.5%, at least or about 99.9%, 100%, or about 100% of the total cells, the total viable cells, the total live cell, the total T cells, the total viable T cells, the total live T cell, the total live CD45+ cells, or CAR-expressing cells thereof in the composition. In particular embodiments, CD3+CD8+ cells account for between at or about 30% and at or about 50%, between at or about 30% and at or about 35%, between at or about 35% and at or about 40%, between at or about 40% and at or about 45%, or between at or about 45% and at or about 50% of the total live CD45+ cells in the composition.

In particular embodiments, CD3+CD4+ cells account for between about 55% and about 65% of the total live CD45+ cells in the composition, while CD3+CD8+ cells account for between about 35% and about 45% of the total live CD45+ cells in the composition. In particular embodiments, CD3+CD4+ cells account for about 60% while CD3+CD8+ cells account for about 40% of the total live CD45+ cells in the composition.

In particular embodiments, CAR+CD3+ cells (e.g., CD3+ cells expressing the anti-CD19 CAR) account for at least or about 20%, at least or about 30%, at least or about 40%, at least or about 50%, at least or about 60%, at least or about 65%, at least or about 70%, at least or about 75%, at least or about 80%, at least or about 85%, at least or about 90%, at least or about 95%, at least or about 96%, at least or about 98%, at least or about 98.5%, at least or about 99%, at least or about 99.5%, at least or about 99.9%, 100%, or about 100% of the total cells, the total viable cells, the total live cell, the total T cells, the total viable T cells, the total live T cell, the total live CD45+ cells, or CAR-expressing cells thereof in the composition. In particular embodiments, CAR+CD3+ cells (e.g., CD3+ cells expressing the anti-CD19 CAR) account for between at or about 40% and at or about 100%, between at or about 40% and at or about 45%, between at or about 45% and at or about 50%, between at or about 50% and at or about 55%, between at or about 55% and at or about 60%, between at or about 60% and at or about 65%, between at or about 65% and at or about 70%, between at or about 70% and at or about 75%, between at or about 75% and at or about 80%, between at or about 80% and at or about 85%, between at or about 85% and at or about 90%, between at or about 90% and at or about 95%, or between at or about 95% and at or about 99% of the total live CD45+ cells in the composition.

In particular embodiments, CAR+CD4+ cells (e.g., CD4+ cells expressing the anti-CD19 CAR) account for at least or about 20%, at least or about 30%, at least or about 40%, at least or about 50%, at least or about 60%, at least or about 65%, at least or about 70%, at least or about 75%, at least or about 80%, at least or about 85%, at least or about 90%, at least or about 95%, at least or about 96%, at least or about 98%, at least or about 98.5%, at least or about 99%, at least or about 99.5%, at least or about 99.9%, 100%, or about 100% of the total cells, the total viable cells, the total live cell, the total T cells, the total viable T cells, the total live T cell, the total live CD45+ cells, or CAR-expressing cells thereof in the composition. In particular embodiments, CAR+CD4+ cells (e.g., CD4+ cells expressing the anti-CD19 CAR) account for between at or about 20% and at or about 60%, between at or about 20% and at or about 25%, between at or about 25% and at or about 30%, between at or about 30% and at or about 35%, between at or about 35% and at or about 40%, between at or about 40% and at or about 45%, between at or about 45% and at or about 50%, between at or about 50% and at or about 55%, or between at or about 55% and at or about 60% of the total live CD45+ cells in the composition.

In particular embodiments, CAR+CD8+ cells (e.g., CD8+ cells expressing the anti-CD19 CAR) account for at least or about 10%, at least or about 20%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 98%, at least or about 98.5%, at least or about 99%, at least or about 99.5%, at least or about 99.9%, 100%, or about 100% of the total cells, the total viable cells, the total live cell, the total T cells, the total viable T cells, the total live T cell, the total live CD45+ cells, or CAR-expressing cells thereof in the composition. In particular embodiments, CAR+CD8+ cells (e.g., CD8+ cells expressing the anti-CD19 CAR) account for between at or about 5% and at or about 35%, between at or about 5% and at or about 10%, between at or about 10% and at or about 15%, between at or about 15% and at or about 20%, between at or about 20% and at or about 25%, between at or about 25% and at or about 30%, between at or about 30% and at or about 35% of the total live CD45+ cells in the composition.

In particular embodiments, CAR+CD3+ cells (e.g., CD3+ cells expressing the anti-CD19 CAR) account for between about 35% and about 65% of the total live CD45+ cells in the composition. In particular embodiments, CAR+CD4+ cells (e.g., CD4+ cells expressing the anti-CD19 CAR) account for between about 25% and about 55% of the total live CD45+ cells in the composition, while CAR+CD8+ cells (e.g., CD8+ cells expressing the anti-CD19 CAR) account for between about 10% and about 30% of the total live CD45+ cells in the composition. In particular embodiments, CD3+ cells expressing the anti-CD19 CAR account for about 50% of the total live CD45+ cells in the composition. In particular embodiments, CAR+CD4+ cells account for about 30% while CAR+CD8+ cells account for about 20% of the total live CD45+ cells in the composition. In particular embodiments, CD3+ cells expressing the anti-CD19 CAR account for about 60% of the total live CD45+ cells in the composition. In particular embodiments, CAR+CD4+ cells account for about 40% while CAR+CD8+ cells account for about 20% of the total live CD45+ cells in the composition.

In particular embodiments, the composition contains a ratio of between 3:1 and 1:3, between 2.5:1 and 1:2.5, between 2:1 and 1:2, between 1.5:1 and 1:1.5, between 1.4:1 and 1:1.4, between 1.3:1 and 1:1.3, between 1.2:1 and 1:1.2, or between 1.1:1 and 1:1.1 CD4+ T cells to CD8+ T cells. In some embodiments, the composition of cells has a ratio of or of about 3:1, of or of about 2.8:1, of or of about 2.5:1, of or of about 2.25:1, of or of about 2:1, of or of about 1.8:1, of or of about 1.7:1, of or of about 1.6:1, of or of about 1.5:1, of or of about 1.4:1, of or of about 1.3:1, of or of about 1.2:1, of or of about 1.1:1, of or of about 1:1, of or of about 1:1.1, of or of about 1:1.2, of or of about 1:1.3, of or of about 1:1.4, of or of about 1:1.5, of or of about 1:1.6, of or of about 1:1.7, of or of about 1:1.8, of or of about 1:2, of or of about 1:2.25, of or of about 1:2.5, of or of about 1:2.8, or of or of about 1:3 CD4+ T cells to CD8+ T cells.

In some embodiments, the output composition contains a ratio of between 3:1 and 1:3, between 2.5:1 and 1:2.5, between 2:1 and 1:2, between 1.5:1 and 1:1.5, between 1.4:1 and 1:1.4, between 1.3:1 and 1:1.3, between 1.2:1 and 1:1.2, or between 1.1:1 and 1:1.1 CD4+ T cells that express the recombinant receptor, e.g., the anti-CD19 CAR, to CD8+ T cells that express the recombinant receptor, e.g., the anti-CD19 CAR. In some embodiments, the ratio of CD4+ T cells that express the recombinant receptor (e.g., the anti-CD19 CAR) to CD8+ T cells that express the recombinant receptor (e.g., the anti-CD19 CAR) in the output composition is of or of about 3:1, of or of about 2.8:1, of or of about 2.5:1, of or of about 2.25:1, of or of about 2:1, of or of about 1.8:1, of or of about 1.7:1, of or of about 1.6:1, of or of about 1.5:1, of or of about 1.4:1, of or of about 1.3:1, of or of about 1.2:1, of or of about 1.1:1, of or of about 1:1, of or of about 1:1.1, of or of about 1:1.2, of or of about 1:1.3, of or of about 1:1.4, of or of about 1:1.5, of or of about 1:1.6, of or of about 1:1.7, of or of about 1:1.8, of or of about 1:2, of or of about 1:2.25, of or of about 1:2.5, of or of about 1:2.8, or of or of about 1:3.

In particular embodiments, the composition contains a ratio of between about 2.5:1 and about 1:2 or between about 2:1 and about 1:1 CD4+ T cells to CD8+ T cells. In some embodiments, the composition of cells has a ratio of or of about 1.5:1 CD4+ T cells to CD8+ T cells. In particular embodiments, the composition contains a ratio of between about 3:1 and about 1:1 CAR+CD4+ cells to CAR+CD8+ cells. In particular embodiments, the composition contains a ratio of between about 2.5:1 and about 1.5:1 CAR+CD4+ cells to CAR+CD8+ cells. In some embodiments, the composition of cells has a ratio of or of about 2:1 CAR+CD4+ cells to CAR+CD8+ cells. In some embodiments, the composition of cells has a ratio of or of about 1.5:1 CD4+ T cells to CD8+ T cells, and a ratio of or of about 2:1 CAR+CD4+ cells to CAR+CD8+ cells.

In particular embodiments, the composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable cells. In some embodiments, the composition contains at least at or about 75% viable cells. In certain embodiments, the composition contains at least at or about 85%, at least at or about 90%, or at least at or about 95% viable cells. In some embodiments, the composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD3+ T cells. In particular embodiments, the composition contains at least at or about 75% viable CD3+ T cells. In certain embodiments, the composition contains at least at or about 85%, at least at or about 90%, or at least at or about 95% viable CD3+ T cells. In some embodiments, the composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD4+ T cells. In certain embodiments, the composition contains at least at or about 75% viable CD4+ T cells. In particular embodiments, the composition contains at least at or about 85%, at least at or about 90%, or at least at or about 95% viable CD4+ T cells. In particular embodiments, the composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD8+ T cells. In some embodiments, the composition contains at least at or about 75% viable CD8+ T cells. In certain embodiments, the composition contains at least at or about 85%, at least at or about 90%, or at least at or about 95% viable CD8+ T cells.

In particular embodiments, the composition has a low portion and/or frequency of cells that are undergoing and/or are prepared, primed, and/or entering apoptosis. In particular embodiments, the composition has a low portion and/or frequency of cells that are positive for an apoptotic marker. In some embodiments, less than at or about 40%, less than at or about 35%, less than at or about 30%, less than at or about 25%, less than at or about 20%, less than at or about 15%, less than at or about 10%, less than at or about 5%, or less than at or about 1% of the cells of the composition express, contain, and/or are positive for an apoptotic marker. In certain embodiments, less than at or about 25% of the cells of the composition express, contain, and/or are positive for a marker of apoptosis. In certain embodiments, less than at or about less than at or about 10% cells of the composition express, contain, and/or are positive for an apoptotic marker.

In particular embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of anti-CD19 CAR-expressing cells of the composition are viable cells, e.g., cells negative for an apoptotic marker, such as a caspase (e.g., an activated caspase-3). In certain embodiments, at least at or about 85%, at least at or about 90%, or at least at or about 95% of anti-CD19 CAR-expressing cells of the composition are negative for an apoptotic marker, such as a caspase (e.g., an activated caspase-3). In some embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of CD3+ T cells of the composition are viable cells, e.g., cells negative for an apoptotic marker, such as a caspase (e.g., an activated caspase-3). In certain embodiments, at least at or about 85%, at least at or about 90%, or at least at or about 95% of CD3+ T cells of the composition are negative for an apoptotic marker, such as a caspase (e.g., an activated caspase-3). In particular embodiments, at least at or about 90% of CD3+ T cells of the composition are viable cells, e.g., cells negative for an apoptotic marker, such as a caspase (e.g., an activated caspase-3). In some embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of CAR+CD3+ T cells of the composition are viable cells, e.g., cells negative for an apoptotic marker, such as a caspase (e.g., an activated caspase-3). In particular embodiments, at least at or about 85%, at least at or about 90%, or at least at or about 95% of the anti-CD19 CAR-expressing CD3+ T cells of the composition are viable cells, e.g., cells negative for an apoptotic marker, such as a caspase (e.g., an activated caspase-3).

In some embodiments, less than or less than about 30%, less than or less than about 25%, less than or less than about 20%, less than or less than about 15%, less than or less than about 10%, or less than or less than about 5% of the total cells, the total T cells, the total CD45+ cells, the total CD3+ cells, the total CD4+ and CD8+ cells, or CAR-expressing cells thereof in the composition, express a marker of apoptosis, optionally Annexin V or active Caspase 3. In some embodiments, between at or about 30% and at or about 25%, between at or about 25% and at or about 20%, between at or about 20% and at or about 15%, between at or about 15% and at or about 10%, between at or about 10% and at or about 5% of the total cells, the total T cells, the total CD45+ cells, the total CD3+ cells, the total CD4+ and CD8+ cells, or CAR-expressing cells thereof in the composition, express a marker of apoptosis, optionally Annexin V or active Caspase 3. In some embodiments, at or about 6%, at or about 8%, at or about 10%, at or about 12%, at or about 14%, at or about 16%, at or about 18%, at or about 20%, at or about 22%, at or about 24%, at or about 26%, at or about 28%, at or about 30% of the CD3+ cells in the composition, express a marker of apoptosis, optionally Annexin V or active Caspase 3.

In some embodiments, expressing the anti-CD19 CAR may include, but is not limited to, having one or more recombinant receptor proteins localized at the cell membrane and/or cell surface, having a detectable amount of recombinant receptor protein, having a detectable amount of mRNA encoding the recombinant receptor, having or containing a recombinant polynucleotide that encodes the recombinant receptor, and/or having or containing an mRNA or protein that is a surrogate marker for recombinant receptor expression.

In some embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the cells of the composition express the recombinant receptor, e.g., the anti-CD19 CAR. In certain embodiments, at least or about 50% of the cells of the composition express the anti-CD19 CAR. In certain embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD3+ T cells of the composition express the anti-CD19 CAR. In some embodiments, at least or about 50% of the CD3+ T cells of the composition express the anti-CD19 CAR. In certain embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the cells of the composition are CD3+ T cells that express the anti-CD19 CAR. In some embodiments, at least or about 50% of the cells of the composition are CD3+ T cells that express the anti-CD19 CAR.

In some embodiments, the composition includes at least or at least about 0.2×10⁶ CD3+CAR+ cells/mL, 0.3×10⁶ CD3+CAR+ cells/mL, 0.4×10⁶ CD3+CAR+ cells/mL, 0.5×10⁶ CD3+CAR+ cells/mL, 0.6×10⁶ CD3+CAR+ cells/mL, 0.7×10⁶ CD3+CAR+ cells/mL, 0.8×10⁶ CD3+CAR+ cells/mL, 0.9×10⁶ CD3+CAR+ cells/mL, 1×10⁶ CD3+CAR+ cells/mL, 1.1×10⁶ CD3+CAR+ cells/mL, 1.2×10⁶ CD3+CAR+ cells/mL, 1.3×10⁶ CD3+CAR+ cells/mL, 1.4×10⁶ CD3+CAR+ cells/mL, 1.5×10⁶ CD3+CAR+ cells/mL, 1.6×10⁶ CD3+CAR+ cells/mL, 1.7×10⁶ CD3+CAR+ cells/mL, 1.8×10⁶ CD3+CAR+ cells/mL, 1.9×10⁶ CD3+CAR+ cells/mL, 2×10⁶ CD3+CAR+ cells/mL, 2.1×10⁶ CD3+CAR+ cells/mL, 2.2×10⁶ CD3+CAR+ cells/mL, 2.3×10⁶ CD3+CAR+ cells/mL, 2.4×10⁶ CD3+CAR+ cells/mL, 2.5×10⁶ CD3+CAR+ cells/mL, 2.6×10⁶ CD3+CAR+ cells/mL, 2.7×10⁶ CD3+CAR+ cells/mL, 2.8×10⁶ CD3+CAR+ cells/mL, 2.9×10⁶ CD3+CAR+ cells/mL, 3×10⁶ CD3+CAR+ cells/mL, 3.1×10⁶ CD3+CAR+ cells/mL, 3.2×10⁶ CD3+CAR+ cells/mL, 3.3×10⁶ CD3+CAR+ cells/mL, 3.4×10⁶ CD3+CAR+ cells/mL, 3.5×10⁶ CD3+CAR+ cells/mL, 3.6×10⁶ CD3+CAR+ cells/mL, 3.7×10⁶ CD3+CAR+ cells/mL, 3.8×10⁶ CD3+CAR+ cells/mL, 3.9×10⁶ CD3+CAR+ cells/mL, 4×10⁶ CD3+CAR+ cells/mL, 4.1×10⁶ CD3+CAR+ cells/mL, 4.2×10⁶ CD3+CAR+ cells/mL, 4.3×10⁶ CD3+CAR+ cells/mL, 4.4×10⁶ CD3+CAR+ cells/mL, 4.5×10⁶ CD3+CAR+ cells/mL, 4.6×10⁶ CD3+CAR+ cells/mL, 4.7×10⁶ CD3+CAR+ cells/mL, 4.8×10⁶ CD3+CAR+ cells/mL, 4.9×10⁶ CD3+CAR+ cells/mL, 5×10⁶ CD3+CAR+ cells/mL, 5.1×10⁶ CD3+CAR+ cells/mL, 5.2×10⁶ CD3+CAR+ cells/mL, 5.3×10⁶ CD3+CAR+ cells/mL, 5.4×10⁶ CD3+CAR+ cells/mL, 5.5×10⁶ CD3+CAR+ cells/mL, 5.6×10⁶ CD3+CAR+ cells/mL, 5.7×10⁶ CD3+CAR+ cells/mL, 5.8×10⁶ CD3+CAR+ cells/mL, 5.9×10⁶ CD3+CAR+ cells/mL, or 6×10⁶ CD3+CAR+ cells/mL, each inclusive. In some embodiments, the composition includes at least or at least about 0.2×10⁶ viable CD3+CAR+ cells/mL, 0.3×10⁶ viable CD3+CAR+ cells/mL, 0.4×10⁶ viable CD3+CAR+ cells/mL, 0.5×10⁶ viable CD3+CAR+ cells/mL, 0.6×10⁶ viable CD3+CAR+ cells/mL, 0.7×10⁶ viable CD3+CAR+ cells/mL, 0.8×10⁶ viable CD3+CAR+ cells/mL, 0.9×10⁶ viable CD3+CAR+ cells/mL, 1×10⁶ viable CD3+CAR+ cells/mL, 1.1×10⁶ viable CD3+CAR+ cells/mL, 1.2×10⁶ viable CD3+CAR+ cells/mL, 1.3×10⁶ viable CD3+CAR+ cells/mL, 1.4×10⁶ viable CD3+CAR+ cells/mL, 1.5×10⁶ viable CD3+CAR+ cells/mL, 1.6×10⁶ viable CD3+CAR+ cells/mL, 1.7×10⁶ viable CD3+CAR+ cells/mL, 1.8×10⁶ viable CD3+CAR+ cells/mL, 1.9×10⁶ viable CD3+CAR+ cells/mL, 2×10⁶ viable CD3+CAR+ cells/mL, 2.1×10⁶ viable CD3+CAR+ cells/mL, 2.2×10⁶ viable CD3+CAR+ cells/mL, 2.3×10⁶ viable CD3+CAR+ cells/mL, 2.4×10⁶ viable CD3+CAR+ cells/mL, 2.5×10⁶ viable CD3+CAR+ cells/mL, 2.6×10⁶ viable CD3+CAR+ cells/mL, 2.7×10⁶ viable CD3+CAR+ cells/mL, 2.8×10⁶ viable CD3+CAR+ cells/mL, 2.9×10⁶ viable CD3+CAR+ cells/mL, 3×10⁶ viable CD3+CAR+ cells/mL, 3.1×10⁶ viable CD3+CAR+ cells/mL, 3.2×10⁶ viable CD3+CAR+ cells/mL, 3.3×10⁶ viable CD3+CAR+ cells/mL, 3.4×10⁶ viable CD3+CAR+ cells/mL, 3.5×10⁶ viable CD3+CAR+ cells/mL, 3.6×10⁶ viable CD3+CAR+ cells/mL, 3.7×10⁶ viable CD3+CAR+ cells/mL, 3.8×10⁶ viable CD3+CAR+ cells/mL, 3.9×10⁶ viable CD3+CAR+ cells/mL, 4×10⁶ viable CD3+CAR+ cells/mL, 4.1×10⁶ viable CD3+CAR+ cells/mL, 4.2×10⁶ viable CD3+CAR+ cells/mL, 4.3×10⁶ viable CD3+CAR+ cells/mL, 4.4×10⁶ viable CD3+CAR+ cells/mL, 4.5×10⁶ viable CD3+CAR+ cells/mL, 4.6×10⁶ viable CD3+CAR+ cells/mL, 4.7×10⁶ viable CD3+CAR+ cells/mL, 4.8×10⁶ viable CD3+CAR+ cells/mL, 4.9×10⁶ viable CD3+CAR+ cells/mL, 5×10⁶ viable CD3+CAR+ cells/mL, 5.1×10⁶ viable CD3+CAR+ cells/mL, 5.2×10⁶ viable CD3+CAR+ cells/mL, 5.3×10⁶ viable CD3+CAR+ cells/mL, 5.4×10⁶ viable CD3+CAR+ cells/mL, 5.5×10⁶ viable CD3+CAR+ cells/mL, 5.6×10⁶ viable CD3+CAR+ cells/mL, 5.7×10⁶ viable CD3+CAR+ cells/mL, 5.8×10⁶ viable CD3+CAR+ cells/mL, 5.9×10⁶ viable CD3+CAR+ cells/mL, or 6×10⁶ viable CD3+CAR+ cells/mL, each inclusive.

In particular embodiments, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD4+ T cells of the composition express the recombinant receptor, e.g., the anti-CD19 CAR. In particular embodiments, at least or about 50% of the CD4+ T cells of the composition express the recombinant receptor, e.g., the anti-CD19 CAR. In some embodiments, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD8+ T cells of the composition express the recombinant receptor, e.g., the anti-CD19 CAR. In certain embodiments, at least or about 50% of the CD8+ T cells of the composition express the recombinant receptor, e.g., the anti-CD19 CAR.

In some embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of live CD45+ cells in the composition are CD3+CAR+(e.g., CD3+ T cells that express the anti-CD19 CAR), CD4+CAR+(e.g., CD4+ T cells that express the anti-CD19 CAR), and/or CD8+CAR+(e.g., CD8+ T cells that express the anti-CD19 CAR). In certain embodiments, at least or about 50% of live CD45+ cells in the composition are CD3+ T cells that express the anti-CD19 CAR. In certain embodiments, between at or about 60% and at or about 65% of live CD45+ cells in the composition are CD3+ T cells that express the anti-CD19 CAR. In certain embodiments, between at or about 35% and at or about 45%, between at or about 35% and at or about 40%, or between at or about 40% and at or about 45% of live CD45+ cells in the composition are CD4+ T cells that express the anti-CD19 CAR. In certain embodiments, between at or about 15% and at or about 25%, between at or about 15% and at or about 20%, or between at or about 20% and at or about 25% of live CD45+ cells in the composition are CD8+ T cells that express the anti-CD19 CAR. In certain embodiments, of the live CD45+ cells in the composition, at least or about 60% are CD3+ T cells that express the anti-CD19 CAR, at least or about 40% are CD4+ T cells that express the anti-CD19 CAR, and at least or about 20% are CD8+ T cells that express the anti-CD19 CAR.

In any of the proceeding embodiments, the composition can comprise about or at least about 10×10⁶, about or at least about 20×10⁶, about or at least about 25×10⁶, about or at least about 50×10⁶, about or at least about 100×10⁶, about or at least about 200×10⁶, about or at least about 400×10⁶, about or at least about 600×10⁶, about or at least about 800×10⁶, about or at least about 1000×10⁶, about or at least about 1200×10⁶, about or at least about 1400×10⁶, about or at least about 1600×10⁶, about or at least about 1800×10⁶, about or at least about 2000×10⁶, about or at least about 2500×10⁶, about or at least about 3000×10⁶, or about or at least about 4000×10⁶ total cells, e.g., total viable cells, in one or more containers such as vials. In any of the proceeding embodiments, the volume of the composition can be between 1.0 mL and 10 mL, inclusive, optionally at or about 2 mL, at or about 3 mL, at or about 4 mL, at or about 5 mL, at or about 6 mL, at or about 7 mL, at or about 8 mL, at or about 9 mL, or at or about 10 mL, or any value between any of the foregoing. In some embodiments, the composition is contained in a plurality of containers, such as 2, 3, 4, 5, 6, 7, 8, 9, 10 or more vials. In any of the proceeding embodiments, the composition can comprise about or at least about 5×10⁶, about or at least about 10×10⁶, about or at least about 20×10⁶, about or at least about 25×10⁶, about or at least about 50×10⁶, about or at least about 100×10⁶, about or at least about 150×10⁶, about or at least about 200×10⁶, about or at least about 250×10⁶, about or at least about 300×10⁶, about or at least about 350×10⁶, about or at least about 400×10⁶, about or at least about 450×10⁶, about or at least about 500×10⁶, about or at least about 550×10⁶, or about or at least about 600×10⁶ total cells, e.g., total viable cells, per unit container such as per vial. In some embodiments, cells of the composition in the one or more containers are at a density of, of about, or at least 5×10⁶ cells/mL, 10×10⁶ cells/mL, 20×10⁶ cells/mL, 30×10⁶ cells/mL, 40×10⁶ cells/mL, 50×10⁶ cells/mL, 60×10⁶ cells/mL, 70×10⁶ cells/mL, 80×10⁶ cells/mL, 90×10⁶ cells/mL, 100×10⁶ cells/mL, 110×10⁶ cells/mL, 120×10⁶ cells/mL, 130×10⁶ cells/mL, 140×10⁶ cells/mL, or 150×10⁶ cells/mL in a solution or buffer, e.g., in a cryopreservation solution or buffer. In some embodiments, about or up to about 900×10⁶ cells (e.g., viable CD4+ T cells and viable CD8+ T cells, or viable CD3+ T cells) are subjected to stimulation, where about or up to about 600×10⁶ cells (e.g., viable CD4+ T cells and viable CD8+ T cells, or viable CD3+ T cells) of the stimulated composition are subjected to genetic engineering such as with a viral vector, e.g. by transduction, or a non-viral method of genetic engineering followed by incubation in a serum-free basal media (e.g., supplemented with one or more supplements) without any recombinant cytokine for about 72 hours or about three days. In some embodiments, the output composition produced comprises between about 100×10⁶ and about 1400×10⁶ total cells, e.g., total viable cells, in one or more containers such as vials.

In particular embodiments, a majority of the cells of the composition are naïve or naïve-like cells, central memory cells, and/or effector memory cells. In particular embodiments, a majority of the cells of the composition are naïve-like or central memory cells. In some embodiments, a majority of the cells of the output composition are central memory cells. In some aspects, less differentiated cells, e.g., central memory cells, are longer lived and exhaust less rapidly, thereby increasing persistence and durability. In some aspects, a responder to a cell therapy, such as a CAR-T cell therapy, has increased expression of central memory genes. See, e.g., Fraietta et al. (2018) Nat Med. 24(5):563-571.

In certain embodiments, the cells of the composition have a high portion and/or frequency of naïve-like T cells or T cells that are surface positive for a marker expressed on naïve-like T cells. In certain embodiments, the cells of the compositions have a greater portion and/or frequency of naïve-like cells than compositions generated from alternative processes, such as processes that involve expansion (e.g. processes that include an expansion unit operation and/or include steps intended to cause expansion of cells). In certain embodiments, naïve-like T cells may include cells in various differentiation states and may be characterized by positive or high expression (e.g., surface expression or intracellular expression) of certain cell markers and/or negative or low expression (e.g., surface expression or intracellular expression) of other cell markers. In some aspects, naïve-like T cells are characterized by positive or high expression of CCR7, CD45RA, CD28, and/or CD27. In some aspects, naïve-like T cells are characterized by negative expression of CD25, CD45RO, CD56, CD62L, and/or KLRG1. In some aspects, naïve-like T cells are characterized by low expression of CD95. In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+CCR7+, where the cells are CD45RA+ or CD45RA−. In certain embodiments, naïve-like T cells or the T cells that are surface positive for a marker expressed on naïve-like T cells are CD62L−CCR7+.

In particular embodiments, the cells of the composition are enriched in CCR7+ cells. CCR7 is a chemokine receptor that is involved in T cell entry into lymph nodes. In particular aspects, CCR7 is expressed by naïve or naïve-like T cells (e.g. CCR7+CD45RA+ or CCR7+CD27+) and central memory T cells (CCR7+CD45RA−). In some embodiments, provided compositions of engineered T cells produced by the provided methods include a population of T cells in which greater than at or about 50%, greater than at or about 55%, greater than or greater than at or about 60%, greater than or greater than at or about 65%, greater than or greater than at or about 70%, greater than or greater than at or about 75%, greater than or greater than at or about 80%, greater than or greater than at or about 85%, or greater than or greater than at or about 90%, of the T cells of the population are central memory and naïve-like T cells. In some embodiments, provided compositions of engineered T cells produced by the provided methods include a population of T cells in which greater than at or about 50%, greater than at or about 55%, greater than or greater than at or about 60%, greater than or greater than at or about 65%, greater than or greater than at or about 70%, greater than or greater than at or about 75%, greater than or greater than at or about 80%, greater than or greater than at or about 85%, or greater than or greater than at or about 90%, of the T cells of the population are CCR7+ T cells. In some embodiments, provided compositions of engineered T cells produced by the provided methods include a population of T cells in which greater than at or about 50%, greater than at or about 55%, greater than or greater than at or about 60%, greater than or greater than at or about 65%, greater than or greater than at or about 70%, greater than or greater than at or about 75%, greater than or greater than at or about 80%, greater than or greater than at or about 85%, or greater than or greater than at or about 90%, of the T cells of the population are CCR7+CD27+. In some embodiments, provided compositions of engineered T cells produced by the provided methods include a population of T cells in which greater than at or about 50%, greater than at or about 55%, greater than or greater than at or about 60%, greater than or greater than at or about 65%, greater than or greater than at or about 70%, greater than or greater than at or about 75%, greater than or greater than at or about 80%, greater than or greater than at or about 85%, or greater than or greater than at or about 90%, of the T cells of the population are CCR7+CD45RA−.

In certain embodiments, the cells of the output composition have a high portion and/or frequency of central memory T cells or T cells that are surface positive for a marker expressed on central memory T cells. In certain embodiments, the cells of the output compositions have a greater portion and/or frequency of central memory cells than output compositions generated from alternative processes, such as processes that involve expansion (e.g. processes that include an expansion unit operation and/or include steps intended to cause expansion of cells). In certain embodiments, central memory T cells may include cells in various differentiation states and may be characterized by positive or high expression (e.g., surface expression) of certain cell markers and/or negative or low expression (e.g., surface expression) of other cell markers. In some aspects, central memory T cells are characterized by positive or high expression of CD45RO, CD62L, CCR7, CD28, CD3, and/or CD127. In some aspects, central memory T cells are characterized by negative or low expression of CD45RA and/or granzyme B. In certain embodiments, central memory T cells or the T cells that are surface positive for a marker expressed on central memory T cells are CCR7+CD45RA−.

In certain embodiments, the cells of the output compositions have a greater portion and/or frequency of naïve-like cells and central memory cells than output compositions generated from alternative processes, such as processes that involve expansion (e.g. processes that include an expansion unit operation and/or include steps intended to cause expansion of cells).

In certain embodiments, the cells of the output composition have a low portion and/or frequency of effector memory and/or effector memory RA T cells or T cells that are surface positive for a marker expressed on effector memory and/or effector memory RA T cells. In certain embodiments, the cells of the output compositions have a lower portion and/or frequency of effector memory and/or effector memory RA T cells than output compositions generated from alternative processes, such as processes that involve expansion (e.g. processes that include an expansion unit operation and/or include steps intended to cause expansion of cells). In certain embodiments, effector memory and/or effector memory RA T cells may include cells in various differentiation states and may be characterized by positive or high expression (e.g., surface expression or intracellular expression) of certain cell markers and/or negative or low expression (e.g., surface expression or intracellular expression) of other cell markers. In certain embodiments, effector memory T cells or the T cells that are surface positive for a marker expressed on effector memory T cells are CCR7−CD45RA−. In certain embodiments, effector memory RA T cells or the T cells that are surface positive for a marker expressed on effector memory RA T cells are CCR7−CD45RA+.

In certain embodiments, the cells of the output compositions have a lower portion and/or frequency of effector memory T cells than output compositions generated from alternative processes, such as processes that involve expansion (e.g. processes that include an expansion unit operation and/or include steps intended to cause expansion of cells). In certain embodiments, the cells of the output compositions have a lower portion and/or frequency of effector memory RA T cells than output compositions generated from alternative processes, such as processes that involve expansion (e.g. processes that include an expansion unit operation and/or include steps intended to cause expansion of cells). In certain embodiments, the cells of the output compositions have a greater portion and/or frequency of naïve-like cells and central memory cells and a lower portion and/or frequency of effector memory and effector memory RA T cells, than output compositions generated from alternative processes, such as processes that involve expansion (e.g. processes that include an expansion unit operation and/or include steps intended to cause expansion of cells).

In certain embodiments, the cells of the output composition have a high portion and/or frequency of naïve-like and/or central memory cells. In certain embodiments, the cells of the output composition have a high portion and/or frequency of central memory cells. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the cells of the output composition are of a memory phenotype, are of a naïve-like or central memory phenotype, or are naïve-like or central memory T cells, or are central memory T cells. In certain embodiments, at least or at or about 50%, at least or at or about 55%, at least or at or about 60%, or at least or at or about 65% of the CD4+ T cells and CD8+ T cells of the output composition are naïve-like or central memory T cells, or are central memory T cells. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CD4+ T cells of the output composition are naïve-like or central memory CD4+ T cells, or are central memory CD4+ T cells. In certain embodiments, at least or at or about 50%, at least or at or about 55%, at least or at or about 60%, or at least or at or about 65% of the CD4+ T cells of the output composition are naïve-like or central memory CD4+ T cells, or are central memory CD4+ T cells. In certain embodiments, between at or about 40% and at or about 65%, between at or about 40% and at or about 45%, between at or about 45% and at or about 50%, between at or about 50% and at or about 55%, between at or about 55% and at or about 60%, or between at or about 60% and at or about 65% of the CD4+ T cells of the output composition are naïve-like or central memory CD4+ T cells. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CD4+CAR+ T cells of the output composition are naïve-like or central memory CD4+CAR+ T cells, or are central memory CD4+CAR+ T cells. In certain embodiments, at least or at or about 50%, at least or at or about 55%, at least or at or about 60%, or at least or at or about 65% of the CD4+CAR+ T cells of the output composition are naïve-like or central memory CD4+CAR+ T cells, or are central memory CD4+CAR+ T cells. In certain embodiments, between at or about 40% and at or about 65%, between at or about 40% and at or about 45%, between at or about 45% and at or about 50%, between at or about 50% and at or about 55%, between at or about 55% and at or about 60%, or between at or about 60% and at or about 65% of the CD4+CAR+ T cells of the output composition are naïve-like or central memory CD4+CAR+ T cells, or are central memory CD4+CAR+ T cells. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CD8+ T cells of the output composition are naïve-like or central memory CD8+ T cells, or are central memory CD8+ T cells. In certain embodiments, at least or at or about 50%, at least or at or about 55%, at least or at or about 60%, or at least or at or about 65% of the CD8+ T cells of the output composition are naïve-like or central memory CD8+ T cells, or are central memory CD8+ T cells. In certain embodiments, between at or about 40% and at or about 65%, between at or about 40% and at or about 45%, between at or about 45% and at or about 50%, between at or about 50% and at or about 55%, between at or about 55% and at or about 60%, or between at or about 60% and at or about 65% of the CD8+ T cells of the output composition are naïve-like or central memory CD8+ T cells, or are central memory CD8+ T cells. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CD8+CAR+ T cells of the output composition are naïve-like or central memory CD8+CAR+ T cells, or are central memory CD8+CAR+ T cells. In certain embodiments, at least or at or about 50%, at least or at or about 55%, at least or at or about 60%, or at least or at or about 65% of the CD8+CAR+ T cells of the output composition are naïve-like or central memory CD8+CAR+ T cells, or are central memory CD8+CAR+ T cells. In certain embodiments, between at or about 40% and at or about 65%, between at or about 40% and at or about 45%, between at or about 45% and at or about 50%, between at or about 50% and at or about 55%, between at or about 55% and at or about 60%, or between at or about 60% and at or about 65% of the CD8+CAR+ T cells of the output composition are naïve-like or central memory CD8+CAR+ T cells, or are central memory CD8+CAR+ T cells. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CAR+ T cells (e.g., the CD4+CAR+ T cells and the CD8+CAR+ T cells) of the output composition are naïve-like or central memory T cells, or are central memory T cells. In certain embodiments, at least or at or about 50%, at least or at or about 55%, at least or at or about 60%, or at least or at or about 65% of the CAR+ T cells (e.g., the CD4+CAR+ T cells and the CD8+CAR+ T cells) of the output composition are naïve-like or central memory T cells, or are central memory T cells. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CAR+ T cells in the composition are CD27+, CD28+, CCR7+, CD45RA−, CD45RO+, CD62L+, CD3+, CD95+, granzyme B−, and/or CD127+. In some embodiments, at least or at or about 50%, at least or at or about 55%, at least or at or about 60%, or at least or at or about 65% of the CAR+ T cells in the composition are CD27+, CD28+, CCR7+, CD45RA−, CD45RO+, CD62L+, CD3+, CD95+, granzyme B−, and/or CD127+.

In certain embodiments, at least or at or about 85% of the cells of the output composition are of a naïve-like or central memory phenotype, or are naïve-like or central memory T cells. In certain embodiments, less than or at or about 15% of the cells of the output composition are of an effector or effector RA phenotype, or are effector or effector RA T cells. In certain embodiments, the cells of the output composition have a low portion and/or frequency of cells that are exhausted and/or senescent. In particular embodiments, the cells of the output composition have a low portion and/or frequency of cells that are exhausted and/or senescent. In some embodiments, less than at or about 40%, less than at or about 35%, less than at or about 30%, less than at or about 25%, less than at or about 20%, less than at or about 15%, less than at or about 10%, less than at or about 5%, or less than at or about 1% of the cells of the output composition are exhausted and/or senescent. In certain embodiments, less than at or about 25% of the cells of the output composition are exhausted and/or senescent. In certain embodiments, less than at or about less than at or about 10% of the cells of the output composition are exhausted and/or senescent.

In some embodiments, the cells of the output composition have a low portion and/or frequency of cells that are negative for CD27 and CD28 expression, e.g., surface expression. In particular embodiments, the cells of the output composition have a low portion and/or frequency of CD27-CD28-cells. In some embodiments, less than at or about 40%, less than at or about 35%, less than at or about 30%, less than at or about 25%, less than at or about 20%, less than at or about 15%, less than at or about 10%, less than at or about 5%, or less than at or about 1% of the cells of the output composition are CD27−CD28− cells. In certain embodiments, less than at or about 25% of the cells of the output composition are CD27−CD28− cells. In certain embodiments, less than at or about less than at or about 10% of the cells of the output composition are CD27−CD28− cells. In embodiments, less than at or about 5% of the cells of the output composition are CD27−CD28− cells.

In certain embodiments, the cells of the output composition have a high portion and/or frequency of cells that are positive for CD27 and CD28 expression, e.g., surface expression. In some embodiments, the cells of the output composition have a high portion and/or frequency of CD27+CD28+ cells. In some embodiments, at least at or about 50%, at least at or about 60%, at least at or about 70%, at least at or about 75%, at least at or about 80%, at least at or about 85%, at least at or about 90%, at least at or about 95%, or greater than at or about 95% of the cells of the output composition are CD27+CD28+ cells. In certain embodiments, less than at or about 25% of the cells of the output composition are CD27−CD28− cells. In certain embodiments, at least at or about 50% of the cells of the output composition are CD27+CD28+ cells. In embodiments, at least at or about 75% of the cells of the output composition are CD27+CD28+ cells.

In particular embodiments, the cells of the output composition have a low portion and/or frequency of cells that are T_{EMRA} cells. In particular embodiments, the cells of the output composition have a low portion and/or frequency of T_{EMRA} cells. In some embodiments, less than at or about 40%, less than at or about 35%, less than at or about 30%, less than at or about 25%, less than at or about 20%, less than at or about 15%, less than at or about 10%, less than at or about 5%, or less than at or about 1% of the cells of the output composition are T_{EMRA} cells. In some embodiments, less than at or about 25% of the cells of the output composition are T_{EMRA} cells. In some embodiments, less than at or about 10% of the cells of the output composition are T_{EMRA} cells. In some embodiments, less than at or about 5% of the cells of the output composition are T_{EMRA} cells.

In certain embodiments, the cells of the output composition have a low portion and/or frequency of cells that are negative for CCR7 and positive for CD45RA expression, e.g., surface expression. In some embodiments, the cells of the output composition have a low portion and/or frequency of CCR7−CD45RA+ cells. In particular embodiments, less than at or about 40%, less than at or about 35%, less than at or about 30%, less than at or about 25%, less than at or about 20%, less than at or about 15%, less than at or about 10%, less than at or about 5%, or less than at or about 1% of the cells of the output composition are CCR7−CD45RA+ cells. In some embodiments, less than at or about 25% of the cells of the output composition are CCR7−CD45RA+ cells. In particular embodiments, less than at or about less than at or about 10% of the cells of the output composition are CCR7−CD45RA+ cells. In certain embodiments, less than at or about 5% of the cells of the output composition are CCR7-CD45RA+ cells.

In certain embodiments, the cells of the output composition have a high portion and/or frequency of T cells in an early stage of differentiation, or T cells that are surface positive for a marker expressed on T cells in an early stage of differentiation. In certain embodiments, the cells of the output compositions have a greater portion and/or frequency of T cells in an early stage of differentiation than output compositions generated from alternative processes, such as processes that involve expansion (e.g. processes that include an expansion unit operation and/or include steps intended to cause expansion of cells). In certain embodiments, T cells in an early stage of differentiation may be characterized by positive or high expression (e.g., surface expression or intracellular expression) of certain cell markers and/or negative or low expression (e.g., surface expression or intracellular expression) of other cell markers. In some aspects, T cells in an early stage of differentiation are characterized by positive or high expression of CCR7 and/or CD27. In certain embodiments, T cells in an early stage of differentiation or the T cells that are surface positive for a marker expressed on T cells in an early stage of differentiation are CCR7+CD27+.

In certain embodiments, the cells of the output composition have a low portion and/or frequency of T cells in an intermediate stage of differentiation, or T cells that are surface positive for a marker expressed on T cells in an intermediate stage of differentiation. In certain embodiments, the cells of the output compositions have a lower portion and/or frequency of T cells in an intermediate stage of differentiation than output compositions generated from alternative processes, such as processes that involve expansion. In certain embodiments, T cells in an intermediate stage of differentiation may be characterized by positive or high expression (e.g., surface expression or intracellular expression) of certain cell markers and/or negative or low expression (e.g., surface expression or intracellular expression) of other cell markers. In certain embodiments, T cells in an intermediate stage of differentiation or the T cells that are surface positive for a marker expressed on T cells in an intermediate stage of differentiation are CCR7+CD27−. In certain embodiments, T cells in an intermediate stage of differentiation or the T cells that are surface positive for a marker expressed on T cells in an intermediate stage of differentiation are CCR7−CD27+. In certain embodiments, T cells in an intermediate stage of differentiation or the T cells that are surface positive for a marker expressed on T cells in an intermediate stage of differentiation include cells that are CCR7+CD27− and cells that are CCR7−CD27+.

In certain embodiments, the cells of the output composition have a low portion and/or frequency of highly differentiated T cells, or T cells that are surface positive for a marker expressed on highly differentiated T cells. In certain embodiments, the cells of the output compositions have a lower portion and/or frequency of highly differentiated T cells than output compositions generated from alternative processes, such as processes that involve expansion. In certain embodiments, highly differentiated T cells may be characterized by positive or high expression (e.g., surface expression or intracellular expression) of certain cell markers and/or negative or low expression (e.g., surface expression or intracellular expression) of other cell markers. In some aspects, highly differentiated T cells are characterized by negative or low expression of CCR7 and/or CD27. In certain embodiments, highly differentiated T cells or the T cells that are surface positive for a marker expressed on highly differentiated T cells are CCR7−CD27−.

In certain embodiments, the cells of the output compositions have a greater portion and/or frequency of T cells in an early stage of differentiation (e.g., cells that are CCR7+CD27+), a lower portion and/or frequency of T cells in an intermediate stage of differentiation (e.g., cells that are CCR7+CD27− and/or cells that are CCR7−CD27+), and a lower portion and/or frequency of highly differentiated T cells (e.g., cells that are CCR7−CD27−), than output compositions generated from alternative processes, such as processes that involve expansion.

In certain embodiments, the cells of the output compositions have a greater portion and/or frequency of naïve-like cells and central memory cells than output compositions generated from alternative processes, such as processes that involve expansion. In certain embodiments, the naïve-like cells and central memory cells include cells in various differentiation states, including T cells in an early stage of differentiation, e.g., cells that are CCR7+CD27+.

In certain embodiments, the cells of the output composition have a high portion and/or frequency of cells that are positive for CCR7 and CD27 expression, e.g., surface expression. In some embodiments, the cells of the output composition have a high portion and/or frequency of CCR7+CD27+ cells. In certain embodiments, less than or less than about 5%, less than or less than about 10%, less than or less than about 15%, less than or less than about 20%, less than or less than about 25%, or less than or less than about 30% of the cells the output composition are CCR7− or CD27− cells. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, at least or at or about 98%, or greater than 98% of the cells of the output composition are CCR7+CD27+. In certain embodiments, at least or at or about 50%, at least or at or about 55%, at least or at or about 60%, or at least or at or about 65% of the CD4+ T cells and CD8+ T cells of the output composition are CCR7+CD27+. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CD4+ T cells of the output composition are CCR7+CD27+. In certain embodiments, at least or at or about 50%, at least or at or about 55%, at least or at or about 60%, or at least or at or about 65% of the CD4+ T cells of the output composition are CCR7+CD27+. In certain embodiments, between at or about 40% and at or about 65%, between at or about 40% and at or about 45%, between at or about 45% and at or about 50%, between at or about 50% and at or about 55%, between at or about 55% and at or about 60%, or between at or about 60% and at or about 65% of the CD4+ T cells of the output composition are CCR7+CD27+. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CD4+CAR+ T cells of the output composition are CCR7+CD27+. In certain embodiments, at least or at or about 50%, at least or at or about 55%, at least or at or about 60%, or at least or at or about 65% of the CD4+CAR+ T cells of the output composition are CCR7+CD27+. In certain embodiments, between at or about 40% and at or about 65%, between at or about 40% and at or about 45%, between at or about 45% and at or about 50%, between at or about 50% and at or about 55%, between at or about 55% and at or about 60%, or between at or about 60% and at or about 65% of the CD4+CAR+ T cells of the output composition are CCR7+CD27+. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CD8+ T cells of the output composition are CCR7+CD27+. In certain embodiments, at least or at or about 50%, at least or at or about 55%, at least or at or about 60%, or at least or at or about 65% of the CD8+ T cells of the output composition are CCR7+CD27+. In certain embodiments, between at or about 40% and at or about 65%, between at or about 40% and at or about 45%, between at or about 45% and at or about 50%, between at or about 50% and at or about 55%, between at or about 55% and at or about 60%, or between at or about 60% and at or about 65% of the CD8+ T cells of the output composition are CCR7+CD27+. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CD8+CAR+ T cells of the output composition are CCR7+CD27+. In certain embodiments, at least or at or about 50%, at least or at or about 55%, at least or at or about 60%, or at least or at or about 65% of the CD8+CAR+ T cells of the output composition are CCR7+CD27+. In certain embodiments, between at or about 40% and at or about 65%, between at or about 40% and at or about 45%, between at or about 45% and at or about 50%, between at or about 50% and at or about 55%, between at or about 55% and at or about 60%, or between at or about 60% and at or about 65% of the CD8+CAR+ T cells of the output composition are CCR7+CD27+. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CAR+ T cells (e.g., the CD4+CAR+ T cells and the CD8+CAR+ T cells) of the output composition are CCR7+CD27+. In certain embodiments, at least or at or about 50%, at least or at or about 55%, at least or at or about 60%, or at least or at or about 65% of the CAR+ T cells (e.g., the CD4+CAR+ T cells and the CD8+CAR+ T cells) of the output composition are CCR7+CD27+. In some embodiments, at least or at or about 30%, at least or at or about 40%, at least or at or about 50%, at least or at or about 60%, at least or at or about 70%, at least or at or about 75%, at least or at or about 80%, at least or at or about 85%, at least or at or about 90%, at least or at or about 95%, or greater than 95% of the CAR+ T cells in the composition are CD27+, CD28+, CCR7+, CD45RA−, CD45RO+, CD62L+, CD3+, CD95+, granzyme B−, and/or CD127+. In some embodiments, at least or at or about 50%, at least or at or about 55%, at least or at or about 60%, or at least or at or about 65% of the CAR+ T cells in the composition are CD27+, CD28+, CCR7+, CD45RA−, CD45RO+, CD62L+, CD3+, CD95+, granzyme B−, and/or CD127+.

In some embodiments, provided herein is a therapeutic T cell composition comprising and/or enriched in CD3+ T cells expressing a recombinant receptor, wherein at least 50%, 60%, 70%, 80% or 90% of the total receptor⁺/CD3+ cells in the composition are CD27+CCR7+. In some embodiments, at least or at least about 80%, at least or at least about 85%, at least or at least about 90%, at least or at least about 95%, at least or at least about 96%, at least or at least about 97%, at least or at least about 98%, at least or at least about 99%, about 100%, or 100% of the cells in the composition are CD3+ T cells. In some embodiments, at least or at least about 90% of the cells in the composition are CD3+ T cells, and at least or at least about 40%, 50%, 60%, 70%, 80% or 90% of the total receptor⁺/CD3+ cells in the composition are CD27+CCR7+. In some embodiments, at least or at least about 95% of the cells in the composition are CD3+ T cells, and at least or at least about 50%, 60%, 70%, 80% or 90% of the total receptor⁺/CD3⁺ cells in the composition are CD27+CCR7+. In some embodiments, at least or at least about 98% of the cells in the composition are CD3+ T cells, and at least or at least about 50%, 60%, 70%, 80% or 90% of the total receptor⁺/CD3⁺ cells in the composition are CD27+CCR7+. In some embodiments, at least 50%, 60%, 70%, 80% or 90% of the cells in the composition are CD3+ T cells, at least 50% of the total receptor⁺/CD8⁺ cells in the composition are CD27+CCR7+ and at least 50% of the total receptor⁺/CD4⁺ cells in the composition are CD27+CCR7+. In some embodiments, at least 90% of the cells in the composition are CD3+ T cells, at least 50%, 60%, 70%, 80% or 90% of the total receptor⁺/CD8⁺ cells in the composition are CD27+CCR7+, and at least 50%, 60%, 70%, 80% or 90% of the total receptor⁺/CD4⁺ cells in the composition are CD27+CCR7+.

In some embodiments, provided herein is a therapeutic T cell composition comprising and/or enriched in CD3+ T cells expressing a recombinant receptor, wherein at least 50%, 60%, 70%, 80% or 90% of the total receptor⁺/CD3⁺ cells in the composition are naïve-like T cells or central memory T cells or are surface positive for a marker expressed on naïve-like T cells or central memory T cells. In some embodiments, at least or at least about 80%, at least or at least about 85%, at least or at least about 90%, at least or at least about 95%, at least or at least about 96%, at least or at least about 97%, at least or at least about 98%, at least or at least about 99%, about 100%, or 100% of the cells in the composition are CD3+ T cells. In some embodiments, at least or at least about 90% of the cells in the composition are CD3+ T cells, and at least or at least about 40%, 50%, 60%, 70%, 80% or 90% of the total receptor⁺/CD3⁺ cells in the composition are naïve-like T cells or central memory T cells or are surface positive for a marker expressed on naïve-like T cells or central memory T cells. In some embodiments, at least or at least about 95% of the cells in the composition are CD3+ T cells, and at least or at least about 50%, 60%, 70%, 80% or 90% of the total receptor⁺/CD3⁺ cells in the composition are naïve-like T cells or central memory T cells or are surface positive for a marker expressed on naïve-like T cells or central memory T cells. In some embodiments, at least or at least about 98% of the cells in the composition are CD3+ T cells, and at least or at least about 50%, 60%, 70%, 80% or 90% of the total receptor⁺/CD3⁺ cells in the composition are naïve-like T cells or central memory T cells or are surface positive for a marker expressed on naïve-like T cells or central memory T cells. In some embodiments, at least 50%, 60%, 70%, 80% or 90% of the cells in the composition are CD3+ T cells, at least 50% of the total receptor⁺/CD8⁺ cells in the composition are naïve-like T cells or central memory T cells or are surface positive for a marker expressed on naïve-like T cells or central memory T cells and at least 50% of the total receptor⁺/CD4⁺ cells in the composition are naïve-like T cells or central memory T cells or are surface positive for a marker expressed on naïve-like T cells or central memory T cells. In some embodiments, at least 90% of the cells in the composition are CD3+ T cells, at least 50%, 60%, 70%, 80% or 90% of the total receptor⁺/CD8⁺ cells in the composition are naïve-like T cells or central memory T cells or are surface positive for a marker expressed on naïve-like T cells or central memory T cells, and at least 50%, 60%, 70%, 80% or 90% of the total receptor⁺/CD4⁺ cells in the composition are naïve-like T cells or central memory T cells or are surface positive for a marker expressed on naïve-like T cells or central memory T cells.

In some embodiments, provided herein is a therapeutic T cell composition comprising and/or enriched in CD4+ T cells and CD8+ T cells expressing a recombinant receptor, wherein at least 50%, 60%, 70%, 80% or 90% of the total receptor⁺/CD4⁺ and receptor⁺/CD8⁺ cells in the composition are CD27+CCR7+. In some embodiments, at least or at least about 80%, at least or at least about 85%, at least or at least about 90%, at least or at least about 95%, at least or at least about 96%, at least or at least about 97%, at least or at least about 98%, at least or at least about 99%, about 100%, or 100% of the cells in the composition are CD4+ T cells and CD8+ T cells.

In some embodiments, provided herein is a therapeutic T cell composition comprising and/or enriched in CD4+ T cells and CD8+ T cells expressing a recombinant receptor, wherein at least 50%, 60%, 70%, 80% or 90% of the total receptor+/CD4+ and receptor+/CD8+ cells in the composition are naïve-like T cells or central memory T cells or are surface positive for a marker expressed on naïve-like T cells or central memory T cells. In some embodiments, at least or at least about 80%, at least or at least about 85%, at least or at least about 90%, at least or at least about 95%, at least or at least about 96%, at least or at least about 97%, at least or at least about 98%, at least or at least about 99%, about 100%, or 100% of the cells in the composition are CD4+ T cells and CD8+ T cells. In some embodiments, at least or at least about 90% of the cells in the composition are CD4+ T cells and CD8+ T cells, and at least or at least about 40%, 50%, 60%, 70%, 80% or 90% of the total receptor+/CD4+ and receptor+/CD8+ cells in the composition are naïve-like T cells or central memory T cells or are surface positive for a marker expressed on naïve-like T cells or central memory T cells. In some embodiments, at least or at least about 95% of the cells in the composition are CD4+ T cells and CD8+ T cells, and at least or at least about 50%, 60%, 70%, 80% or 90% of the total receptor+/CD4+ and receptor+/CD8+ cells in the composition are naïve-like T cells or central memory T cells or are surface positive for a marker expressed on naïve-like T cells or central memory T cells. In some embodiments, at least or at least about 98% of the cells in the composition are CD4+ T cells and CD8+ T cells, and at least or at least about 50%, 60%, 70%, 80% or 90% of the total receptor+/CD4+ and receptor+/CD8+ cells in the composition are naïve-like T cells or central memory T cells or are surface positive for a marker expressed on naïve-like T cells or central memory T cells. In some embodiments, at least 50%, 60%, 70%, 80% or 90% of the cells in the composition are CD4+ T cells and CD8+ T cells, at least 50% of the total receptor⁺/CD8⁺ cells in the composition are naïve-like T cells or central memory T cells or are surface positive for a marker expressed on naïve-like T cells or central memory T cells and at least 50% of the total receptor⁺/CD4⁺ cells in the composition are naïve-like T cells or central memory T cells or are surface positive for a marker expressed on naïve-like T cells or central memory T cells. In some embodiments, at least 90% of the cells in the composition are CD4+ T cells and CD8+ T cells, at least 50%, 60%, 70%, 80% or 90% of the total receptor⁺/CD8⁺ cells in the composition are naïve-like T cells or central memory T cells or are surface positive for a marker expressed on naïve-like T cells or central memory T cells, and at least 50%, 60%, 70%, 80% or 90% of the total receptor⁺/CD4⁺ cells in the composition are naïve-like T cells or central memory T cells or are surface positive for a marker expressed on naïve-like T cells or central memory T cells.

In certain embodiments, disclosed herein is a therapeutic T cell composition comprising CD4+ T cells expressing a recombinant receptor and CD8+ T cells expressing a recombinant receptor, wherein at least 50%, 60%, 70%, 80% or 90% of the total receptor⁺/CD8⁺ cells in the composition are CD27+CCR7+ and at least 30%, 40%, 50%, 60%, 70%, 80% or 90% of the total receptor⁺/CD4⁺ cells in the composition are CD27+CCR7+, wherein at least or at least about 80%, at least or at least about 85%, at least or at least about 90%, at least or at least about 95%, at least or at least about 96%, at least or at least about 97%, at least or at least about 98%, at least or at least about 99%, about 100%, or 100% of the cells in the composition are CD3+ T cells. In certain embodiments, disclosed herein is a therapeutic T cell composition comprising CD4+ T cells expressing a recombinant receptor and CD8+ T cells expressing a recombinant receptor, wherein at least 50%, 60%, 70%, 80% or 90% of the total receptor⁺/CD8⁺ cells in the composition are CD27+CCR7+ and at least 30%, 40%, 50%, 60%, 70%, 80% or 90% of the total receptor⁺/CD4⁺ cells in the composition are CD27+CCR7+, wherein at least or at least about 80%, at least or at least about 85%, at least or at least about 90%, at least or at least about 95%, at least or at least about 96%, at least or at least about 97%, at least or at least about 98%, at least or at least about 99%, about 100%, or 100% of the cells in the composition are CD4+ T cells and CD8+ T cells. In some aspects, at least or at least about 90% of the cells in the composition are CD4+ T cells and CD8+ T cells, at least or at least about 60% of the total receptor⁺/CD8⁺ cells in the composition are CD27+CCR7+, and at least or at least about 40% of the total receptor⁺/CD4⁺ cells in the therapeutic T cell composition are CD27+CCR7+. In some aspects, at least or at least about 95% of the cells in the composition are CD4+ T cells and CD8+ T cells, at least or at least about 65% of the total receptor⁺/CD8⁺ cells in the composition are CD27+CCR7+, and at least or at least about 45% of the total receptor⁺/CD4⁺ cells in the therapeutic T cell composition are CD27+CCR7+. In some aspects, at least or at least about 98% of the cells in the composition are CD4+ T cells and CD8+ T cells, at least or at least about 70%, at least or at least about 75%, at least or at least about 80%, or at least or at least about 85% of the total receptor⁺/CD8⁺ cells in the composition are CD27+CCR7+, and at least or at least about 50%, at least or at least about 55%, at least or at least about 60%, or at least or at least about 65% of the total receptor⁺/CD4⁺ cells in the therapeutic T cell composition are CD27+CCR7+. In some aspects, at least or at least about 98% of the cells in the composition are CD4+ T cells and CD8+ T cells, at least or at least about 75% of the total receptor⁺/CD8⁺ cells in the composition are CD27+CCR7+, and at least or at least about 55% of the total receptor⁺/CD4⁺ cells in the therapeutic T cell composition are CD27+CCR7+. In some aspects, at least or at least about 98% of the cells in the composition are CD4+ T cells and CD8+ T cells, at least or at least about 80% of the total receptor⁺/CD8⁺ cells in the composition are CD27+CCR7+, and at least or at least about 60% of the total receptor⁺/CD4⁺ cells in the composition are CD27+CCR7+. In some aspects, at least or at least about 98% of the cells in the composition are CD4+ T cells and CD8+ T cells, at least or at least about 85% of the total receptor⁺/CD8⁺ cells in the composition are CD27+CCR7+, and at least or at least about 65% of the total receptor⁺/CD4⁺ cells in the composition are CD27+CCR7+. In some aspects, at least or at least about 98% of the cells in the composition are CD4+ T cells and CD8+ T cells, at least or at least about 90% of the total receptor⁺/CD8⁺ cells in the composition are CD27+CCR7+, and at least or at least about 70% of the total receptor⁺/CD4⁺ cells in the composition are CD27+CCR7+. In some aspects, at least 90% of the cells in the composition are CD4+ T cells and CD8+ T cells, at least 60% of the total receptor⁺/CD8+ cells in the composition are CD27+CCR7+ and at least 40% of the total receptor⁺/CD4⁺ cells in the composition are CD27+CCR7+. In some aspects, at least 90% of the cells in the composition are CD4+ T cells and CD8+ T cells, at least 70% of the total receptor⁺/CD8⁺ cells in the composition are CD27+CCR7+ and at least 50% of the total receptor⁺/CD4⁺ cells in the composition are CD27+CCR7+. In some aspects, at least 90% of the cells in the composition are CD4+ T cells and CD8+ T cells, at least 70% of the total receptor⁺/CD8⁺ cells in the composition are CD27+CCR7+ and at least 60% of the total receptor⁺/CD4⁺ cells in the composition are CD27+CCR7+. In some aspects, at least 95% of the cells in the composition are CD4+ T cells and CD8+ T cells, at least 70% of the total receptor⁺/CD8⁺ cells in the composition are CD27+CCR7+ and at least 70% of the total receptor⁺/CD4⁺ cells in the composition are CD27+CCR7+. In some aspects, at least 95% of the cells in the composition are CD4+ T cells and CD8+ T cells, at least 80% of the total receptor⁺/CD8⁺ cells in the composition are CD27+CCR7+ and at least 80% of the total receptor⁺/CD4⁺ cells in the composition are CD27+CCR7+.

In any of the preceding embodiments, a percentage of cells positive/negative for one or more markers (e.g., CD3, CD4, CD8, CD27, CD28, CCR7, or CD45RA, etc.) within a cell population or composition can be an average, mean, or median percentage from a plurality of output compositions produced by the method disclosed herein. In some embodiments, a percentage of cells positive for a marker within a cell population or composition is an average of such percentages from a plurality of output compositions produced by the method disclosed herein. In some embodiments, the plurality of output compositions are produced by the method disclosed herein from a plurality of input compositions, which may be originated from the same biological sample or different biological samples (e.g., PBMCs or an apheresis or leukapheresis sample), e.g., from the same donor or different donors. In some aspects, the average is based on the plurality of about or at least about 5, about or at least about 10, about or at least about 15, about or at least about 20, about or at least about 25, about or at least about 30, about or at least about 35, about or at least about 40, about or at least about 45, about or at least about 50, about or at least about 55, about or at least about 60, about or at least about 100, or more than about 100 output compositions produced by the method disclosed herein.

In some embodiments, wherein on average in a plurality of output compositions (e.g., about or at least about 5) produced by the method, at least at or about, or at or about, 80%, 85%, 90%, 95%, 96%, 97%, 98%, or 99% of the total number of T cells in the composition or of the total number of T cells in the composition expressing the recombinant protein, are CD4+ T cells and CD8+ T cells. In some embodiments, on average in a plurality of output compositions (e.g., about or at least about 5) produced by the method, at least 50%, 60%, 70%, 80% or 90% of the total receptor⁺/CD4⁺ and receptor⁺/CD8⁺ cells in the composition are naïve-like T cells or are surface positive for a marker expressed on naïve-like T cells (e.g., CD27+CCR7+ cells). In some embodiments, a plurality of (e.g., about or at least about 5) output compositions produced by the method disclosed herein, on average, comprise at least 50%, 60%, 70%, 80% or 90% naïve-like T cells or central memory T cells or are surface positive for a marker expressed on naïve-like T cells or central memory T cells, of the total receptor⁺/CD4⁺ and receptor⁺/CD8⁺ cells in the composition. In some embodiments, a plurality of (e.g., about or at least about 5) output compositions produced by the method disclosed herein, on average, comprise at least 50%, 60%, 70%, 80% or 90% CD27+CCR7+ cells of the total receptor⁺/CD4⁺ and receptor⁺/CD8⁺ cells in the composition. In some embodiments, a plurality of (e.g., about or at least about 5) output compositions produced by the method disclosed herein, on average, comprise at least or at least about 80%, 85%, 90%, 95%, 96%, 97%, 98%, or 99% CD3+ T cells. In some embodiments, a plurality of (e.g., about or at least about 5) output compositions produced by the method disclosed herein, on average, comprise at least 50%, 60%, 70%, 80% or 90% naïve-like T cells or are surface positive for a marker expressed on naïve-like T cells (e.g., CD27+CCR7+ cells), of the total receptor⁺/CD3⁺ cells in the composition. In some embodiments, a plurality of (e.g., about or at least about 5) output compositions produced by the method disclosed herein, on average, comprise at least 50%, 60%, 70%, 80% or 90% naïve-like T cells or central memory T cells or are surface positive for a marker expressed on naïve-like T cells or central memory T cells, of the total receptor⁺/CD3⁺ cells in the composition. In some embodiments, a plurality of (e.g., about or at least about 5) output compositions produced by the method disclosed herein, on average, comprise at least 50%, 60%, 70%, 80% or 90% CD27+CCR7+ cells of the total receptor⁺/CD3⁺ cells in the composition.

In some embodiments, the composition comprises T cells having the heterologous or recombinant polynucleotide encoding the anti-CD19 CAR integrated into the T cell genomes. In particular embodiments, the integrated vector copy number (iVCN) of the cells in the composition, on average, is of about, or of at least 0.1, 0.5, 1, 2, 3, 4, 5, or greater than 5 per diploid genome. In particular embodiments, iVCN of the CAR+ cells in the composition, on average, is between or between about 0.4 copies per diploid genome and 3.0 copies per diploid genome, inclusive. In particular embodiments, iVCN of the CAR+ cells in the composition, on average, is about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 copies per diploid genome, inclusive.

In certain embodiments, the fraction of iVCN to total vector copy number (VCN) in the diploid genome of the population of transformed cells, on average, is less than or less than about 0.9, for example, is at least or is about 0.4, 0.5, 0.6, 0.7, 0.8, 0.9, or is within a tolerated error thereof, e.g., ±25%, ±20%, 15%, 10%, 5%, or ±1%. In certain embodiments, the fraction of iVCN to total vector copy number (VCN) in the diploid genome of the population of transformed cells, on average, is or is about 0.8, or is within a tolerated error thereof.

In some embodiments, the total vector copy number (VCN) of the cells in the composition is, on average, less than or less than about 20, 18, 16, 14, 12, 10, 9, 8, 7, 6, 5, 4, 3, or 2 copies, inclusive. In some embodiments, the total vector copy number (VCN) of the CD3+ cells in the composition is, on average, less than or less than about 20, 18, 16, 14, 12, 10, 9, 8, 7, 6, 5, 4, 3, or 2 copies, inclusive. In some embodiments, the total vector copy number (VCN) of the CD3+CAR+ cells in the composition is, on average, less than or less than about 20, 18, 16, 14, 12, 10, 9, 8, 7, 6, 5, 4, 3, or 2 copies, inclusive.

In some embodiments, the composition includes residual stimulatory reagents, e.g., stimulatory reagents not removed following any of the methods described in Section II-C-6. In some embodiments, the residual stimulatory reagents include any of the oligomeric stimulatory reagents described in Section II-C-2. In some embodiments, the residual stimulatory reagents include any of the oligomeric streptavidin mutein reagents described in Section II-C-2. In some embodiments, the composition contains between or between about 50 and 2000 ng/mL of residual stimulatory reagent, such as between or between about 50 and 1900 ng/mL, 50 and 1800 ng/mL, 50 and 1700 ng/mL, 50 and 1600 ng/mL, 50 and 1500 ng/mL, 50 and 1400 ng/mL, 50 and 1300 ng/mL, 50 and 1200 ng/mL, 50 and 1100 ng/mL, 50 and 1000 ng/mL, 50 and 900 ng/mL, 50 and 800 ng/mL, 50 and 700 ng/mL, 50 and 600 ng/mL, 50 and 500 ng/mL, 50 and 400 ng/mL, 50 and 300 ng/mL, 50 and 200 ng/mL, 50 and 100 ng/mL, 100 and 2000 ng/mL, 100 and 1900 ng/mL, 100 and 1800 ng/mL, 100 and 1700 ng/mL, 100 and 1600 ng/mL, 100 and 1500 ng/mL, 100 and 1400 ng/mL, 100 and 1300 ng/mL, 100 and 1200 ng/mL, 100 and 1100 ng/mL, 100 and 1000 ng/mL, 100 and 900 ng/mL, 100 and 800 ng/mL, 100 and 700 ng/mL, 100 and 600 ng/mL, 100 and 500 ng/mL, 100 and 400 ng/mL, 100 and 300 ng/mL, 100 and 200 ng/mL, 200 and 2000 ng/mL, 200 and 1900 ng/mL, 200 and 1800 ng/mL, 200 and 1700 ng/mL, 200 and 1600 ng/mL, 200 and 1500 ng/mL, 200 and 1400 ng/mL, 200 and 1300 ng/mL, 200 and 1200 ng/mL, 200 and 1100 ng/mL, 200 and 1000 ng/mL, 200 and 900 ng/mL, 200 and 800 ng/mL, 200 and 700 ng/mL, 200 and 600 ng/mL, 200 and 500 ng/mL, 200 and 400 ng/mL, 200 and 300 ng/mL, 300 and 2000 ng/mL, 300 and 1900 ng/mL, 300 and 1800 ng/mL, 300 and 1700 ng/mL, 300 and 1600 ng/mL, 300 and 1500 ng/mL, 300 and 1400 ng/mL, 300 and 1300 ng/mL, 300 and 1200 ng/mL, 300 and 1100 ng/mL, 300 and 1000 ng/mL, 300 and 900 ng/mL, 300 and 800 ng/mL, 300 and 700 ng/mL, 300 and 600 ng/mL, 300 and 500 ng/mL, 300 and 400 ng/mL, 400 and 2000 ng/mL, 400 and 1900 ng/mL, 400 and 1800 ng/mL, 400 and 1700 ng/mL, 400 and 1600 ng/mL, 400 and 1500 ng/mL, 400 and 1400 ng/mL, 400 and 1300 ng/mL, 400 and 1200 ng/mL, 400 and 1100 ng/mL, 400 and 1000 ng/mL, 400 and 900 ng/mL, 400 and 800 ng/mL, 400 and 700 ng/mL, 400 and 600 ng/mL, 400 and 500 ng/mL, 500 and 2000 ng/mL, 500 and 1900 ng/mL, 500 and 1800 ng/mL, 500 and 1700 ng/mL, 500 and 1600 ng/mL, 500 and 1500 ng/mL, 500 and 1400 ng/mL, 500 and 1300 ng/mL, 500 and 1200 ng/mL, 500 and 1100 ng/mL, 500 and 1000 ng/mL, 500 and 900 ng/mL, 500 and 800 ng/mL, 500 and 700 ng/mL, 500 and 600 ng/mL, 600 and 2000 ng/mL, 600 and 1900 ng/mL, 600 and 1800 ng/mL, 600 and 1700 ng/mL, 600 and 1600 ng/mL, 600 and 1500 ng/mL, 600 and 1400 ng/mL, 600 and 1300 ng/mL, 600 and 1200 ng/mL, 600 and 1100 ng/mL, 600 and 1000 ng/mL, 600 and 900 ng/mL, 600 and 800 ng/mL, 600 and 700 ng/mL, 700 and 2000 ng/mL, 700 and 1900 ng/mL, 700 and 1800 ng/mL, 700 and 1700 ng/mL, 700 and 1600 ng/mL, 700 and 1500 ng/mL, 700 and 1400 ng/mL, 700 and 1300 ng/mL, 700 and 1200 ng/mL, 700 and 1100 ng/mL, 700 and 1000 ng/mL, 700 and 900 ng/mL, 700 and 800 ng/mL, 800 and 2000 ng/mL, 800 and 1900 ng/mL, 800 and 1800 ng/mL, 800 and 1700 ng/mL, 800 and 1600 ng/mL, 800 and 1500 ng/mL, 800 and 1400 ng/mL, 800 and 1300 ng/mL, 800 and 1200 ng/mL, 800 and 1100 ng/mL, 800 and 1000 ng/mL, 800 and 900 ng/mL, 900 and 2000 ng/mL, 900 and 1900 ng/mL, 900 and 1800 ng/mL, 900 and 1700 ng/mL, 900 and 1600 ng/mL, 900 and 1500 ng/mL, 900 and 1400 ng/mL, 900 and 1300 ng/mL, 900 and 1200 ng/mL, 900 and 1100 ng/mL, 900 and 1000 ng/mL, 1000 and 2000 ng/mL, 1000 and 1900 ng/mL, 1000 and 1800 ng/mL, 1000 and 1700 ng/mL, 1000 and 1600 ng/mL, 1000 and 1500 ng/mL, 1000 and 1400 ng/mL, 1000 and 1300 ng/mL, 1000 and 1200 ng/mL, 1000 and 1100 ng/mL, 1100 and 2000 ng/mL, 1100 and 1900 ng/mL, 1100 and 1800 ng/mL, 1100 and 1700 ng/mL, 1100 and 1600 ng/mL, 1100 and 1500 ng/mL, 1100 and 1400 ng/mL, 1100 and 1300 ng/mL, 1100 and 1200 ng/mL, 1200 and 2000 ng/mL, 1200 and 1900 ng/mL, 1200 and 1800 ng/mL, 1200 and 1700 ng/mL, 1200 and 1600 ng/mL, 1200 and 1500 ng/mL, 1200 and 1400 ng/mL, 1200 and 1300 ng/mL, 1300 and 2000 ng/mL, 1300 and 1900 ng/mL, 1300 and 1800 ng/mL, 1300 and 1700 ng/mL, 1300 and 1600 ng/mL, 1300 and 1500 ng/mL, 1300 and 1400 ng/mL, 1400 and 2000 ng/mL, 1400 and 1900 ng/mL, 1400 and 1800 ng/mL, 1400 and 1700 ng/mL, 1400 and 1600 ng/mL, 1400 and 1500 ng/mL, 1500 and 2000 ng/mL, 1500 and 1900 ng/mL, 1500 and 1800 ng/mL, 1500 and 1700 ng/mL, 1500 and 1600 ng/mL, 1600 and 2000 ng/mL, 1600 and 1900 ng/mL, 1600 and 1800 ng/mL, 1600 and 1700 ng/mL, 1700 and 2000 ng/mL, 1700 and 1900 ng/mL, 1700 and 1800 ng/mL, 1800 and 2000 ng/mL, 1800 and 1900 ng/mL, or 1900 and 2000 ng/mL, each inclusive.

Also disclosed herein is a method of treating a B-cell non-Hodgkin lymphoma (B-cell NHL), the method comprising administering to a subject having or suspected of having a B-cell NHL a composition comprising engineered T cells expressing a CAR that targets CD19, wherein the administered composition is produced by a manufacturing process to produce an output composition exhibiting a predetermined feature, wherein iterations of the manufacturing process produce a plurality of the output compositions, optionally from human biological samples, when carried out among a plurality of different individual subjects, in which the predetermined feature of the output composition among the plurality of output compositions is selected from the features of the composition disclosed in Section III in any combination, including the percentage of CD3+ cells, ratios of CD4+/CD8+ or CD4CAR+/CD8+CD8+ cells, percentage of cells expressing an apoptosis marker, percentage of less differentiated cells, and iVCN and iVCN/VCN values.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. In some embodiments, sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### V. Exemplary Embodiments

Among the provided embodiments are:

1. A method of treating a B-cell non-Hodgkin lymphoma (B-cell NHL), the method comprising administering to a subject having or suspected of having a B-cell NHL a composition comprising engineered T cells expressing a chimeric antigen receptor (CAR) that targets CD19, wherein:

the composition comprises CD4⁺ T cells expressing the CAR and CD8⁺ T cells expressing the CAR;

the composition comprises between at or about 5×10⁶ CAR-expressing T cells and at or about 25×10⁶ CAR-expressing T cells, inclusive; and

at least or at least about 80% of the cells in the composition are CD3⁺ cells.

2. A method of treating a B-cell non-Hodgkin lymphoma (B-cell NHL), the method comprising administering to a subject having or suspected of having a B-cell NHL a composition comprising engineered T cells expressing a chimeric antigen receptor (CAR) that targets CD19, wherein:

the composition comprises CD4⁺ T cells expressing the CAR and CD8⁺ T cells expressing the CAR at a ratio between about 1:2.5 and about 2.5:1;

the composition comprises between at or about 5×10⁶ CAR-expressing T cells and at or about 100×10⁶ CAR-expressing T cells, inclusive;

at least or at least about 90% of the cells in the composition are CD3⁺ cells.

3. A method of treating a B-cell non-Hodgkin lymphoma (B-cell NHL), the method comprising administering to a subject having or suspected of having a B-cell NHL a composition comprising engineered T cells expressing a chimeric antigen receptor (CAR) that targets CD19, wherein:

the composition comprises CD4⁺ T cells expressing the CAR and CD8⁺ T cells expressing the CAR at a ratio between about 1:1 and about 2.5:1;

the composition comprises between at or about 5×10⁶ CAR-expressing T cells and at or about 50×10⁶ CAR-expressing T cells, inclusive; and

at least or at least about 80% of the cells in the composition are CD3⁺ cells.

4. A method of treating a B-cell non-Hodgkin lymphoma (r/r B-cell NHL), the method comprising administering to a subject having or suspected of having a B-cell NHL a composition comprising engineered T cells expressing a chimeric antigen receptor (CAR) that targets CD19, wherein:

the composition comprises CD4⁺ T cells expressing the CAR and CD8⁺ T cells expressing the CAR;

the composition comprises between at or about 5×10⁶ CAR-expressing T cells and at or about 100×10⁶ CAR-expressing T cells, inclusive;

at least or at least about 80% of the cells in the composition are CD3⁺ cells; and at least or at least about 80% of the CAR⁺ T cells in the composition are of a naïve-like or central memory phenotype.

5. A method of treating a B-cell non-Hodgkin lymphoma (r/r B-cell NHL), the method comprising administering to a subject having or suspected of having a B-cell NHL a composition comprising engineered T cells expressing a chimeric antigen receptor (CAR) that targets CD19, wherein:

the composition comprises CD4⁺ T cells expressing the CAR and CD8⁺ T cells expressing the CAR;

the composition comprises between at or about 5×10⁶ CAR-expressing T cells and at or about 100×10⁶ CAR-expressing T cells, inclusive;

at least or at least about 80% of the cells in the composition are CD3⁺ cells; and

at least or at least about 50% of the CD4⁺CAR⁺ T cells in the composition are CD27⁺CCR7⁺ and/or at least or at least about 50% of the CD8⁺CAR⁺ T cells in the composition are CD27⁺CCR7⁺.

6. A method of treating a B-cell non-Hodgkin lymphoma (B-cell NHL), the method comprising administering to a subject having or suspected of having a B-cell NHL a composition comprising engineered T cells expressing a chimeric antigen receptor (CAR) that targets CD19, wherein:

the composition comprises CD4⁺ T cells expressing the CAR and CD8⁺ T cells expressing the CAR;

the composition comprises between at or about 5×10⁶ CAR-expressing T cells and at or about 50×10⁶ CAR-expressing T cells, inclusive;

at least or at least about 96% of the cells in the composition are CD3⁺ cells; and

at least or at least about 80% of the CAR⁺ T cells in the composition are of a naïve-like or central memory phenotype.

7. A method of treating a B-cell non-Hodgkin lymphoma (r/r B-cell NHL), the method comprising administering to a subject having or suspected of having a B-cell NHL a composition comprising engineered T cells expressing a chimeric antigen receptor (CAR) that targets CD19, wherein:

the composition comprises CD4⁺ T cells expressing the CAR and CD8⁺ T cells expressing the CAR;

the composition comprises between at or about 5×10⁶ CAR-expressing T cells and at or about 100×10⁶ CAR-expressing T cells, inclusive;

at least or at least about 80% of the cells in the composition are CD3⁺ cells; and

the fraction of integrated vector copy number (iVCN) to total VCN in the CAR⁺ T cells in the composition, on average, is less than or less than about 0.9.

8. A method of treating a B-cell non-Hodgkin lymphoma (r/r B-cell NHL), the method comprising administering to a subject having or suspected of having a B-cell NHL a composition comprising engineered T cells expressing a chimeric antigen receptor (CAR) that targets CD19, wherein:

the composition comprises CD4⁺ T cells expressing the CAR and CD8⁺ T cells expressing the CAR;

the composition comprises between at or about 5×10⁶ CAR-expressing T cells and at or about 100×10⁶ CAR-expressing T cells, inclusive;

at least or at least about 80% of the cells in the composition are CD3⁺ cells; and

the integrated vector copy number (iVCN) of the CAR⁺ T cells in the composition, on average, is between or between about 0.4 copies per diploid genome and 3.0 copies per diploid genome, inclusive.

9. The method of any of embodiments 1-8, wherein the composition comprises CD4⁺ T cells expressing the CAR and CD8⁺ T cells expressing the CAR at a ratio between about 1:2 and about 2:1, between about 1:1.5 and about 1.5:1, or at or at about 1:1.

10. The method of any of embodiments 1-8, wherein the composition comprises CD4⁺ T cells expressing the CAR and CD8⁺ T cells expressing the CAR at a ratio between about 1:1 and about 2.5:1, between about 1.5:1 and about 2:1, at or at about 1.5:1, or at or at about 2:1.

11. The method of any of embodiments 1-10, wherein the composition comprises between at or about 5×10⁶ CAR-expressing T cells and at or about 10×10⁶ CAR-expressing T cells, inclusive.

12. The method of any of embodiments 1-10, wherein the composition comprises between at or about 10×10⁶ CAR-expressing T cells and at or about 25×10⁶ CAR-expressing T cells, inclusive.

13. The method of any of embodiments 1-10, wherein the composition comprises at or about 5×10⁶ CAR-expressing T cells.

14. The method of any of embodiments 1-10, wherein the composition comprises at or about 10×10⁶ CAR-expressing T cells.

15. The method of any of embodiments 1-10, wherein the composition comprises at or about 25×10⁶ CAR-expressing T cells.

16. The method of any of embodiments 1-15, wherein at least or at least about 91%, at least or at least about 92%, at least or at least about 93%, at least or at least about 94%, at least or at least about 95%, or at least or at least about 96% of the cells in the composition are CD3⁺ cells.

17. The method of any of embodiments 1-16, wherein between at or about 5% and at or about 30%, optionally between at or about 5% and at or about 30%, of the CAR⁺ T cells in the composition express a marker of apoptosis, optionally Annexin V or active Caspase 3.

18. The method of any of embodiments 1-16, wherein between at or about 10% and at or about 15% of the CAR⁺ T cells in the composition express a marker of apoptosis, optionally Annexin V or active Caspase 3.

19. The method of any of embodiments 1-16, wherein between at or about 15% and at or about 20% of the CAR⁺ T cells in the composition express a marker of apoptosis, optionally Annexin V or active Caspase 3.

20. The method of any of embodiments 1-16, wherein between at or about 20% and at or about 25% of the CAR⁺ T cells in the composition express a marker of apoptosis, optionally Annexin V or active Caspase 3.

21. The method of any of embodiments 1-16, wherein between at or about 25% and at or about 30% of the CAR⁺ T cells in the composition express a marker of apoptosis, optionally Annexin V or active Caspase 3.

22. The method of any of embodiments 1-16, wherein at or about 5%, at or about 10%, at or about 15%, at or about 20%, at or about 25%, or at or about 30% of the CAR⁺ T cells in the composition express a marker of apoptosis, optionally Annexin V or active Caspase 3.

23. The method of any of embodiments 1-22, wherein between at or about 80% and at or about 85% of the CAR⁺ T cells in the composition are of a naïve-like or central memory phenotype.

24. The method of any of embodiments 1-22, wherein between at or about 85% and at or about 90% of the CAR⁺ T cells in the composition are of a naïve-like or central memory phenotype.

25. The method of any of embodiments 1-22, wherein between at or about 90% and at or about 95% of the CAR⁺ T cells in the composition are of a naïve-like or central memory phenotype.

26. The method of any of embodiments 1-22, wherein between at or about 95% and at or about 99% of the CAR⁺ T cells in the composition are of a naïve-like or central memory phenotype.

27. The method of any of embodiments 1-22, wherein at or about 85%, at or about 90%, at or about 95%, or at or about 99% of the CAR⁺ T cells in the composition are of a naïve-like or central memory phenotype.

28. The method of any of embodiments 1-27, wherein at least or at least about 80% of the CAR⁺ T cells in the composition are surface positive for a marker expressed on naïve-like or central memory T cells.

29. The method of embodiment 28, wherein the marker expressed on naïve-like or central memory T cell is selected from the group consisting of CD45RA, CD27, CD28, and CCR7.

30. The method of any of embodiments 1-29, wherein at least or at least about 80% of the CAR⁺ T cells in the composition are CCR7⁺CD45RA⁺, CD27⁺CCR7⁺, and/or CD62L⁻CCR7⁺.

31. The method of any of embodiments 1-30, wherein between at or about 80% and at or about 85%, between at or about 85% and at or about 90%, between at or about 90% and at or about 95%, between at or about 95% and at or about 99% of the CAR⁺ T cells in the composition are CCR7⁺CD45RA⁺, CD27⁺CCR7⁺, and/or CD62L⁻CCR7⁺.

32. The method of any of embodiments 1-31, wherein at or about 80%, at or about 85%, at or about 90%, at or about 95%, or at or about 99% of the CAR⁺ T cells in the composition are CCR7⁺CD45RA⁺, CD27⁺CCR7⁺, and/or CD62L⁻CCR7⁺.

33. The method of any of embodiments 1-32, wherein at or about 80%, at or about 85%, at or about 90%, at or about 95%, or at or about 99% of the CAR⁺ T cells in the composition are CD27⁺CCR7⁺.

34. The method of any of embodiments 1-33, wherein at least or at least about 50% of the CD4⁺CAR⁺ T cells in the composition are CCR7⁺CD45RA⁺ or CCR7⁺CD45RA⁻.

35. The method of any of embodiments 1-33, wherein at least or at least about 60% of the CD4⁺CAR⁺ T cells in the composition are CCR7⁺CD45RA⁺ or CCR7⁺CD45RA⁻.

36. The method of any of embodiments 1-33, wherein at least or at least about 70% of the CD4⁺CAR⁺ T cells in the composition are CCR7⁺CD45RA⁺ or CCR7⁺CD45RA⁻.

37. The method of any of embodiments 1-33, wherein at least or at least about 80% of the CD4⁺CAR⁺ T cells in the composition are CCR7⁺CD45RA⁺ or CCR7⁺CD45RA⁻.

38. The method of any of embodiments 1-33, wherein at least or at least about 85% of the CD4⁺CAR⁺ T cells in the composition are CCR7⁺CD45RA⁺ or CCR7⁺CD45RA⁻.

39. The method of any of embodiments 1-38, wherein at least or at least about 50% of the CD4⁺CAR⁺ T cells in the composition are CD27⁺CCR7⁺.

40. The method of any of embodiments 1-38, wherein at least or at least about 60% of the CD4⁺CAR⁺ T cells in the composition are CD27⁺CCR7⁺.

41. The method of any of embodiments 1-38, wherein at least or at least about 70% of the CD4⁺CAR⁺ T cells in the composition are CD27⁺CCR7⁺.

42. The method of any of embodiments 1-38, wherein at least or at least about 80% of the CD4⁺CAR⁺ T cells in the composition are CD27⁺CCR7⁺.

43. The method of any of embodiments 1-38, wherein at least or at least about 85% of the CD4⁺CAR⁺ T cells in the composition are CD27⁺CCR7⁺.

44. The method of any of embodiments 1-43, wherein at least or at least about 50% of the CD8⁺CAR⁺ T cells in the composition are CCR7⁺CD45RA⁺ or CCR7⁺CD45RA⁻.

45. The method of any of embodiments 1-43, wherein at least or at least about 60% of the CD8⁺CAR⁺ T cells in the composition are CCR7⁺CD45RA⁺ or CCR7⁺CD45RA⁻.

46. The method of any of embodiments 1-43, wherein at least or at least about 70% of the CD8⁺CAR⁺ T cells in the composition are CCR7⁺CD45RA⁺ or CCR7⁺CD45RA⁻.

47. The method of any of embodiments 1-43, wherein at least or at least about 80% of the CD8⁺CAR⁺ T cells in the composition are CCR7⁺CD45RA⁺ or CCR7⁺CD45RA⁻.

48. The method of any of embodiments 1-43, wherein at least or at least about 85% of the CD8⁺CAR⁺ T cells in the composition are CCR7⁺CD45RA⁺ or CCR7⁺CD45RA⁻.

49. The method of any of embodiments 1-48, wherein at least or at least about 50% of the CD8⁺CAR⁺ T cells in the composition are CD27⁺CCR7⁺.

50. The method of any of embodiments 1-48, wherein at least or at least about 60% of the CD8⁺CAR⁺ T cells in the composition are CD27⁺CCR7⁺.

51. The method of any of embodiments 1-48, wherein at least or at least about 70% of the CD8⁺CAR⁺ T cells in the composition are CD27⁺CCR7⁺.

52. The method of any of embodiments 1-48, wherein at least or at least about 80% of the CD8⁺CAR⁺ T cells in the composition are CD27⁺CCR7⁺.

53. The method of any of embodiments 1-48, wherein at least or at least about 85% of the CD8⁺CAR⁺ T cells in the composition are CD27⁺CCR7⁺.

54. The method of any of embodiments 1-53, wherein the fraction of integrated vector copy number (iVCN) to total VCN in the CAR⁺ T cells in the composition, on average, is between at or about 0.9 and at or about 0.8.

55. The method of any of embodiments 1-53, wherein the fraction of integrated vector copy number (iVCN) to total VCN in the CAR⁺ T cells in the composition, on average, is less than or less than about 0.8.

56. The method of any of embodiments 1-53, wherein the fraction of integrated vector copy number (iVCN) to total VCN in the CAR⁺ T cells in the composition, on average, is between at or about 0.8 and at or about 0.7.

57. The method of any of embodiments 1-53, wherein the fraction of integrated vector copy number (iVCN) to total VCN in the CAR⁺ T cells in the composition, on average, is between at or about 0.7 and at or about 0.6.

58. The method of any of embodiments 1-53, wherein the fraction of integrated vector copy number (iVCN) to total VCN in the CAR⁺ T cells in the composition, on average, is between at or about 0.6 and at or about 0.5.

59. The method of any of embodiments 1-53, wherein the fraction of integrated vector copy number (iVCN) to total VCN in the CAR⁺ T cells in the composition, on average, is between at or about 0.5 and at or about 0.4.

60. The method of any of embodiments 1-59, wherein the integrated vector copy number (iVCN) of the CAR⁺ T cells in the composition, on average, is between or between about 0.8 copies per diploid genome and 2.0 copies per diploid genome, inclusive.

61. The method of any of embodiments 1-59, wherein the integrated vector copy number (iVCN) of the CAR⁺ T cells in the composition, on average, is between or between about 0.8 copies per diploid genome and 1.0 copies per diploid genome, inclusive.

62. The method of any of embodiments 1-59, wherein the integrated vector copy number (iVCN) of the CAR⁺ T cells in the composition, on average, is between or between about 1.0 copies per diploid genome and 1.5 copies per diploid genome, inclusive.

63. The method of any of embodiments 1-59, wherein the integrated vector copy number (iVCN) of the CAR⁺ T cells in the composition, on average, is between or between about 1.5 copies per diploid genome and 2.0 copies per diploid genome, inclusive.

64. The method of any of embodiments 1-63, wherein the r/r B-cell NHL is selected from the group consisting of: diffuse large B-cell lymphoma (DLBCL), optionally DLBCL not otherwise specified (DLBCL NOS; including de novo or transformed DLBCL, e.g., from follicular lymphoma or marginal zone lymphoma); high grade B-cell lymphoma (HGBCL), optionally HGBCL with MYC and BCL2 and/or BCL6 rearrangements with DLBCL histology; primary mediastinal large B cell lymphoma (PMBCL); and follicular lymphoma (FL), optionally follicular lymphoma grade 3b (FL3B).

65. The method of any of embodiments 1-64, wherein the r/r B-cell NHL is a diffuse large B-cell lymphoma.

66. The method of any of embodiments 1-64, wherein the r/r B-cell NHL is a diffuse large B-cell lymphoma not otherwise specified.

67. The method of any of embodiments 1-64, wherein the r/r B-cell NHL is a de novo diffuse large B-cell lymphoma.

68. The method of any of embodiments 1-64, wherein the r/r B-cell NHL is a diffuse large B-cell lymphoma transformed from a follicular lymphoma.

69. The method of any of embodiments 1-64, wherein the r/r B-cell NHL is a diffuse large B-cell lymphoma transformed from a marginal zone lymphoma.

70. The method of any of embodiments 1-64, wherein the r/r B-cell NHL is a high grade B-cell lymphoma (HGBCL).

71. The method of any of embodiments 1-64, wherein the r/r B-cell NHL is a high grade B-cell lymphoma with MYC and BCL2 and/or BCL6 rearrangements.

72. The method of any of embodiments 1-64, wherein the r/r B-cell NHL is a high grade B-cell lymphoma with DLBCL histology.

73. The method of any of embodiments 1-64, wherein the r/r B-cell NHL is a double-hit lymphoma or triple-hit lymphoma.

74. The method of any of embodiments 1-64, wherein the r/r B-cell NHL is a primary mediastinal large B cell lymphoma.

75. The method of any of embodiments 1-64, wherein the r/r B-cell NHL is a follicular lymphoma.

76. The method of any of embodiments 1-64, wherein the r/r B-cell NHL is a follicular lymphoma grade 3b.

77. The method of any of embodiments 1-64, wherein the r/r B-cell NHL is histologically confirmed.

78. The method of any of embodiments 1-77, wherein at or immediately prior to the time of the administration of the composition of engineered T cells, the subject has relapsed following remission after treatment with, or become refractory to: (i) two or more prior therapies for the r/r B-cell NHL and/or (ii) an autologous stem cell transplant (ASCT) therapy.

79. The method of embodiment 78, wherein at or immediately prior to the time of the administration of the composition of engineered T cells, the subject has relapsed following remission after treatment with, or become refractory to, two or more prior therapies for the r/r B-cell NHL.

80. The method of embodiment 78, wherein at or immediately prior to the time of the administration of the composition of engineered T cells, the subject has relapsed following remission after treatment with, or become refractory to, three or more prior therapies for the r/r B-cell NHL.

81. The method of any of embodiments 78-80, wherein the two or more prior therapies for the r/r B-cell NHL does not comprise another dose of cells expressing the CAR.

82. The method of any of embodiments 78-81, wherein the two or more prior therapies comprise an anthracycline and a CD20-targeted agent.

83. The method of any of embodiments 78-82, wherein the two or more prior therapies do not include those given for prior indolent lymphoma.

84. The method of any of embodiments 78-83, wherein the two or more prior therapies do not include anthracycline given for indolent DLBCL.

85. The method of any of embodiments 78-84, wherein the two or more prior therapies comprise a CD20-targeted agent, and the two or more prior therapies may exclude an anthracycline if it is given for prior indolent lymphoma.

86. The method of any of embodiments 82-85, wherein the CD20-targeted agent comprises anti-CD20 monoclonal antibody.

87. The method of any of embodiments 82-86, wherein the CD20-targeted agent comprises rituximab.

88. The method of any of embodiments 1-77, wherein at or immediately prior to the time of the administration of the composition of engineered T cells, the subject has relapsed following remission after treatment with, or become refractory to an autologous stem cell transplant (ASCT) therapy.

89. The method of embodiment 88, wherein the subject has relapsed and/or refractory DLBCL.

90. The method of embodiment 88 or 89, wherein the ASCT fails to achieve an objective response (partial response (PR) or better).

91. The method of any of embodiments 78-90, wherein after the ASCT the disease of the subject has progressed.

92. The method of any of embodiments 1-91, wherein, at or prior to the administration of the composition of cells, the subject has been identified as having an aggressive disease or high-risk disease or as having poor prognosis.

93. The method of any of embodiments 1-92, wherein, at or prior to the administration of the composition of cells, the subject has been identified as having a chemorefractory disease or having a persistent or relapsed disease following chemotherapy.

94. The method of any of embodiments 1-93, wherein the subject has a pathologically-confirmed secondary central nervous system (CNS) involvement by malignancy.

95. The method of any of embodiments 1-94, wherein the subject does not have a central nervous system (CNS)-only involvement by malignancy.

96. The method of any of embodiments 1-95, wherein the subject has not received prior CAR T cell or genetically-modified T cell therapy.

97. The method of any of embodiments 1-96, wherein the subject has not received prior CD19-targeted therapy such as an anti-CD19 monoclonal antibody or bispecific antibody.

98. The method of any of embodiments 1-97, further comprising obtaining a leukapheresis sample from the subject for manufacturing the composition of engineered T cells.

99. The method of embodiment 98, wherein the subject has not received a therapeutic dose of a corticosteroid, optionally within at or about 14 days prior to the time of leukapheresis.

100. The method of embodiment 98 or 99, wherein the subject has not received a cytotoxic chemotherapeutic agent, which is not a lymphotoxic chemotherapeutic agent or intrathecal therapy, within at or about 7 days prior to the time of leukapheresis.

101. The method of any of embodiments 98-100, wherein the subject has not received a lymphotoxic chemotherapeutic agent within at or about 4 weeks prior to the time of leukapheresis.

102. The method of any of embodiments 98-101, wherein the subject has not received an immunosuppressive therapy within at or about 4 weeks prior to the time of leukapheresis.

103. The method of any of embodiments 98-102, wherein the subject has not received radiation within at or about 6 weeks prior to the time of leukapheresis.

104. The method of any of embodiments 98-103, wherein the subject has not received autologous stem-cell transplant within at or about 3 months prior to the time of leukapheresis.

105. The method of any of embodiments 1-104, wherein the subject has not achieved complete remission (CR) in response to a prior therapy.

106. The method of any of embodiments 1-105, wherein the subject has not achieved an objective response (partial response (PR) or better) in response to a prior therapy.

107. The method of any of embodiments 1-106, wherein, at or prior to the administration of the composition of cells, the subject is evaluated for a lymphoma associated with or involving central nervous system (CNS) involvement or a secondary CNS lymphoma.

108. The method of any of embodiments 1-107, wherein, at or prior to the administration of the composition of cells, the subject is or has been identified as having:

an adequate cardiac function, optionally with a left ventricular ejection fraction (LVEF) at or about 40%, greater than 40%, or great than about 40%; and/or

an adequate renal function, optionally with a calculated creatinine clearance of at or about 45 mL/min, greater than 45 mL/min, or great than about 45 mL/min; and/or

an adequate hepatic function, optionally with an aspartate aminotransferase (AST) and alanine aminotransferase (ALT) of or less than 2.5 times the upper limit of normal (ULN) and total bilirubin less than 1.5 times the ULN; and/or

an adequate pulmonary function, optionally with ≤CTCAE Grade 1 dyspnea and saturated oxygen (e.g., SaO₂≥92%) on room air.

109. The method of any of embodiments 1-108, wherein, at or prior to the administration of the composition of cells, the subject is or has been identified as having an absolute neutrophil count (ANC) of at or about 1.0×10⁹ cells/L, greater than 1.0×10⁹ cells/L, or great than about 1.0×10⁹ cells/L without growth factor support.

110. The method of any of embodiments 1-109, wherein, at or prior to the administration of the composition of cells, the subject is or has been identified as having platelets of at or about 50×10⁹ cells/L, greater than 50×10⁹ cells/L, or great than about 50×10⁹ cells/L without transfusion support.

111. The method of any of embodiments 1-110, wherein, at or prior to the administration of the composition of cells, the subject has received a bridging chemotherapy between the time of leukapheresis to produce the composition of engineered T cells and the administration of the composition of engineered cells.

112. The method of any of embodiments 1-111, wherein, at or prior to the administration of the composition of cells, the subject has received a bridging chemotherapy for disease control after a prior therapy.

113. The method of any of embodiments 1-112, wherein the subject is or has been identified as having an Eastern Cooperative Oncology Group Performance Status (ECOG PS) of 0 or 1.

114. The method of any of embodiments 1-113, wherein, prior to the administration of the composition of cells, the subject is or has been identified as having a high baseline tumor burden, such as measured by the sum of product of the perpendicular diameters (SPD), or high serum lactate dehydrogenase (LDH), such as LDH≥500 U/L.

115. The method of any of embodiments 1-114, wherein, at or prior to the administration of the composition of cells, the subject has a positron emission tomography (PET)-positive disease.

116. The method of any of embodiments 1-115, further comprising, prior to administration of the composition of cells, identifying or selecting the subject for administration of the composition of cells.

117. The method of any of embodiments 1-116, wherein, prior to the administration, the subject has been preconditioned with a lymphodepleting therapy.

118. The method of any of embodiments 1-117, wherein the method further comprises, immediately prior to the administration of a dose of the cells, administering a lymphodepleting therapy to the subject comprising the administration of fludarabine and/or cyclophosphamide.

119. The method of embodiment 117 or 118, wherein the administration of the composition of cells and/or the lymphodepleting therapy is carried out via outpatient delivery, e.g., in a non-tertiary care center.

120. The method of any of embodiments 117-119, wherein the lymphodepleting therapy comprises the administration of fludarabine at 30 mg/m² body surface area of the subject, daily, and cyclophosphamide at 300 mg/m² body surface area of the subject, daily, for 3 days.

121. The method of any of embodiments 117-120, wherein the composition of engineered T cells is administered between at or about 48 hours and at or about 9 days, inclusive, after completion of the lymphodepleting therapy.

122. The method of any of embodiments 117-121, wherein the subject has a creatinine clearance of at least or at least about 30 mL/min when receiving the lymphodepleting therapy.

123. The method of any of embodiments 1-122, wherein prior to initiation of administration of the composition of cells, the subject has not been administered an agent or treatment for the treatment or prevention or reduction or attenuation of a neurotoxicity and/or a cytokine release syndrome or risk thereof.

124. The method of any of embodiments 1-123, further comprising administering to the subject an agent or treatment for the treatment or prevention or reduction or attenuation of a neurotoxicity and/or a cytokine release syndrome or risk thereof.

125. The method of embodiment 123 or 124, wherein the agent is or comprises an anti-IL-6 antibody, anti-IL-6 receptor antibody or a steroid.

126. The method of any of embodiments 123-125, wherein the agent is or comprises tocilizumab or methylprednisolone.

127. The method of any of embodiments 1-126, wherein the T cells are primary T cells obtained from a subject.

128. The method of any of embodiments 1-127, wherein the T cells are autologous to the subject.

129. The method of any of embodiments 1-128, wherein:

at least 35%, at least 40% or at least 50% of subjects treated according to the method achieve a complete response (CR);

at least 60%, 70%, 80%, 90%, or 95% of subjects achieving a CR exhibit a CR that is durable for at or greater than 3 months or at or greater than 6 months; and/or

at least 60%, 70%, 80%, 90%, or 95% of subjects achieving a CR by one month and/or by 3 months remain in response, remain in CR, and/or survive or survive without progression, for at or greater than 3 months and/or at or greater than 6 months and/or at greater than 9 months after achieving the CR; and/or

at least 50%, at least 60% or at least 70% of the subjects treated according to the method achieve objective response (OR);

at least 60%, 70%, 80%, 90%, or 95% of subjects achieving an OR exhibit an OR that is durable for at or greater than 3 months or at or greater than 6 months; and/or

at least 35%, at least 40%, or at least 50% of subjects achieving an OR remain in response or survive for at or greater than 3 months and/or at or greater than 6 months after achieving the OR.

130. The method of any of embodiments 1-129, wherein:

at least 50% of subjects treated according to the method achieve a complete response (CR);

at least 60% of subjects achieving a CR exhibit a CR that is durable for at or greater than 6 months; and/or

at least 60% of subjects achieving a CR by 1 month and/or by 3 months remain in response, remain in CR, and/or survive or survive without progression, for at or greater than 6 months after achieving the CR; and/or

at least 70% of the subjects treated according to the method achieve objective response (OR);

at least 60%, of subjects achieving an OR exhibit an OR that is durable for at or greater than 6 months; and/or

at least 50% of subjects achieving an OR remain in response or survive for at or greater than 6 months after achieving the OR.

131. The method of embodiment 129 or 130, wherein:

the CR or the OR is durable for greater than 3 months or greater than 6 months; and/or

at least 20%, at least 25%, at least 35%, at least 40% or at least 50% of subjects treated according to the method achieve a CR that is durable for greater than 3 months or greater than 6 months; and/or

at least 60%, 70%, 80%, 90%, or 95% of subjects treated with the method and who achieve a CR, remain in CR or remain in response or remain surviving for at or greater than 3 months or at or greater than 6 months or at or greater than 9 months; and/or

at least 60%, 70%, 80%, 90%, or 95% of subjects treated with the method who achieve a CR by one month and/or by 3 months remain in response, remain in CR, and/or survive or survive without progression, for greater at or greater than 3 months and/or at or greater than 6 months and/or at greater than 9 months; and/or

at least 50%, at least 60% or at least 70% of the subjects treated according to the method achieve objective response (OR);

at least 60%, 70%, 80%, 90%, or 95% of subjects achieve an OR that is durable for at or greater than 3 months or at or greater than 6 months; and/or

at least at least 35%, at least 40%, or at least 50% of subjects treated with the method and achieving an OR remain in response or survive for at or greater than 3 months and/or at or greater than 6 months.

132. The method of any of embodiments 1-131, wherein the cells are autologous to the subject, and

no minimum absolute lymphocyte count (ALC) for apheresis is required and/or specified for production of the therapy; and/or

the cells are produced by a process which, for at least 90%, 91%, 92%, 93%, 94%, 95%, 96%, 97%, 98%, 99%, or 100% of subjects having the disease or condition or of the selected population of subjects, is capable of generating a cell product for administration according to the method.

133. The method of any of embodiments 1-132, wherein:

at least 35%, at least 40% or at least 50% of subjects treated according to the method achieve a complete response (CR) or remission of CNS disease;

at least 60%, 70%, 80%, 90%, or 95% of subjects who achieve a CR remain in CR for at or greater than 3 months or at or greater than 6 months; and/or

at least 60%, 70%, 80%, 90%, or 95% of subjects achieving a CR or remission of CNS disease by one month and/or by 3 months remain in response, remain in CR, and/or survive or survive without progression, for greater at or greater than 3 months and/or at or greater than 6 months and/or at greater than 9 months; and/or

at least 50%, at least 60% or at least 70% of the subjects treated according to the method achieve objective response (OR) or remission of CNS disease;

at least 60%, 70%, 80%, 90%, or 95% of subjects achieving the OR, for at or greater than 3 months or at or greater than 6 months; and/or

at least 60%, 70%, 80%, 90%, or 95% of subjects achieving OR or remission of CNS disease remain in response or survive for at or greater than 3 months and/or at or greater than 6 months; and/or

the brain lesion is reduced in size or volume by greater than or greater than about 25%, 50%, 75% or more; and/or

reduction or remission or clearance of CNS disease is achieved in at least 35%, at least 40% or at least 50% of subjects treated according to the method.

134. The method of any of embodiment 1-133, wherein:

greater than or greater than about 50%, about 60%, about 70%, or about 80% of the subjects treated according to the method do not exhibit a grade 3 or greater cytokine release syndrome (CRS) and/or do not exhibit a grade 3 or greater neurotoxicity and/or greater than 40% or 50% or 55% do not exhibit any neurotoxicity or CRS.

135. The method of any of embodiment 1-134, wherein greater than or greater than about 80% of the subjects treated according to the method do not exhibit a grade 3 or greater cytokine release syndrome (CRS) and/or do not exhibit a grade 3 or greater neurotoxicity.

136. The method of any of embodiments 1-135, wherein greater than 95% of the subjects treated according to the method do not exhibit grade 3 or greater CRS.

137. The method of any of embodiments 1-136, wherein greater than 85% of the subjects treated according to the method do not exhibit grade 3 or greater neurotoxicity.

138. The method of any of embodiments 1-137, wherein:

greater than or greater than about 30%, 35%, 40%, or 50% of the subjects treated according to the method do not exhibit any grade of cytokine release syndrome (CRS) or neurotoxicity; and/or

at least at or about 45%, 50%, 60%, 65%, 70%, 75%, 80%, 85%, 90%, or 95% of subjects treated according to the method do not exhibit onset of CRS earlier than 3 days following initiation of the administration and/or do not exhibit onset of neurotoxicity earlier than 5 days following initiation of the administration; and/or

the median onset of neurotoxicity among subjects treated according to the method is at or after the median peak of, or median time to resolution of, CRS in subjects treated according to the method and/or the median onset of neurotoxicity among subjects treated according to the method is greater than at or about 8, 9, 10, or 11 days.

139. The method of any of embodiments 1-138, wherein:

greater than or greater than about 50% of the subjects treated according to the method do not exhibit any grade of cytokine release syndrome (CRS) or neurotoxicity; and/or

at least at or about 45% of subjects treated according to the method do not exhibit onset of CRS earlier than 3 days following initiation of the administration and/or do not exhibit onset of neurotoxicity earlier than 5 days following initiation of the administration; and/or

the median onset of neurotoxicity among subjects treated according to the method is at or after the median peak of, or median time to resolution of, CRS in subjects treated according to the method and/or the median onset of neurotoxicity among subjects treated according to the method is greater than at or about 8 days.

140. The method of any of embodiments 1-139, wherein:

at least 50% of subjects treated according to the method achieve a complete response (CR);

at least 70% of the subjects treated according to the method achieve objective response (OR); and

greater than or greater than about 50% of the subjects treated according to the method do not exhibit any grade of cytokine release syndrome (CRS) or neurotoxicity; and

greater than or greater than about 80% of the subjects treated according to the method do not exhibit a grade 3 or greater cytokine release syndrome (CRS) and/or do not exhibit a grade 3 or greater neurotoxicity.

141. The method of any of embodiments 1-140, wherein:

the CAR comprises an extracellular antigen-binding domain specific for the antigen, a transmembrane domain, a cytoplasmic signaling domain derived from a costimulatory molecule, which optionally is a 4-1BB, and a cytoplasmic signaling domain derived from a primary signaling ITAM-containing molecule, which optionally is a CD3zeta;

the CAR comprises, in order, an extracellular antigen-binding domain specific for the antigen, a transmembrane domain, a cytoplasmic signaling domain derived from a costimulatory molecule, and a cytoplasmic signaling domain derived from a primary signaling ITAM-containing molecule; or

the CAR comprises an extracellular antigen-recognition domain that specifically binds to the antigen and an intracellular signaling domain comprising a CD3-zeta (CD3ζ) chain and a costimulatory signaling region that is a signaling domain of 4-1BB.

142. The method of any of embodiments 1-141, wherein the CAR comprises an extracellular antigen-binding domain specific for CD19, a transmembrane domain, a cytoplasmic signaling domain derived from a 4-1BB, and a cytoplasmic signaling domain derived from a CD3zeta.

143. The method of embodiment 142, wherein the antigen-binding domain is an scFv.

144. The method of embodiment 143, wherein the scFv comprises an amino acid sequence of RASQDISKYLN (SEQ ID NO: 35), an amino acid sequence of SRLHSGV (SEQ ID NO: 36), and/or an amino acid sequence of GNTLPYTFG (SEQ ID NO: 37) and/or an amino acid sequence of DYGVS (SEQ ID NO: 38), an amino acid sequence of VIWGSETTYYNSALKS (SEQ ID NO: 39), and/or an amino acid sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any one of the foregoing, and optionally wherein the scFv comprises, in order, a VH, a linker, optionally comprising SEQ ID NO: 24, and a VL, and/or the scFv comprises a flexible linker and/or comprises the amino acid sequence set forth as SEQ ID NO: 43.

145. The method of embodiment 143 or 144, wherein the scFv comprises a variable heavy chain region of FMC63 and a variable light chain region of FMC63.

146. The method of any of embodiments 141-145, wherein the costimulatory signaling region is a signaling domain of 4-1BB.

147. The method of any of embodiments 141-146, wherein the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto.

148. The method of any of embodiments 141-147, wherein the primary signaling domain is a CD3zeta signaling domain.

149. The method of any of embodiments 141-148, wherein the primary signaling domain comprises SEQ ID NO: 13, 14 or 15 having at least 85%, 86%, 87%, 88%, 89%, 90%, 91%, 92%, 93%, 94%, 95%, 96%, 97%, 98%, 99% or more sequence identity thereto.

150. The method of any of embodiments 141-149, wherein the CAR further comprises a spacer between the transmembrane domain and the scFv.

151. The method of embodiment 150, wherein the spacer is a polypeptide spacer that comprises or consists of all or a portion of an immunoglobulin hinge or a modified version thereof, optionally an IgG4 hinge, or a modified version thereof.

152. The method of embodiment 150 or 151, wherein the spacer is at or about 12 amino acids in length.

153. The method of any of embodiments 150-152, wherein:

the spacer has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any one of the foregoing having at least 85%, 86%, 87%, 88%, 89%, 90%, 91%, 92%, 93%, 94%, 95%, 96%, 97%, 98%, 99% or more sequence identity thereto; and/or

the spacer comprises or consists of the formula X₁PPX₂P, where X₁ is glycine, cysteine or arginine and X₂ is cysteine or threonine.

154. The method of any of embodiments 150-153, wherein:

the spacer is a polypeptide spacer that (a) comprises or consists of all or a portion of an immunoglobulin hinge or a modified version thereof or comprises about 15 amino acids or less, and does not comprise a CD28 extracellular region or a CD8 extracellular region, (b) comprises or consists of all or a portion of an immunoglobulin hinge, optionally an IgG4 hinge, or a modified version thereof and/or comprises about 15 amino acids or less, and does not comprise a CD28 extracellular region or a CD8 extracellular region, or (c) is at or about 12 amino acids in length and/or comprises or consists of all or a portion of an immunoglobulin hinge, optionally an IgG4, or a modified version thereof; or (d) has or consists of the sequence of SEQ ID NO: 1, a sequence encoded by SEQ ID NO: 2, SEQ ID NO: 30, SEQ ID NO: 31, SEQ ID NO: 32, SEQ ID NO: 33, SEQ ID NO: 34, or a variant of any one of the foregoing having at least 85%, 86%, 87%, 88%, 89%, 90%, 91%, 92%, 93%, 94%, 95%, 96%, 97%, 98%, 99% or more sequence identity thereto, or (e) comprises or consists of the formula X₁PPX₂P, where X₁ is glycine, cysteine or arginine and X₂ is cysteine or threonine; and/or

the costimulatory domain comprises SEQ ID NO: 12 or a variant thereof having at least 85%, 86%, 87%, 88%, 89%, 90%, 91%, 92%, 93%, 94%, 95%, 96%, 97%, 98%, 99% or more sequence identity thereto; and/or

the primary signaling domain comprises SEQ ID NO: 13, 14 or 15 having at least 85%, 86%, 87%, 88%, 89%, 90%, 91%, 92%, 93%, 94%, 95%, 96%, 97%, 98%, 99% or more sequence identity thereto; and/or

the scFv comprises an amino acid sequence of RASQDISKYLN (SEQ ID NO: 35), an amino acid sequence of SRLHSGV (SEQ ID NO: 36), and/or an amino acid sequence of GNTLPYTFG (SEQ ID NO: 37) and/or an amino acid sequence of DYGVS (SEQ ID NO: 38), an amino acid sequence of VIWGSETTYYNSALKS (SEQ ID NO: 39), and/or an amino acid sequence of YAMDYWG (SEQ ID NO: 40) or wherein the scFv comprises a variable heavy chain region of FMC63 and a variable light chain region of FMC63 and/or a CDRL1 sequence of FMC63, a CDRL2 sequence of FMC63, a CDRL3 sequence of FMC63, a CDRH1 sequence of FMC63, a CDRH2 sequence of FMC63, and a CDRH3 sequence of FMC63 or binds to the same epitope as or competes for binding with any one of the foregoing, and optionally wherein the scFv comprises, in order, a VH, a linker, optionally comprising SEQ ID NO: 24, and a VL, and/or the scFv comprises a flexible linker and/or comprises the amino acid sequence set forth as SEQ ID NO: 43.

155. The method of any of embodiments 150-154, wherein:

the spacer is a polypeptide spacer that comprises the sequence of SEQ ID NO: 1;

the costimulatory domain comprises SEQ ID NO: 12;

the primary signaling domain comprises SEQ ID NO: 13, 14 or 15;

the antigen binding domain comprises an scFv that comprises a variable heavy chain region of FMC63 and a variable light chain region of FMC63.

156. The method of any of embodiments 1-155, wherein the composition of cells is administered parenterally, optionally intravenously.

157. The method of any of embodiments 1-156, wherein the subject is a human subject.

158. The method of any of embodiments 1-157, wherein the composition comprising engineered T cells is produced by a manufacturing process comprising exposing an input composition comprising primary T cells with a stimulatory reagent comprising an oligomeric particle reagent comprising a plurality of avidin, streptavidin, avidin mutein, or streptavidin mutein molecules under conditions to stimulate T cells, thereby generating a stimulated population, wherein the stimulatory reagent is capable of activating one or more intracellular signaling domains of one or more components of a TCR complex and one or more intracellular signaling domains of one or more costimulatory molecules.

159. The method of embodiment 158, wherein the manufacturing process further comprises introducing into T cells of the stimulated population, a heterologous polynucleotide encoding the CAR that targets CD19, thereby generating a population of transformed cells.

160. The method of embodiment 159, wherein the manufacturing process further comprises incubating the population of transformed cells for up to 96 hours.

161. The method of embodiment 160, wherein the incubating is carried out in basal media lacking one or more recombinant cytokines.

162. The method of embodiment 159 or 161, wherein the manufacturing process further comprises harvesting T cells of the transformed population, thereby producing a composition of engineered cells.

163. The method of embodiment 162, wherein the harvesting is carried out at a time between 24 and 120 hours, inclusive, after the exposing to the stimulatory reagent is initiated.

164. The method of embodiment 162 or 163, wherein the harvesting is carried out at a time between 48 and 120 hours, inclusive, after the exposing to the stimulatory reagent is initiated.

165. The method of any of embodiments 162-164, wherein the harvesting is carried out at a time when integrated vector is detected in the genome but prior to achieving a stable integrated vector copy number (iVCN) per diploid genome.

166. The method of any of embodiments 162-165, wherein the harvesting is carried out at a time before the total number of viable cells at the harvesting is more than or more than about three times as the number of total viable cells of the stimulated population.

167. The method of embodiment 166, wherein the harvesting is carried out at a time when the total number of viable cells at the harvesting is at or about three times, at or about two times, or the same or about the same as the number of total viable cells of the stimulated population.

168. The method of any of embodiments 162-167, wherein the harvesting is carried out at a time when the percentage of CD27⁺CCR7⁺ cells is greater than or greater than about 50% among total T cells in the population, total CD3⁺ T cells in the population, total CD4⁺ T cells in the population, or total CD8⁺ T cells in the population, or of CAR-expressing cells thereof, in the population.

169. The method of any of embodiments 162-168, wherein the harvesting is carried out at a time when the percentage of CD45RA⁺CCR7⁺ and CD45RA-CCR7⁺ cells is greater than or greater than about 60% among total T cells in the population, total CD3⁺ T cells in the population, total CD4⁺ T cells in the population, or total CD8⁺ T cells, or of CAR-expressing cells thereof, in the population.

170. The method of any of embodiments 1-169, wherein the cells in the administered composition are produced by a manufacturing process to produce an output composition exhibiting a predetermined feature, wherein iterations of the manufacturing process produce a plurality of the output compositions, optionally from human biological samples, when carried out among a plurality of different individual subjects, in which the predetermined feature of the output composition among the plurality of output compositions is selected from:

the mean percentage of cells of a memory phenotype in the plurality of the output compositions is between about 40% and about 65%, between about 40% and about 45%, between about 45% and about 50%, between about 50% and about 55%, between about 55% and about 60%, or between about 60% and about 65%;

the mean percentage of cells of a central memory phenotype in the plurality of the output compositions is between about 40% and about 65%, between about 40% and about 45%, between about 45% and about 50%, between about 50% and about 55%, between about 55% and about 60%, or between about 60% and about 65%;

the mean percentage of cells that are CD27+, CD28+, CCR7+, CD45RA−, CD45RO+, CD62L+, CD3+, CD95+, granzyme B−, and/or CD127+ in the plurality of the output compositions is between about 40% and about 65%, between about 40% and about 45%, between about 45% and about 50%, between about 50% and about 55%, between about 55% and about 60%, or between about 60% and about 65%;

the mean percentage of cells that are CCR7+/CD45RA− or CCR7+/CD45RO+ in the plurality of the output compositions is between about 40% and about 65%, between about 40% and about 45%, between about 45% and about 50%, between about 50% and about 55%, between about 55% and about 60%, or between about 60% and about 65%;

the mean percentage of central memory CD4+ T cells in the engineered CD4+ T cells, optionally CAR+CD4+ T cells, of the plurality of the output compositions is between about 40% and about 65%, between about 40% and about 45%, between about 45% and about 50%, between about 50% and about 55%, between about 55% and about 60%, or between about 60% and about 65%;

the mean percentage of central memory CD8+ T cells in the engineered CD8+ T cells, optionally CAR+CD8+ T cells, of the plurality of the output compositions is between about 40% and about 65%, between about 40% and about 45%, between about 45% and about 50%, between about 50% and about 55%, between about 55% and about 60%, or between about 60% and about 65%; and/or

the mean percentage of central memory T cells, optionally CD4+ central memory T cells and CD8+ central memory T cells, in the engineered T cells, optionally CAR+ T cells, of the plurality of the output compositions is between about 40% and about 65%, between about 40% and about 45%, between about 45% and about 50%, between about 50% and about 55%, between about 55% and about 60%, or between about 60% and about 65%.

171. The method of embodiment 170, wherein the administered composition is produced by a manufacturing process to produce an output composition exhibiting a predetermined feature, optionally a threshold number of cells expressing the CAR in the output composition, in at least about 80%, about 90%, about 95%, about 97%, about 99%, about 100%, or is 100% of the human biological samples in which it is carried out among a plurality of different individual subjects.

172. The method of any of embodiments 158-171, wherein the composition comprising genetically engineered cells does not contain residual beads from the manufacturing process.

173. The method of any of embodiments 1-172, wherein the B-cell NHL is a relapsed and/or refractory B-cell non-Hodgkin lymphoma (B-cell NHL).

174. An article of manufacture comprising a composition comprising genetically engineered cells expressing a chimeric antigen receptor (CAR) that targets CD19, and instructions for administering the composition of the cells in accordance with the method of any of embodiments 1-173.

### VI. EXAMPLES

The following examples are included for illustrative purposes only and are not intended to limit the scope of the invention.

### Example 1: T Cell Compositions Containing CAR⁺ T Cells Generated Using Non-Expanded Processes

Engineered compositions of primary T cells containing T cells expressing an anti-CD19 chimeric antigen receptor (CAR) were produced by processes that utilized a stimulatory reagent composed of an anti-CD3/anti-CD28 Fab conjugated oligomeric reagent to activate T cells prior to transduction with a viral vector but that did not involve a subsequent step for expansion of the transduced cells. Two similar non-expanded processes, designated non-expanded Process A and non-expanded process B, were used to generate the CAR+ T cell compositions. The non-expanded processes did not contain a cultivation step after transduction for the purpose of increasing (e.g., expanding) the total number of viable cells at the end of the cultivation step compared to the beginning of the cultivation step. Although the incubating conditions were not carried out for purposes of expanding the cell population, the composition at harvested might have undergone expansion or exhibit an increase in cell number compared to the beginning of the incubation. For comparison, T cells from the same donor were engineered by a process in which cells were cultivated for expansion after transduction. The generated CD19-targeted CAR therapeutic T cell compositions were assessed for phenotype and activity.

All processes including engineering of T cells with the same CD19-targeted CAR. The CAR contained an anti-CD19 scFv, an immunoglobulin-derived spacer, a transmembrane domain derived from CD28, a costimulatory region derived from 4-1BB, and a CD3-zeta intracellular signaling domain. The viral vector further contained sequences encoding a truncated receptor, which served as a surrogate marker for CAR expression; separated from the CAR sequence by a T2A ribosome skip sequence.

A. Generation of CD19-Targeted CAR T Cell Compositions.

In non-expanded Process A, leukapheresis samples were collected from human donors, washed, and subjected to immunoaffinity-based selection for CD4+ and CD8+ T cell compositions. After selection, the separate CD4+ and CD8+ T cell compositions were cryofrozen, and then thawed and mixed at an approximate 1:1 ratio of viable CD4+ T cells to viable CD8+ T cells (about 300×10⁶ CD4+ and 300×10⁶ CD8+ cells) prior to stimulation by incubation for 18-30 hours with anti-CD3/anti-CD28 Fab conjugated oligomeric streptavidin mutein reagents in a serum-free complete media containing recombinant IL-2 (100 IU/mL) recombinant IL-7 (600 IU/mL) and recombinant IL-15 (100 IU/mL). After stimulation, cells were transduced by spinoculation with a lentiviral vector encoding the anti-CD19 CAR. After the spinoculation, the cells were washed and resuspended in a basal serum-free media without the addition of recombinant cytokines, and incubated at about 37.0° C. in an incubator. After about 48 hours after initiation of stimulation, D-biotin was then added and mixed with the cells to dissociate anti-CD3 and anti-CD28 Fab reagents from oligomeric streptavidin reagent. The cells were further incubated for another 48 hours (until about 4 days after initiation of stimulation), and then were formulated with a cryoprotectant.

In non-expanded Process B, leukapheresis samples were collected from human donors, washed, and cryopreserved. The cryopreserved samples were thawed before separate compositions of CD4+ and CD8+ cells were selected from each sample by immunoaffinity-based selection. The selected CD4+ and CD8+ T cell compositions were mixed at up to 900×10⁶ total viable CD4+ and CD8+ T cells, and typically at an approximate 1:1 ratio, prior to stimulation incubation for 16-24 hours with anti-CD3/anti-CD28 Fab conjugated oligomeric streptavidin mutein reagents in a serum-free complete media containing recombinant IL-2 (100 IU/mL) recombinant IL-7 (600 IU/mL) and recombinant IL-15 (100 IU/mL). After stimulation, cells were transduced by spinoculation with a lentiviral vector encoding the anti-CD19 CAR. After the spinoculation, the cells were washed and resuspended in a basal serum-free media without the addition of recombinant cytokines, and incubated at about 37.0° C. in an incubator. After about 48 hours after initiation of stimulation, D-biotin was then added and mixed with the cells to dissociate anti-CD3 and anti-CD28 Fab reagents from oligomeric streptavidin reagent. The cells were further incubated for another 48 hours (until about 4 days after initiation of stimulation), and then were formulated with a cryoprotectant.

In the expanded process, engineered CD4+ T cells and engineered CD8+ T cells each expressing the same anti-CD19 CAR were produced by a process involving subjecting enriched CD4+ and enriched CD8+ cell populations, separately, to process steps, including separate selection, cryopreservation, stimulation, transduction, expansion, and harvesting steps. Leukapheresis samples were collected from human donors, washed, and subjected to immunoaffinity-based selection for CD4+ and CD8+ T cell compositions. After selection, the separate CD4+ and CD8+ T cell compositions were activated with anti-CD3/anti-CD28 paramagnetic beads in serum-free complete media containing recombinant IL-2 and recombinant IL-15 (and additionally recombinant IL-7 for the CD4+ T cell composition), and then were separately subjected to lentiviral transduction with a vector encoding the anti-CD19 CAR. Paramagnetic beads were removed, and then transduced populations were separately incubated in the presence of recombinant IL-2 and recombinant IL-14 (and additionally recombinant IL-7 for the CD4+ T cell composition) and were cultivated in a rocking motion bioreactor for cell expansion until reaching a threshold of expansion, such as between 9 and 13 days from start of expansion. The cells were then separately harvested, formulated, and cryofrozen.

B. Purity of Engineered T Cell Compositions.

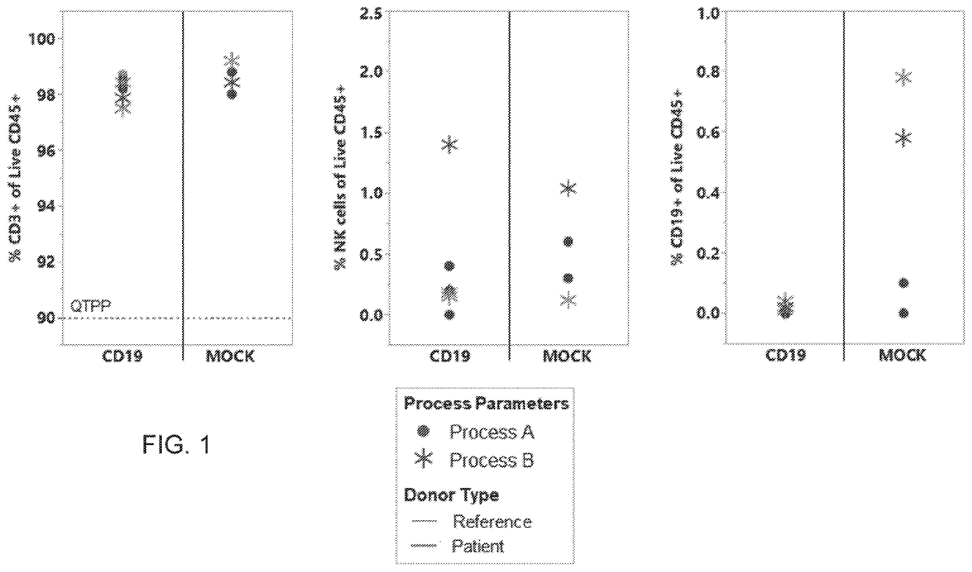

T cell compositions produced from the non-expanded engineering processes were stained with antibodies recognizing surface markers including CD3, an NK cell marker, and CD19, and quantified by flow cytometry as shown in FIG. 1. Mock transduced T cell compositions were generated and used as controls. Cell compositions generated using the non-expanded processes generally exhibited high percentages of CD3+ T cells (>96%), with low percentages of NK cells and CD19+ cells.

C. CD4/CD8 Frequencies and CD4/CD8 Ratio.

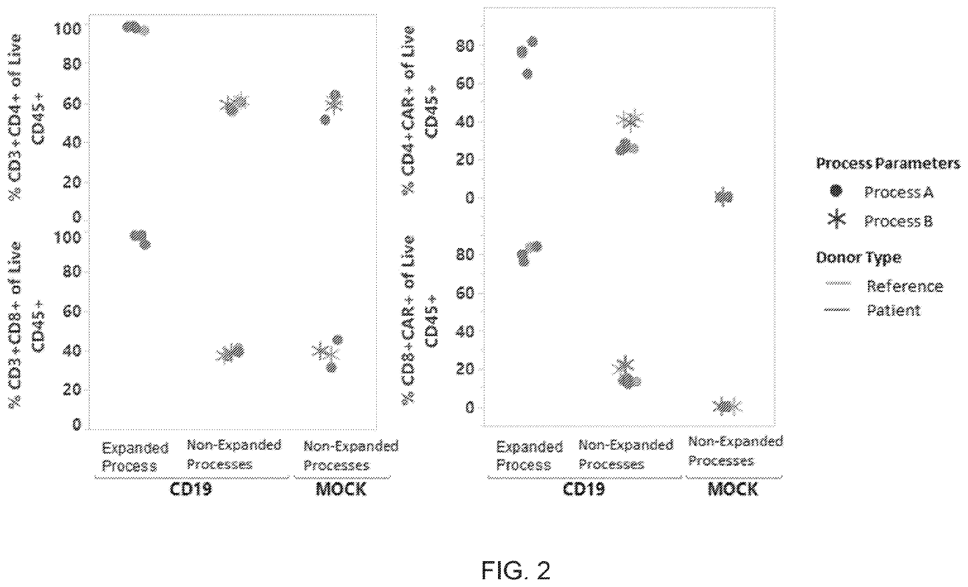

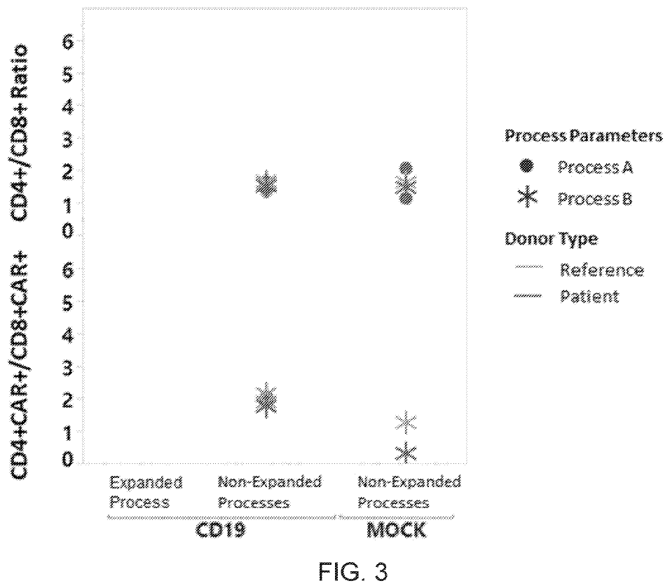

T cell compositions produced from expanded and non-expanded engineering processes were stained with antibodies recognizing surface markers including CD3, CD4, and CD8, and quantified by flow cytometry as shown in FIG. 2 and FIG. 3. Mock transduced T cell compositions were generated and used as controls. Because the expanded process produced separate CD4 and CD8 compositions, each of the CD4+ or CD8+ T cells were present at ˜100% in the respective generated compositions by that process.

D. Cell Viability.

T cell compositions produced from the non-expanded engineering processes were stained for expression of a factor indicative of apoptosis, such as surface staining with Annexin V or activated caspase 3 (aCas3) as a measure of cell health and with antibodies recognizing surface markers including CD3. FIG. 4 shows percentages of aCas3+ cells in CD3+ cells of the cell compositions produced from the non-expanded engineering processes, as compared to the mock transduced T cell compositions.

E. Vector Copy Number and Surface Expression of CAR.

T cell compositions produced from non-expanded and expanded engineering processes essentially as described in this example were assayed using a standard vector copy number (VCN) assay and an integrated vector copy number (iVCN) assay that included separation of the high- and low-molecular weight DNA species by pulse field gel electrophoresis (PFGE). Vector copy numbers of a CAR as determined by the VCN and iVCN assays were also correlated to surface expression of the CAR. Exemplary methods and compositions for the VCN and iVCN assays are disclosed in PCT/US2019/046048, which is incorporated herein by reference in its entirety.

Specifically, genomic DNA was prepared from the cells and subjected to assessment of transgene sequence copy number by either (1) an iVCN method using a threshold value for separation of >15 kb (“iVCN”) higher molecular weight DNA species from low molecular weight, non-chromosomal species below the threshold of 15 kb, in which separation was performed by automated PFGE (e.g. PippinHT (Sage Science, Beverly, Mass.) device), or (2) a standard VCN method in which genomic DNA was not first separated by PFGE (“VCN”). In both assays, transgene copy number was determined by ddPCR using primers specific for a sequence unique to the transgene (e.g. primers specific to a regulatory element of the recombinant protein), and normalized to a diploid genome, as determined using primers specific for a reference gene (e.g., albumin (ALB) gene).

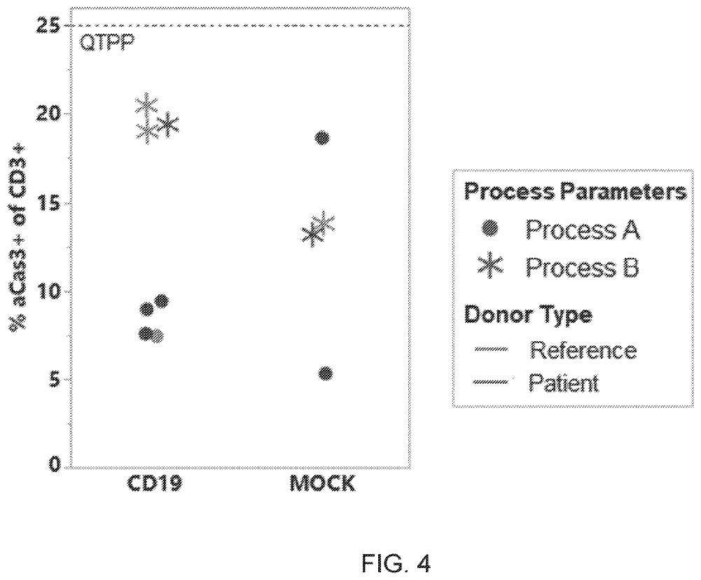

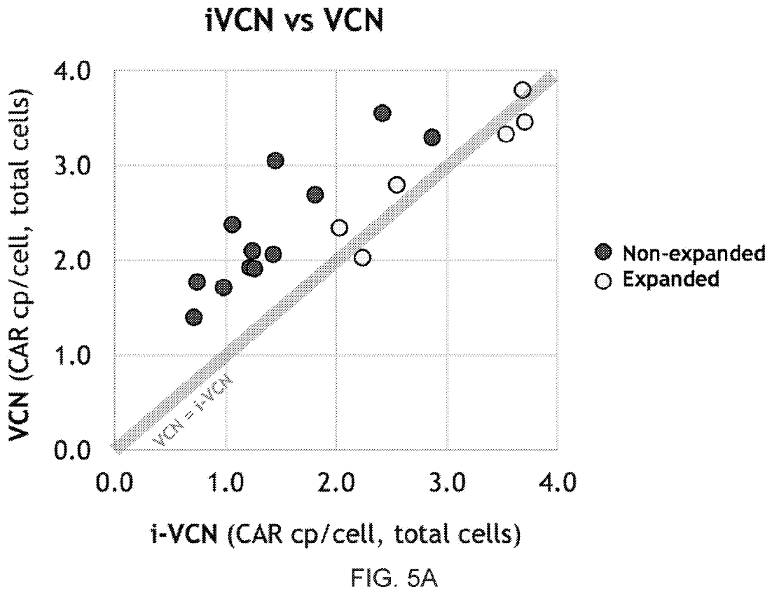

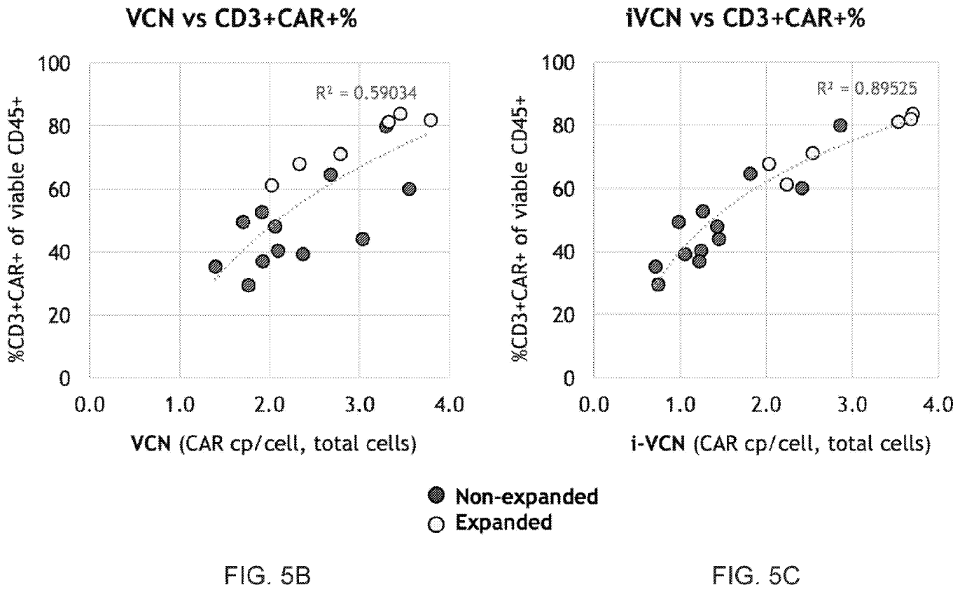

The results showed that transgene copy number assessed using the VCN assay generally correlated with the transgene copy number assessed by iVCN (FIG. 5A). However, for cells manufactured using the non-expanded process, the values obtained by VCN were higher than the values obtained by iVCN, consistent with the VCN assay detecting non-integrated transgene sequences that could be present in samples containing cells generated using the non-expanded process. In contrast, for cells manufactured using the expanded process, the value obtained by VCN and iVCN were nearly identical (near the VCN=iVCN line). These differences are likely due to the presence of a greater amount of free, non-integrated copies of transgene sequences in samples in the shorter non-expanded process compared to the expanded process. These results are consistent with the observation that a standard VCN assay that is able to detect both high and low molecular weight DNA has limitations compared to an iVCN assay, particularly when used to assess cells early after transgene introduction, such as in a shortened process for engineering T cells, where free, non-integrated copies of transgene sequences may still be present in the sample.

To assess the degree of correlation of the iVCN or VCN assay to surface expression of the CAR, cell samples from the non-expanded or expanded process were assessed by flow cytometry for expression of CD3, CD45 and the CAR to determine the percentage of CD3+CAR+ cells among viable CD45+ cells. As shown in FIG. 5B, the VCN assay exhibited better correlation to the percentage of CAR+ cells for samples engineered by the expanded process than by the non-expanded process, likely due to the presence of non-integrated CAR DNA sequences that did not contribute to surface CAR expression. As shown in FIG. 5C, the iVCN showed similar correlation to expression of the CAR among cells that had been engineered by either the non-expanded or expanded process. For all samples, the correlation of CAR expression with the copy number per cell was higher by the iVCN assay (R²=0.8952) compared to the copy number per cell as determined by the VCN assay (R²=0.5903).

F. Memory Profiles of the T Cell Compositions.

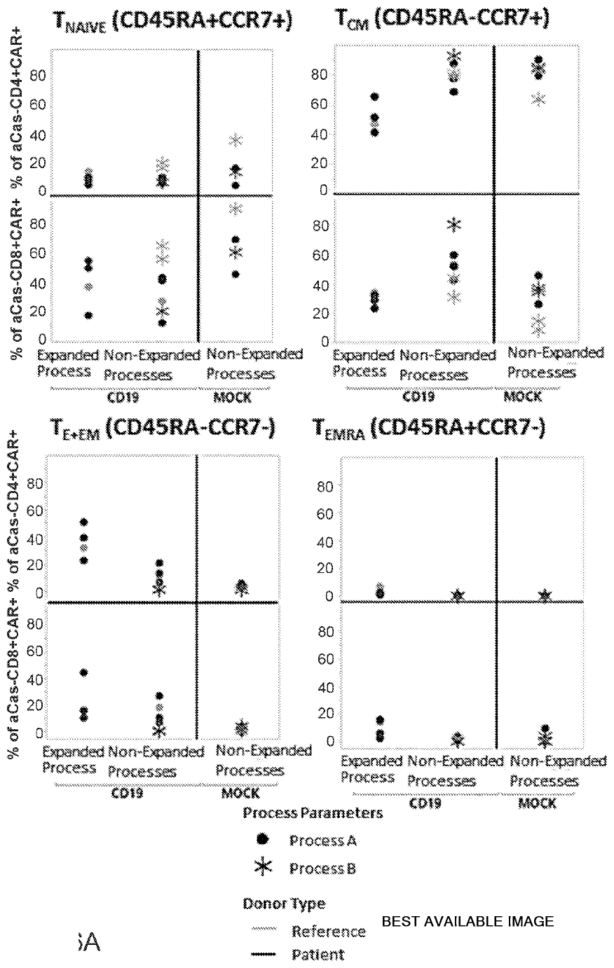

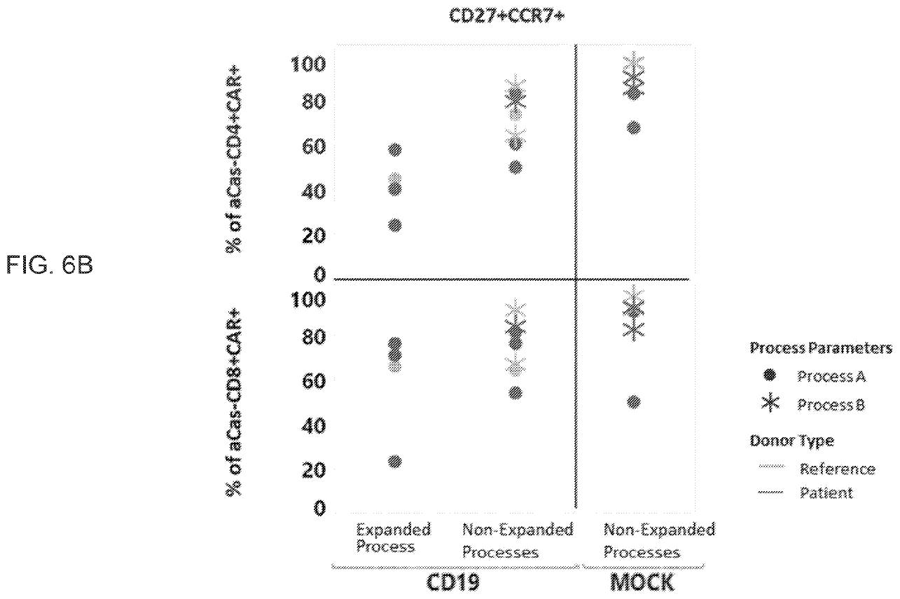

T cell compositions produced from the non-expanded engineering processes were stained for activated caspase 3 (aCas3) and with antibodies recognizing surface markers including CD4, CD8, CCR7, CD27 and CD45RA. The percentage of T cells that were indicative of naïve/naïve-like T cells (CD45RA+CCR7+), central memory (CD45RA-CCR7+), effector memory (CD45RA−CCR7−; T_{E+EM}) and effector memory CD45RA+ cells (CCR7−CD45RA+; T_{EMRA}) was determined. As shown in FIG. 6A, the engineered compositions are enriched in central memory/naïve-like T cells.

The percentage of CD4+CAR+ and CD8+CAR+ T cells among aCas-T cells positive for both CCR7 and CD27 staining are shown in FIG. 6B. As shown, the engineered compositions are enriched for CCR7+CD27+ cells.

These results support that the engineered cell compositions generated from the non-expanded processes have a greater portion of cells with a naïve-like, less differentiated phenotype than cell compositions generated from the expanded process.

### Example 2: In Vitro Activities of T Cell Compositions Generated Using Non-Expanded Processes

Engineered T cell compositions of primary T cells containing T cells expressing an anti-CD19 chimeric antigen receptor (CAR) were produced from matched donors using non-expanded and expanded processes for manufacturing engineered T cells, and attributes of the cell compositions were compared.

Leukapheresis samples from 1 healthy donor and three patients with Non-Hodgkin Lymphoma (NHL) (two patients with diffuse large B-cell lymphoma, DLBCL; and one patient with mantel cell lymphoma, MCL) were collected and manufacturing runs were carried out to engineer T cells with an anti-CD19 CAR using a non-expanded process substantially as described as Process A in Example 1. The engineered T cell compositions were compared to T cell compositions produced from donor-matched process runs using an expanded process as described in Example 1. The anti-CD19 CAR contained an anti-CD19 scFv, an immunoglobulin-derived spacer, a transmembrane domain derived from CD28, a costimulatory region derived from 4-1BB, and a CD3-zeta intracellular signaling domain. The viral vector further contained sequences encoding a truncated receptor, which served as a surrogate marker for CAR expression; separated from the CAR sequence by a T2A ribosome skip sequence.

1) Memory Profile.

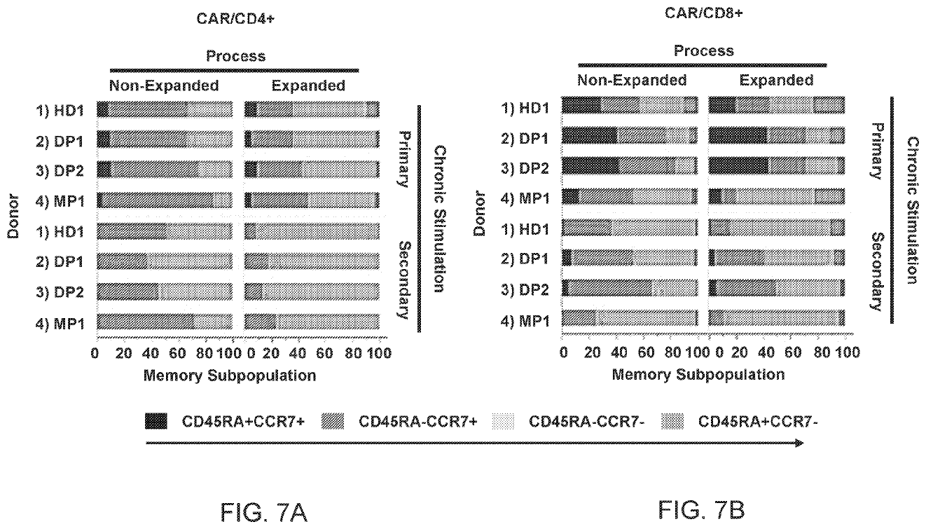

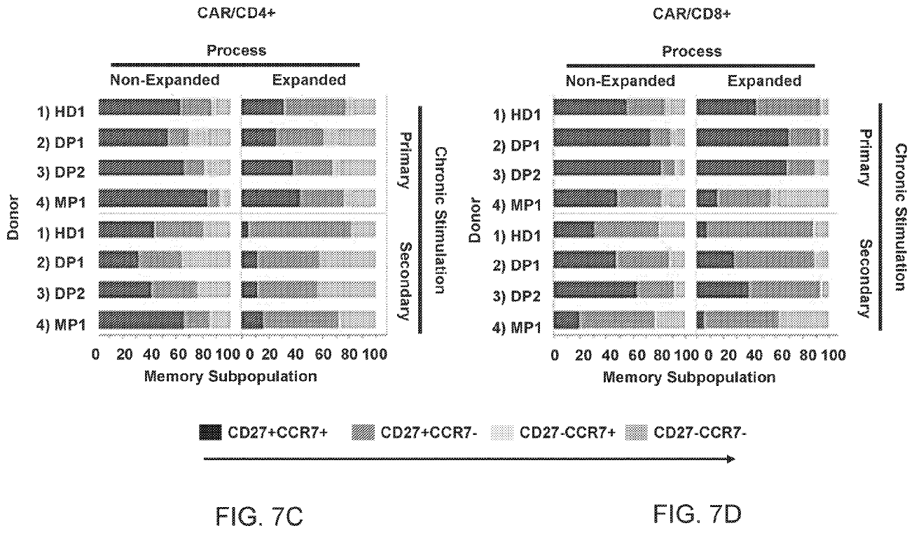

Cells from the engineered T cell compositions generated from matched donors by the non-expanded and expanded processes were analyzed in a long-term stimulation assay involving continuous incubation for 9 to 14 days in the presence of microbeads conjugated with an agonistic anti-idiotypic antibody against the anti-CD19 CAR to provide a CAR-dependent stimulus. This assay mimics the fitness and ability of cells to survive upon long-term exposure to antigen as may occur in vivo upon administration. Specifically, cells were assessed by flow cytometry for surface markers including CD4, CD8, CCR7, CD27, CD45RA, and for the CAR (surrogate marker+ or anti-idiotype+) before the long-term CAR-dependent stimulation (primary) and after the long-term CAR-dependent stimulation (secondary). Cells were gated on live, CD3+ cells that were dual-positive for CAR expression. CAR+ cells were then gated either as CD4+(FIG. 7A and FIG. 7C) or CD8+(FIG. 7B and FIG. 7D) cells prior to memory subtyping.

Before CAR-specific stimulation (primary), cell compositions generated using the non-expanded process exhibited higher proportions of naïve-like and central memory T cells and lower proportions of effector memory subtypes compared with cell compositions produced using the expanded process (FIG. 7A and FIG. 7B). Compositions generated using the non-expanded process had higher proportions of early, less-differentiated CD4+ and CD8+CAR T cells, lower proportions of intermediate-differentiated CD4+ and CD8+CAR T cells, and a lower proportion of late, more-differentiated CD4+CAR T cells compared with compositions produced using the expanded process (FIG. 7C and FIG. 7D).

After chronic CAR-specific stimulation (secondary), both compositions generated from the non-expanded and the expanded processes were more differentiated in that there were fewer less-differentiated naïve-like (e.g., CD45RA+CCR7+), central memory (e.g., CD45RA−CCR7+), or early (CD27+CCR7+) cells after expansion (FIGS. 7A-7D). Compositions generated from the non-expanded process showed preservation of less-differentiated memory subtypes after stimulation compared with compositions generated from the expanded process, including better retention of the chemokine receptor, CCR7, which is involved in trafficking to secondary lymphoid tissues in vivo and may suggest the potential for prolonged function.

2) Proliferative Capacity.

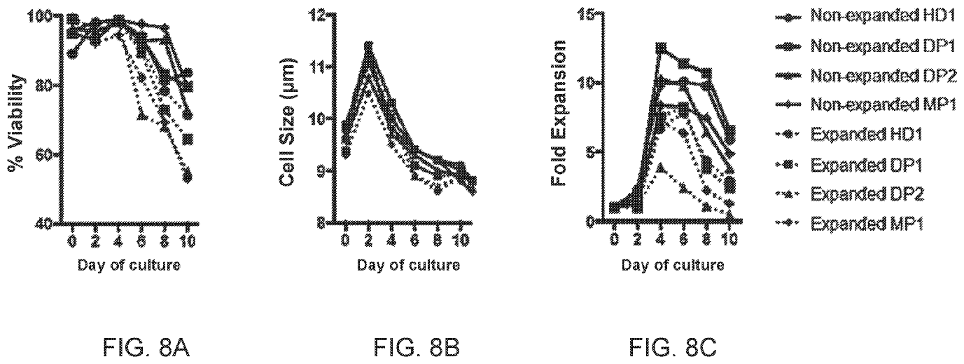

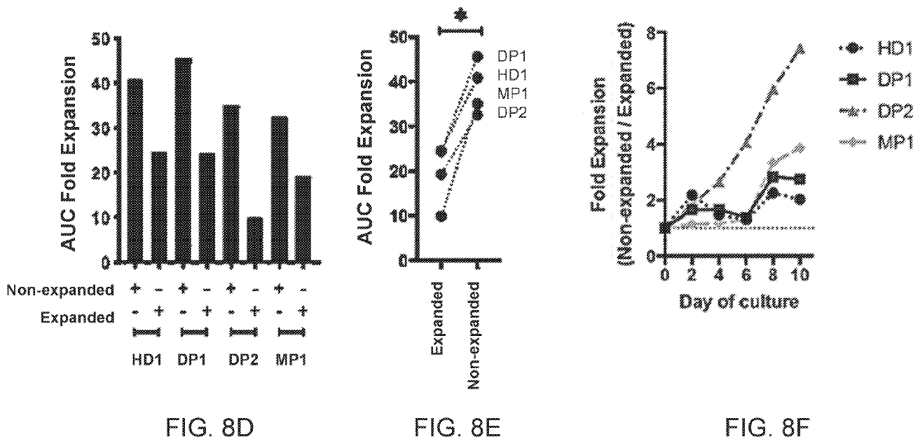

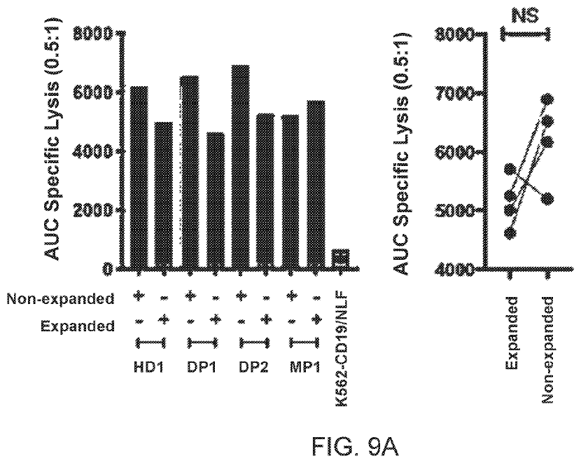

Proliferative capacity was assessed following long-term CAR-dependent stimulation with an anti-ID antibody specific for the anti-CD19 CAR as described above for 10 days. After the long-term stimulation, live cells in the compositions were analyzed. As shown in FIGS. 8A-8F, cells from the anti-CD19 non-expanded cell products exhibited substantially enhanced proliferative capacity (2- to 7.4-fold) compared to engineered cells in a donor-matched expanded cell product. These results are consistent with the observation that non-expanded cell products contains a higher relative proportion of less-differentiated T cells, including cells that maintain characteristics similar to central memory and stem cell memory T cells, and thus have the ability to undergo additional rounds of division in response to signaling through the CAR.

3) Cytolytic Activity.

For assessment of cytolytic potential, anti-CD19 CAR T cells engineered as described above in a non-expanded or expanded process were stimulated for 10-14 days with an anti-ID antibody specific for the CAR and expansion data were summarized based on the proliferation observed through day 10. K562 cells transduced to express CD19 (K562-CD19) were labeled with NucLight Red (NLR) to permit their tracking by microscopy. Engineered CAR-T cell compositions from the non-expanded or expanded process before or after the long term stimulation were co-cultured with K562-CD19 at a low effector to target cell (E:T) ratio 0.5:1. Cytolytic activity was assessed by measuring the loss of viable target cells over a period of 96 hours as determined by red fluorescent signal (using the IncuCyte® Live Cell Analysis System, Essen Bioscience). Data were transformed into an area under the curve (AUC) for comparison either for individual donors/process or by manufacturing process.

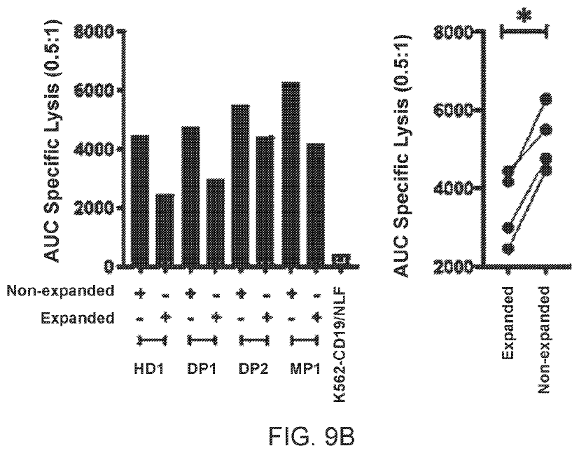

Immediately following manufacturing and before chronic stimulation (FIG. 9A), engineered CAR-T cell compositions generated using the non-expanded process exhibited better or similar cytolytic activity as compared to T cell compositions generated using the expanded process. However, following expansion in the chronic stimulation assay (FIG. 9B), compositions generated using the non-expanded process exhibited better ability to retain functional cytolytic activity at the low effector to target ratio (0.5:1), compared to compositions generated using the expanded process (Mann-Whitney test; *p<0.05).

4) Cytokine Production.

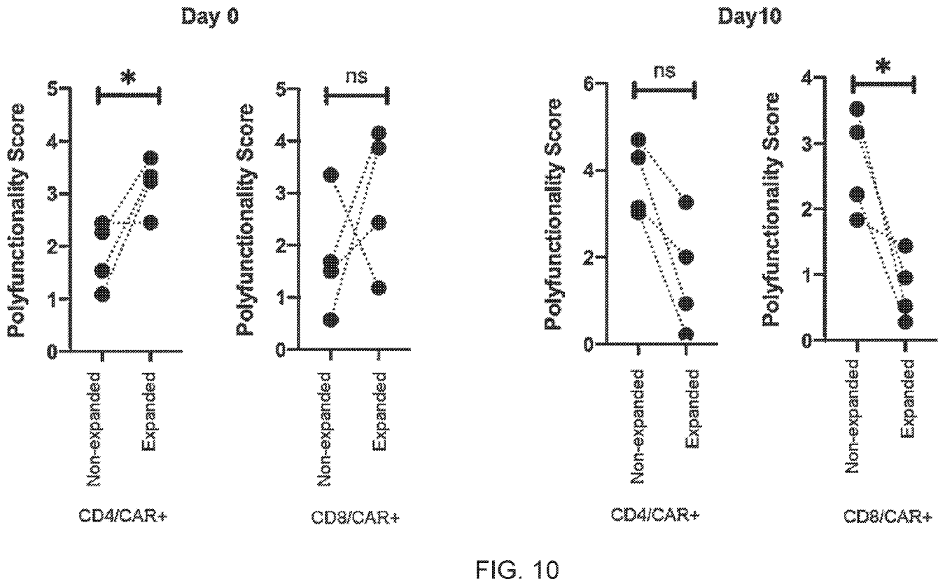

For assessment of cytokine production, anti-CD19 CAR T cells engineered as described above in a non-expanded or expanded process were stimulated for 9 to 14 days with an anti-ID antibody specific for the CAR. Engineered CAR-T cell compositions from the non-expanded or expanded process before or after the long term stimulation were incubated on plate-bound anti-ID for 5 hours, and cytokine production was measured by flow cytometry. A polyfunctional score based on simultaneous production of three cytokines (IL-2, IFNγ and TNFα) in CD4+CAR+ and CD8+CAR+ cells was determined by Boolean logic gating of triple-positive cells on a per cell basis. Statistical significance was assessed by Mann-Whitney, *p<0.05. Cells generated from the non-expanded processes showed greater polyfunctionality than cells produced from the expanded process, after a secondary antigen stimulus (FIG. 10).

### Example 3: In Vivo Anti-Tumor Activity of T Cell Compositions Generated Using Non-Expanded Processes

1) CD19+ Nalm-6 Leukemia Model.

Anti-CD19 CAR-T cell compositions generated from non-expanded and expanded matched-donor engineering processes were evaluated in the Nalm-6 acute lymphocytic leukemia (ALL) tumor model. NOD.Cg-Prkdc^{scid}IL-2rg^{tm1Wjl}/SzJ mice were injected intravenously with 5.0×10⁵ Nalm-6 firefly luciferase-green fluorescent protein (FfLuc-GFP) lymphocytic leukemia cells, and 3 days later, mice were randomized into groups to balance tumor burden. This model is a slow-growing tumor model with mice reaching end-stage disease by approximately Day 24 to Day 26 in mice that were not administered CAR-T cells. At day 4 after engraftment, CAR-T cells were intravenously administered to mice. Disseminated tumor growth was assessed by imaging Nalm-6 FfLuc-positive bioluminescence (BLI).

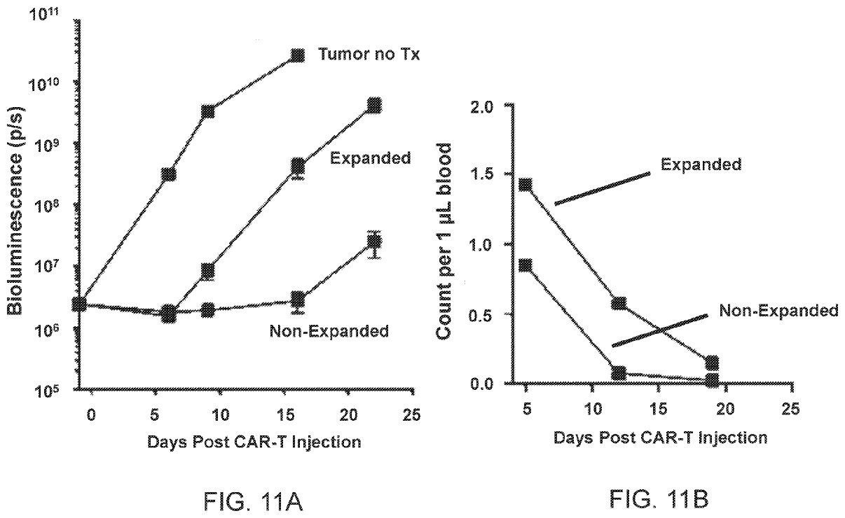

FIG. 11A shows that in mice treated with the engineered CAR-T cells generated from the expanded engineering process, tumor burden was detectably increased by around Day 10 post CAR-T cell injection. In contrast, the reduction of tumor burden persisted to around Day 15 in mice treated with CAR-T cells produced from the non-expanded engineering process. This was despite the observation that between Day 5 and around Day 20, CD19 CAR-T cells produced from the non-expanded process exhibited decrease in circulating CAR-T cells in the mice as compared to cells generated from the expanded process (FIG. 11B).

2) CD19+ Raji Burkitt's Lymphoma Model.

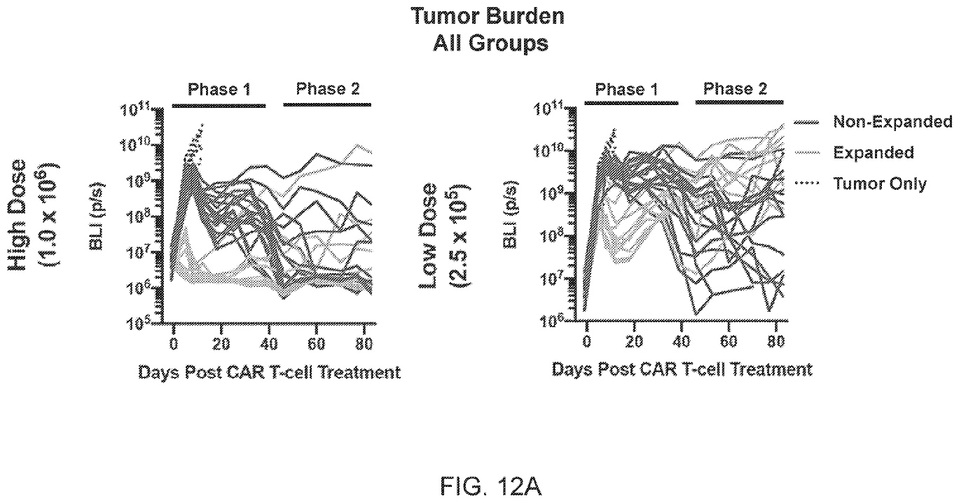

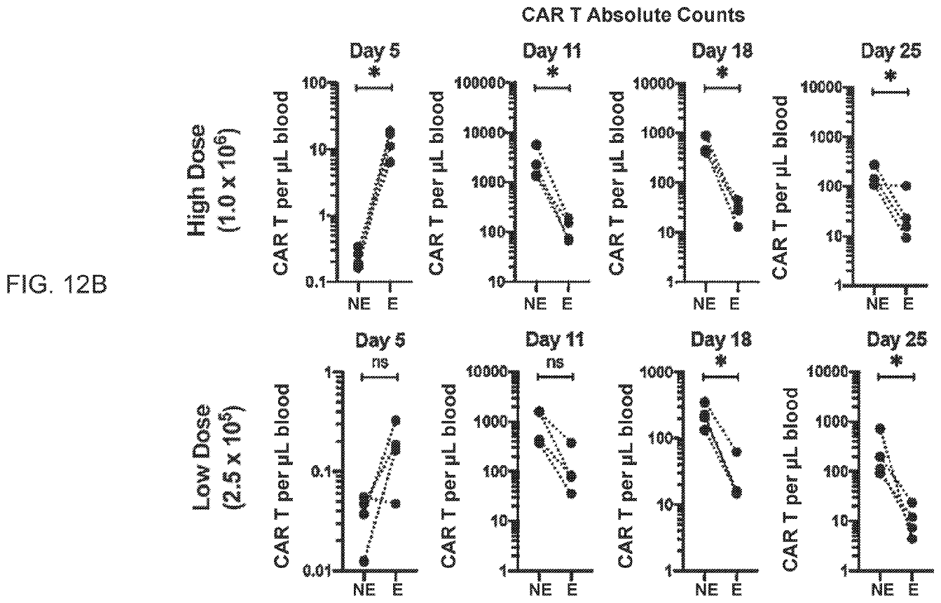

Anti-CD19 CAR-T cell compositions generated from non-expanded and expanded matched-donor engineering processes were evaluated in the Raji Burkitt's lymphoma model. NOD.Cg-Prkdc^{scid}IL-2rg^{tm1Wjl}/SzJ mice were injected intravenously with 5.0×10⁵ Raji firefly luciferase-green fluorescent protein (FfLuc-GFP) Burkitt's lymphoma cells, and 6 days later, mice were randomized into groups to balance tumor burden (n=8 per group). The following day (Day 7 post tumor engraftment), mice were treated intravenously with vehicle or anti-CD19 chimeric antigen receptor (CAR) human CD4+ and CD8+ T cells at a high dose of 1.0×10⁶ cells (FIG. 12, left panel) or a low dose of 2.5×10⁵ cells (FIG. 12, right panel) per mouse. Anti-CD19 CAR T cells were produced using T cells from one healthy donor (HD1), two patients with diffuse large B cell lymphoma (DP1, DP2), or one patient with mantle cell lymphoma (MP1) using the non-expanded process and the expanded process. Control mice did not receive treatment (tumor only). Disseminated tumor growth was assessed by imaging Raji FfLuc-positive bioluminescence (BLI).

The Raji Burkitt's lymphoma model is a rapid growing tumor, with end-stage disease characterized by hind limb paralysis occurring by Day 18 to 20 post tumor engraftment in mice that were not administered CAR-T cells. At Day 7 post tumor engraftment, the mice had a moderate tumor burden when CAR-T treatment was given. Mice treated with compositions generated using the non-expanded process had delayed responses in which the peak tumor inhibition occurred around 40 days after CAR-T cell administration (FIG. 12A, Phase 1). In contrast, treatment with compositions generated using the expanded process at doses that are typically sub-curative in this model, resulted in an early antitumor effect that started to diminish after 50 days (FIG. 12A, Phase 2). The numbers of circulating CAR-T cells were higher in mice treated with compositions generated using the expanded process on Day 5 post-treatment. However, beyond Day 5, mice treated with compositions generated using the non-expanded process showed greater expansion of CAR-T cells, with on average, 10- to 15-fold higher expansion compared to the expanded process compositions, with fold expansion reaching as high as 30-fold for some time points (FIG. 12B).

CAR-T cells with an effector memory phenotype may mediate rapid tumor clearance, whereas less-differentiated naïve-like and central memory T cells likely mediate long-term anti-tumor activity and memory formation (Gattinoni et al., Nat Med. 2017, 23(1):18-27; Klebanoff et al., Trends Immunol. 2005, 26(2):111-117). The slower initial rate of expansion of CAR-T cells of the non-expanded process compositions may be due to a less differentiated phenotype and possibly delayed expansion kinetics, while contributing to longer-term persistence. The higher tumor burden at treatment and the aggressive growth rate of the Raji tumor model may initially outpace the in vivo differentiation of CAR-T cells of the non-expanded process compositions into effector cells, leading to the lag in anti-tumor function. In contrast, in the slower growing Nalm-6 tumor model with a lower tumor burden at treatment, CAR-T cells of the non-expanded process compositions had a trend towards greater anti-tumor efficacy than CAR-T cells of the expanded process compositions. Taken together, these in vivo data are consistent with the greater proliferative capacity and potency of CAR-T cells of the non-expanded process compositions, which may be an indicator of enhanced anti-tumor activity in humans.

### Example 4: Administration of Anti-CD19 CAR-Expressing Cells to Subjects with Relapsed or Refractory B-Cell Non-Hodgkin Lymphoma

An anti-CD19 CAR-expressing therapeutic T cell composition containing CD4+CAR-expressing T cell compositions and CD8+CAR-expressing T cell compositions was administered to subjects with relapsed or refractory B-cell non-Hodgkin lymphoma (r/r B-cell NHL).

Subjects with r/r B-cell NHL were enrolled, including subjects with histologically confirmed DLBCL not otherwise specified, high-grade B-cell lymphoma with MYC and BCL2 and/or BCL6 rearrangements with DLBCL histology (HGBCL), transformed DLBCL from follicular (tFL) or marginal zone lymphoma (tMZL), primary mediastinal B-cell lymphoma (PMBCL), or FL grade 3b (FL3B). The subjects had relapsed and/or refractory disease after at least 2 lines of systemic therapy which included at least one anthracycline and rituximab (or other anti-CD20 monoclonal antibody), and/or the subjects had relapsed and/or refractory DLBCL that had failed autologous stem cell transplant (ASCT) treatment.

The anti-CD19 CAR-expressing therapeutic T cell compositions administered were generated by a process in which the cells were not subjected to cultivation steps for the purpose of expanding (proliferating) the cells, which resulted in a CAR T cell product with a phenotypic profile containing a high proportion of cell types that were less differentiated. The process included immunoaffinity-based (e.g., immunomagnetic selection) enrichment of CD4+ and CD8+ cells from previously cryopreserved leukapheresis samples from the individual subjects to be treated.

The selected CD4+ and CD8+ T cell compositions were mixed at up to 900×10⁶ total viable CD4+ and CD8+ T cells, and typically at an approximate 1:1 ratio, prior to stimulation incubation for 16-24 hours with anti-CD3/anti-CD28 Fab conjugated oligomeric streptavidin mutein reagents in a serum-free complete media containing recombinant IL-2 (100 IU/mL) recombinant IL-7 (600 IU/mL) and recombinant IL-15 (100 IU/mL). After stimulation, cells were transduced by spinoculation with a lentiviral vector encoding the anti-CD19 CAR. The CAR contained an anti-CD19 scFv, an immunoglobulin-derived spacer, a transmembrane domain derived from CD28, a costimulatory region derived from 4-1BB, and a CD3-zeta intracellular signaling domain. The viral vector further contained sequences encoding a truncated receptor, which served as a surrogate marker for CAR expression; separated from the CAR sequence by a T2A ribosome skip sequence.

After the spinoculation, the cells were washed and resuspended in a basal serum-free media without the addition of recombinant cytokines, and incubated at about 37.0° C. in an incubator. After about 48 hours after initiation of stimulation, D-biotin was then added and mixed with the cells to dissociate anti-CD3 and anti-CD28 Fab reagents from oligomeric streptavidin reagent. The cells were further incubated for another 48 hours (until about 4 days after initiation of stimulation), and then formulated with a cryoprotectant. The process did not involve a subsequent step for expansion of transduced cells and was generally shorter than a process that involved expansion of transduced cells. The process resulted in a cell composition in which greater than 80% percent of the cells were T cells, containing both CD4+CAR+ and CD8+CAR+ T cells enriched for a central memory phenotype as compared to the starting samples and as compared to cell compositions generated using a manufacturing process that involved a cultivation step aimed to expand transduced cells.

The cryopreserved cell compositions containing both CD4+CAR+ and CD8+CAR+ T cells were thawed prior to intravenous administration. Prior to CAR+ T cell infusion, subjects received a lymphodepleting chemotherapy with fludarabine (flu, 30 mg/m²/day) IV and cyclophosphamide (Cy, 300 mg/m²/day) IV for three (3) days with reduced dose for subjects with decreased renal function.

The subjects received CAR-expressing T cells 2-7 days after completion of lymphodepletion. Each subject was administered a single dose of 10×10⁶ total CAR-expressing T cells. It is contemplated that subjects in this study are to be administered a dose from among the following dose levels: 5×10⁶ total CAR+ T cells, 10×10⁶ total CAR+ T cells, 25×10⁶ total CAR+ T cells, 50×10⁶ total CAR+ T cells, and 100×10⁶ total CAR+ T cells. Each dose contained CD3+CAR+ T cells (≥80% CD3+ cells; ≥10% CD3+CAR+ cells).

Response to treatment was assessed based on radiographic tumor assessment by positron emission tomography (PET) and/or computed tomography (CT) and/or magnetic resonance imaging (MRI) scans at baseline prior to treatment and at various times following treatment (e.g. based on Lugano classification, see, e.g., Cheson et al., (2014) JCO 32(27):3059-3067).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID NO. | SEQUENCE | DESCRIPTION |
| 1 | ESKYGPPCPPCP | spacer |
| | | (IgG4hinge) (aa) |
| | | Homo sapiens |
| 2 | GAATCTAAGTACGGACCGCCCTGCCCCCCTTGCCCT | spacer |
| | | (IgG4hinge) (nt) |
| | | homo sapiens |
| 3 | ESKYGPPCPPCPGQPREPQVYTLPPSQEEMTKNQVSLTCLVKGFY | Hinge-CH3 spacer |
| | PSDIAVEWESNGQPENNYKTTPPVLDSDGSFFLYSRLTVDKSRWQ | Homo sapiens |
| | EGNVFSCSVMHEALHNHYTQKSLSLSLGK | |
| 4 | ESKYGPPCPPCPAPEFLGGPSVFLFPPKPKDTLMISRTPEVTCVVVD | Hinge-CH2-CH3 |
| | VSQEDPEVQFNWYVDGVEVHNAKTKPREEQFNSTYRVVSVLTVL | spacer |
| | HQDWLNGKEYKCKVSNKGLPSSIEKTISKAKGQPREPQVYTLPPS | Homo sapiens |
| | QEEMTKNQVSLTCLVKGFYPSDIAVEWESNGQPENNYKTTPPVL | |
| | DSDGSFFLYSRLTVDKSRWQEGNVFSCSVMHEALHNHYTQKSLS | |
| | LSLGK | |
| 5 | RWPESPKAQASSVPTAQPQAEGSLAKATTAPATTRNTGRGGEEK | IgD-hinge-Fc |
| | KKEKEKEEQEERETKTPECPSHTQPLGVYLLTPAVQDLWLRDKA | Homo sapiens |
| | TFTCFVVGSDLKDAHLTWEVAGKVPTGGVEEGLLERHSNGSQSQ | |
| | HSRLTLPRSLWNAGTSVTCTLNHPSLPPQRLMALREPAAQAPVKL | |
| | SLNLLASSDPPEAASWLLCEVSGFSPPNILLMWLEDQREVNTSGF | |
| | APARPPPQPGSTTFWAWSVLRVPAPPSPQPATYTCVVSHEDSRTL | |
| | LNASRSLEVSYVTDH | |
| 6 | LEGGGEGRGSLLTCGDVEENPGPR | T2A |
| | | artificial |
| 7 | MLLLVTSLLLCELPHPAFLLIPRKVCNGIGIGEFKDSLSINATNIKHF | tEGFR |
| | KNCTSISGDLHILPVAFRGDSFTHTPPLDPQELDILKTVKEITGFLLI | artificial |
| | QAWPENRTDLHAFENLEIIRGRTKQHGQFSLAVVSLNITSLGLRSL | |
| | KEISDGDVIISGNKNLCYANTINWKKLFGTSGQKTKIISNRGENSC | |
| | KATGQVCHALCSPEGCWGPEPRDCVSCRNVSRGRECVDKCNLLE | |
| | GEPREFVENSECIQCHPECLPQAMNITCTGRGPDNCIQCAHYIDGP | |
| | HCVKTCPAGVMGENNTLVWKYADAGHVCHLCHPNCTYGCTGP | |
| | GLEGCPTNGPKIPSIATGMVGALLLLLVVALGIGLFM | |
| 8 | FWVLVVVGGVLACYSLLVTVAFIIFWV | CD28 (amino |
| | | acids 153-179 of |
| | | Accession No. |
| | | P10747) |
| | | Homo sapiens |
| 9 | IEVMYPPPYLDNEKSNGTIIHVKGKHLCPSPLFPGPSKPFWVLVVV | CD28 (amino |
| | GGVLACYSLLVTVAFIIFWV | acids 114-179 of |
| | | Accession No. |
| | | P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | P10747) |
| | | Homo sapiens |
| 11 | RSKRSRGGHSDYMNMTPRRPGPTRKHYQPYAPPRDFAAYRS | CD28 (LL to GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) |
| | | Homo sapiens |
| 13 | RVKFSRSADAPAYQQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 14 | RVKFSRSAEPPAYQQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 15 | RVKFSRSADAPAYKQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 16 | RKVCNGIGIGEFKDSLSINATNIKHFKNCTSISGDLHILPVAFRGDS | tEGFR |
| | FTHTPPLDPQELDILKTVKEITGFLLIQAWPENRTDLHAFENLEIIR | artificial |
| | GRTKQHGQFSLAVVSLNITSLGLRSLKEISDGDVIISGNKNLCYAN | |
| | TINWKKLFGTSGQKTKIISNRGENSCKATGQVCHALCSPEGCWGP | |
| | EPRDCVSCRNVSRGRECVDKCNLLEGEPREFVENSECIQCHPECLP | |
| | QAMNITCTGRGPDNCIQCAHYIDGPHCVKTCPAGVMGENNTLVW | |
| | KYADAGHVCHLCHPNCTYGCTGPGLEGCPTNGPKIPSIATGMVG | |
| | ALLLLLVVALGIGLFM | |
| 17 | EGRGSLLTCGDVEENPGP | T2A artificial |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | PGGG-(SGGGG)5-P- | linker |
| | wherein P is proline, G is glycine and S is serine | |
| 23 | GSADDAKKDAAKKDGKS | Linker |
| 24 | GSTSGSGKPGSGEGSTKG | Linker |
| 25 | gacatccagatgacccagaccacctccagcctgagcgccagcct | Sequence |
| | gggcgaccgggtgaccatcagctgccgggccagccaggacatca | encoding scFv |
| | gcaagtacctgaactggtatcagcagaagcccgacggcaccgtc | |
| | aagctgctgatctaccacaccagccggctgcacagcggcgtgcc | |
| | cagccggtttagcggcagcggctccggcaccgactacagcctga | |
| | ccatctccaacctggaacaggaagatatcgccacctacttttgc | |
| | cagcagggcaacacactgccctacacctttggcggcggaacaaa | |
| | gctggaaatcaccggcagcacctccggcagcggcaagcctggca | |
| | gcggcgagggcagcaccaagggcgaggtgaagctgcaggaaagc | |
| | ggccctggcctggtggcccccagccagagcctgagcgtgacctg | |
| | caccgtgagcggcgtgagcctgcccgactacggcgtgagctgga | |
| | tccggcagccccccaggaagggcctggaatggctgggcgtgatc | |
| | tggggcagcgagaccacctactacaacagcgccctgaagagccg | |
| | gctgaccatcatcaaggacaacagcaagagccaggtgttcctga | |
| | agatgaacagcctgcagaccgacgacaccgccatctactactgc | |
| | gccaagcactactactacggcggcagctacgccatggactactg | |
| | gggccagggcaccagcgtgaccgtgagcagc | |
| 26 | X₁PPX₂P | Hinge |
| | X₁ is glycine, cysteine or arginine | |
| | X₂ is cysteine or threonine | |
| 27 | Glu Pro Lys Ser Cys Asp Lys Thr His Thr Cys Pro Pro Cys Pro | Hinge |
| 28 | Glu Arg Lys Cys Cys Val Glu Cys Pro Pro Cys Pro | Hinge |
| 29 | ELKTPLGDTHTCPRCPEPKSCDTPPPCPRCPEPKSCDTPPPCPRCPE | Hinge |
| | PKSCDTPPPCPRCP | |
| 30 | Glu Ser Lys Tyr Gly Pro Pro Cys Pro Ser Cys Pro | Hinge |
| 31 | Glu Ser Lys Tyr Gly Pro Pro Cys Pro Pro Cys Pro | Hinge |
| 32 | Tyr Gly Pro Pro Cys Pro Pro Cys Pro | Hinge |
| 33 | Lys Tyr Gly Pro Pro Cys Pro Pro Cys Pro | Hinge |
| 34 | Glu Val Val Val Lys Tyr Gly Pro Pro Cys Pro Pro Cys Pro | Hinge |
| 35 | RASQDISKYLN | CDR L1 |
| 36 | SRLHSGV | CDR L2 |
| 37 | GNTLPYTFG | CDR L3 |
| 38 | DYGVS | CDR H1 |
| 39 | VIWGSETTYYNSALKS | CDR H2 |
| 40 | YAMDYWG | CDR H3 |
| 41 | EVKLQESGPGLVAPSQSLSVTCTVSGVSLPDYGVSWIRQPPRKGL | VH |
| | EWLGVIWGSETTYYNSALKSRLTIIKDNSKSQVFLKMNSLQTDDT | |
| | AIYYCAKHYYYGGSYAMDYWGQGTSVTVSS | |
| 42 | DIQMTQTTSSLSASLGDRVTISCRASQDISKYLNWYQQKPDGTVK | VL |
| | LLIYHTSRLHSGVPSRFSGSGSGTDYSLTISNLEQEDIATYFCQQ | |
| | GNTLPYTFGGGTKLEIT | |
| 43 | DIQMTQTTSSLSASLGDRVTISCRASQDISKYLNWYQQKPDGTVKL | scFv |
| | LIYHTSRLHSGVPSRFSGSGSGTDYSLTISNLEQEDIATYFCQQGN | |
| | TLPYTFGGGTKLEITGSTSGSGKPGSGEGSTKGEVKLQESGPGLVA | |
| | PSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGG | |
| | SYAMDYWGQGTSVTVSS | |
| 44 | KASQNVGTNVA | CDR L1 |
| 45 | SATYRNS | CDR L2 |
| 46 | QQYNRYPYT | CDR L3 |
| 47 | SYWMN | CDR H1 |
| 48 | QIYPGDGDTNYNGKFKG | CDR H2 |
| 49 | KTISSVVDFYFDY | CDR H3 |
| 50 | EVKLQQSGAELVRPGSSVKISCKASGYAFSSYWMNWVKQRPGQ | VH |
| | GLEWIGQIYPGDGDTNYNGKFKGQATLTADKSSSTAYMQLSGLT | |
| | SEDSAVYFCARKTISSVVDFYFDYWGQGTTVTVSS | |
| 51 | DIELTQSPKFMSTSVGDRVSVTCKASQNVGTNVAWYQQKPGQSP | VL |
| | KPLIYSATYRNSGVPDRFTGSGSGTDFTLTITNVQSKDLADYFC | |
| | QQYNRYPYTSGGGTKLEIKR | |
| 52 | GGGGSGGGGSGGGGS | Linker |
| 53 | EVKLQQSGAELVRPGSSVKISCKASGYAFSSYWMNWVKQRPGQ | scFv |
| | GLEWIGQIYPGDGDTNYNGKFKGQATLTADKSSSTAYMQLSGLT | |
| | SEDSAVYFCARKTISSVVDFYFDYWGQGTTVTVSSGGGGSGGGG | |
| | SGGGGSDIELTQSPKFMSTSVGDRVSVTCKASQNVGTNVAWYQQ | |
| | KPGQSPKPLIYSATYRNSGVPDRFTGSGSGTDFTLTITNVQSKD | |
| | LADYFCQQYNRYPYTSGGGTKLEIKR | |
| 54 | HYYYGGSYAMDY | CDR H3 |
| 55 | HTSRLHS | CDRL2 |
| 56 | QQGNTLPYT | CDR L3 |
| 57 | ACACGGCCTCGTGTATTACTGT | IGH primer |
| 58 | ACCTGAGGAGACGGTGACC | IGH Primer |
| 59 | MEAGITGTWYNQLGSTFIVTAGADGALTGTYIGARGNAESRYV | Mutein |
| | LTGRYDSAPATDGSGTALGWTVAWKNNYRNAHSATTWSGQYVG | Streptavidin Ile⁴⁴- |
| | GAEARINTQWLLTSGTTEANAWKSTLVGHDTFTKVKPSAAS | Gly⁴⁵-Ala-⁴⁶-Arg⁴⁷ |
| | | Species: |
| | | Streptomyces |
| | | avidinii |
| 60 | Gln Val Gln Leu Gln Gln Ser Gly Ala Glu Leu | Variable Ileavy |
| | Ala Arg Pro Gly Ala Ser Val Lys Met Ser Cys | chain of anti-CD3 |
| | Lys Ala Ser Gly Tyr Thr Phe Thr Arg Tyr Thr | antibody OKT3 |
| | Met His Trp Val Lys Gln Arg Pro Gly Gln Gly | |
| | Leu Glu Trp Ile Gly Tyr Ile Asn Pro Ser Arg | |
| | Gly Tyr Thr Asn Tyr Asn Gln Lys Phe Lys Asp | |
| | Lys Ala Thr Leu Thr Thr Asp Lys Ser Ser Ser | |
| | Thr Ala Tyr Met Gln Leu Ser Ser Leu Thr Ser | |
| | Glu Asp Ser Ala Val Tyr Tyr Cys Ala Arg Tyr | |
| | Tyr Asp Asp His Tyr Cys Leu Asp Tyr Trp Gly | |
| | Gln Gly Thr Thr Leu Thr Val Ser Ser | |
| 61 | Gln Ile Val Leu Thr Gln Ser Pro Ala Ile Met | Variable Light |
| | Ser Ala Ser Pro Gly Glu Lys Val Thr Met Thr | chain of anti-CD3 |
| | Cys Ser Ala Ser Ser Ser Val Ser Tyr Met Asn | antibody OKT3 |
| | Trp Tyr Gln Gln Lys Ser Gly Thr Ser Pro Lys | |
| | Arg Trp Ile Tyr Asp Thr Ser Lys Leu Ala Ser | |
| | Gly Val Pro Ala His Phe Arg Gly Ser Gly Ser | |
| | Gly Thr Ser Tyr Ser Leu Thr Ile Ser Gly Met | |
| | Glu Ala Glu Asp Ala Ala Thr Tyr Tyr Cys Gln | |
| | Gln Trp Ser Ser Asn Pro Phe Thr Phe Gly Ser | |
| | Gly Thr Lys Leu Glu Ile Asn | |
| 62 | Leu Gln Gln Ser Gly Ala Glu Leu Val Lys Pro | Variable Ileavy |
| | Gly Ala Ser Val Arg Leu Ser Cys Lys Ala Ser | chain of anti-CD28 |
| | Gly Tyr Thr Phe Thr Glu Tyr Ile Ile His Trp | antibody CD28.3 |
| | Ile Lys Leu Arg Ser Gly Gln Gly Leu Glu Trp | |
| | Ile Gly Trp Phe Tyr Pro Gly Ser Asn Asp Ile | |
| | Gln Tyr Asn Ala Lys Phe Lys Gly Lys Ala Thr | |
| | Leu Thr Ala Asp Lys Ser Ser Ser Thr Val Tyr | |
| | Met Glu Leu Thr Gly Leu Thr Ser Glu Asp Ser | |
| | Ala Val Tyr Phe Cys Ala Arg Arg Asp Asp Phe | |
| | Ser Gly Tyr Asp Ala Leu Pro Tyr Trp Gly Gln | |
| | Gly Thr Met Val Thr Val | |
| 63 | Asp Ile Gln Met Thr Gln Ser Pro Ala Ser Leu | Variable Light |
| | Ser Val Ser Val Gly Glu Thr Val Thr Ile Thr | chain of anti-CD28 |
| | Cys Arg Thr Asn Glu Asn Ile Tyr Ser Asn Leu | antibody CD28.3 |
| | Ala Trp Tyr Gln Gln Lys Gln Gly Lys Ser Pro | |
| | Gln Leu Leu Ile Tyr Ala Ala Thr His Leu Val | |
| | Glu Gly Val Pro Ser Arg Phe Ser Gly Ser Gly | |
| | Ser Gly Thr Gln Tyr Ser Leu Lys Ile Thr Ser | |
| | Leu Gln Ser Glu Asp Phe Gly Asn Tyr Tyr Cys | |
| | Gln His Phe Trp Gly Thr Pro Cys Thr Phe Gly | |
| | Gly Gly Thr Lys Leu Glu Ile Lys Arg | |
| 64 | SAWSHPQFEKGGGSGGGSGGSAWSHPQFEK | Twin-Strep-tag |
| 65 | MLLLVTSLLLCELPHPAFLLIP | GMCSFR alpha |
| | | chainsignal |
| | | sequence |
| 66 | atgcttctcctggtgacaagccttctgctctgtgagttaccacacc | GMCSFR alpha |
| | cagcattcctcctgatccca | chainsignal |
| | | sequence |
| 67 | MALPVTALLLPLALLLHA | CD8 alpha signal |
| | | peptide |
| 68 | DPSKDSKAQVSAAEAGITGTWYNQLGSTFIVTAGADGALTGTYES | Streptavidin |
| | AVGNAESRYVLTGRYDSAPATDGSGTALGWTVAWKNNYRNAH | Species: |
| | SATTWSGQYVGGAEARINTQWLLTSGTTEANAWKSTLVGHDTFT | Streptomyces |
| | KVKPSAASIDAAKKAGVNNGNPLDAVQQ | avidinii |
| | | UniProt No. |
| | | P22629 |
| 69 | EAGITGTWYNQLGSTFIVTAGADGALTGTYESAVGNAESRYVLT | Minimal |
| | GRYDSAPATDGSGTALGWTVAWKNNYRNAHSATTWSGQYVGG | streptavidin |
| | AEARINTQWLLTSGTTEANAWKSTLVGHDTFTKVKPSAAS | Species: |
| | | Streptomyces |
| | | avidinii |
| 70 | His-Pro-GIn-Phe | Streptavidin- |
| | | binding peptide |
| 71 | His-Pro-Xaa | Streptavidin |
| | | Binding peptide |
| | | Xaa is selected |
| | | from Gln, Asp, |
| | | and Met |
| 72 | Oaa-Xaa-His-Pro-Gln-Phe-Y aa-Zaa | Streptavidin- |
| | | binding peptide |
| | | Oaa is Trp, Lys or |
| | | Arg; |
| | | Xaa is any amino |
| | | acid; |
| | | Yaa is Gly or Glu |
| | | Zaa is Gly, Lys or |
| | | Arg |
| 73 | -Trp-Xaa-His-Pro-Gln-Phe-Yaa-Zaa- | Streptavidin- |
| | | binding peptide |
| | | Xaa is any amino |
| | | acid; |
| | | Yaa is Gly or Glu |
| | | Zaa is Gly, Lys or |
| | | Arg |
| 74 | Trp-Arg-His-Pro-Gln-Phe-Gly-Gly | Streptavidin |
| | | binding peptide, |
| | | Strep-tag® |
| 75 | WSHPQFEK | Strep-tag® 11 |
| 76 | Trp-Ser-His-Pro-Gln-Phe-Glu-Lys-(Xaa)n-Trp-Ser- | Sequential |
| | His-Pro-Gln-Phe-Glu-Lys- | modules of |
| | | streptavidin- |
| | | binding peptide |
| | | Xaa is any amino |
| | | acid; |
| | | n is either 8 or 12 |
| 77 | Trp-Ser-His-Pro-Gln-Phe-Glu-Lys-(GlyGlyGlySer)n- | Sequential |
| | Trp-Ser-His-Pro-Gln-Phe-Glu-Lys | modules of |
| | | streptavidin- |
| | | binding peptide |
| | | n is 2 or 3 |
| 78 | WSHPQFEKGGGSGGGSGGGSWSHPQFEK | Twin-Strep-tag |
| 79 | WSHPQFEKGGGSGGGSWSHPQFEK | Twin-Strep-tag |
| 80 | WSHPQFEKGGGSGGGSGGSAWSHPQFEK | Twin-Strep-tag |
| 81 | SAWSHPQFEKGGGSGGGSGGGSWSHPQFEK | Twin-Strep-tag |
| 82 | SAWSHPQFEKGGGSGGGSGGSAWSHPQFEK | Twin-Strep-tag |
| 83 | DPSKDSKAQVSAAEAGITGTWYNQLGSTFIVTAGADGALTGTYV | Mutein |
| | TARGNAESRYVLTGRYDSAPATDGSGTALGWTVAWKNNYRNA | Streptavidin |
| | HSATTWSGQYVGGAEARINTQWLLTSGTTEANAWKSTLVGHDT | Val44-Thr45- |
| | FTKVKPSAASIDAAKKAGVNNGNPLDAVQQ | Ala46-Arg47 |
| | | Species: |
| | | Streptomyces |
| | | avidinii |
| 84 | EAGITGTWYNQLGSTFIVTAGADGALTGTYVTARGNAESRYVLT | Mutein |
| | GRYDSAPATDGSGTALGWTVAWKNNYRNAHSATTWSGQYVGG | Streptavidin |
| | AEARINTQWLLTSGTTEANAWKSTLVGHDTFTKVKPSAAS | Val44-Thr45- |
| | | Ala46-Arg47 |
| | | Species: |
| | | Streptomyces |
| | | avidinii |
| 85 | MEAGITGTWYNQLGSTFIVTAGADGALTGTYVTARGNAESRYVL | Mutein |
| | TGRYDSAPATDGSGTALGWTVAWKNNYRNAHSATTWSGQYVG | Streptavidin |
| | GAEARINTQWLLTSGTTEANAWKSTLVGHDTFTKVKPSAAS | Val44-Thr45- |
| | | Ala46-Arg47 |
| | | Species: |
| | | Streptomyces |
| | | avidinii |
| 86 | DPSKDSKAQVSAAEAGITGTWYNQLGSTFIVTAGADGALTGTYIG | Mutein |
| | ARGNAESRYVLTGRYDSAPATDGSGTALGWTVAWKNNYRNAH | Streptavidin Ile44- |
| | SATTWSGQYVGGAEARINTQWLLTSGTTEANAWKSTLVGHDTFT | Gly45-Ala-46- |
| | KVKPSAASIDAAKKAGVNNGNPLDAVQQ | Arg47 |
| | | Species: |
| | | Streptomyces |
| | | avidinii |
| 87 | EAGITGTWYNQLGSTFIVTAGADGALTGTYIGARGNAESRYVLT | Mutein |
| | GRYDSAPATDGSGTALGWTVAWKNNYRNAHSATTWSGQYVGG | Streptavidin Ile44- |
| | AEARINTQWLLTSGTTEANAWKSTLVGHDTFTKVKPSAAS | Gly45-Ala-46- |
| | | Arg47 |
| | | Species: |
| | | Streptomyces |
| | | avidinii |
| 88 | EAGITGTWYNQLGSTFIVTAGADGALTGTYVTARGNAESRYVLT | Mutein |
| | GRYDSAPATDGSGTALGWTVAWKNNYRNAHSATTWSGQYVGG | Streptavidin |
| | AEARINTQWLLTSGTTEENAGYSTLVGHDTFTKVKPSAAS | Val44-Thr45- |
| | | Ala46-Arg47 and |
| | | Glull7, Gly120, |
| | | Try 121 (mutein |
| | | ml-9) |
| | | Species: |
| | | Streptomyces |
| | | avidinii |
| 89 | DPSKDSKAQVSAAEAGITGTWYNQLGSTFIVTAGADGALTGTYV | Mutein |
| | TARGNAESRYVLTGRYDSAPATDGSGTALGWTVAWKNNYRNA | Streptavidin |
| | HSATTWSGQYVGGAEARINTQWLLTSGTTEENAGYSTLVGHDTF | Val44-Thr45- |
| | TKVKPSAAS | Ala46-Arg47 and |
| | | Glu117, Gly120, |
| | | Try121 (mutein |
| | | mi-9) |
| | | Species: |
| | | Streptomyces |
| | | avidinii |
| 90 | MEAGITGTWYNQLGSTFIVTAGADGALTGTYESAVGNAESRYVL | Minimal |
| | TGRYDSAPATDGSGTALGWTVAWKNNYRNAHSATTWSGQYVG | streptavidin |
| | GAEARINTQWLLTSGTTEANAWKSTLVGHDTFTKVKPSAAS | Species: |
| | | Streptomyces |
| | | avidinii |

1. A method of treating a B-cell non-Hodgkin lymphoma (r/r B-cell NHL), the method comprising administering to a subject having or suspected of having a B-cell NHL a composition comprising engineered T cells expressing a chimeric antigen receptor (CAR) that targets CD19, wherein:
the composition comprises CD4+ T cells expressing the CAR and CD8+ T cells expressing the CAR;
the composition comprises between at or about 5×10⁶ CAR-expressing T cells and at or about 100×10⁶ CAR-expressing T cells, inclusive;
at least or at least about 80% of the cells in the composition are CD3⁺ cells; and
at least or at least about 80% of the CAR⁺ T cells in the composition are of a naïve-like or central memory phenotype.

2. A method of treating a B-cell non-Hodgkin lymphoma (r/r B-cell NHL), the method comprising administering to a subject having or suspected of having a B-cell NHL a composition comprising engineered T cells expressing a chimeric antigen receptor (CAR) that targets CD19, wherein:
the composition comprises CD4+ T cells expressing the CAR and CD8+ T cells expressing the CAR;
the composition comprises between at or about 5×10⁶ CAR-expressing T cells and at or about 100×10⁶ CAR-expressing T cells, inclusive;
at least or at least about 80% of the cells in the composition are CD3⁺ cells; and
at least or at least about 50% of the CD4⁺CAR⁺ T cells in the composition are CD27⁺CCR7⁺ and/or at least or at least about 50% of the CD8⁺CAR⁺ T cells in the composition are CD27⁺CCR7⁺.

3. The method of claim 1, wherein the composition comprises CD4⁺ T cells expressing the CAR and CD8⁺ T cells expressing the CAR at a ratio between about 1:2.5 and about 2.5:1.

4-5. (canceled)

6. The method of claim 1, wherein the composition comprises between at or about 5×10⁶ CAR-expressing T cells and at or about 50×10⁶ CAR-expressing T cells, inclusive.

7-12. (canceled)

13. The method of claim 1, wherein at least or at least about 90% of the cells in the composition are CD3⁺ cells.

14. (canceled)

15. The method of claim 1, wherein between at or about 5% and at or about 30% of the CAR⁺ T cells in the composition express a marker of apoptosis.

16-20. (canceled)

21. The method of claim 2, wherein at least or at least about 80% of the CAR⁺ T cells in the composition are of a naïve-like or central memory phenotype.

22-26. (canceled)

27. The method of claim 1, wherein the at least or at least about 80% of the CAR⁺ T cells in the composition that are of a naïve-like or central memory phenotype are surface positive for a marker expressed on naïve-like or central memory T cells.

28. The method of claim 27, wherein the marker expressed on naïve-like or central memory T cell is selected from the group consisting of CD45RA, CD27, CD28, and CCR7.

29. The method of claim 1, wherein the at least or at least about 80% of the CAR⁺ T cells in the composition that are of a naïve-like or central memory phenotype have a phenotype selected from CCR7⁺CD45RA⁺, CD27⁺CCR7⁺, or CD62L⁻CCR7⁺.

30-32. (canceled)

33. The method of claim 1, wherein:
at least or at least about 50% of the CD4⁺CAR⁺ T cells in the composition are of a naïve-like or central memory phenotype that is CCR7⁺CD45RA⁺ or CCR7⁺CD45RA⁻;
at least or at least about 50% of the CD4⁺CAR⁺ T cells in the composition are of a naïve-like or central memory phenotype that is CD27⁺CCR7⁺;
at least or at least about 50% of the CD8⁺CAR⁺ T cells in the composition are of a naïve-like or central memory phenotype that is CCR7⁺CD45RA⁺ or CCR7⁺CD45RA⁻; and/or
at least or at least about 50% of the CD8⁺CAR⁺ T cells in the composition are of a naïve-like or central memory phenotype that is CD27⁺CCR7⁺.

34-52. (canceled)

53. The method of claim 1, wherein the fraction of integrated vector copy number (iVCN) to total VCN in the CAR⁺ T cells in the composition, on average, is less than or less than about 0.9.

54-59. (canceled)

60. The method of claim 1, wherein the integrated vector copy number (iVCN) of the CAR⁺ T cells in the composition, on average, is between or between about 0.4 copies per diploid genome and 3.0 copies per diploid genome, inclusive.

61-64. (canceled)

65. The method of claim 1, wherein the B-cell NHL is selected from the group consisting of: diffuse large B-cell lymphoma (DLBCL); transformed DLBCL; high grade B-cell lymphoma (HGBCL); primary mediastinal large B cell lymphoma (PMBCL); and follicular lymphoma (FL).

66. The method of claim 1, wherein at or immediately prior to the time of the administration of the composition comprising engineered T cells, the subject has relapsed following remission after treatment with, or become refractory to: (i) two or more prior therapies for the B-cell NHL and/or (ii) an autologous stem cell transplant (ASCT) therapy.

67. The method of claim 66, wherein the two or more prior therapies for the B-cell NHL comprise an anthracycline and a CD20-targeted agent.

68-69. (canceled)

70. The method of claim 1, wherein prior to the administration, the subject has been preconditioned with a lymphodepleting therapy.

71. (canceled)

72. The method of claim 1, wherein the administration of the composition comprising engineered T cells and/or the lymphodepleting therapy is carried out via outpatient delivery.

73. The method of claim 70, wherein the lymphodepleting therapy comprises the administration of fludarabine at 30 mg/m² body surface area of the subject, daily, and cyclophosphamide at 300 mg/m² body surface area of the subject, daily, each for 3 days.

74-77. (canceled)

78. The method of claim 1, wherein the T cells are autologous to the subject.

79. The method of claim 1, wherein:
at least 35% of subjects treated according to the method achieve a complete response (CR);
at least 60% of subjects achieving a CR exhibit a CR that is durable for at or greater than 3 months or at or greater than 6 months; and/or
at least 60% of subjects achieving a CR by one month and/or by 3 months remain in response, remain in CR, and/or survive or survive without progression, for at or greater than 3 months and/or at or greater than 6 months and/or at greater than 9 months after achieving the CR; and/or
at least 50% of the subjects treated according to the method achieve objective response (OR);
at least 60% of subjects achieving an OR exhibit an OR that is durable for at or greater than 3 months or at or greater than 6 months; and/or
at least 35% of subjects achieving an OR remain in response or survive for at or greater than 3 months and/or at or greater than 6 months after achieving the OR.

80. The method of claim 1, wherein the cells are autologous to the subject, and
no minimum absolute lymphocyte count (ALC) for apheresis is required and/or specified for production of the therapy; and/or
the cells are produced by a process which, for at least 90% of subjects having the B-cell NHL, is capable of generating a cell product for administration according to the method.

81-84. (canceled)

85. The method of claim 1, wherein:
greater than or greater than about 50%, about 60%, about 70%, or about 80% of the subjects treated according to the method do not exhibit a grade 3 or greater cytokine release syndrome (CRS) and/or do not exhibit a grade 3 or greater neurotoxicity and/or greater than 40% or 50% or 55% of the subjects treated according to the method do not exhibit any neurotoxicity or CRS; and/or
greater than or greater than about 30%, 35%, 40%, or 50% of the subjects treated according to the method do not exhibit any grade of cytokine release syndrome (CRS) or neurotoxicity; and/or
at least at or about 45%, 50%, 60%, 65%, 70%, 75%, 80%, 85%, 90%, or 95% of subjects treated according to the method do not exhibit onset of CRS earlier than 3 days following initiation of the administration and/or do not exhibit onset of neurotoxicity earlier than 5 days following initiation of the administration; and/or
the median onset of neurotoxicity among subjects treated according to the method is at or after the median peak of, or median time to resolution of, CRS in subjects treated according to the method and/or the median onset of neurotoxicity among subjects treated according to the method is greater than at or about 8, 9, 10, or 11 days.

86. The method of claim 1, wherein:
at least 50% of subjects treated according to the method achieve a complete response (CR);
at least 70% of the subjects treated according to the method achieve objective response (OR); and
greater than or greater than about 50% of the subjects treated according to the method do not exhibit any grade of cytokine release syndrome (CRS) or neurotoxicity; and
greater than or greater than about 80% of the subjects treated according to the method do not exhibit a grade 3 or greater cytokine release syndrome (CRS) and/or do not exhibit a grade 3 or greater neurotoxicity.

87. The method of claim 1, wherein:
the CAR comprises an extracellular antigen-binding domain specific for CD19, a transmembrane domain, a cytoplasmic signaling domain derived from a costimulatory molecule, and a cytoplasmic signaling domain derived from a primary signaling ITAM-containing molecule;
the CAR comprises, in order, an extracellular antigen-binding domain specific for CD19, a transmembrane domain, a cytoplasmic signaling domain derived from a costimulatory molecule, and a cytoplasmic signaling domain derived from a primary signaling ITAM-containing molecule; or
the CAR comprises an extracellular antigen-recognition domain that specifically binds to CD19, a transmembrane domain, and an intracellular signaling domain comprising a CD3-zeta (CD3ζ) chain and a costimulatory signaling region that is a signaling domain of 4-1BB.

88. (canceled)

89. The method of claim 87, wherein the extracellular antigen-binding domain is an scFv.

90. (canceled)

91. The method of claim 89, wherein:
the scFv comprises a variable heavy chain region of FMC63 and a variable light chain region of FMC63; or
the scFv is set forth as SEQ ID NO: 43.

92. (canceled)

93. The method of claim 87, wherein:
the cytoplasmic signaling domain derived from a costimulatory molecule is a signaling domain of 4-1BB; and/or
the cytoplasmic signaling domain derived from a primary signaling ITAM-containing molecule is a CD3zeta signaling domain; and/or
the transmembrane domain is a transmembrane domain from CD28.

94. (canceled)

95. The method of claim 87, wherein the CAR further comprises a spacer between the transmembrane domain and the extracellular antigen-binding domain.

96. The method of claim 95, wherein the spacer is a polypeptide spacer that comprises or consists of all or a portion of an immunoglobulin hinge or a modified version thereof.

97. The method of claim 95, wherein the spacer:
is at or about 12 amino acids in length; or
comprises or consists of the sequence of SEQ ID NO: 1, or a sequence encoded by SEQ ID NO: 2; or
is a polypeptide spacer that comprises the sequence of SEQ ID NO: 1.

98-100. (canceled)

101. The method of claim 96, wherein the CAR contains in order from N-terminus to C-terminus: an extracellular antigen-binding domain that is the scFv set forth in SEQ ID NO: 43, the spacer set forth in SEQ ID NO:1, the transmembrane domain set forth in SEQ ID NO:8, the 4-1BB costimulatory signaling domain set forth in SEQ ID NO:12, and the signaling domain of a CD3-zeta (CD3ζ) chain set forth in SEQ ID NO:13.

102. The method of claim 1, wherein the composition comprising engineered T cells is produced by a manufacturing process comprising:
(i) exposing an input composition comprising primary T cells, optionally an input composition comprising autologous T cells selected from the subject, with a stimulatory reagent comprising an oligomeric particle reagent comprising a plurality of streptavidin mutein molecules under conditions to stimulate T cells, thereby generating a stimulated population, wherein:
the oligomeric particle reagent comprises a first agent comprising an anti-CD3 antibody or antigen binding fragment thereof and a second agent comprising an anti-CD28 antibody or antigen binding fragment thereof;
(ii) introducing into T cells of the stimulated population, a heterologous polynucleotide encoding the CAR that targets CD19, thereby generating a population of transformed cells;
(iii) incubating the population of transformed cells for up to 96 hours; and
(iv) harvesting T cells of the population of transformed cells, thereby producing a composition of engineered cells, wherein the harvesting is carried out at a time between 24 and 120 hours, inclusive, after the exposing to the stimulatory reagent is initiated.

103. (canceled)

104. The method of claim 102, wherein:
the first agent and the second agent each comprise a streptavidin-binding peptide that reversibly binds the first agent and the second agent to the oligomeric particle reagent; and/or
the streptavidin mutein molecule is a tetramer of a streptavidin mutein comprising amino acid residues Val44-Thr45-Ala46-Arg47 or Ile44-Gly45-Ala46-Arg47.

105. (canceled)

106. The method of claim 102, wherein the oligomeric particle reagent comprises between 1,000 and 5,000 streptavidin mutein tetramers, inclusive.

107. The method of claim 102, wherein the method further comprises, prior to harvesting the cells, adding biotin or a biotin analog after or during the incubation.

108. The method of claim 102, wherein the harvesting is carried out:
at a time between 48 and 120 hours, inclusive, after the exposing to the stimulatory reagent is initiated; and/or
at a time when integrated vector is detected in the genome but prior to achieving a stable integrated vector copy number (iVCN) per diploid genome; and/or
at a time before the total number of viable cells at the harvesting is more than or more than about three times the number of total viable cells of the stimulated population; and/or
at a time when the total number of viable cells at the harvesting is at or about three times, at or about two times, or the same or about the same as the number of total viable cells of the stimulated population; and/or
at a time when the percentage of CD27⁺CCR7⁺ cells is greater than or greater than about 50% among total T cells in the population of transformed cells, total CD3⁺ T cells in the population of transformed cells, total CD4⁺ T cells in the population of transformed cells, or total CD8⁺ T cells in the population of transformed cells, or of CAR-expressing cells thereof, in the population of transformed cells; and/or
at a time when the percentage of CD45RA⁺CCR7⁺ and CD45RA⁻CCR7⁺ cells is greater than or greater than about 60% among total T cells in the population of transformed cells, total CD3⁺ T cells in the population of transformed cells, total CD4⁺ T cells in the population of transformed cells, or total CD8⁺ T cells, or of CAR-expressing cells thereof, in the population of transformed cells.

109-113. (canceled)

114. The method of claim 1, wherein the cells in the administered composition are produced by a manufacturing process to produce an output composition (i) comprising engineered CD4+ T cells and engineered CD8+ T cells and (ii) exhibiting a predetermined feature, wherein iterations of the manufacturing process produce a plurality of the output compositions, optionally from human biological samples, when carried out among a plurality of different individual subjects, in which the predetermined feature of the output composition among the plurality of output compositions is selected from:
the mean percentage of cells of a memory phenotype in the plurality of the output compositions is between about 40% and about 65%, between about 40% and about 45%, between about 45% and about 50%, between about 50% and about 55%, between about 55% and about 60%, or between about 60% and about 65%;
the mean percentage of cells of a central memory phenotype in the plurality of the output compositions is between about 40% and about 65%, between about 40% and about 45%, between about 45% and about 50%, between about 50% and about 55%, between about 55% and about 60%, or between about 60% and about 65%;
the mean percentage of cells that are CD27+, CD28+, CCR7+, CD45RA−, CD45RO+, CD62L+, CD3+, CD95+, granzyme B−, and/or CD127+ in the plurality of the output compositions is between about 40% and about 65%, between about 40% and about 45%, between about 45% and about 50%, between about 50% and about 55%, between about 55% and about 60%, or between about 60% and about 65%;
the mean percentage of cells that are CCR7+/CD45RA− or CCR7+/CD45RO+ in the plurality of the output compositions is between about 40% and about 65%, between about 40% and about 45%, between about 45% and about 50%, between about 50% and about 55%, between about 55% and about 60%, or between about 60% and about 65%;
the mean percentage of central memory CD4+ T cells in the engineered CD4+ T cells, optionally CAR+CD4+ T cells, of the plurality of the output compositions is between about 40% and about 65%, between about 40% and about 45%, between about 45% and about 50%, between about 50% and about 55%, between about 55% and about 60%, or between about 60% and about 65%;
the mean percentage of central memory CD8+ T cells in the engineered CD8+ T cells, optionally CAR+CD8+ T cells, of the plurality of the output compositions is between about 40% and about 65%, between about 40% and about 45%, between about 45% and about 50%, between about 50% and about 55%, between about 55% and about 60%, or between about 60% and about 65%; and/or
the mean percentage of central memory T cells, optionally CD4+ central memory T cells and CD8+ central memory T cells, in the engineered T cells, optionally CAR+ T cells, of the plurality of the output compositions is between about 40% and about 65%, between about 40% and about 45%, between about 45% and about 50%, between about 50% and about 55%, between about 55% and about 60%, or between about 60% and about 65%.

115-116. (canceled)

117. The method of claim 1, wherein the B-cell NHL is a relapsed and/or refractory B-cell non-Hodgkin lymphoma (B-cell NHL).

118. An article of manufacture comprising a composition comprising genetically engineered cells expressing a chimeric antigen receptor (CAR) that targets CD19, and instructions for administering the composition of cells in accordance with the method of claim 1.


SEQUENCE LISTING (90 sequences)

SEQ ID NO: 1 - protein, 12 aa, Homo sapiens
  spacer (IgG4hinge)
ESKYGPPCPPCP

SEQ ID NO: 2 - DNA, 36 bp, Homo sapiens
  spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28 (amino acids 153-179 of Accession No. P10747)
FWVLVVVGGVLACYSLLVTVAFIIFWV

SEQ ID NO: 9 - protein, 66 aa, Homo sapiens
  CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28 (amino acids 180-220 of P10747)
RSKRSRLLHSDYMNMTPRRPGPTRKHYQPYAPPRDFAAYRS

SEQ ID NO: 11 - protein, 41 aa, Homo sapiens
  CD28 (LL to GG)
RSKRSRGGHSDYMNMTPRRPGPTRKHYQPYAPPRDFAAYRS

SEQ ID NO: 12 - protein, 42 aa, Homo sapiens
  4-1BB (amino acids 214-255 of Q07011.1)
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 22 aa, Artificial Sequence
  F2A
VKQTLNFDLLKLAGDVESNPGP

SEQ ID NO: 22 - protein, 35 aa, Artificial Sequence
  Linker
PGGGSGGGGSGGGGSGGGGSGGGGSGGGGSGGGGP

SEQ ID NO: 23 - protein, 17 aa, Artificial Sequence
  Linker
GSADDAKKDAAKKDGKS

SEQ ID NO: 24 - protein, 18 aa, Artificial Sequence
  Linker
GSTSGSGKPGSGEGSTKG

SEQ ID NO: 25 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 26 - protein, 5 aa, Artificial Sequence
  Hinge
  VARIANT (1)...(1): Xaa is glycine, cysteine or arginine
  VARIANT (4)...(4): Xaa is cysteine or threonine
XPPXP

SEQ ID NO: 27 - protein, 15 aa, Artificial Sequence
  Hinge
EPKSCDKTHTCPPCP

SEQ ID NO: 28 - protein, 12 aa, Artificial Sequence
  Hinge
ERKCCVECPPCP

SEQ ID NO: 29 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 30 - protein, 12 aa, Artificial Sequence
  Hinge
ESKYGPPCPSCP

SEQ ID NO: 31 - protein, 12 aa, Artificial Sequence
  Hinge
ESKYGPPCPPCP

SEQ ID NO: 32 - protein, 9 aa, Artificial Sequence
  Hinge
YGPPCPPCP

SEQ ID NO: 33 - protein, 10 aa, Artificial Sequence
  Hinge
KYGPPCPPCP

SEQ ID NO: 34 - protein, 14 aa, Artificial Sequence
  Hinge
EVVVKYGPPCPPCP

SEQ ID NO: 35 - protein, 11 aa, Artificial Sequence
  CDR L1
RASQDISKYLN

SEQ ID NO: 36 - protein, 7 aa, Artificial Sequence
  CDR L2
SRLHSGV

SEQ ID NO: 37 - protein, 9 aa, Artificial Sequence
  CDR L3
GNTLPYTFG

SEQ ID NO: 38 - protein, 5 aa, Artificial Sequence
  CDR H1
DYGVS

SEQ ID NO: 39 - protein, 16 aa, Artificial Sequence
  CDR H2
VIWGSETTYYNSALKS

SEQ ID NO: 40 - protein, 7 aa, Artificial Sequence
  CDR H3
YAMDYWG

SEQ ID NO: 41 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 43 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 5 aa, Artificial Sequence
  CDR H1
SYWMN

SEQ ID NO: 48 - protein, 17 aa, Artificial Sequence
  CDR H2
QIYPGDGDTNYNGKFKG

SEQ ID NO: 49 - protein, 13 aa, Artificial Sequence
  CDR H3
KTISSVVDFYFDY

SEQ ID NO: 50 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 51 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 52 - protein, 15 aa, Artificial Sequence
  Linker
GGGGSGGGGSGGGGS

SEQ ID NO: 53 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 54 - protein, 12 aa, Artificial Sequence
  CDR H3
HYYYGGSYAMDY

SEQ ID NO: 55 - protein, 7 aa, Artificial Sequence
  CDR L2
HTSRLHS

SEQ ID NO: 56 - protein, 9 aa, Artificial Sequence
  CDR L3
QQGNTLPYT

SEQ ID NO: 57 - DNA, 22 bp, Artificial Sequence
  IGH primer
acacggcctcgtgtattactgt

SEQ ID NO: 58 - DNA, 19 bp, Artificial Sequence
  IGH primer
acctgaggagacggtgacc

SEQ ID NO: 59 - protein, 127 aa, Artificial Sequence
  Mutein Streptavidin Ile44-Gly45-Ala-46-Arg47
MEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 60 - protein, 119 aa, Artificial Sequence [sequence omitted]

SEQ ID NO: 61 - protein, 106 aa, Artificial Sequence [sequence omitted]

SEQ ID NO: 62 - protein, 116 aa, Artificial Sequence
  Variable Heavy chain of anti-CD28 antibody CD28.3
LQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTV

SEQ ID NO: 63 - protein, 108 aa, Artificial Sequence
  Variable Light chain of anti-CD28 antibody CD28.3
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKR

SEQ ID NO: 64 - protein, 30 aa, Artificial Sequence
  Twin-Strep-tag
SAWSHPQFEKGGGSGGGSGGSAWSHPQFEK

SEQ ID NO: 65 - protein, 22 aa, Artificial Sequence
  GMCSFR alpha chain signal sequence
MLLLVTSLLLCELPHPAFLLIP

SEQ ID NO: 66 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 67 - protein, 18 aa, Artificial Sequence
  CD8 alpha signal peptide
MALPVTALLLPLALLLHA

SEQ ID NO: 68 - protein, 159 aa, Streptomyces avidinii
  Streptomyces avidinii (UniProt No. P22629)
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 69 - protein, 126 aa, Streptomyces avidinii
  Minimal streptavidin
EAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 70 - protein, 4 aa, Artificial Sequence
  Streptavidin-binding peptide
HPQF

SEQ ID NO: 71 - protein, 3 aa, Artificial Sequence
  Streptavidin-binding peptide
  VARIANT (3)...(3): Xaa is selected from Gln, Asp, and Met
HPX

SEQ ID NO: 72 - protein, 8 aa, Artificial Sequence
  Streptavidin-binding peptide
  VARIANT (1)...(1): Xaa is Trp, Lys or Arg
  VARIANT (2)...(2): Xaa is any amino acid
  VARIANT (7)...(7): Xaa is Gly or Glu
  VARIANT (8)...(8): Xaa is Gly, Lys or Arg
XXHPQFXX

SEQ ID NO: 73 - protein, 8 aa, Artificial Sequence
  Streptavidin-binding peptide
  VARIANT (2)...(2): Xaa is any amino acid
  VARIANT (7)...(7): Xaa is Gly or Glu
  VARIANT (8)...(8): Xaa is Gly, Lys or Arg
WXHPQFXX

SEQ ID NO: 74 - protein, 8 aa, Artificial Sequence
  Streptavidin-binding peptide, Strep-tag
WRHPQFGG

SEQ ID NO: 75 - protein, 8 aa, Artificial Sequence
  Streptavidin-binding peptide, Strep-tag II
WSHPQFEK

SEQ ID NO: 76 - protein, 29 aa, Artificial Sequence
  Sequential modules of streptavidin-binding peptide
  VARIANT (9)...(21): Xaa is any amino acid
  VARIANT (18)...(21): may be present or absent
WSHPQFEKXXXXXXXXXXXXXWSHPQFEK

SEQ ID NO: 77 - protein, 32 aa, Artificial Sequence
  Sequential modules of streptavidin-binding peptide
  VARIANT (21)...(23): Xaa is Gly
  VARIANT (24)...(24): Xaa is Ser
  VARIANT (21)...(24): may be present or absent
WSHPQFEKGGGSGGGSGGGSXXXXWSHPQFEK

SEQ ID NO: 78 - protein, 28 aa, Artificial Sequence
  Twin-Strep-tag
WSHPQFEKGGGSGGGSGGGSWSHPQFEK

SEQ ID NO: 79 - protein, 24 aa, Artificial Sequence
  Twin-Strep-tag
WSHPQFEKGGGSGGGSWSHPQFEK

SEQ ID NO: 80 - protein, 28 aa, Artificial Sequence
  Twin-Strep-tag
WSHPQFEKGGGSGGGSGGSAWSHPQFEK

SEQ ID NO: 81 - protein, 30 aa, Artificial Sequence
  Twin-Strep-tag
SAWSHPQFEKGGGSGGGSGGGSWSHPQFEK

SEQ ID NO: 82 - protein, 30 aa, Artificial Sequence; identical to SEQ ID NO: 64
  Twin-Strep-tag

SEQ ID NO: 83 - protein, 159 aa, Artificial Sequence
  Mutein Streptavidin Val44-Thr45-Ala46-Arg47
DPSKDSKAQVSAAEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 84 - protein, 126 aa, Artificial Sequence
  Mutein Streptavidin Val44-Thr45-Ala46-Arg47
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 85 - protein, 127 aa, Artificial Sequence
  Mutein Streptavidin Val44-Thr45-Ala46-Arg47
MEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 86 - protein, 159 aa, Artificial Sequence
  Mutein Streptavidin Ile44-Gly45-Ala-46-Arg47
DPSKDSKAQVSAAEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 87 - protein, 126 aa, Artificial Sequence
  Mutein Streptavidin Ile44-Gly45-Ala-46-Arg47
EAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 88 - protein, 126 aa, Artificial Sequence
  Mutein Streptavidin Val44-Thr45-Ala46-Arg47 and Glu117, Gly120, Try121 (mutein m1-9)
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 89 - protein, 139 aa, Artificial Sequence
  Mutein Streptavidin Val44-Thr45-Ala46-Arg47 and Glu117, Gly120, Try121 (mutein m1-9)
DPSKDSKAQVSAAEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 90 - protein, 127 aa, Artificial Sequence
  Minimal streptavidin
MEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS